SEQUENCE LISTING (87 sequences)

SEQ ID NO: 1 - protein, 967 aa, Arabidopsis thaliana
MYSNNRVEVFHGDGRLGELEIYPSRELNQQQDDVMKQRKKKQREVMELAKMGIRISHFSQSGERCPPLAILTTISSCGLCFKLEASPSPAQESLSLFYSSCLRDNKTAVMLLGGEELHLVAMYSENIKNDRPCFWAFSVAPGIYDSCLVMLNLRCLGIVFDLDETLVVANTMRSFEDKIDGFQRRINNEMDPQRLAVIVAEMKRYQDDKNLLKQYIESDQVVENGEVIKVQSEIVPALSDNHQPLVRPLIRLQEKNIILTRINPMIRDTSVLVRMRPSWEELRSYLTAKGRKRFEVYVCTMAERDYALEMWRLLDPEGNLINTNDLLARIVCVKSGFKKSLFNVFLDGTCHPKMALVIDDRLKVWDEKDQPRVHVVPAFAPYYSPQAEAAATPVLCVARNVACGVRGGFFRDFDDSLLPRIAEISYENDAEDIPSPPDVSHYLVSEDDTSGLNGNKDPLSFDGMADTEVERRLKEAISASSAVLPAANIDPRIAAPVQFPMASASSVSVPVPVQVVQQAIQPSAMAFPSIPFQQPQQPTSIAKHLVPSEPSLQSSPAREEGEVPESELDPDTRRRLLILQHGQDTRDPAPSEPSFPQRPPVQAPPSHVQSRNGWFPVEEEMDPAQIRRAVSKEYPLDSEMIHMEKHRPRHPSFFSKIDNSTQSDRMLHENRRPPKESLRRDEQLRSNNNLPDSHPFYGEDASWNQSSSRNSDLDFLPERSVSATETSADVLHGIAIKCGAKVEYKPSLVSSTDLRFSVEAWLSNQKIGEGIGKSRREALHKAAEASIQNLADGYMRANGDPGPSHRDATPFTNENISMGNANALNNQPFARDETALPVSSRPTDPRLEGSMRHTGSITALRELCASEGLEMAFQSQRQLPSDMVHRDELHAQVEIDGRVVGEGVGSTWDEARMQAAERALSSVRSMLGQPLHKRQGSPRSFGGMSNKRLKPDFQRSLQRMPSSGRYS

SEQ ID NO: 2 - protein, 932 aa, Zea mays
MFKSMVYYVNTSIGEVEVWPKGEASAGLTMAAWAREIRVERISPPSERCPPLAVMHTVAVGARCLVMESRPPVVADVVPPLVVMHTACLRENKTAVVPLGDEELHLVAMTSRRNLTNHACFWGYKLPFGLYNSCLTMLNLRCLGIVFDLDETLIVANTSRSFEDRIDALQRKLSNETDPQRRNGMLSEIKRYQDDKSILKQYIEGDQVYDDGKVYKAQPEIVPPLSDNQQPMTRPVIRLQDKNIILTRINPLIRDTSVLVCLRPAWEDLRSYLIARGRKRFEVYVCTMAERDYALEMWRLLDPDSRLINSVQLHDRMVCVKSGLKKSLLNVFHDGSCHPGMALVIDDRLKVWDEKDQLRVHVVPAFTPYYAPQAEANCSIPVLCVARNVACNVRGGFFKDFDEGLLPRISNVHYEDEVNEISAPDVGNYLITDDENVALVNGNRDSLPFDGMADAEVERRMKEANAQSFHQTAGDFVMPVAPAQNFVSTSVASLAPPLGMMPSPFSQPVAPPGFSDSLQGSPAREEGEVPESELDPDTRRRLLILQHGQDTRDPTSPLPAIPPVQVPVPPVQPHGNWFPTEDGINQSNLNRGSAGFTVESDSIVYEKKQPPHPSFFHGGDSPMPSDRFGYQNQRFPSQLPHEDHPMMQNHAPPKYRSFSGEELASWHVPSSQRNNQIESGRHFAQYAGTSAGILEGIALKCGSKVEYKSALCDTAELQFSIEVWIVGEKVGEGIGRTRREAQRQAAEMSLRNLANKYLSSDPNKLSDMKENDFSSNRNVFGYSGNTRDDMLPLSSTSEESRFMKMENNNSRKTGSSVAALKELCTVEGYNLVFQACPSSADGLVGKESYAQVQVGGQILGKGVGLTWEEAKLQAAAEALGTLRSMLGQLGHKRSGSPRSLAPNFNKRFKPDFPRTVQRVPYGTYSRIEGHVP

SEQ ID NO: 3 - protein, 934 aa, Zea mays
MFKSMVYFVNISIGEVEVWPKGQASAGLAVAAWARDIRVDRLSPPSERCPPLAVMHTVAVAARCLVMESRPPVAADVASLPLVDMHAACLRDNKTAVVALGDEELHLVAMTSRRNLTNHACFWGYKLPFGLYNSCLTMLNLRCLGIVFDLDETLVVANTSRSFEDRIDALQRKLSNETDPQRRNGMLSEIKRYQDDKSILKQYIEGDQVYDDGKVYKAQPEIVPPLSDNQQTMTRPVIRLQEKDIILTRINPLIRDTSVLVRLRPAWEDLRSYLIARGRKRFEVYVCTMAERDYALEMWRLLDPDSRLINSVRLHDRMVCVKSGLKKSLLNVFHDGSCHPGMALVIDDRLKVWDEKDQLRVHVVPAFTPYYAPQAEANCSIPVLCVARNVACNVRGGFFKDFDEGLLPRISNVHYEDEVNDISSAPDVGNYLITEDENVALVNGNRDSLPFDGMSDAEIERRMKEANAQAFHQTATNFVMPVAPAQNFVSSSVAPLAPPLSMMPPPFSQPVVQPGFSDPLQGSPAREEGEVPESELDPDTRRRLLILQHGQDTRDPTPPHPAIPPVQVPVPPVQPHGNWFPTEDGINPSNLSRGSAGFTVESDSMPYEKKQPPHPSFFHGGDSPMSSDRFGYQNQRFPSQLPHTEDHHMLQNHAPPKYRSFSGEELATRHVPSSQRNHIESGRHFSQYAGTSAGVLEGIAVKCGSKVEYRSTLCDTAELQFSIEVWIVGEKFGEGIGRTRREAQRQAAEMSLRNLANKYLSSDPNKLTDMKQDAFGSNRNIFGYSGNTRDDMLPLSSTSEESRFMKMEENNSRKTGDSVTALKELCTVEGYNLVFQACPSPADGLVGKESYAQVQIGRQILGKGVGLTWEEAKLQAADEALGTLRSMLGQLGHRRSGSPRSLAPNFNKRFKPDFPRTVQRVPYGTYSRIEGHAP

SEQ ID NO: 4 - protein, 934 aa, Sorghum bicolor
MFKSMVYYGNTSIGEVEVWPKGEASAGLAVAAWAREIRVDRLSPPSERCPPLAVMHTVAVGARCLVMESRPPVAADVAPMPLVAMHAACLRDNKTAVVPLGDGELHLVAMTSRRNLTNHACFWGYKLPFGLYNSCLTMLNLRCLGIVFDLDETLIVANTSRSFEDRIDGLQRKLSNETDPQRRNGMLSEIKRYQDDKSILKQYIEGDQVYDDGKVYKAQPEIVPPLSDNQQPMTRPVIRLQDKNIILTRINPLIRDTSVLVRLRPAWEDLRSYLIARGRKRFEVYVCTMAERDYALEMWRLLDPDSRLINSVQLHDRMVCVKSGLKKSLLNVFHDGSCHPGMALVIDDRLKVWDEKDQLRVHVVPAFTPYYAPQAEANCSIPVLCVARNVACNVRGGFFKDFDEGLLPRISNVHYEDEVNDISSAPDVGNYLITEDENAALVNGNRDSLPFDGMADAEVERRMKEANAQAFHQTAGNFVMPVAPAQNFVSSSVAPLAPPLGVMPPTFSQPVVQPGFSDSLQGSPAREEGEVPESELDPDTRRRLLILQHGQDIRDPTPPLPAIPPVQVPVPPVQPHGNWFPTEDGLNPSNLNRGSAGFTVESDPMLYEKKQPPHPSFFHGGDSPMSSDRFGYQNQRFPSQLPHTEDHHMLQNHAPPKYRSFSGEELAARHVPSSQRNNQIESGRHFAQYAGTSAGILDGIALKCGSKVEYRSTLCDTAELQFSIEVWIVGEKVGEGIGRTRREAQHKAAEMSLRNLANKYLSSDPNKLTDMKENGFSGNRNVFGYSGNTRDDMLPLSSTSEESRFMKMENNSRKTGGSVAALKELCTVEGYNLVFQERPSPADGLVGKESYAQVEVGGQILGKGVGLTWEEAKLQAADEALGTLRSMLGQLAHKRSGSPRSLAPNFNKRFKPDFPRTVQRVPYGTYSRIEGHVP

SEQ ID NO: 5 - protein, 942 aa, Sorghum bicolor
MIKSLVYYGNTPVGEVEVWPKGQTDLAWAREIRVDRLSPASERCPPLAVLHVVAAGARCLVMESKSTATAHEPPPPLVTMHTTCLKDNKTAVFPLGAEEIHLVAMTSKRNMPNGACFWGYKVPLGLYNSCLSMLNLRCLGIVFDLDETLIVANTTRSFEDRIDAIQRKLNNEADPQRISGMLAEIKRYQEDKSILKQYIESDQVTDGGELYKVQSEVIPPLDDNHQQPMTRPIIRLQEKNIILTRINPSIRDTSVLVRLRPAWDDLRSYLIARGRKRFEIYVCTMAERDYALEMWRLLDPDSKLINSVQLLDRLVCVKSGSRKSLLNVFHDGSCHPGMALVIDDRLKVWDEWDQRRVHVVPAFAPYYAPQAEANFPIPVLCVARNVACNVRAGFFKEFDEGIIPRITEVCYEDELDDIASAPDVCNYFVSEDENAAVSNVNKNPLAFDGMADAEVEKRMKEASSSFQSANPITTNVDVMSVAANQHFGTPISSSTPVAPPLGMLLNNDQDPQPPSLRWPVAQSGHVDSSQGSPAREEGEVPESELDPDTRRRLLILQHGQDTRDPAAPFPAGSPAQVSVPPVQSHENWFCLEDEMNPRNLNKVSTEFHLESDSVHYDKKQLQHTSYIPIGDNPMSYDRYDYQNQRYPSQPPHSEGHHRFHNHAPTAYRSFSGEDMATWYAPSGQRSSHMESGRHFARYGGIPGVLEEIALKCGFKVEYRSTLCDTTELQFSTEVLIFGEKVGEGVGKTRKEAQWQAADTSLRNLADKFLSWDPDKVTVLKENDFNRHPKSHRYPGSNIYDTLPVASTSDESRYMNDRIDTLRKPGASVAALKELCAVEGYNLDFHAQPSADGSVGKEIRAQVEIGGKVLGKGVGVTWEEAKLQAAYEAYGTLKSMLGQFVPRQSASPRSMVPNFNKRFNPDFSEALQRIPSGRYSRNDSRFP

SEQ ID NO: 6 - protein, 948 aa, Triticum aestivum
MIKSMVYFGHISIGEVELSPKGETNVAAAPWVREIRVDRLSPPSERCLPLAVLHTVSSGALCFVMESRPSPATADNEPPSSLVAMHTACLRDNKTAVFPLGAEEIHLVAMKPKSNLPNHACFWGYKVPLGLYSSCLSMLNLRCLGIVFDLDETLIVANTTRSFEDRIDALQRKLSKETDPQRISGMLAEIKRYQEDRTMLKQYIDGDQVIDGGKMYKVQSEVVPPLADNHQPMIRPVIRLQDKSIILTRINPSIRDTSVLVRLRPAWDDLRSYLIARGRKRFEVYVCTMAERDYALEMWRLLDPDSRLINSVQLPHRLVCVKSGSKKSLLNVFHDGSCHPGMALVIDDRLKVWEEKDQCRVHVVPAFSPYYAPQAEANFPIPVLCVARNVACNVRGSFFKEFDEGLLPSISEVHFDDELDHVPSSPDVGNYLISEDENAASLNVNKDPMAFDGMADAEVERRMKEAVCSVQAADPVTTNVDVISVAANQQFATSSSIPLAPPLGVVPLNNDQGPQPPSVSWPDAQSGMVDPLQGSPAREEGEVPESELDPDTRRRLLILQHGQDTRDPTPPFAAEPSVQASVPPVQSQGNWFPVEDEMDPRNLNRTSTDFHLESDAVHSDKSQPPHQPYFPARDNPIFSDRLNHQNQRYSSQLPHSEDRQMLQNQAPTTYRSFSGEDMATQRFHPGNRSSQMESGRQFVQYTETSGAVLEEIAAKCGFKVEYRSTLCDTTELRFSIQIWIVGEKVGEGMGRTRKEAQRQAANISLRNLADRFLSFDPDKMTVPVDDGFSSNPNSFKYRGIDGDNIVPVASTSDGSRYMHERVDNSTKSAGSVAALKELCTAEGYNLVFQAQPSPSDSLRREEVHAQIEIGGQILGKGVGVTWEEAKVQAADGALGTLRYMLGQRPQKRSGSPRSFASNYNNKRYKPDFQPMVQRIPSGRYSRNDSRVP

SEQ ID NO: 7 - protein, 948 aa, Triticum aestivum
MIKSMVYFGHISIGEVELWPKGETNVAAAPWVREIRVDRLSPPSERCLPLAVLHTVSSGALCFVMESRPSPATADDEPPSSLVAMHTACLRDNKTAVFPLGAEEIHLVAMKPKSSLPNHACFWGYKVPLGLYNSCLSMLNLRCLGIVFDLDETLIVANTTRSFEDRIDALQRKLSKEIDPQRISGMLAEIKRYQEDRSMLKQYIDGDQVTDGGKVYKVQSEVVPPLADNHQPMIRPVIRLQEKSIILTRINPSIRDTSVLVRLRPAWDDLRSYLIARGRKRFEVYVCTMAERDYALEMWRLLDPDSRLINSVQLPHRLVCVKSGSKKSLLNVFHDGSCHPGMALVIDDRLKVWEEKDQCRVHVVPAFSPYYAPQAEANFPIPVLCVARNVACNVRGSFFKEFDEGLLPSISEVHFDDELNHVPSSPDVGNYLIPEDENAASLNVNKDQMAFDGMADAEVERRMKEAVCSVQAADPVTTNVDVIPVAANQQFATSSSIPLAPPPGMVPLNNDQGPQPPSVSWPDAQSGMVDPLQGSPAREEGEVPESELDPDTRRRLLILQHGQDTRDPTPPFAAEPSVQASVPPVQSQGNWFPVEDEMDPRNLNRTSTDFHLESDAVHSDKSQPPHQPYFPAHDNPIFSDRLNHQNQRYSSQLPHSEDRQMLQNQAPTTYRSFSGEDMATQRFHPGNRSSQMESGRQFVQYTETSGAVLEEIAAKCGFKVEYRSTLCDTTELRFSIQIWIVGEKVGEGMGRTRKEAQRQAANISLRNLADRFLSFDPDKMTVPMDDGFSSNPNSFKYRGIDGDNIVPVASTSDGSRYMHERVDNSTKSAGSVAALKELCTAEGYNLVFQAQPSPLDSLRREEVHAQIEIGGQILGKGVGVTWEEAKEQAADGALRTLRYMLGQRPQKRPGSPRSFASNYNNKRYKPDFQPMVQRIPSGRYSRNDSRVP

SEQ ID NO: 8 - protein, 947 aa, Triticum aestivum
MIKSMVYFGHISIGEVELWPKGETNVAAAPWVREIRVDRLSPPSERCLPLAVLHTVSSGALCFVMESRPSPATADDEPPSSLVAMHTACLRDNKTAVFPLGAEEIHLVAMKPKSNLPNHACFWGYKVPLGLYNSCLSMLNLRCLGIVFDLDETLIVANTTRSFEDRIDALQRKLSKETDPQRISGMLAEIKRYQEDRTMLKQYIDGDQVIDGGKMYKVQSEVVPPLADNHQPMIRPVIRLQEKSIILTRINPSIRDTSVLVRLRPAWDDLRSYLIARGRKRFEVYVCTMAERDYALEMWRLLDPDSRLINSVQLPHRLVCVKSGFKKSLLNVFHDGSCHPGMALVIDDRLKVWEEKDQCRVHVVPAFSPYYAPQAEANFPIPVLCVARNVACNVRGSFFKEFDEGLLPSISEVHFDDELDHVPSSPDVGNYLISEDENAASLNVNKDPMAFDGMADAEVERRMKEAVCSVQAADPVTTNVDVISAAANQQFATSSSIPLAPPLGMVPLNNDQGPQPPSVSWSDAQSGMVDPLQGSPAREEGEVPESELDPDTRRRLLILQHGQDTRDPTPPFAAEPSVQASVPPVQSQGNWFPVEDEMDPRNLNRTSTDFHVESDAVHSDKSQPPHQPYFPARDNPVFSDRFNHQNQRYSSQLPHSEDRQMLQNQAPTTYRSFSGEDMATQRFHPGNRSSQMESGRQFVQYTETSGAVLEEIAAKCGFKVEYRSTLCDTTELRFSIQIWIVGEKVGEGMGRTRKEAQRQAANISLRNLADKFLSFDPDKMTVPMDDGFSSNPNSFKYTGIDGDNIVPVASTSDGSRYMHERVDNSTKSAGSVAALKELCTAEGYNLVFQAQPSPLDSLTRKEVHAQIEIGGQILGKGVGVTWEEAKVQAADGALGTLRYMLGQRPQKRSGSPRSFASNYNKRYKPDFQPMAQRIPSGRYSRNDSRVP

SEQ ID NO: 9 - protein, 934 aa, Triticum aestivum
MIKSMVYYGNTSIGEVEVWPKGDTNLGAAAWAREIRVDRLSPPSERCLPLAVMHTVAVGARCLVMESRPPKAADEPPPPLVAMHAACLRDNKTAVVPLGEEELHLVAMTSGRNLTNHACFWGYKVPFGLYNSCLTMLNLRCLGIVFDLDETLIVANTTRSFEDRIDSLQRKLSNETDPQRMNGMLAEIKRYQDDRSILKQYIEGDQVYDDGKMYKVQPEIVPPLSDNHQSLTRPVIRLQEKNIILTRINPLIRDTSVLVRLRPAWEDLRSYLIARGRKRFEVYVCTMAERDYALEMWRLLDPDSRLINSVQLSDRMVCVKSGLKKSLLNVFHDGSCHPGMALVIDDRLKVWDEKDQSRVHVVPAFTPYYAPQAEANCSIPVLCVARNVACNVRGGFFKDFDEGLLPRITSVLYEDEIQDISSAPDVGNYLISEDENVAVVNGNRDSLAFDGMADAEVERRMKEASGSGSVLNPTMANMVMPVAPSQSFAPSSVAPFAPPLGMMPLSNNQVPPPAFSQPVVQPVVLDPLQASPGREEGEVPESELDPDTRRRLLILQHGQDTRDPTPPLPAVPPAQVSVPPVQSHGNWFPIEDGIGMNSNNLNMGSAGFPSESDTMHYDKKQPPQPSYFHGGDNNPVPSDRFSYQSQRFPSQVTHTEDHRMLQNHAPPRYRSFPGQRNNLIESGQSYARNVGSVGILEEIALKSGSKVEYRSTLCDTAELQFSIEIWIVGEKVGEGIGSSRKEAQRQAAEISLRNLANKYLLSDPNKMTDVNEDGFGSNPNFFGYSENTRNDILSVASTSEESRFTKTGENNSRITGGSIAALKQLCTVEGYNLVFQARPSPLDGSGGKETYAQVEVGGQTLGKGVGITWEEAKLQAADEALGTLRSMLGQLAQKRSSSPRSLAPNYNKRFKPDFPRAVQRPPYGRYSRIEGHVP

SEQ ID NO: 10 - protein, 935 aa, Triticum aestivum
MIKSMVYYGNTSIGEVEVWPKGDTNLGAAAWAREIRVDRLSPPSERCLPLAVMHTVAVGARCLVMESRPPKAADEPPPPLVAMHAACLRDNKTAVVPLGEEELHLVAMTSGRNLTNHACFWGYKVPFGLYNSCLTMLNLRCLGIVFDLDETLIVANTTRTFEDRIDSLQRKLSNETDPQRMNGMLAEIKRYQDDRSILKQYIEGDQVYDDGKMYKVQPEIVPPLSDNHQSLTRPVIRLQEKNIILTRINPLIRDTSVLVRLRPAWEDLRSYLIARGRKRFEVYVCTMAERDYALEMWRLLDPDSRLINSVQLSDRMVCVKSGLKKSLLNVFHDRSCHPGMALVIDDRLKVWDEKDQSRVHVVPAFTPYYAPQAEANCSIPVLCVARNVACNVRGGFFKDFDEGLLPRITSVLYEDEIQDISSAPDVGNYLISEDENVAVVNGNRDSLAFDGMADAEGERRMKEASGSCSVLNPTMANMVMPVAPSQSFVPSSVAPFAPPLGMMPLSNNQVPPPVFSQPVVQPVVLDPLQASPGREEGEVPESELDPDTRRRLLILQHGQDTRDPTPPLPAVPPAQVSVPPVQSHGNWFPVEDGIGMNSNNLNRGSAGFPSESDTMHYDKKQPPQPSYFHGGDNNPVSSDRFSYQSQRFPSQVTHTEDHRMLQNHAPPRYRSFPGQRDNLIESGQSYARNVGTSVGILEEIALKSGSKVEYRSKLCDTAELQFSIEVWIVGEKVGEGIGSSRKEAQRQAAEISLRNLANKYLLSDPNKMTDVNEDGFGSNPNFFGYSENTRNDILPVASTSEESRFTKTGENNSRITGGSIAALKQLCTVEGYNLVFQARPSPLDGSGGKETYAQVEVGGQTLGKGVGITWEEAKLQAADEALGTLRSMLGQLAQKRSSSPRSLAPNYNKRFKPDFPRAVQRPPYGRYSRIEGHVP

SEQ ID NO: 11 - protein, 935 aa, Triticum aestivum
MIKSMVYYGNTSIGEVEVWPKGDTNLGAAAWAREIRVDRLSPPSERCLPLAVMHTVAVGARCLDMESRPPKAADEPPPPLVAMHAACLRDNKTAVVPLGEEELHLVAMTSGRNLTNHACFWGYKVPFGLYNSCLTMLNLRCLGIVFDLDETLIVANTTRSFEDRIDSLQRKLSNETDPQRMNGMLAEIKRYQDDRSILKQYIEGDQVYDDGKMYKVQPEIVPPLSDNHQSLTRPVIRLQEKNIILTRINPLIRDTSVLVRLRPAWEDLRSYLIARGRKRFEVYVCTMAERDYALEMWRLLDPDSRLINSVQLSDRMVCVKSGLKKSLLNVFHDGSCHPGMALVIDDRLKVWDEKDQSRVHVVPAFTPYYAPQAEANCSIPVLCVARNVACNVRGGFFKDFDEGLLPRITSVLYEDEIQDISSAPDVGNYLISEDENVAVVNGNRDSLAFDGMADAEVERRMKEASGSGSVLNPTMANMVMPVAPSQSFVPSSVAPFAPPLGMMPLSNNQVPPPALSQPVVQPVVLDPLQASPGREEGEVPESELDPDTRRRLLILQHGQDTRDPTPPLPAVPPAQVSVPPVQSHGNWFPIEDGIGMNSNNLNMGSAGFPSESDTMHYDKKQPPQPSYFHGGDNNPVSSGRFSYQSQRFPSQVTHTEDHRMLQNHAPPRYRSFPGQRNNLIESGQSYARNVGTSVGILEEIALKSGCKVEYRSTLCDTAELQFSIEVWIVGEKVGEGIGSSRKEAQRQAAEISLRNLANKYLLSDPNKMTDGNEDGFGSNPNFFGYSENTRNDILPVASTSEESRFTKTGENNSRITGGSIAALKQLCTVEGYNLVFQARPSPLDGSGGKETYAQVEVGGQTLGKGVGITWEEAKLQAADEALGTLRSMLGQLAQKRSSSPRSLVPNYNKRFKPDFPRAVQRPPYGRYSRIEGHVP

SEQ ID NO: 12 - protein, 956 aa, Glycine max
MRMYKSVVYQGEVVVGEVDVYPEENNNYKNFHVKEIRISHFSQPSERCPPLAVLHTVTSCGVCFKMESKTQQQDGLFQLHSLCIRENKTAVMPLGGEEIHLVAMHSRNVDRPCFWGFIVALGLYDSCLVMLNLRCLGIVFDLDETLIVANTMRSFEDRIDALQRKINSEVDPQRISGMQAEVKRYQDDKNILKQYAENDQVVDNGRVIKVQSEIVPALSDSHQPIVRPLIRLQDKNIILTRINPQIRDTSVLVRLRPAWEDLRSYLTARGRKRFEVYVCTMAERDYALEMWRLLDPDSNLINSKELLGRIVCVKSGLKKSLFNVFQDGLCHPKMALVIDDRLKVWDEKDQPRVHVVPAFAPYYAPQAEASNTIPVLCVARNVACNVRGGFFKDFDDGLLQKIPQIAYEDDIKDIPSPPDVSNYLVSEDDGSISNGHRDPFLFDGMADAEVERKLKDALSAASTIPVTTANLDPRLTSLQYTMVPSGSVPPPTAQASMMPFPHVQFPQPATLVKPMGQAAPSEPSLHSSPAREEGEVPESELDPDTRRRLLILQHGQDTRDHASAEPPFPVRHPVQTSAPHVPSSRGVWFPAEEEIGSQPLNRVVPKEFPVDSGPLGIAKPRPHHPSFFSKVESSISSDRILHDSHQRLPKEMYHRDDRPRLNHMLSSYRSFSGDDIPFSRSFSSHRDLDSESGHSVLHADTPVAVLQEIALKCGTKVDFISSLVASTELQFSMEAWFSGKKIGHRVGRTRKEAQNKAAEDSIKHLADIYLSSAKDEPGSTYGDVSGFPNVNDSGYMGIASSLGNQPLSKEDSASFSTASPSRVLDPRLDVSKRSMGSISSLKELCMMEGLDVNFLSAPAPVSTNSVQKDEVHAQVEIDGKVFGKGIGLTWDEAKMQAAEKALGSLRSKLGQSIQKRQSSPRPHQGFSNKRLKQEYPRPMQRMPSSARYPRNAPPIP

SEQ ID NO: 13 - protein, 960 aa, Glycine max
MYKSVVYQGEVVVGEVDVYPEENNNNNNKNYNKNFHVKEIRISHFSQPSERCPPLAVLHTVTSCGVCFKMESKTQQQDGLFQLHSLCIRENKTAVMPLGGEEIHLVAMHSRNDDRPCFWGFIVTLGLYDSCLVMLNLRCLGIVFDLDETLIVANTMRSFEDRIDALQRKINSEVDPQRISGMQAEVKRYLDDKNILKQYAENDQVVDNGRVIKVQSEIVPALSDSHQPIVRPLIRLQDKNIILTRINPQIRDTSVLVRLRPAWEDLRSYLTARGRKRFEVYVCTMAERDYALEMWRLLDPDSNLINSKELLGRIVCVKSGLKKSLFNVFQDGSCDPKMALVIDDRLKVWDERDQPRVHVVPAFAPYYAPQAEASNTIPVLCVARNVACNVRGGFFKDFDDGLLQKIPQIAYEDDIKDVPSPPDVSNYLVSEDDGSISNGNRDPFLFDGMADAEVERKLKDALAAASTFPVTTANLDPRLTSLQYTMVPSGSVPPPTAQASMMPFPHVQFPQPATLVKPMGQAAPSDPSLHSSPAREEGEVPESELDPDTRRRLLILQHGQDTRDHASAEPPFPVRHPVQASAPRVPSSRGVWFPVEEEIGSQPLNRVVPKEFPVDSGPLGIEKPRLHHPSFFNKVESSISSDRILHDSHQRLPKEMYHRDDRPRLNHMLSSYRSFSGDDIPFSRSSSSHRDLDSESGHSVLHADTPVAVLHEIALKCGTKVDFMSSLVASTELKFSLEAWFSGKKIGHGFGRTRKEAQNKAAKDSIEHLADIYLSSAKDEPGSTYGDVSGFPNVNDNGYMGIASSLGNQPLSKEDSASFSSASPSRALDPRLDVSKRSMGSISALKELCMMEGLGVNFLSTPAPVSTNSVQKDEVHAQVEIDGKIFGKGIGLTWDEAKMQAAEKALGNLRSKLGQSIQKMQSSPRPHQGFSNKRLKQEYPRTMQRMPSSARYPRNAPPIP

SEQ ID NO: 14 - protein, 960 aa, Glycine max
MPTSMVYHGEMAVGEVKIYPEENKNMDLKEIRISHFSQPSERCPPLAVLHTITSFGICFKMESSTSQKRQQQDALFHLHSSCIRENKTAVMPVRGEEIHLVAMYSRNNDRPCFWGFIVASGLYNSCLTMLNLRCLGIVFDLDETLVVANTMRSFEDKIEVLHRKMNSEVNPQQISAMQAEIKRYLDDKNILKEYAENDQVVDNGKVIKIQSESVPALSDSHQPIVRPLIRLQEKNIILTRINPQIRDTSVLVRLRPAWEDLRSYLTARGRKRFEVFVCTMAERDYALEMWRLLDPELNLINSKELLDRIVCVKSGLKKSLFNVFQNGLCHLKMALVIDDRLKVWDEKDQPRVHVVPAFAPYYTPQAEASNAVPFLCLARNVACNVRGGFFKDFDDGLLQKIPLIAYEDDIKDIPSPDVSNYLVSEDDASASNGNKNLLLFDGMADAEVERRLKDAISASSTILALTANIDPRLAFTSSLQYTMVSSSGTVPPPTAQASVVQFGNVQFPQPNTLVKPMSQVTHPGLSLHSSPAREEGELPESELDLDTRRRFLILQHGQDTRERMASEPPFPVRHPAQVSAPASSVPSRRGWFSVEEEMGPQQLNLPVPKEFPVDSEPFHIEKRWPRHPSFFSKVGDSISSDRVFHESHQRLPKEVHHRDDRSRLSQSLSSYHSLPGDDIPLSGSSYSNRDFDSESGRSLFHADTTAGVLQEIALNCGTKVEFLSSLVASTELQFSIEAWFAGKKIGEGFGRTRREAQSKAAGCSIKQLADIYMSHAKDDSGSTYGDVSGFHGSNNDGFVSSGNSLGNQLLPKEESGSFSTASESSRVSDSRLEVSKRSTDSISALKELCMMEGLAASFQSPPASASTHLTQKDEVHAQVEIDGQIFGKGFGVTWEEAKMQAAKKALGSLRTMFNQGSLKRHGSPRSMQGLANKRLKPEYPPTLQRVPYSARYPRNAPLVP

SEQ ID NO: 15 - protein, 958 aa, Glycine max
MKRSMVYHGEMEVGEVEIYPEEKKNIDLKEIRISHFSQPSERCPPLAVLHTITSFGICFKMESSTSQTRQQQDVLFHLHSSCIRENKTAVMPLRGEEIHLVAMYSRNNDRPCFWGFIVASGLYNSCLTMLNLRCLGIVFDLDETLVVANTMRSFEDKIEVLHRKMNSEVNPQRISTMQAEIKRYLDDKNILKEYAENDQVVDNGKVIKIQSEIVPALSDSHQPIVRPLIRLQEKNIILTRINPQIRDTSVLVRLRPAWEDLRSYLTARGRKRFEVFVCTMAERDYALEMWRLLDPELNLINSKELLDRIVCVKSGLKKSLFNVFQNGLCHLKMALVIDDRLKVWDEKDQPQVHVVPAFAPYYAPQAEASNAVPTLCLARSVACNVRGGFFKDFDDGLLQKIPLIAYEDDIKDIPSPPDVSNYLVSEDDASASNGNKNLLLFDGMADAEVERRLKDAISASSTVPAMTTNLDPRLAFNSSLQYTMVSSSGTVPPPTAQASIVQFGNVQFPQPNTLVKPICQVTPPGPSLHSSPAREEGEVPESELDLDTRRRLLILQHGQDTREHTSSEPPLPVRHPTQVSAPSVPSRRGWFSVEEEMGPQQLNQLVPKEFPVGSEPLHIEKRWPRHPSLFSKVDDSVSSDRVFHESHQRLPKEVHHRDDHSRLSQSLSSYHSFPGDDIPLSGSSYSNRDFDSESGRSLFHADITAGVLQEIALKCGTKVEFLSSLVASTALQFSIEAWFAGKKVGEGFGRTRREAQNKAAECSIKQLADIYMSHAKDDSGSTYGDVSGFHGSNNNGFVSSGNSLGNQLLPKESVSFSTSSDSSRVSDPRLEVSKRSTDSISALKEFCMMEGLAANFQSSPAPASTHFAQKDEVHAQVEIDGQIFGKGFGLTWEEAKMQAAKKALESLRTMFNQGTRKRHGSPRSMQGLANKRLKQEYPRTLQRIPYSARYPRNAPLVP

SEQ ID NO: 16 - protein, 1023 aa, Beta vulgaris
MIKSVVYEGENLLGEVEIYFQNNNNNKNLELMKGMRISHYSEMSERCPPLAVLHTITKSSGGICFKMMESSSHTSSNNNNNNKFYFQQQQQESQLLAMHSNCIRDNKTAVVPLGEQEIHLVALRSRRMAGVTPCFWGFSVAPGLYESCLGLLNLRCLGIVFDLDETLIVANTLRSFEDRIEALQRKISVEADPQRIAGMVAEVKRYQEDKSILKQYAETDQVVDNGKVHKIQAEVIPALSDNHQTVIRPLIRLQDKNIVLTRINPQIRDTSVLVRLRPAWEDLRSYLTARGRKRFEVYVCTMAERDYALEMWRLLDPDSNLICARELLDRIVCVKSGSKKSLFNVFHGGICHPKMALVIDDRLKVWDEKDQPRVHVVPAFAPYYAPQAEANNAIPVLCVARNVACNVRGGFFKEFDEGLLQRVSEVSFEDDPRDIPSPPDVSNYLVSEDDGSGSNGIKESMTFDGMADAEVERRLKEAVLSSSSASPLPSANTPATVNFDHRLASSLPFAVATSALAIPQPAPQATITPYHNNLFSQAGPLARPLGNIGPQDIGLHNSPAREEGEVPESELDPDTRRRLLILQHGQDMREGPPNEPPFPARTPVQAPVTGPVPVSVPGPVPVPGPGPVSVPVPGPIPSPVPVPVSGTVPGPGPRVQSRGSWFPVEDHISQGPLSRVAAKEFPVAPDASPVEKQRPPPPSFPRKVESLGWSDRNYAEKQRLPREALRRDDRLRSNYSLPSHQSFRGDEISLSRSASSNKDFEVEPERGSSFAESPSIALHDIAMKCGTKVEFKTGLVATPELKFLLEAYFAGDKIGEGTGTTRREAQHRAAEAALMNLADKYLTHIKSDSSTPQSDTSRGHSPIDTGFVSDANSHGDGISRKEDIIPSSEMTGLDDSNVDGSKNSMGSVSVLKELCLREGLGVDFKGQSPTSTNSVDRDEIHAEVEINGQVLGKGTGLTWDEAKMQAAEMALTSLNSMIGQFNKRPSSPRLLQGMPNKRLKPEYPRVVDHLPSSRYPRNASPVP

SEQ ID NO: 17 - protein, 881 aa, Solanum tuberosum
MFKSTVVLYEGERLVGEVEIYCEKGVLWGEKVIRISHYSPSSERCPPLAVLHTVTTGLSFKLEPTKSKPLTQDSPLTLLHSTCLRDNKTAVMSLGREELHLVAMQSKNIGGQCPCFWGFKVASGLYDSCLTMLNLRCLGIVFDLDETLIVANTMRSFEDRIEALQRKINSESDPQRASVMLAEVKRYQEDKIILKQYAENDQVVDNGKVIKSQSEVFPALSDNHQPIVRPLIRLQDRNIILTRINPMIRDTSVLVRLRPAWEDLRSYLTARGRKRFEVYVCTMAERDYALEMWRLLDPDSNLINSQELLDRIVCVKSGLRKSLFNVFQDGNCHPKMALVIDDRLKVWDDKDQPRVHVVPAFAPYFAPQAEGNNSVPVLCVARNVACNVRGGFFKDFDEGLLQRISEVAYEDDIKQVPSAPDVSNYLISEDDPSAVNGNKDSLGFDGMADSEVERRLKEAMLASTSVPSQMTNLDPRLVPALQYPVPPVISQPSIQSPVVPFPTQHLPQVTSVLKSSVTQISPQDTSLQSSPAREEGEVPESELDPDTRRRLLILQHGQDTRDQVSSEPKFPMGTPLQVSVPPRVQPHGWFPAEEEMSPRQLNRPLPPKEFPLNPESMHINKHRPPHPPFLPKMETSMPSDRVLFENQRLPKEVIPRDDRMRFSQSQPSFRPPGEEVPLGRSSSSNRVLDLEPGHYDPYLETPAGALQDIAFKCGAKVEFRSSFLSSPELQFSLEVLFAGEKVGEGTGRTRREAQRRAAEESLMYLADKYLSCIKPDSSSTQGDGFRFPNASDNGFVDNMSPFGYQDRVSHSFASEPPRVLDPRLEVFKKSVGSVGALRELCAIEGLGLAFQTQPQLSANPGQKSEIYAQVAHLLPFLLCYC

SEQ ID NO: 18 - DNA, 2904 bp, Artificial Sequence
  CDS of Arabidopsis_thaliana_AtCPL1
atgtatagtaataatagagtagaagtgtttcatggtgatggaagacttggagaattggagatatacccttcaagggaattgaatcagcaacaagatgatgtgatgaagcagaggaagaagaaacagagggaagtaatggagctagccaagatgggaatcagaataagccacttttctcaatctggcgagaggtgtcctcctcttgcaatacttactacaatttcatcttgtggcctttgtttcaaattagaagcttcaccttctccagctcaggagtcactcagtttattctactcgtcctgtctcagggacaacaagacagcagtaatgctcttgggtggagaagagctccatttggttgctatgtactcggaaaatatcaagaatgaccgtccttgtttctgggcatttagtgttgctcctggaatttacgattcctgtcttgttatgttgaatcttagatgtctgggtattgtctttgatcttgatgaaacccttgtagtggcgaataccatgcgctcatttgaggataagattgacgggtttcagcggagaataaacaacgagatggaccctcaacgccttgccgttatagtggctgagatgaagcgttatcaagatgacaaaaatctattgaagcaatatattgaaagtgaccaggttgttgaaaacggggaggtgataaaggtgcaatctgaaattgttcctgccttgtctgacaaccatcagcctcttgttcgccctctgataaggttgcaagagaagaatattattctgactcgcattaacccaatgattcgtgatacaagtgttcttgtgagaatgaggccctcatgggaggaacttcgaagctatttgacagcaaaagggcgtaagcgttttgaagtatatgtttgcacgatggctgaaagagattacgccttagagatgtggaggctccttgatccagaagggaatttgataaacacaaatgacttgcttgctcgcatcgtttgtgtgaaatctggttttaaaaagtcactgttcaatgtgtttctcgatggaacctgccatccaaagatggcattggtaattgatgatcgattgaaagtttgggatgagaaggatcagccgagggtacatgtggttcctgcattcgctccctattattctcctcaagctgaagctgctgcaacaccagtactatgtgttgccaggaacgttgcctgtggtgtcagaggtggatttttcagggattttgatgatagtctgctaccaaggattgctgaaatttcttatgagaatgatgctgaggatattccttctccgcctgatgtcagccattatttggtgtcggaggatgatacatcgggtttaaatgggaacaaagatccactttcctttgacgggatggctgatactgaagtggagagaagactgaaggaggcaatttctgcatcttcagctgtccttccggcggcaaatatagatccgaggatagctgctcccgttcagttccccatggcttctgcttcttctgtttcagttccagtaccagtacaagtcgtgcaacaagcaatacaaccttcagctatggcctttccaagtattccatttcaacaacctcaacaaccgacatcaatagctaaacacttggttccttcagaaccaagcttgcagagttctcctgctagagaggaaggtgaggtacctgaatcagaattagatccagatactaggaggagactcctcatattgcagcatggacaagatactagggatcctgctccaagtgaaccttcatttcctcagagacctccagttcaagctccaccctcacatgtgcaatcaagaaatggctggtttcctgttgaggaggagatggatcctgctcaaattcgtcgagcagtctcaaaagaatatccgttggattctgaaatgattcatatggaaaagcacaggcctcgtcatccatcatttttttctaagattgataactcaactcagtctgacaggatgcttcatgagaatcgcaggccgccaaaggagtctctccggagagatgaacagttacgttcaaataacaatctacctgactctcatcctttctatggggaggatgcgtcttggaatcaatcttcctctaggaacagtgatcttgacttcctacctgaacgaagtgtctcagcaacggagacttcagctgatgttctacacggaattgctatcaaatgtggagctaaggtggagtacaaaccaagtttagtttctagtacagatttgcggttctctgttgaggcttggctttctaatcaaaaaattggagaagggattggcaaatcgagaagagaagccctgcataaggctgctgaagcttctatacagaatttagctgatggatatatgcgtgcaaatggtgacccagggcccagccacagagatgctacccccttcaccaatgaaaatataagtatgggaaacgcaaatgcgcttaataatcagccatttgctagagatgaaacagcgttgccagtttcttctagacctacagatccgagattagaaggttctatgaggcacactggctccattactgcactcagggaattgtgtgcatcggagggtcttgagatggcttttcaatctcagcgtcagcttccatctgacatggtccacagagatgaattacatgctcaggttgaaatagatgggcgtgttgtaggggaaggagttggatcgacatgggacgaagctagaatgcaggctgctgagagagcactgtccagtgtgagatcaatgcttggtcaacctctgcataaacgacaaggatctccacgatcatttggtgggatgtcaaacaagcgattaaagccggactttcaacggtctctgcaacggatgccatcttcgggaagatactcttaa

SEQ ID NO: 19 - DNA, 2799 bp, Artificial Sequence
  CDS of Zea mays ZmCPL1.1
atgttcaagtcgatggtttattacgtgaacacctcaatcggagaggtggaggtgtggcccaagggcgaggcgagcgcgggcctgaccatggcggcgtgggcgcgagaaatccgcgttgaacgcatttccccgcccagcgagcggtgccctccgctggccgtcatgcacaccgtggccgtcggcgcccgatgcctcgtcatggagtccaggccgcccgttgtcgccgacgttgtgccgcctctcgtcgtcatgcacaccgcttgcctcagggagaacaagactgcggttgttccacttggagatgaagagttgcatttagttgcgatgacatccagaagaaacttgacaaatcatgcatgtttctggggctataaattgccatttggtttgtataattcttgcttgaccatgttaaatcttcggtgcctgggtattgtatttgaccttgatgagacattgattgtcgccaatacatcacggtcttttgaggacagaattgacgcacttcaaagaaagctgagtaatgagactgatccacaacgtaggaatggtatgctatcagagatcaagaggtaccaggatgataagtccatcctaaagcaatatatagaaggtgatcaggtctatgatgatggaaaagtgtataaagcacaacctgagattgttccaccattgtctgataaccagcaaccaatgacacgtccagttataagattacaagataaaaacattatcctgacaagaataaatcctctgattagggataccagtgtgcttgtatgtttaaggccagcctgggaggatcttcgcagctacttaattgccagaggtcgcaagcgttttgaggtctatgtgtgtacgatggctgaaagagactatgctttagaaatgtggagattgcttgatccagattcaagattgattaattctgttcaactccatgatcggatggtgtgtgtaaaatctggtttaaaaaagtccttgctaaatgtcttccatgatggttcttgccatcctggtatggcattagtaattgatgatcgcctgaaagtttgggatgagaaggatcaattacgagttcatgtggttcctgcatttactccatattatgctcctcaggcggaggcaaattgttctatcccagttctgtgtgtagccagaaacgttgcatgcaatgttaggggtggtttcttcaaagactttgatgaaggcctcttaccaaggattagcaatgttcactatgaggatgaagtaaatgagatatctgcgccagatgttggcaattatttgataacagatgatgaaaatgtcgcattagtgaatgggaatagagattcattgccttttgatggtatggcagatgcagaggttgagcggagaatgaaggaagccaatgctcaatcattccatcaaacagcgggagactttgtcatgccagtagcccctgcccagaatttcgtttcgacttcagttgcatcattagccccacctcttggcatgatgccatctccatttagccagccagttgctccaccaggtttttcagattcactgcaaggttctccagctagagaagagggtgaagttccagagtctgagttggatccggacacaaggagaaggcttcttatattacagcatggccaagacacaagagatcctacatctccactaccagcaataccacccgtccaagttccagttcctccagtgcaaccccatgggaattggtttcccacagaggatgggataaaccaaagtaacctgaatagaggctcagcaggattcactgtagaatctgattctattgtctatgagaaaaagcaaccacctcatccttcattctttcatggtggggatagtcctatgccgtctgatagatttggctatcagaaccagaggtttccctctcagctaccacacgaggatcaccccatgatgcagaaccatgcacctccaaaatacagatccttttctggtgaggaactagcatcttggcatgttccctcaagccagagaaacaaccagatagaatcaggaagacactttgcacaatatgctgggacctctgctggcatattagaggggattgctctgaaatgtggttctaaggtggagtacaagtcagcattatgtgatactgcagaactacaattctctattgaggtttggattgttggggaaaaggtcggtgaaggaattggtaggacaaggagagaagcacaacgccaagctgcagaaatgtctttaagaaacttggccaataaatacttgtcatctgatccaaataagttgtctgatatgaaagaaaatgatttcagtagcaacagaaatgtctttggttactctggaaatacgagggatgatatgttgccactttcaagtacttccgaggaatctcgctttatgaaaatggagaacaataattcccggaaaacaggaagttctgttgctgctctcaaagaactttgcactgttgagggatataacttagtttttcaagcctgtccatcttcagcagatggtttagttgggaaagaatcttatgctcaggtacaagttggtggacaaattctaggcaaaggagttgggttaacatgggaagaggccaagctccaggctgctgctgaggctcttggaactttaagatccatgctaggtcaacttggtcataaacgatctggctctccaaggtcattggcaccaaattttaacaagcggttcaagccagattttccaaggaccgtacaaagagttccttacggaacatattctaggatcgaaggtcatgttccttaa

SEQ ID NO: 20 - DNA, 2805 bp, Artificial Sequence
  CDS of Zea mays ZmCPL1.2
atgttcaagtcgatggtttatttcgtgaacatctcaatcggggaggttgaggtgtggcccaagggccaggcgagcgcaggcctggccgtggcggcgtgggcgcgagatatccgcgttgaccgcctttccccgcccagcgagcggtgccctccgctggccgtcatgcacaccgtggccgtcgccgcccgatgcctcgtcatggagtccaggccgcccgtcgccgccgacgtggcgtcgctgcctctcgtcgacatgcacgccgcttgcctcagggacaacaagaccgcggttgttgcacttggagatgaagaattgcatttagttgcgatgacatccagaagaaacttgacaaatcatgcatgtttctggggctacaagttgccatttggtttgtataattcttgcttgaccatgttaaatctacggtgcctgggaatcgtgtttgaccttgatgagacattggttgtcgccaatacatcacggtctttcgaggacagaattgatgcacttcaaagaaagctgagcaatgagactgatccacagcgtagaaatggtatgctatcagagatcaagaggtaccaagatgataaatccatcttaaagcaatatatagaaggtgatcaggtctatgatgacggtaaagtgtataaagcacaacctgagattgttccaccattgtctgataaccagcaaacaatgacacgcccagttataagattacaagaaaaagacattatcctgacaagaataaatcctctgattagggataccagtgtgcttgtacgtttaaggccagcctgggaggatcttcgcagctacttaattgccagaggtcgcaaacgttttgaggtctatgtgtgtacgatggctgaaagagactatgctttagaaatgtggagattgcttgatccagattcaagattaattaattctgttcgacttcatgataggatggtgtgcgtaaaatctggtttaaagaagtccttgctaaatgtcttccatgatggttcttgtcatcctggtatggcattagtaattgatgatcgtctgaaagtttgggatgagaaggatcaattacgagtccatgtggttcctgcatttactccatattatgctccacaggcagaggcaaattgttctatcccggttctttgtgtagccagaaacgttgcatgcaatgttaggggtggtttcttcaaagacttcgatgaaggactcttaccaaggattagtaatgttcattatgaagatgaagtaaatgatatctcatctgcgccagatgttggcaattatttgataacagaggatgagaatgtcgcattagtgaatggaaatagagattcattgccttttgatggtatgtcagatgcagagattgaacggagaatgaaggaagccaatgctcaagcattccatcaaacagcgactaactttgtcatgccagtagcccctgctcagaattttgtttcgtcttcagttgcaccattagcgccacctcttagcatgatgccgcctccattcagccagccagttgttcagccaggtttttcagatccactgcaaggttctccagctagagaagagggtgaagttcctgagtctgagttggatccagacacaaggagaaggcttctcatattgcagcatggtcaagacacaagagatcctacacctccacacccagcgataccaccagtgcaagttccagttcctccagtgcaacctcatgggaattggtttcccacagaggatgggataaacccaagtaacctgagtagaggttcagcaggtttcaccgttgaatccgattctatgccctatgagaaaaaacaaccacctcatccttcattctttcatggtggggatagtcctatgtcgtctgatagatttggctatcagaaccagaggtttccctctcagctaccgcacaccgaggatcaccacatgcttcagaaccatgcacctccaaaatacagatccttttctggtgaggagctagcaactcggcatgttccctcaagccaaagaaaccatatagaatcaggaagacacttttcacaatatgctgggacctctgctggcgtattagagggcattgctgtgaaatgcggatctaaggtggagtacaggtcaacattatgtgataccgcagaactacaattctctattgaggtttggattgttggagaaaagtttggtgaaggaattggtaggacaaggagagaagcacagcgccaagctgcagaaatgtctttaagaaacttggccaataaatacttgtcatctgatccaaataagttgactgatatgaaacaagatgctttcggtagcaacagaaacatctttggttactctggaaatactagggatgatatgttgccactttcaagtacttctgaggaatctcgctttatgaaaatggaggagaacaattccaggaaaacaggagattctgtcactgctctcaaggaactttgcacagttgagggatataacttagtttttcaagcctgtccatctccagcagatggtttagttgggaaagaatcttatgctcaggtacaaattggtaggcaaattctaggcaaaggagttgggttaacatgggaagaggccaagctccaggctgctgatgaggctcttggaactttaagatccatgctaggtcaacttggtcatagacgatctggctctccaaggtcattggcaccgaattttaacaagcggttcaagccagattttccaaggaccgtgcaaagagttccttatggaacatattctagaatcgaaggccatgctccttag

SEQ ID NO: 21 - DNA, 2805 bp, Artificial Sequence
  CDS of Sorghum bicolor Sobic.004G227600.1
atgttcaagtcgatggtttattacgggaacacctcaatcggagaagttgaggtgtggcccaaaggcgaggcgagcgcgggcctggccgtggcggcgtgggcgcgagaaatccgcgttgaccgcctttccccgcccagcgagcggtgccctccgctggcggtcatgcacaccgtggccgtcggcgcccgatgcctcgtcatggagtccaggccgcccgtcgccgccgacgtggcgccgatgcctctcgtcgccatgcacgccgcttgcctcagggacaacaagaccgcggttgttccacttggagatggagaattgcatttagttgcgatgacatccagaagaaacttgacaaatcatgcatgtttctggggctataaattgccatttggcctgtataattcttgcttgaccatgttaaatcttcggtgcctgggtatcgtgtttgaccttgatgagacgttgattgtcgccaatacatcacggtcttttgaggacagaattgatggccttcaaagaaagctgagtaatgagactgatccacagcgtaggaatggtatgctatcagagatcaaaaggtaccaggatgataagtccatcctaaagcaatatatagaaggtgatcaggtctatgatgatggaaaagtgtataaagcacaacctgagattgttccaccattgtctgataaccagcaaccaatgacacgtccagttataagattacaagataaaaacattatcctgacaagaataaatcctctgattagggataccagtgtacttgtacgtttaaggccggcctgggaggatcttcgcagctacttaattgcaagaggtcgcaagcgttttgaggtgtatgtgtgtacgatggctgaaagagactatgctttagaaatgtggagattgcttgatccagattcaagattgattaattctgttcaacttcatgatcggatggtgtgtgtaaaatctggtttaaagaagtccttgctaaatgtcttccatgatggttcttgccatcctggtatggcattagtaattgacgatcgtctgaaagtttgggatgagaaggatcaattacgagttcatgtggttcctgcatttactccatattatgctcctcaggcagaggcaaattgttctatcccggttctgtgtgtagccagaaacgttgcatgcaatgttaggggtggtttcttcaaagacttcgatgaagggctcttaccaaggattagcaatgttcactatgaggatgaagtaaatgatatctcatctgcgccagatgttggcaattatttgataacagaggatgagaatgccgcattagtgaatggaaatcgagattcattgccttttgatggtatggcagatgcagaggttgagaggagaatgaaggaagctaatgctcaagcattccatcaaacagcggggaactttgtcatgccagtagcccctgctcagaattttgtttcgtcttcagttgcaccattagccccacctcttggcgttatgccaccaacattcagccaaccagttgttcaaccaggtttttcagattcactgcaaggttctccagctagagaagagggcgaagttccagagtctgagttggatccggacacaaggagaagacttctcatattacagcatggtcaagacataagagatcctacacctccactcccagcaataccaccagtgcaagttccagttcctccagtgcaacctcatgggaattggtttcccacagaggatgggctaaacccaagtaacctgaatagaggttcagcaggtttcactgtagaatctgatcctatgctctacgagaaaaaacaaccacctcatccttcattctttcatggtggggacagtcctatgtcgtctgatagatttggctatcagaaccagaggtttccctctcagctaccacacaccgaggatcaccacatgcttcagaaccatgcacctccaaaatacagatccttttctggtgaggagctagcagctcggcatgttccctcaagccagagaaacaaccagatagaatcaggaagacactttgcacaatatgctgggacctcagctggcatattagacggcattgctctgaaatgtggatctaaggtggagtacaggtcaacattatgtgatactgcagaactacaattctctattgaggtttggattgttggggaaaaggttggtgaaggaattggtaggacaaggagagaagcacagcacaaagctgcagaaatgtctttaagaaacttggccaataaatacttgtcatctgatccaaataagttgactgatatgaaagaaaatggtttcagtggcaacagaaatgtctttggttactctggaaataccagggatgatatgttgcctctttcaagtacttcagaggaatctcgctttatgaaaatggagaacaattccaggaaaacaggaggttctgttgctgctctcaaggaactttgcacggttgagggatataacttagtttttcaagagcgtccatctccagcagatggtttagttgggaaagaatcttatgctcaggtagaagttggtggacaaattctaggcaaaggagttggattaacatgggaagaggccaagctccaggctgctgatgaggctcttggaactttaagatccatgctaggtcaacttgctcataagcgatctggctctccaaggtcattggcaccaaattttaacaagcggttcaagccagattttccaaggaccgtacaaagagttccttatggaacgtattctaggatcgaaggtcatgttccgtag

SEQ ID NO: 22 - DNA, 2829 bp, Artificial Sequence
  CDS of Sorghum bicolor Sobic.006G156400.1
atgatcaagtcgctggtttattacgggaacacccctgttggggaggtggaggtgtggcccaagggccagacggacctggcatgggcgcgcgagatccgggtggaccggctgtccccggccagcgagcggtgcccgccgctcgccgtgctgcacgtcgtcgcggccggtgcccgatgcctcgtgatggagtcgaagtccactgccacagctcacgagcccccgccgccgctcgtcaccatgcacaccacttgtctcaaggataacaagacggctgtttttccacttggagcagaagagatacacttagtggcgatgacttctaagagaaacatgccaaacggtgcatgtttttggggctataaagtgccattgggcttatataattcttgcctgagcatgctgaatcttcgatgccttggtattgtgtttgaccttgatgaaacattaattgttgccaataccacacgatcttttgaagacaggatagatgctattcagagaaaattgaataatgaggctgatccgcagcgcattagtggtatgttggcagagatcaagaggtaccaagaagacaagtctatcctaaaacagtatatagaaagtgaccaggttactgatggaggagaattgtacaaagtacaatcagaggtcattccacccttagatgataatcatcaacaacctatgacacgcccgattataaggttgcaagagaaaaatattatcttgacacgtataaatccatcaataagagatactagtgttctggtgcggttaagacctgcatgggacgaccttcggagctacttaattgcaagaggtcgcaaacgttttgagatttatgtgtgcacaatggctgagagggactatgctttggaaatgtggaggttgcttgatccagattcaaaattgataaactctgttcaacttcttgacagacttgtatgtgtcaaatctggctcaagaaagtctttgctaaatgtattccatgatgggtcttgtcaccccggaatggcccttgtcattgacgaccgtttgaaagtttgggatgagtgggaccaacgtagagttcacgttgttcctgcatttgctccatattacgctccccaggcagaggcaaactttcctattccagttctatgcgtcgcacggaatgttgcttgcaatgttagggctggtttcttcaaagaatttgatgagggcatcattccaaggattacagaggtttgttatgaggacgaactggatgacattgcttctgctcctgatgtttgcaattattttgtttcagaggacgagaatgctgctgtttcaaatgtaaacaaaaatccactagcttttgatggtatggcagatgcagaggttgagaagagaatgaaggaagcttcttccagctttcaatctgcgaatccaataacaactaatgttgatgtgatgtcagtagccgctaaccaacactttggtacacctatatcctcatctactccagtagcaccaccactgggtatgcttttgaataatgatcaggatcctcagccaccttcactcagatggcctgttgctcaatctggccatgtggactcttcccaaggttctccggctagagaagagggtgaagttcctgagtctgaattggatcctgacactcggagaaggcttctcatattacaacatggtcaagacacaagagatcctgcagcgccgtttcctgctggatctcctgcacaagtctcagtaccacctgtgcaatctcatgaaaattggttttgcttagaggatgagatgaaccctagaaacctgaacaaggtttcaacagaatttcatttagaatctgattctgtacattatgataagaagcaattgcagcatacatcatacattcctattggggataatcctatgtcttatgatagatacgattatcagaatcagagatatccttctcagccacctcacagtgaaggtcatcatagatttcataaccatgcgcctacagcttatcgatccttttctggtgaggacatggcaacttggtatgctccttcagggcaaagaagcagccatatggaatcaggacgacattttgcacgatatggaggaattccaggtgtattagaggaaattgcattgaagtgtggatttaaggtggagtaccgatcaactttatgtgatacgacagagttgcaattctccactgaggttttaatttttggagaaaaggtaggtgaaggagttgggaaaacaaggaaagaagcacagtggcaagctgctgatacatccttaagaaatctggcagacaagtttctatcatgggacccagataaggtgacagttctaaaggagaatgactttaataggcatccaaagtcacacagatatccaggaagtaatatatatgacacattaccagttgcaagcacttcagatgagtctagatatatgaatgacagaattgatactttaagaaaacctggtgcttctgttgctgctcttaaggagctttgtgcggttgaagggtataatttggatttccatgctcagccatctgcggatggttcagtaggtaaagaaattcgtgctcaggtagaaataggaggaaaagtcctgggtaaaggagttggagtaacatgggaggaagctaagctgcaggctgcttatgaggcatatggaacgttgaaatccatgctcggtcaatttgttccgaggcaatctgcatctccaaggtcaatggtgccaaattttaataagcgcttcaatccagatttctccgaggcactacaaaggattccttcaggcagatattctagaaatgacagtcgttttccttga

SEQ ID NO: 23 - DNA, 2847 bp, Artificial Sequence
  CDS of Triticum aestivum TraesCS2A02G373400.3
atgatcaaatcgatggtgtatttcgggcacatctccatcggggaggtggagctgtctcccaagggggagacgaacgtggcggcggcgccgtgggtgcgggagatccgggtggaccgcctctccccgcccagcgagcggtgcctgccgctcgccgtgctgcacactgtgtcctccggcgccctctgcttcgtaatggagtcgaggccaagcccagcaaccgctgacaacgagcccccgtcgtcgcttgttgccatgcacaccgcttgcctcagagacaataagacagcagtttttccacttggagcagaagaaatccatttagttgcaatgaaacccaagagtaacttgcccaaccacgcatgtttttggggctataaagtaccattaggcctgtatagttcttgcttgagtatgttgaatcttcgatgccttggtattgtgtttgaccttgatgaaacactaattgttgccaataccacacggtcttttgaagacagaattgatgcactccagagaaaattgagtaaagagactgatccacagcgcattagtggtatgttggcagagatcaagaggtaccaagaggacaggactatgctaaagcagtatatagatggtgatcaagttattgatggtgggaaaatgtataaagtacaatccgaggttgttccacctttagctgataatcatcaacctatgatccgtcctgttataaggctacaagacaaaagtattatattgacacgtataaatccatcgataagagataccagtgttctggtgcggttaaggcctgcttgggatgatcttcggagctacttgattgcaagaggtcggaaacgttttgaggtgtatgtgtgcacaatggctgagagggactatgccttggaaatgtggaggttgcttgatcctgattctagactgataaactctgttcaacttcctcacaggcttgtctgtgtcaaatctggctctaaaaagtcattgctaaatgtgttccatgatggatcttgtcaccctggtatggccctagtgattgatgaccgtttgaaagtttgggaggagaaggaccaatgtcgagttcatgttgttcctgccttctctccgtattatgctccgcaagcagaggcgaacttccctattccagttctttgtgttgccagaaatgttgcatgcaatgttaggggtagcttcttcaaggaattcgatgagggtcttctaccatcgatcagtgaagttcattttgatgatgaattagatcatgttccgtcttctcctgatgtcggaaattatttgatttcagaggatgagaatgcagcaagtttgaatgtgaacaaagatccgatggcttttgacggtatggcagatgcagaggttgagaggagaatgaaggaagcagtttgcagtgtgcaagctgcagatccagtgacaacaaatgttgacgtgatatcagtagctgctaaccagcaatttgctacatcttcatctattccgttagcaccaccactgggcgtggtgcctttgaataacgatcaaggtcctcagcctccttcagtcagctggcctgatgctcaatctggtatggtggatcctttgcaaggttctccagctagagaagagggtgaggttcctgagtctgagttagatcctgacacaaggagaaggcttctgatattacagcatggccaagacacaagagatcccacaccaccctttgctgcagaaccttctgtacaagcctcagttcctccagtgcaatctcagggaaattggtttcctgtagaggatgagatggacccaagaaacttaaataggacttcaacggactttcatctagaatctgatgccgtacatagtgataaaagccaaccaccacatcaaccatacttccctgctcgtgataatcctatattttccgatagattaaatcatcagaaccagagatattcttctcagctacctcacagtgaggatcgtcaaatgcttcagaaccaggcacctacaacatatagatcattttctggcgaggatatggcaacccagcgttttcatccaggtaacagaagcagccaaatggagtcaggacgtcaatttgtacaatacacagagacatctggtgctgtattggaggagattgcagcgaagtgtggatttaaggtggagtacaggtcaactttatgtgatactacagagttgcgattctccattcagatttggattgttggagaaaaagtaggtgaaggaatgggaagaacaaggaaagaagctcaacgacaagctgctaatatatcattaagaaatctggcagatagatttctgtcatttgatccagataagatgacagttccggtggatgatggttttagtagcaatccaaactcattcaaatatagagggattgatggagataatatagtaccagttgcaagcacgtcggatgggtctagatatatgcacgagagagttgataactcaaccaaatctgctggttctgttgctgctcttaaggagctttgtacagctgagggttataacttagttttccaagctcagccatctccatcagatagtttgagaagggaagaagttcatgctcagattgaaataggcgggcaaatcctgggaaaaggagttggagtaacatgggaggaagctaaggtgcaggctgctgatggggctctcggaactctgagatacatgcttggtcaacgtccacagaaacggtctggatctccaaggtcattcgcatccaattataataataagcgctacaagccagatttccagccaatggtacaaaggattccttctggcagatactctaggaatgacagtcgtgttccctga

SEQ ID NO: 24 - DNA, 2847 bp, Artificial Sequence
  CDS of Triticum aestivum TraesCS2B02G390500.1
atgatcaaatcgatggtgtatttcgggcacatctccatcggggaggtggagctgtggcccaagggggagacgaacgtggcggcggcgccgtgggtgcgggagatccgggtggaccgcctctccccgcccagcgagcggtgcctgccgctcgccgtgctgcacactgtgtcctccggcgccctctgcttcgtaatggagtcgaggccaagcccagcaaccgctgacgacgagcccccgtcgtcgcttgttgccatgcacaccgcttgcctcagagacaataagacagcagtttttccacttggagcagaagaaatccatttagttgcaatgaaacccaagagtagcttgcccaaccacgcatgtttttggggctataaagtaccattaggcttgtataattcttgcttgagtatgttgaatcttcgatgccttggtattgtgtttgaccttgatgaaacactaattgttgccaataccacacggtcttttgaagacagaattgatgcactccagagaaaattgagtaaagagattgatccacagcgtattagtggtatgttggcagagatcaagaggtaccaagaggacaggtctatgctaaagcagtatatagatggtgatcaagttactgatggtgggaaagtgtataaagtacaatctgaggttgttccacctttagctgataatcatcaacctatgatacgtcctgttatcaggctacaagagaaaagtattatattgacacgtataaatccatcgataagagacaccagtgttctggtgcggttaaggcctgcttgggatgatcttcggagctacttgattgcaagaggtcggaaacgtttcgaggtgtatgtgtgcacaatggctgagagggactatgccttggaaatgtggaggttgcttgatcctgattctagactgataaactctgttcaacttcctcacaggcttgtctgtgtcaaatctggctctaaaaagtcattgctaaatgtattccatgatggatcttgccaccctggtatggccctagtgattgatgaccgtttgaaagtttgggaggagaaggaccaatgtcgagttcatgttgttcctgccttctctccgtattacgctccgcaagcagaggcgaacttccctattccagttctttgcgttgccagaaatgttgcatgcaatgttaggggtagcttcttcaaggaattcgatgagggtcttctaccgtcgatcagtgaagttcattttgacgatgaattaaatcatgtcccgtcttctcctgatgttggaaattatttgattccagaggatgagaatgcagcaagtttgaatgtgaacaaagatcagatggcttttgatggtatggcagatgcagaggttgagaggagaatgaaggaagcagtttgcagtgtgcaagctgcagatccagtgacaacaaatgttgacgtgataccagtagctgctaaccagcaatttgctacatcttcatctattccgttagcaccaccaccgggcatggtgcctttgaataacgatcaaggtcctcagcctccttcagtcagctggcctgatgctcaatctggtatggtggatcctttgcaaggttctccagctagggaagagggtgaggttcccgagtctgagttagatcctgacacaaggagaaggcttctgatattacagcatggccaagacacaagagatcccacaccaccctttgctgcagaaccttctgtacaagcctcagttcctccagtgcaatctcagggaaattggtttcctgtagaggatgaaatggacccaagaaacttaaataggacttcaacggactttcatctagaatctgatgctgtacatagtgataaaagtcaaccaccacatcagccatacttccctgctcatgataatcctatattttccgatagattaaaccatcagaaccagagatattcttctcagctacctcacagtgaggatcgtcaaatgcttcagaaccaggcacctacaacatatagatcattttctggcgaggatatggcaacccagcgttttcatccaggtaacagaagcagccaaatggagtcaggacgtcaatttgtacaatacacagagacatctggtgctgtattggaggagattgcagcgaagtgtggatttaaggtggagtacaggtcaactttatgtgatactacagagttgcgattctccattcagatttggattgttggagaaaaagtaggtgaaggaatgggaagaacaaggaaagaagctcaacgacaagctgctaatatatcattaagaaatctggcagatagatttctgtcatttgatccagataagatgacagttccgatggatgatggttttagtagcaatccaaactcattcaaatatagagggattgatggagataatatagtaccagttgcaagcacgtcggatgggtctagatatatgcacgagagagttgataactcaacaaaatctgctggttctgttgctgctcttaaggagctttgtacagctgagggttataacttagttttccaagctcagccatctccattagatagtttgagaagggaagaagttcatgctcagattgaaataggcgggcaaatcctgggaaaaggagttggagtaacatgggaggaagctaaggagcaggctgctgatggggctctcaggactctgagatacatgcttggtcaacgtccacagaaacggcctggatctccaaggtcatttgcatccaattataataataagcgctacaagccagatttccagccaatggtacaaaggattccttctggcagatactctaggaatgacagtcgtgttccctga

SEQ ID NO: 25 - DNA, 2844 bp, Artificial Sequence
  CDS of Triticum aestivum TraesCS2D02G369600.6
atgatcaaatcgatggtgtatttcgggcacatctccatcggggaggtggagctgtggcccaagggggagacgaacgtggcggcggcaccgtgggtgcgggagatccgggtggaccgcctctccccgcccagcgagcggtgcctgccgctcgccgtgctgcacactgtgtcctccggcgccctctgcttcgtaatggagtcgaggccaagcccagcaacagctgacgacgagcccccgtcgtcgcttgttgccatgcacaccgcttgcctcagagacaataagacagcagtttttccacttggagcagaagaaatccatttagttgcaatgaaacccaagagtaacttgcccaaccacgcatgtttttggggctataaagtaccattaggcctgtataattcttgcttgagtatgttgaatcttcgatgccttggtattgtgtttgaccttgatgaaacactaattgttgccaataccacacggtcttttgaagacagaattgatgcactccagagaaaattgagtaaagagactgatccacagcgtattagtggtatgttggcagagatcaagaggtaccaagaggacaggactatgctaaagcagtatatagatggtgatcaagttattgatggtgggaaaatgtataaagtacaatccgaggttgttccacctttagctgataatcatcaacctatgatccgtcctgttataaggctacaagagaaaagtattatattgacacgtataaatccatcgataagagataccagtgttctggtgcggttaaggcctgcttgggatgatcttcggagctacttgattgcaagaggtcggaaacgttttgaggtgtatgtgtgcacaatggctgagagggactatgccttggaaatgtggaggttgcttgatcctgattctagactgataaactctgttcaacttcctcacaggcttgtctgtgtcaaatctggctttaaaaagtcattgctaaatgtgttccatgatggatcttgtcaccctggtatggctctagtgattgatgaccgtttgaaagtttgggaggagaaggaccaatgtcgagttcatgttgttcctgccttctctccgtattacgctccgcaagcagaggcgaacttccctattccagttctttgcgttgccagaaatgttgcatgcaatgttaggggtagcttcttcaaggaattcgatgagggtcttctaccatcgatcagtgaagttcattttgacgatgaattagatcatgttccgtcttctcctgatgttggaaattatttgatttcagaggatgagaatgcagcaagtttgaatgtgaacaaagatccgatggcttttgacggtatggcagatgcagaggttgagaggagaatgaaggaagcagtttgcagtgtgcaagctgcagatccagtgacaacaaatgttgacgtgatatcagcagctgctaaccagcaatttgctacatcttcatctattccgttagcaccaccactgggcatggtgcctttgaataacgatcaaggtcctcagcctccttcagtcagctggtctgatgctcaatctggtatggtggatcctttgcaaggttctccagctagagaagagggtgaggttcctgagtctgagttagatcctgacacaaggagaaggcttctgatattacagcatggccaagacacaagagatcccacaccacccttcgctgcagaaccttctgtacaagcctcagttcctccagtgcaatctcagggaaattggtttcctgtagaggatgagatggacccaagaaacttaaataggacttcaacggactttcatgtagaatctgatgctgtacatagtgataaaagtcaaccaccacatcaaccatacttccctgctcgtgataatcctgtattttccgatagatttaaccatcagaaccagagatattcttctcagctacctcacagtgaggatcgtcaaatgcttcagaaccaggcacctacaacatatagatcattttctggcgaggatatggcaacccagcgttttcatccaggtaacagaagcagccaaatggagtcaggacgtcaatttgtacaatacacagagacatctggtgctgtattggaggagattgcagcgaagtgtggatttaaggtggagtacaggtcaactttatgtgatactacagagttgcgattctccattcagatttggattgttggagaaaaagtaggcgaaggaatgggaagaacaaggaaagaagctcaacgacaagctgctaatatatcattaagaaatctggcagataaatttctgtcatttgatccagataagatgacagttcctatggatgatggttttagtagcaatccaaactcattcaaatatacagggattgatggagataatatagtaccagttgcaagcacgtcggatgggtctagatatatgcacgagagagttgataactcaaccaaatctgctggttctgttgctgctcttaaggagctttgtacagctgaggggtataacttagttttccaagctcagccatctccattagatagtttgacaaggaaagaagttcatgctcagattgaaataggcgggcaaatcctgggaaaaggagttggagtaacatgggaggaagctaaggtgcaggctgctgatggggctctgggaactctgagatacatgcttggtcaacgtccacagaaacggtctggatctccaaggtcatttgcatccaattataataagcgctacaagccagatttccagccgatggcacaaaggattccttctggcagatattctaggaatgacagtcgtgttccctga

SEQ ID NO: 26 - DNA, 2805 bp, Artificial Sequence
  CDS of Triticum aestivum TraesCS6A02G235300.1
atgatcaagtccatggtttattacgggaacacctccatcggggaggtggaggtgtggcccaagggcgacacgaacctgggcgcggcggcgtgggcgcgagagatccgcgtggaccgcctttccccgcccagcgagcgctgcctgccgctcgccgtcatgcacaccgtggcggttggagctcgctgcctcgtcatggagtccaggccgcccaaggccgccgacgagccaccaccgccactcgtcgccatgcacgctgcttgcctcagggacaataagactgcagttgttccgttaggagaggaagaattgcatttagtggcaatgacatcagggagaaacttgacaaaccatgcatgtttctggggctataaagtgccatttggtctgtataattcttgcttgaccatgttaaatctccgctgcctcggtattgtgtttgaccttgatgagacattgattgttgccaataccacacgctcttttgaggacagaattgattcacttcaaagaaagctgagtaatgagactgatccacaacgcatgaatggtatgttagcagagatcaagaggtaccaagatgatagatctatcctaaagcagtatatagaaggtgatcaggtctatgatgatggaaaaatgtataaagtgcaacctgagattgttccaccattgtctgataaccatcaatcattgacacgcccagttataagattacaagagaaaaacattatcttgaccagaataaatcctttgataagggatacaagtgttcttgttcggttaaggccagcatgggaggatctccgaagctacttgattgcaagaggtcgcaagcgttttgaggtctatgtgtgtacaatggcggagagagactatgctttagaaatgtggagattgcttgatccagactcaagattgattaactctgttcaacttagtgacagaatggtgtgtgtaaaatctggcttaaaaaagtccttgctaaatgttttccatgatggatcttgccatcctggaatggcattagtaattgatgatcgtctgaaagtttgggatgagaaagaccaatctagagttcatgtggttcctgctttcaccccatactatgcacctcaagccgaggcaaactgttccatcccagttctatgtgttgccagaaatgtcgcgtgcaatgttaggggcggcttcttcaaggactttgatgaaggcctcttgccaaggattacgagtgttctttatgaggatgaaatacaggatatctcatcagccccagatgtgggcaattatttgatttcagaggatgagaatgttgcagtagtgaatgggaacagagattcactggcttttgatggcatggctgatgctgaggttgagaggagaatgaaggaagcatctggcagtggcagtgttttgaatccaacaatggctaacatggtgatgccagtagcccctagtcaaagttttgctccatcttcagtagcgccatttgcaccacctcttggcatgatgcctttgagtaacaatcaagttccgccacctgcattcagccaaccagtcgttcaaccagttgtcttagatccactgcaagcttctccaggtagagaagagggcgaggttccagagtctgagttagatcctgacacaaggagaaggcttctcatattacaacatggccaagacacaagagatcctacaccacctcttccagcagtacctcctgcgcaagtttcagttcctccggtgcaatctcatgggaattggttccctatcgaggatggcattggcatgaactcaaataatttgaacatgggttcagcagggttcccttcagaatctgataccatgcattatgataaaaaacaaccaccacagccatcttacttccatggtggcgataataatccagtgccctctgatagatttagttatcagagccagaggtttccctctcaggtaacacatactgaggaccaccgcatgcttcagaaccatgcacctccaagatacagatcctttcctggtcagagaaacaacttgattgagtctggacaaagctatgcacgaaatgtaggttctgttggcatactagaggagatcgcactgaagtctggatctaaggtggagtacaggtcaacattatgtgataccgcagaactacagttctctattgagatttggatagtgggagaaaaagtgggtgaaggcattggtagctcaaggaaagaagcacagcgtcaagctgcagaaatatctttaagaaatttggccaataaatacctgttgtctgatccaaataagatgactgatgtgaacgaagatggtttcggtagcaatccaaacttttttggatactctgaaaataccaggaacgatatattgtcagtcgcaagcacttcagaggaatcccgattcacgaaaacaggggagaacaattccagaataacaggaggctctattgctgctctgaagcaactttgcacagttgagggatataacttagtttttcaagctcggccaagtcctctagatggttcaggtggcaaggagacttacgctcaggtagaagttggtgggcaaactcttggcaaaggagttggaataacatgggaagaagctaagcttcaggcggctgatgaggctcttggaactttgagatccatgcttggtcaacttgctcagaaacggtctagttctccaaggtctttggcgcccaattataataagcgcttcaagccagatttcccccgggcagtgcaaaggcctccttatggtagatattccaggattgaaggccatgttccttga

SEQ ID NO: 27 - DNA, 2808 bp, Artificial Sequence
  CDS of Triticum aestivum TraesCS6B02G264000.4
atgatcaagtccatggtttattacgggaacacctccatcggggaggtggaggtgtggcccaagggcgacacgaacctgggcgcggcggcgtgggcgcgagagatccgcgtggaccgcctttccccgcccagcgagcgctgcctgccgctcgccgtcatgcacaccgtggcggtcggggctcgctgcctcgtcatggagtccaggccgcccaaggccgccgacgagccaccaccgccactcgtcgccatgcacgctgcttgcctcagggacaataagactgcagttgttccgttaggagaggaagaattgcatttagtggcaatgacatcagggagaaacttgacaaaccatgcatgtttctggggctataaagtgccatttggtctgtataattcttgcttgaccatgttaaatctccgctgcctcggtattgtgtttgaccttgatgagacattgattgttgccaataccacacgcacttttgaggacagaattgattcacttcaaagaaagctgagtaatgagactgatccacaacgcatgaatggtatgttagcagagatcaagaggtaccaagatgatagatctatcctaaagcagtatatagaaggtgatcaggtctatgatgatggaaaaatgtataaagtgcaacctgagattgttccaccattgtctgataaccatcaatcattgacacgcccagttataagattacaagagaaaaacattatcttgaccagaataaatcctttgataagggatacaagtgttcttgtccggttaagaccagcatgggaggatctccgaagctacttgattgcaagaggccgcaagcggtttgaggtctatgtgtgtacaatggcggagagagactatgctttagaaatgtggagattgcttgatccagactcaagattgattaactctgttcaacttagtgacagaatggtgtgtgtaaaatctggcttaaaaaagtccttgctaaatgttttccatgatagatcttgccatcctggaatggcattagtaattgatgatcgtctgaaagtttgggatgagaaagaccaatctagagttcatgtggttcctgctttcaccccatactatgcacctcaagccgaggcaaactgttccatcccagttctatgtgttgccagaaatgtcgcgtgcaatgttaggggcggcttcttcaaggactttgatgaaggcctcttgccaaggattacgagtgttctttatgaggatgaaatacaggatatctcatcagccccagatgtgggcaattatttgatttcagaggatgagaatgttgcagtagtgaatgggaacagagattcactggcttttgatggcatggctgatgctgagggtgagaggagaatgaaggaagcatctggcagctgcagtgttttgaatccaacaatggctaacatggtgatgccagtagcccctagtcaaagttttgttccatcttcagtagcgccatttgcaccacctcttggcatgatgcctttgagtaacaatcaagttccgccacctgtattcagccaaccagtcgttcaaccagttgtcttagatccactgcaagcttctccaggtagagaagagggcgaggttccagagtctgagttagaccctgacacaaggagaaggcttctcatattacaacatggccaagacacaagagatcctacaccacctcttccagcagtacctcctgcacaagtttcagttcctccggtgcaatctcatgggaattggttccctgtcgaggatggcattggcatgaactcaaataatttgaacaggggttcagcagggttcccttcagaatcagataccatgcattatgataaaaaacaaccaccacagccatcttacttccatggtggcgataataatccagtgtcctccgatagatttagttatcagagccagaggtttccctctcaggtaacacatactgaggaccaccgcatgcttcagaaccatgcacctccaagatacagatcctttcctggtcagagagacaacttgattgagtctggacaaagctatgcacgaaatgtaggtacctctgttggcatactagaggagatcgcactgaagtctggatctaaggtggagtacaggtcaaaattatgtgataccgcagaactacagttctctattgaggtttggatagtgggagaaaaagtgggtgaaggcattggtagctcaaggaaagaagcacagcgtcaagctgcagaaatatctttaagaaatttggccaataaatacctgttgtctgatccaaataagatgactgatgtgaacgaagatggtttcggtagcaatccaaacttttttggatactctgaaaataccaggaacgatatattgccagtcgcaagcacttcagaggaatcccgattcacaaaaacaggggagaacaattccagaataacaggaggctctattgctgctctgaagcaactttgcacagttgagggatataacttagtttttcaagctcggccaagtcctctagatggttcaggtggcaaggagacttacgctcaggtagaagttggtgggcaaactcttggcaaaggagttggaataacatgggaagaagctaagcttcaggctgctgatgaggctcttggaactttgagatccatgcttggtcaacttgctcagaaacggtctagttctccaaggtctttggcgcccaattataataagcgcttcaagccagatttcccccgggcagtgcaaaggcctccttatggtagatattccaggattgaaggccatgttccttga

SEQ ID NO: 28 - DNA, 2808 bp, Artificial Sequence
  CDS of Triticum aestivum TraesCS6D02G218000.4
atgatcaagtccatggtttattacgggaacacctccatcggggaggtggaggtgtggcccaagggcgacacgaacctgggcgcggcggcgtgggcgcgagagatccgcgtggaccgcctttccccgcccagcgagcgctgcctgccgctcgccgtcatgcacaccgtggcggttggggctcgctgcctcgacatggagtccaggccgcccaaggccgccgacgagccaccaccgccactcgtcgccatgcacgctgcttgcctcagggacaataagactgcagttgttccgttaggagaggaagaattgcatttagtggcaatgacatcagggagaaacttaacaaaccatgcatgtttctggggctataaagtaccatttggtctgtataattcttgcttgaccatgttaaatctccgctgcctcggtattgtgtttgaccttgatgagacattgattgttgccaataccacacgctcttttgaggacagaattgattcacttcaaagaaagttgagtaatgagactgatccacaacgcatgaatggtatgttagcagagatcaagaggtaccaagatgatagatctatcctaaagcagtatatagaaggtgatcaggtctatgatgatggaaaaatgtataaagtgcaacctgagattgttccaccattgtctgataaccatcaatcattgacacgcccagttataagattacaagagaaaaacattatcttgaccagaataaatcctttgataagggatacaagtgttcttgttcggttaaggccagcatgggaggatctccgaagctacttgattgcaagaggccgcaagcgttttgaggtctatgtgtgtacaatggcggagagagactatgctttagaaatgtggagattgcttgatccagactcaagattgattaactctgttcaacttagtgacagaatggtgtgtgtaaaatctggcttaaaaaagtccttgctaaatgttttccatgatggatcttgccatcctggaatggcattagtaattgatgatcgtctgaaagtttgggatgagaaagaccaatctagagttcatgtggttcctgctttcaccccatactatgcacctcaagccgaggcaaactgttccatcccagttctatgtgttgccagaaatgtcgcgtgcaatgtcaggggcggcttcttcaaggactttgatgaaggcctcttgccaaggattacgagtgttctttatgaggatgaaatacaggatatctcatcagccccagatgtgggcaattatttgatttcagaggatgagaatgttgcagtggtgaatgggaacagagattcgctggcttttgatggcatggctgatgctgaggttgagaggagaatgaaggaagcatctggcagtggcagtgttttgaatccaacaatggctaacatggtgatgccagtagcccctagtcaaagttttgttccatcttcagtagcgccatttgcaccacctcttggcatgatgcctttgagtaacaatcaagttccgccacctgcactcagccaaccagtcgttcaaccagttgtcttagatccactgcaagcttctccaggtagagaagagggcgaggttccagagtctgagttagatcctgacacaaggagaaggcttctcatattacaacatggccaagacacaagagatcctacaccacctcttccagcagtacctcctgcacaagtttcagttcctccggtgcaatctcatgggaattggttccctatcgaggatggcattggcatgaactcaaataatttgaacatgggttcagcagggttcccttcagaatctgataccatgcattatgataaaaagcaaccaccacagccatcttacttccatggtggcgataataatccagtgtcctctggtagatttagttatcagagccagaggtttccctctcaggtaacacatacagaggaccaccgcatgcttcagaaccatgcacctccaagatacagatcctttcctggtcagagaaacaacttgattgagtctggacaaagctatgcacgaaatgtaggtacctctgttggcatactagaggagatcgcactgaagtctggatgtaaggtggagtacaggtcaacattatgtgataccgcagaactacagttctctattgaggtttggatagtgggagaaaaagtgggtgaaggcattggtagctcaaggaaagaagcacagcgtcaagctgcagaaatatctttaagaaatttggccaataaatacctgttgtctgatccaaataagatgactgatgggaacgaagatggtttcggtagcaatccaaacttttttggatactctgaaaataccagaaacgatatattgccagtcgcaagcacttcagaggaatcccgattcacgaaaacaggggagaacaattccagaataacaggaggctctattgctgctctgaagcaactttgcacagttgagggatataacttagttttccaagctcggccaagtcctctagatggttcaggtggcaaggagacttacgctcaggtagaagttggtgggcaaactcttggcaaaggagttggaataacatgggaggaagctaagcttcaggctgctgatgaggctcttggaactttgagatccatgcttggtcaacttgctcagaaacggtctagttctccgaggtctttggtgcccaattataataagcgcttcaagccagatttcccccgggcagtgcaaaggcctccttatggtagatattccaggattgaaggccatgttccttga

SEQ ID NO: 29 - DNA, 2871 bp, Artificial Sequence
  CDS of Glycine max Glyma.07g194800.1
atgagaatgtataaatcggtggtgtaccagggggaggtggtggtgggtgaggtagatgtatacccagaagagaacaacaactacaagaacttccatgtgaaggaaatcagaataagccacttctcgcaacctagtgagaggtgtcccccacttgctgtgcttcacactgttacttcgtgtggtgtttgcttcaaaatggagtcaaagactcagcagcaggacggcctctttcaattgcactccttgtgtatcagagagaacaagactgctgtaatgccactgggtggggaggaaatacatttggttgcaatgcattcacggaatgtcgatagaccatgcttctggggatttattgttgccttgggactttacgattcatgtcttgtgatgctaaatcttagatgtttgggtatagtgtttgatctggacgaaacactcattgtagcaaatacaatgcgatcatttgaggatcgaattgatgcactccagagaaaaataaactctgaagtagatccacagagaatttccggtatgcaggcagaggtcaagcgctaccaagatgacaagaatatattgaagcaatatgctgaaaatgatcaggttgttgataatggtagagtgataaaagttcaatctgagattgttccggcattatctgacagtcatcagcctatagttcggccactgatacggttacaggataagaacattattctgacgcgcatcaatccacagattcgtgatacgagtgttcttgtgaggttgagacctgcatgggaagatcttcggagctacttgactgcaagagggcgcaagcgttttgaggtttatgtctgcacaatggctgaaagggactatgcactagaaatgtggagacttcttgatccagattcaaatttgataaattctaaagaactattaggtcgcattgtatgcgttaagtctggtttgaagaagtcattgttcaatgtcttccaagatggtttatgccatccaaagatggcattggtaattgatgatcgcttgaaagtgtgggatgagaaggatcaacctcgggtgcatgttgtccctgcatttgctccatactatgctcctcaagctgaagcaagcaatactatccctgtcctatgtgttgcaagaaatgttgcttgcaatgttaggggtggcttctttaaggattttgatgatggtcttttacaaaagattcctcaaattgcctatgaagatgatatcaaagatataccttctcctcctgatgtgagcaattatctagtttcagaggatgatggctctatttccaatggacaccgagatccattcttgtttgatggcatggcagatgccgaggtagaaagaaaattaaaggatgcactatcggcagcttcaactatccctgtgacaactgctaatctagatcccaggctcacttctcttcagtacacaatggtgccttctggttcagttcctcctccaacagcacaagcatctatgatgccatttccccatgtacagttccctcaacctgctacactagtaaagccaatgggtcaagctgccccttccgaacctagcttgcatagctctcctgctagagaagaaggtgaagtacctgaatcagaattagacccagatacaaggcgtaggcttctcatattgcaacatggacaagacaccagggatcatgcatctgctgagcctccatttcctgttagacatcctgtgcaaacctctgctcctcatgttccatcatcacggggggtttggtttcctgcagaagaggagattggttcacagccactaaaccgggtagtacctaaagaatttcctgtagattctggtccattgggtattgcaaagccccggcctcatcatccctcctttttctctaaggttgaaagttctatttcatctgatagaattctccatgacagccaccaaagattgccaaaggagatgtatcacagagatgatcgcccaagattaaaccatatgctctctagttatcgttctttttctggtgatgatatcccctttagtaggtcattttccagccacagggatcttgactctgaatctggccattctgtattgcatgcagatactccagttgcagtactacaggaaattgcactgaagtgtggaactaaggtggactttatttcatctctggttgcaagcactgaactgcagttctccatggaggcatggttttctggaaaaaaaattggtcacagagttggcagaactagaaaggaagctcaaaataaggctgctgaggattctatcaaacatttggcagatatatatttgtctagtgccaaggatgagcctggttctacatatggcgatgtgagtgggtttcctaatgtaaatgacagtggttacatgggtattgccagctctcttggtaatcagccattgtctaaagaggattcagcttcattttcaactgcatctccatcaagagttttagatcctaggttggatgtctctaagaggtcaatgggttcaatctcttccctgaaagaattgtgcatgatggagggtcttgatgtcaattttctatctgcacctgctccagtgtcaacaaattcagttcagaaagatgaagtacacgcacaggttgaaatagatggcaaggtctttggtaaaggtattggattaacatgggatgaggctaaaatgcaggctgccgagaaggcacttggaagtctaagatcaaagcttggccaaagcattcagaaacggcagagctctcccaggccacatcagggattttcaaataaacgtttaaagcaagaataccctcgaccaatgcagcggatgccctcatcagctagatatcccaggaatgctccaccaattccttga

SEQ ID NO: 30 - DNA, 2883 bp, Artificial Sequence
  CDS of Glycine max Glyma.13G181700.1
atgtataaatcggtggtgtaccagggggaggtggtggtgggtgaggtagatgtatacccagaagagaacaacaacaacaacaacaagaactacaacaagaacttccatgtgaaggaaatcagaataagccacttctcgcaacctagtgagagatgtcccccacttgctgtgcttcacactgttacctcatgtggtgtttgcttcaaaatggagtcaaagactcagcagcaggacggcctctttcaattgcactccttgtgtatcagagagaacaagactgctgtaatgccactgggtggggaagaaatacatttggttgcaatgcattctcggaatgatgatagaccatgcttttggggatttattgttaccttgggactttacgattcatgtctcgtgatgctaaatcttagatgtttgggtatagtgtttgatctggatgaaacactcattgtagcaaacacaatgcgatcatttgaggatagaattgatgcactccagagaaaaataaactctgaggtagatccacagcgaatttctggtatgcaggcggaggtcaagcgctacctagatgacaagaatatattgaagcaatatgctgaaaatgatcaggttgttgataatgggagagtgataaaagttcaatctgagattgttccggcattatcagacagtcatcagcctatagttcggccactgatccggttacaggataagaacattattctgacacgcatcaatccacagattcgtgatacgagtgttcttgtgaggttgagacctgcatgggaagatcttcggagctaccttactgcaagaggacgcaagcgttttgaggtttatgtctgcacaatggctgaaagggactatgcactagaaatgtggagacttcttgatccagattcaaatttgataaattctaaagaactattaggtcgcattgtatgcgttaagtctggtttgaagaagtcattgttcaatgtcttccaagatggttcatgtgatccaaagatggcattggtaattgatgatcgcttgaaagtgtgggatgagagggatcaacctcgggtgcatgttgttcctgcatttgctccatactatgctcctcaagctgaagcaagcaatactatccctgttctgtgtgttgcaagaaatgttgcttgcaatgttaggggtggcttctttaaggattttgatgatggtcttttacaaaagattcctcaaattgcatatgaagatgatatcaaagatgtaccttctcctcctgatgtgagcaattatctagtttcagaggatgatggttctatttccaatggaaacagagatccgttcttgtttgatggcatggcagatgctgaggtagaaagaaaattgaaggatgcactagcggcagcttcaactttccctgtgacaactgctaatctagatcccaggctcacttctcttcagtacacaatggtgccttctggttcagttcctcctccaacagcacaagcatctatgatgccatttccccatgtacagttccctcaacctgctacactagtaaagccaatgggtcaagctgccccttccgatcctagcttgcatagctctcctgctagagaagaaggtgaagtacctgaatcagaattagatccagatacaaggcgtaggcttctcatattgcaacatggacaagataccagggatcatgcatccgctgagcctccatttcctgttagacatcctgtgcaagcctctgctcctcgtgttccatcatcacggggggtttggtttcctgtagaagaggagattggttcacagccactaaaccgggtagtacctaaagaatttcctgtggattctggtccattgggtattgaaaagccccggcttcatcatccatcctttttcaataaggttgaaagttctatttcatctgatagaattctccatgacagccaccaaagattaccaaaggagatgtatcacagagatgatcgccccagactaaatcatatgctctctagttatcgttctttttctggtgatgatatcccctttagtaggtcatcttccagccacagggatcttgactctgaatctggccattctgtattgcatgcagatactccagttgcagtattgcacgaaattgcactgaagtgtggaactaaggtggactttatgtcatctttggttgcaagcactgaactgaagttctccttggaggcatggttttctggaaaaaaaattggtcatggatttggcaggactaggaaggaagctcaaaataaggctgctaaggattctatcgaacatttggcagatatatatttgtctagtgccaaggatgagcctggttccacatatggcgatgtgagtgggtttcccaatgtaaatgacaatggttacatgggtattgccagctctcttggtaatcaaccattgtctaaagaggattcagcttcattttcatctgcatctccatcaagagctttagatcctaggttagatgtctctaagaggtcaatgggttcgatctctgccctgaaagaattgtgcatgatggagggtcttggtgtcaattttctatctacacctgctccagtgtcaacaaattcagttcaaaaagatgaagtacatgcacaggtcgaaatagatggcaagatctttggtaaaggcattggattaacatgggatgaggctaaaatgcaggctgccgagaaggcacttggaaatctaagatcaaagcttggccaaagcattcagaaaatgcagagctctcccaggccccatcagggattttcaaataagcgtttaaagcaagaataccctcgaacaatgcagcggatgccctcatcagctcgatatcccaggaatgctccaccaattccttga

SEQ ID NO: 31 - DNA, 2883 bp, Artificial Sequence
  CDS of Glycine max Glyma.13G241400.1
atgccgacgtctatggtgtaccatggagagatggcagtgggagaggtaaagatatacccagaagagaacaagaacatggatctgaaagaaatcagaataagtcacttctcacaacctagtgaaaggtgtccaccgcttgcagtgcttcacacaattacttcttttggtatttgcttcaaaatggagtcatcgacctcacagaaacgacaacagcaagatgcactctttcatttgcactcctcatgtatcagagagaacaagactgctgtaatgccagtgcgtggggaagaaatacacttggttgcaatgtattcacggaataatgataggccttgtttctggggatttattgttgcctctggactttacaattcatgtcttacaatgttaaatctgagatgtttgggtatagtgtttgatctggacgaaacccttgtagtggcaaatacaatgcgttcttttgaggataaaattgaggtgctccatagaaaaatgaactctgaggtcaatccgcaacaaatttctgctatgcaggcagagatcaagcgatatttagatgacaagaatatattgaaggaatatgctgaaaatgatcaagttgttgataatgggaaagtgataaaaattcaatctgagagtgttccagcattgtctgatagtcatcagcctatagttcgaccactgatccgattacaagaaaagaatattattctgacacgcatcaatccacagattcgtgatactagtgttcttgtgaggttgagacctgcatgggaagatctccggagctacctgactgcaagagggcgcaagcgttttgaggtttttgtttgcacaatggctgagagggactatgcactagaaatgtggaggcttcttgatccagaattgaatttgataaattcgaaagaactgctagatcgtattgtttgtgtcaagtcaggtttaaagaaatcattgtttaatgtattccaaaatggcttatgccatctgaagatggcattggtaattgatgatcgcttgaaagtgtgggatgagaaagaccagcctcgggtgcatgttgtccctgcatttgctccatattacactcctcaagctgaagcaagcaatgcagtccctttcttgtgtcttgcaagaaatgttgcttgcaatgttaggggtggcttcttcaaggattttgatgatggtcttttacagaagatccctctaattgcttacgaagatgatatcaaagatataccttctcccgatgtgagcaattatctagtttcggaggatgatgcatctgcttccaatggaaataaaaatttgcttctatttgatggcatggcagatgctgaggtagaaagaagattaaaggatgcaatatcagcttcttcaactatccttgcattgactgctaatatagatcctaggcttgctttcacttcttctcttcaatacacaatggtgtcttcttccggaacagttccacccccaacagcacaagcatctgtagtgcaatttggcaatgttcagtttcctcaacctaatactctagttaagcctatgagtcaagttacccatccaggactgagcttgcatagttctcctgccagagaagagggtgaattacctgaatcagaattagacctagataccaggcgtaggtttctcatattgcaacatggtcaagatacaagggagcgcatggcaagtgaacccccatttcctgttagacatcctgcacaagtttctgctcctgcttccagtgtcccatcacgtaggggatggttttctgtagaagaggaaatgggtccacaacaactaaatttgccagtacccaaagaattccctgtagattctgaaccatttcatattgagaagcgctggccccgtcatccatcctttttctctaaggttggcgattctatttcctctgatagagttttccatgagagccatcaaagattgccaaaggaggtgcatcatagggatgatcgctcaagattaagccaatcactctctagttatcattctttaccaggtgatgacattcccttgagtggttcatcttacagcaacagggattttgactctgaatctggacgttctctattccatgctgatactacagctggagtattacaggaaattgcactgaattgtggaactaaagtggaatttttgtcttcgttagtggctagcacagaactgcaattctccattgaggcatggtttgctggaaaaaagattggtgaaggatttggtagaactagaagggaagctcaaagtaaggctgctgggtgttcaatcaaacaattggctgatatatatatgtctcatgctaaggatgattctggttccacttatggtgatgttagtgggtttcatggttcaaacaacgatggttttgtgagtagtggtaattctcttggtaatcagctattgcctaaagaagagtcaggctcattttcaactgcatctgagtcatctagggtttcagattctaggttggaagtctctaaaaggtcaacagattcaatttctgccctcaaagaattgtgcatgatggagggtcttgctgccagttttcaatctccaccagcttcagcatcaacacatttaactcagaaagatgaagtgcatgcacaggttgaaatagatggtcagatctttggtaaagggtttggagtaacatgggaagaggctaaaatgcaggctgccaagaaggcacttggaagtctaagaacaatgtttaatcaaggttctctaaagaggcatggttcgcccaggtcaatgcaaggattggcaaataaacgtctaaagccagaatatcctccaactctgcagcgggttccctattctgctagatatccccggaatgctcctcttgtcccttga

SEQ ID NO: 32 - DNA, 2877 bp, Artificial Sequence
  CDS of Glycine max Glyma.15G072000.1
atgaagaggtctatggtgtaccatggagagatggaagtgggagaggtggagatatacccagaagagaaaaagaacatagatctgaaagaaatcagaataagtcacttctcgcaacctagtgaaaggtgtccaccgcttgctgtgcttcacaccattacttcttttggtatttgcttcaaaatggagtcatcgacctcacagacacgacaacagcaggatgtcctctttcacttgcactcctcatgtatcagagagaacaagactgctgtaatgccacttcgtggggaagaaatacacttggttgcaatgtattcacggaacaatgatagaccatgtttctggggatttattgttgcctctggactttacaattcatgcctaacaatgttaaatctgagatgtttgggtatagtgtttgatctggatgaaacccttgtagttgcaaacacaatgcgttcttttgaggataaaattgaggtactccatagaaaaatgaactctgaggtcaatccgcaacgaatttctactatgcaggcggagataaagcgatacttagatgacaagaatatattgaaggaatatgctgaaaatgatcaagttgttgataatgggaaagtgataaaaattcaatctgagattgttccagcattgtctgatagtcatcagcctatagttcgaccactgatccgattacaagaaaagaatattattttgacacgcatcaatccacagattcgtgatactagtgttcttgtgaggttaagacctgcatgggaagatcttcggagctacctgactgcaagagggcgcaagcgttttgaggtttttgtttgcacaatggctgagagggactatgcactagaaatgtggaggcttcttgatccagaattgaatttgataaattcgaaagaactgttagatcgtattgtttgtgtcaagtctggtttaaagaaatcgttgtttaatgtattccaaaatggcttatgccatctgaagatggcattggtaattgatgatcgcctgaaagtgtgggatgagaaagaccagccgcaggtgcatgttgtccctgcatttgctccatactatgctcctcaagctgaagcaagcaatgcagtccctaccttgtgtcttgcaagaagtgttgcttgcaatgttaggggtggcttcttcaaggattttgatgatggtcttttacagaagatccctctaattgcttatgaagatgatatcaaagatataccttctcctcccgatgtgagcaattatctagtttcggaggatgatgcatctgcttccaatggaaataaaaatttgcttctatttgatggcatggcagatgctgaggtagaaagaagattaaaggatgcaatatcagcttcttcaactgtccctgcaatgaccactaatcttgatcctaggcttgctttcaattcttctcttcaatacacgatggtgtcttcttctggaactgtcccacccccaacagcacaagcatctatagtgcaatttggcaatgttcagtttcctcaacctaatactctagttaagccaatatgtcaagttacccctccaggaccgagcttgcatagttctccggccagagaagagggtgaagtgcctgaatcagaattagacctagataccaggcgtaggcttctcatattgcaacatgggcaagatacaagggagcacacgtcaagtgaacccccacttcccgtcagacaccccacacaagtttctgctcccagtgtcccatcacgtaggggatggttttctgtagaagaggagatgggtccacaacaactaaatcagttagtacccaaagaatttcctgtaggttctgaaccattgcatattgagaagcgctggccccgtcatccatcccttttctctaaggttgacgattctgtttcctctgatagagttttccatgagagccaccaaagattgccaaaggaggtgcatcatagggatgatcactcaagattaagccaatcactctctagttatcattctttcccaggtgatgacattcccttgagtggttcatcttacagcaacagggattttgactctgaatctggacgttctctatttcatgctgatattacagctggagtattacaggaaattgcactgaagtgtggaactaaagtggaatttttgtcttcgttagtggctagcacagcactgcaattctccattgaggcatggtttgctggaaaaaaagttggtgaaggatttggtagaactagaagggaagcccaaaataaggctgctgagtgttcaatcaaacaattggctgatatatatatgtctcatgctaaggatgactctggttccacttatggtgatgttagtgggtttcatggttcaaacaacaatggttttgtgagtagtggtaattctcttggtaatcagctattgcctaaagagtcagtctcattttcaacttcatctgactcatctagggtttcagatcctaggttggaagtctctaaaaggtcaacagattcaatttctgccctcaaagaattttgcatgatggagggtcttgctgccaattttcaatcttcaccagctccagcatcaacacattttgctcagaaagatgaagtgcacgcacaggttgaaatagatggtcagatctttggtaaagggtttggattaacatgggaagaggctaaaatgcaggctgccaagaaggcacttgaaagtctaagaacaatgtttaatcaaggcactcggaagaggcatggttctcccaggtcaatgcaaggattggcaaataaacgtctaaagcaagaatatcctcgaactctgcagcggattccctattctgctagatatcccaggaatgctcctcttgttccttga

SEQ ID NO: 33 - DNA, 3072 bp, Artificial Sequence
  CDS of Beta vulgaris g16374.t1
atgatcaaatcggtagtatatgaaggtgaaaatctacttggagaagtagagatatattttcagaacaacaacaacaacaagaatttagaattgatgaaggggatgaggataagtcattattcagagatgagtgaaagatgtccaccacttgcagttcttcatacaattacaaaatcatcaggtggaatttgcttcaaaatgatggagtcatcttctcatactagttccaacaacaacaacaacaacaagttctattttcaacaacaacaacaagaatcgcagcttttagctatgcactccaactgtatcagagataacaagacggctgtggtgccgcttggtgagcaagagatccatttggtggcattgcgctctagaagaatggccggtgtaactccttgcttctggggtttcagtgtcgccccagggttatatgaatcttgtcttggcttgttaaatcttagatgtctgggcattgtgtttgatctagacgagacactgattgttgcaaacacactgcggtcattcgaggatagaattgaggccttgcaaaggaaaatcagtgttgaagctgacccacaacgaatagcgggtatggtggccgaagtgaaacgctaccaagaagacaagagtatactgaagcaatatgctgaaactgatcaggtggtggacaatggaaaagtccacaaaattcaagcagaagttattccagccctatctgacaaccaccaaacagttattcgccccctcattcggttacaggataagaacattgtcctaacacgaattaatcctcagattcgtgatacaagtgttcttgtaaggctaagacctgcatgggaagatttacgcagctatttaactgccagaggtcggaaacgctttgaggtttatgtttgtacaatggctgaaagagactatgctctagaaatgtggaggcttcttgatccagactcaaacttgatatgtgcgagggaacttttggatcgcattgtctgtgtcaagtctggatcaaagaagtcgctgtttaatgttttccatggtggcatttgtcatcccaaaatggctttggttattgatgatcgtctaaaggtgtgggatgagaaagatcagccgcgagtgcatgttgttcctgcatttgctccttattatgctccccaagccgaggcaaataatgccatcccagttctatgtgtggctagaaatgtagcatgcaatgttcgaggtggttttttcaaagaatttgatgagggtctcttgcaacgggtttctgaggtttcttttgaagatgatcctagagatatcccttctccacctgatgtgagcaactacttggtatcagaggatgatggttctggttctaatggaattaaagaatcaatgacttttgacgggatggctgatgcagaggttgaaagaaggcttaaggaagcagttttatcctcttcttcagcttctccacttccatctgcaaatactccggcgactgttaatttcgatcataggctggcatcgtctcttccattcgctgttgctacttctgccctggcaattccacagcctgcacctcaagcaacaatcacaccttaccataataacctattctcgcaggcagggcctttggctagacctttgggtaatatcggtcctcaagacattggcctgcacaattcccccgcccgagaagaaggtgaagtacctgaatctgagctagatcctgatacaaggagacggcttcttatattgcagcatggtcaagatatgagagaaggcccaccaaatgagcctccatttccagcacgaaccccagttcaagctcctgttacaggtcctgttcctgtttcagttcctggtccagtgcctgttccaggtcctggcccagtttcagtcccagttcctggtcccattcccagtcctgttccagttcctgtatctggtaccgttccaggtcctggtccacgtgtacaatcacgtgggagctggtttccagttgaggatcacatcagccagggtcccttgagtcgagtagcagctaaagagtttcctgtagctcctgatgcttcccctgttgagaagcagcggccacctccaccctcttttcctcgaaaagtggagagtttaggttggtcagatcgaaattatgctgaaaaacaaagactgccgagggaggctctgcgcagagatgacagattgcggtcaaactattcattgcccagccatcagtcatttcgaggtgatgaaatttcattgagccgatcagcttccagcaacaaagattttgaagttgaacctgaacgaggtagttcatttgcagagagtccttcaattgctttacacgacattgcaatgaagtgtggaaccaaggttgaatttaagacagggttggttgctaccccagagttgaagttcttactggaggcttattttgctggagataaaattggtgaaggcactggtacaaccagaagggaagcccagcatcgcgctgcagaggctgctttgatgaatctggctgataagtatttgacccacataaagtctgattctagcactccgcaaagtgatacaagcaggggtcatagtccaattgacacgggatttgtaagtgatgcaaactctcacggtgatggcatttcaaggaaagaggatataattccttcatctgagatgaccgggttggatgactctaatgtagatgggtcaaagaactccatgggttcagtttccgttcttaaagaattgtgtttgagagagggcctaggtgtcgattttaaaggtcaatctccaacttcaactaattcagtcgacagagatgaaatacatgcggaggtggaaataaatgggcaagttctgggcaagggtacaggattgacatgggatgaggcgaaaatgcaggctgctgagatggctcttacaagtcttaattctatgatcggtcaatttaataagcgtccaagttctccgcggttgttgcaagggatgccaaataaacgcttgaagccagagtatcctagagtagtagaccatttgccgtcttcaagatatccaaggaatgcatcacctgtaccttga

SEQ ID NO: 34 - DNA, 2646 bp, Artificial Sequence
  CDS of Solanum tuberosum PGSC0003DMT400057635
atgtttaaatcgacggtggtgttgtatgaaggagaaagattggtgggagaagttgaaatatactgtgagaagggtgtgttatggggggagaaagtaataaggatatctcactattcgccgtcaagtgagagatgtccaccactggcagtgctacacacagttactactggattaagcttcaaattggaacctacaaaatcaaagccgctgacccaagactcgccgctcactcttttgcattccacatgtctcagagataacaagactgcggtgatgtcacttggaagagaggaacttcatttagtcgccatgcagtctaagaatattggcggacagtgtccttgcttttggggatttaaggttgcatcagggctctatgattcttgtctaacaatgctaaacctcaggtgtcttggcattgtatttgatcttgatgagacactcatagttgccaacacaatgcgttcatttgaggatagaatagaggccttgcagcggaaaataaattcagagtcagatccgcaacgtgcctctgttatgctggctgaggtcaagcgttatcaggaggataaaattattttaaagcaatatgccgaaaatgatcaggttgttgacaatggaaaggtgatcaaatctcagtctgaggttttccccgcattgtctgataatcaccaacctattgtcagacccctgattaggttgcaagatagaaacattattcttactcgtattaatccaatgatccgtgatactagtgttcttgtgagattgagacctgcttgggaggatctccgaagctatctgactgcaaggggtcggaagcgctttgaagtttatgtgtgcacaatggctgaaagagattatgctttagaaatgtggcgccttcttgatccagattcaaacttgattaactcacaagaactcttggatcgtattgtctgtgtcaagtctggcttgaggaaatctttgttcaatgtattccaagatggaaattgtcatcctaagatggcattggtgattgatgatcgcttgaaagtgtgggatgataaggatcaaccccgagtgcacgttgttcctgcatttgctccatattttgcccctcaagctgaaggcaacaattctgttcctgtcctttgtgttgccagaaatgtagcctgcaatgttagaggtggttttttcaaagattttgatgaaggcctattacagcgaatatctgaagttgcatatgaagatgatattaaacaagttccttctgctcccgatgttagcaactatttgatttcggaggatgatccttcagctgtaaacgggaataaagattcacttggatttgatggaatggctgatagtgaggttgaaaggagactgaaggaagcaatgttggcttcaacaagtgttccttctcaaatgacaaatttggatccaagacttgtaccagctctccagtaccctgtcccacctgttatttcacaaccaagcattcaaagcccagtggtgccttttcctactcaacatctccctcaagtgacttctgtacttaaatcatcagtgactcaaataagtcctcaggatacaagcttgcaaagttctcctgctagggaagagggtgaggtaccagaatctgaattagatcctgatacaaggaggagattacttatattgcagcatggtcaagacacgagagatcaagtatcaagtgaacctaagttccctatggggactccattacaagtatctgtgccaccacgggtgcagccacatggttggtttccggctgaggaagagatgagccctaggcaacttaatcgacccttacctcccaaggaatttcctttaaatccagagtctatgcatattaacaaacatcggcctccacatccaccttttcttcccaaaatggagacttctatgccatctgatagagttctttttgagaaccagaggctgccaaaagaggtaatccctcgggatgacaggatgagattttcccaatcacaacctagctttcgtccaccaggtgaggaagttcccttgggtcggtcatcttccagcaatagggttcttgaccttgaacctggacattatgatccatatctagaaacacctgctggagctttacaagatattgcattcaagtgtggagcaaaggtcgaattcagatcaagttttctttcgagtccagagctgcagttctccttggaggtgttgtttgcaggagaaaaagttggagaaggaactggccggacgagaagggaagcgcaacgacgtgctgctgaggaatctcttatgtatctggccgacaaatacttgtcatgtatcaaacctgattccagttccacacagggtgatggatttaggtttccaaatgcaagtgataatgggtttgttgacaacatgagtccttttgggtatcaagatcgtgtctcacattcttttgcatcagagcctcccagagtgttggatccaaggcttgaggtttttaagaagtcagtgggttcagttggcgccctaagagaactatgcgcaattgaaggacttggtttggcattccaaactcagcctcaactttcagctaatccgggccagaaaagtgaaatatacgcccaggttgcccatttacttccctttcttctttgctattgctga

SEQ ID NO: 35 - DNA, 200 bp, Artificial Sequence
  ZmCPL1 silencing sequence
atggtatgctatcagagatcaagaggtaccaggatgataagtccatcctaaagcaatatatagaaggtgatcaggtctatgatgatggaaaagtgtataaagcacaacctgagattgttccaccattgtctgataaccagcaaccaatgacacgtccagttataagattacaagataaaaacattatcctgacaagaata

SEQ ID NO: 36 - protein, 237 aa, Oryza sativa
MSLSLGFSAGAGVGADRLAAAPALQAAGALPPRVDVSLSLARAANGQPSSYLPLNENDSLDMVLFDVLREASAVAALSSSSSSSPELGARTTAPVVAGHPAGRKGGGGGGGGRGAAARGGAAGGRHYRGVRRRPWGKYAAEIRDPTRHGARLWLGTFGTAEEAAAAYDRAAFRMRGAKALLNFPPAVAGDGARRGAAAAAKQVGMSDVVPRACHVVCYSVLSAHIFYDIFRGVIPIP

SEQ ID NO: 37 - protein, 231 aa, Zea mays
MSTYISLSLERSSSVVRDAAKSAPPGSGVSAPYPRAGAGAGAGTSLSLSGAAAAHGHGQYLPLNEDDSLDMVLFDVLREASGSAPGATYQPPSSSLPTLKALASGASRKGGGNNGNGSRAAHAPKAATGRHYRGVRRRPWGKYAAEIRDPARHGARLWLGTFATAEEAAAAYDRAAFGMRGAKALLNFPPPAAGCGNAATATAKQAAGSQVNNGECDLLEENGSSHDSECF

SEQ ID NO: 38 - protein, 225 aa, Sorghum bicolor
MNTYMSLSLESSSSTTTSSVVRDVATPAPLSSGASASSYPRAGAGMGMGTSLSLASAAAAAHGHGQYLPLNEDDSLEMVLFDVLREASGSAAAPTTYQPPASSLPTLKKALASGAAARKGGGGGVGCSSRAAHAPKAATTGRHYRGVRRRPWGKYAAEIRDPARHGARLWLGTFATAEEAAAAYDRAAFRMRGAKALLNFPPAVAGCGNAATATAEQAAGVTEAK

SEQ ID NO: 39 - protein, 273 aa, Beta vulgaris
MSIISNFETDLAVLESIKRHLLEDWDPRIAGASFMATGSGPIYRRNSSFSRLYPCLTDNWGELPLKEDDSEDMVVFGVLRDAVHTGWSPESGSEIDSGSPAPVVVKSEPVESPVRVSGGEAPAATVAMAAPIRGKHYRGVRRRPWGKFAAEIRDPAKNGARVWLGTFETAEDAALAYDRAAFRMRGSKALLNFPLRINSGEPDPVRITSKRSSPERSTSSLSSSSCTSSDNGSPKRRKKGSVVGQVGNGVEGLQVGCQVGVGSMPLGDQLLVS

SEQ ID NO: 40 - protein, 247 aa, Solanum tuberosum
MYQLPISTELPPTFFPAEFPVYCRSSSFSSLMPCLTESWGDLPLKVNDSEDMVIYGLLQDAFSIGWTPSNLTSSEVKPEPREEIEPVMSTSVSPPTETAAAPAALQPKGRHYRGVRQRPWGKFAAEIRDPAKNGARVWLGTYESAEEAALAYDKAAFRMRGTKALLNFPHRIGLNEPEPVRVTVKRRLPEPASSSVSSASESGSPKRRRKGVAAKQAELEVQSRGPNVIQVGCQMEQFPVGEQLLVN

SEQ ID NO: 41 - protein, 293 aa, Hordeum vulgare
MLLNPASEALVLDSIRQHLMEDTAAAAPALAEARRQRPAYCRSASFGSLVADQWSESLPFRADDSDDMVVYGALRDAFSCGWLPDGSFAAVKPEPLPSPESCYGGFLYPDTPPATSAGDGEGDGEEEEAAALMGEAAAAAVARGKHYRGVRQRPWGKFAAEIRDPAKNGARVWLGTYDTAEDAAVAYDRAAYRMRGSRALLNFPLRIGSEIAAAHAAAAAAGDKRPSPEPATSDSSSSSGSPKRRKRGEAAAASMAMALVPPPSQLSRPAQAWYPAAPAEQVAMAPRVQQLVS

SEQ ID NO: 42 - protein, 279 aa, Hordeum vulgare
MLLLNPAVEAAVLDSIRQQLLEEPTAPARPAYCRSASFGSLVADQWSESLPFRPNDADDMVVYGALRDAFSCGWLPDGSFAAVKPEPLPSPDPYDGSCLGSFLASPGPATPDEAASTATWTEEAEVAATASRGRHFRGVRQRPWGKFAAEIRDPAKNGARVWLGTFDSAEDAAVAYDRAAYRMRGSRALLNFPLRIGSEIAAAAAAAGQKRASPEPASSDSASPSSSAPGSSKRRKRGEAAAASMAMALVPPTPVQAPVQLILPAQPWLATGAVQQLVS

SEQ ID NO: 43 - protein, 186 aa, Triticum aestivum
MPLSLGADHLAAPGARAPPARVGAAMGMGMGMGMDTSLSLGGAAAYLPLNENDSLDMILLDVLREASAVAAASPSSSSPPTKTARAIAASSPDASEGGGACAAGRAAERPHYRGVRRRPWGKYAAEIRDPSRPGTRLWLGTFGTAEEAAVAYDRAAFRLRGAKALLNFPPAVGADGARRGGADGAR

SEQ ID NO: 44 - protein, 182 aa, Triticum aestivum
MPLSLGADHLATPGARAPPARVGAAMGMDTSLSLGGAAAYLPLNENDSLDMLLFDVLREASAVAAASPSSSSSSPPTKMAAAIAASGPATVEGGRACAAGRAAERPHYRGVRRRPWGKYAAEIRDPSRPGTRLWLGTFGTAEEAAVAYDRAAFRLRGAKALLNFPPAVGAHEARRGGADGAR

SEQ ID NO: 45 - protein, 181 aa, Triticum aestivum
MPLSLGADHLAAPGARAPPARVGTGMGMDTSLSLGGAAAYLPLNENDSLDMLLFDVLREASAVAAASPSSSSSPPTKTATAIAAAGADAIEGGGACAAGRAAERPHYRGVRRRPWGKYAAEIRDPSRPGTRLWLGTFGTAEEAAVAYDRAAFRLRGAKALLNFPPAVGAHEARRGGANEAR

SEQ ID NO: 46 - protein, 298 aa, Triticum aestivum
MLLNPASEALVLDSIRQHLMEDTAAAAAPVAEARRQRPAYCRSASFGSLVADQWSESLPFRADDSDDMVVYGALRDAFSCGWLPDGSFAAVKPEPLLPSPDSAYACDGPSCFGFLEPETPPATSPGEGEEEGAAFMGEAAAAVARGKHYRGVRQRPWGKFAAEIRDPAKNGARVWLGTYDTAEDAAVAYDRAAYRMRGSRALLNFPLRIGSEIAAAHAAAAAAGDKRPSPEPATSDSSSSSSSSGSPKRRKRGEAAAASMAMALVPPPSQLSRPAHAWYPAAPVEQVAMAPRAQQLVS

SEQ ID NO: 47 - protein, 290 aa, Triticum aestivum
MLLNPASEAMVLDSIRQHLMEDTAAPAPEARRQRPAYCRSASFGSLVADQWSESLPFRADDSDDMVVYGALRDAFSCGWLPDGSFAAVKPEPLLPSPDSCYVGFLEPETPPATSPGGSEEEAAAAFMGEEAAAAVARGKHYRGVRQRPWGKFAAEIRDPAKNGARVWLGTYDTAEDAAVAYDRAAYRMRGSRALLNFPLRIGSEIAAAHAAAAAAGDKRPSPEPATSDSSSSSSSSGSPKRRKRGEAAAASMAMALVPPSSQLSRPAHAWYPAVPAEQVAMAPCVQQLVS

SEQ ID NO: 48 - protein, 289 aa, Triticum aestivum
MLLNPASEALVLDSIRQHLMEDTAAPAPEARRQRPAYCRSASFGSLVADQWSESLPFRADDSDDMVVYGALRDAFSCGWLPDGSFAAVKPEPLLPSPDSCYVGFLEPETPPATSPGESEEEAAAAFMGEAAAAAARGKHYRGVRQRPWGKFAAEIRDPAKNGARVWLGTYDTAEDAAVAYDRAAYRMRGSRALLNFPLRIGSEIAAAHAAAAAAGDKRPSPEPATSDSSSSSSSSGSPKRRKRGEAAAASMAMALVPPPSQLSRPAQAWYPAAPVEQVAMAPRAQQLVS

SEQ ID NO: 49 - protein, 185 aa, Secale cereale
MPLSLGADHLATPGAPARVGAGMCMGMDTPLSLGGAATHGHGQYYLPLNENDSLDMLLFDVLREASAVATAPPSSSSPSTKMATAIAASDPATIEGGRACAPGRAAERPHYRGVRRRPWGKYAAEIRDPSRAGARLWLGTFGTAEEAAVAYDRAAFRLRGAKALLNFPPVVAADGGRRGGADGAR

SEQ ID NO: 50 - protein, 297 aa, Secale cereale
MLLNPASEAMVLDSIRQHLMEDTAAAAPAAEARRQRPAYCRSASFGSLVADQWSESLPFRADDSDDMVVYGALRDAFSCGWLPDGSFAAVKPEPLLPSPDSAYACGGPSCFGFLEPETAPATPQGEGEEEEEAAAFMGEAAAAVARGKHYRGVRQRPWGKFAAEIRDPAKNGARVWLGTYDTAEDAAVAYDRAAYRMRGSRALLNFPLRIGSEIAAAHAAAAAAGDKRPSPEPATSDTSSSSSGSPKRRKRGEAAAASMAMALVPPPSLLSRPAQAWYPAAPVEQVAMAPRAQQLVS

SEQ ID NO: 51 - DNA, 714 bp, Artificial Sequence
  CDS of OsERF922
atgtctctctccttggggtttagcgccggcgccggcgtcggtgccgatcgcctcgccgccgcgcccgcgctccaggccgccggtgctctgccgccacgcgtggacgtgtctctgtcgctggcgcgcgcggccaacgggcagccgtcgtcgtacctcccgctcaacgagaacgactcgctggacatggtcctcttcgacgtcctccgcgaggcctccgcggtggcggcgctgtcgtcgtcgtcgtcgtcgtcgccggagctgggcgcgcggacgacggcgccggtggtcgccggccaccccgccgggcgcaagggtggcggcggcggaggcggaggcaggggagcggcggcgaggggcggcgccgctggcgggcggcactaccgcggtgtgaggcggcggccgtgggggaagtacgccgcggagatccgtgacccgacgcggcacggcgcgcggctgtggctcggcaccttcggcacggccgaggaggccgccgcggcgtacgaccgcgccgcgttccgcatgcggggcgccaaggcgctgctcaacttcccgccggctgtcgccggtgacggcgcccgtcgcggcgccgccgcggccgcgaagcaggtcggcatgagcgacgtggtaccaagagcatgtcatgtagtttgttactctgtgcttagtgctcacattttttatgatatctttcgtggtgtgataccaattccgtga

SEQ ID NO: 52 - DNA, 696 bp, Artificial Sequence
  CDS of ZmERF922-I
atgagcacgtacatatctctctccttagagcgcagcagcagcgtggtccgcgacgccgccaagtccgcgcctccgggcagtggtgtcagtgccccgtatccacgcgcgggcgcgggcgcgggcgcgggcacgtcgctttccctgtccggcgcagcggcggcgcacgggcacggccagtacctccccctcaacgaggacgactccctggacatggtcctcttcgacgtgctccgcgaggcatccggctccgcacccggcgccacctaccagccgccctcgtcgtcgttgcctacgctgaaggcgctcgcttccggcgcctcgcgcaagggcggtggcaacaacggcaacggcagtagggccgcgcacgcacccaaggcggccacggggcggcactaccgcggggtgaggcggcggccgtggggcaagtacgccgcagagatccgtgacccggcgcggcacggggcgcggctctggctcggcaccttcgccacggccgaggaggccgccgcggcgtacgaccgcgccgcgttcggcatgcggggcgccaaggcgctgctcaacttcccgccgccggccgcgggctgcgggaacgccgcaaccgccacggcgaagcaggctgctggaagccaagtgaacaacggcgaatgcgatctcctcgaagaaaatgggtcatcgcatgatagtgaatgtttctaa

SEQ ID NO: 53 - DNA, 678 bp, Artificial Sequence
  CDS of SobicERF922-I
atgaacacgtacatgtctctctccttggagagcagcagcagcaccaccaccagcagcgtggtccgcgacgtcgccacgcccgcgcctctgtccagcggtgccagtgcctcgtcgtatccacgggcgggcgcgggcatgggcatgggcacgtcgctctccctggccagcgccgcggcggcggcgcacgggcacggacagtacctccccctcaacgaggatgactccctggagatggtcctcttcgacgtgctccgcgaggcatccggctccgctgcggcgcccaccacctaccagccgccggcctcgtcgttgcctacgctgaagaaggcgctcgcttccggcgccgccgcgcgcaagggcggcggcggcggcgtcggttgcagcagcagggcagcgcacgcgccgaaggcggccacaacggggcggcactaccgcggggtgaggcggcggccgtggggcaagtacgccgcggagatccgtgaccccgcgcggcacggggcgcggctctggctcggcaccttcgccacggccgaggaggccgccgcggcgtacgaccgcgccgcgttccgcatgcggggcgccaaggcgctgctcaacttcccgccggcggtcgcgggctgcgggaacgccgcaaccgccacggcggagcaggctgctggggtgacggaagccaagtga

SEQ ID NO: 54 - DNA, 822 bp, Artificial Sequence
  CDS of BvERF922-I
atgagtataatcagtaattttgaaaccgacttagcagttcttgagtcgatcaagcgtcacttactagaagattgggatccaagaatcgccggagcttcgtttatggcgacaggatcaggcccaatttatcgccggaattccagctttagtaggttatacccttgtttaactgataattggggtgaattaccattaaaagaagatgattctgaagatatggttgtttttggggttctccgagacgccgttcacaccggatggtcgccggaatccgggtcggaaatcgactcaggttcgccggcgccagtggtggtcaagtcggagccggtggaatctccggtgagagtttccggcggtgaagccccggcggccaccgtggcgatggcggctccgataagggggaagcattatcggggtgtccgaaggcggccgtgggggaagtttgcggcggagatccgagacccggccaagaacggggctcgggtttggctcggaacgttcgagacagccgaggacgcggccctggcctacgacagggccgcgttccggatgcgcggctcgaaggcgttgctaaatttcccactccgaatcaactcgggtgagcccgacccggttcggatcacttcaaagcgatcatcacccgagagaagcacgtcatcactttcgtcatcatcgtgtacgtcatcagataatgggtcccccaaaagaagaaagaaagggagtgtggttggacaagttggaaatggagttgagggtttacaagtgggatgccaagttggtgtgggctcaatgccacttggcgatcaactattagttagttaa

SEQ ID NO: 55 - DNA, 741 bp, Artificial Sequence
  CDS of StERF922-I
atgtaccagttacccatcagtaccgagttgccccccacattctttcctgcagagttcccagtgtattgtcgatcctcctcatttagttccctgatgccttgtctcacggaatcgtggggggatcttcccttgaaagttaatgacagcgaagacatggttatttacggactactacaagacgcattcagtattggttggaccccgagtaatctaacaagttcagaggtgaaaccggagccccgtgaggaaattgagccagtaatgtccactagtgtcagtccgccaacggaaacggctgcagcgcccgccgcgttgcagccaaaaggccggcattatcggggagtcagacaacggccgtggggtaagttcgcagctgaaataagagacccggcaaagaacggtgcccgggtctggttaggtacctatgaatctgccgaggaagcagcgcttgcctatgataaggcagcattccgcatgcgggggacgaaagctctacttaacttcccgcataggataggcctcaatgaacctgaacccgtgagagttaccgtcaagaggcgactgccggaaccggcttcttcctccgtgtcgagcgcatctgaatcagggagtcctaagaggaggcgaaagggcgtcgctgcgaaacaggcagaactcgaggtccagtcgcgcggtcccaatgtaatacaggttggttgtcagatggaacaatttccagtcggggagcagctgttagtgaat

SEQ ID NO: 56 - DNA, 879 bp, Artificial Sequence
  CDS of HvERF922-IIa
atgctgctgaacccggcatcggaggcgctggtgctggacagcatcaggcagcacctcatggaggacacggcggccgcggcgccggcgctggcggaggcgaggaggcagaggccggcctactgccggagcgccagcttcggaagcctggtggcggaccagtggagcgagtcgctcccgttccgcgccgacgactcggacgacatggtcgtctacggcgccctccgcgacgccttctcctgcggctggctccccgacggctccttcgccgccgtcaagccggagcccctcccttccccggagtcctgctacggcggtttcctctacccggacacgcctccggccacgtctgccggcgacggcgagggcgacggcgaggaggaggaggccgcggcgctcatgggcgaggcggcggccgcggccgtggccagggggaagcactaccgcggggtgaggcagcggccgtggggcaagttcgcggcggagatccgggacccggccaagaacggcgcgcgcgtgtggctcggcacctacgacaccgccgaggacgccgccgtcgcctacgaccgcgccgcgtaccgcatgcgcggctcccgcgcgctcctcaacttcccgctccgcatcggctccgagatcgccgccgcccacgccgcagccgcggccgccggcgacaagcgtccgtcccccgagccggcgacctctgactcgtcctcctcgtccgggtccccgaaaaggaggaagaggggcgaggccgcggccgcgtccatggccatggcgctggtgccgccgccctcccagctgagccggccggcccaggcgtggtaccccgccgcgccggccgagcaggtggccatggcgccgcgcgtgcagcagctggtcagc

SEQ ID NO: 57 - DNA, 837 bp, Artificial Sequence
  CDS of HvERF922-IIb
atgctgctgcttaatccggcggtcgaggcggcggtgctggacagcatccggcagcagctcctggaggagccgacggcgccggcgcggcctgcgtactgccggagcgcgagcttcggcagcctcgtggcggaccagtggagcgagtcgctcccgttccggcccaacgacgccgacgacatggtcgtctacggcgccctccgcgacgccttctcttgcggctggctccccgacggctccttcgccgccgtcaagcccgagcccctgccttcccctgacccgtacgacggctcctgcctcggcagcttcctcgcgtcgcccgggcccgccacgccggacgaggcggcctcgacggcaacgtggacggaggaggccgaggtggcggcgacggcatcgagggggaggcacttcagaggcgtgaggcagcggccgtggggcaagttcgcggcggagatccgggacccggccaagaacggcgcgcgcgtgtggctcggcacctttgacagcgccgaggacgccgccgtggcgtacgaccgcgccgcctaccgcatgcgcggctcccgcgcgctcctcaacttcccgctccgcatcggctccgagatcgccgccgcagcagcagccgcgggtcagaagcgcgcgtctcccgagcctgcgagctccgactctgcttctccctcctccagcgcgcccgggtcgtcgaagcggaggaagagaggcgaggcggcggcagcgtccatggccatggctctggtgccgcccacgccggtgcaggctccggtccagctcatcctcccagcccagccgtggctcgccaccggcgccgtccagcagctagtgagc

SEQ ID NO: 58 - DNA, 561 bp, Artificial Sequence
  CDS of TaERF922-3A-I
atgcctctctcgctcggcgccgaccacctcgccgcgcccggcgcgcgcgctccgcctgcgcgggtgggcgcggccatgggcatgggcatgggcatgggcatggacacgtccctctcgctgggcggtgcagccgcctacctcccgctcaacgagaacgactccctcgacatgatcctcctcgacgtcctccgcgaggcctccgccgtggccgccgcctcgccctcctcctcctctcccccgacgaagacggcgagggccattgccgcctctagccccgacgcgagcgagggcggcggggcatgcgcggccgggagggccgcggagcggccgcactaccgcggggtgaggcggcggccgtggggcaagtacgcggcggagatccgggacccgtcgcggcccggcacgcgcctgtggctcggcaccttcggcacggccgaggaggcggccgtcgcctacgaccgcgccgcgttccgcctgcgcggcgccaaggcgctgctcaacttcccgccggccgtgggggcggacggggcgcgccgcgggggcgccgatggggcaaggtga

SEQ ID NO: 59 - DNA, 549 bp, Artificial Sequence
  CDS of TaERF922-3B-I
atgcctctctcgctcggcgccgaccatctcgccacgcccggcgcgcgcgctccgcctgctcgggtgggcgcggccatgggcatggacacgtccctctcgctgggcggtgccgccgcctacctcccgctcaacgagaacgactccctcgacatgctcctcttcgacgtcctccgcgaggcctccgccgtggccgccgcctcgccctcctcctcctcctcctctcccccgacgaagatggcggcggccattgccgcctctggtcccgccacggtcgagggcggcagggcatgcgcggccgggagggccgcggagcgcccgcactaccgcggggtgaggcggcggccgtgggggaagtacgcggcggagatccgggacccatcgcggcccggcacgcgcctgtggctcggcaccttcggcacggccgaggaggcggccgtcgcctacgaccgcgccgcgttccgcctgcgcggcgccaaggcgctgctcaacttcccgccggccgtgggggcgcacgaggcgcgccgcggcggcgccgatggggctaggtga

SEQ ID NO: 60 - DNA, 546 bp, Artificial Sequence
  CDS of TaERF922-3D-I
atgcctctctcgctcggcgccgaccacctcgccgcgcccggcgcgcgcgctccgcctgcgcgggtgggcacgggcatgggcatggacacgtccctctcgctgggcggtgccgccgcctacctcccgctcaacgagaacgactccctcgacatgctcctcttcgacgtcctccgcgaggcctccgccgtggccgccgcctcgccctcctcgtcctcctctcccccgacgaagacggcgacggccattgccgccgctggcgccgacgcgatcgagggcggcggggcatgcgcggccgggagggccgcggagcgcccgcactaccgcggggtgaggcggcggccgtggggcaagtacgcggcggagatccgggacccgtcgcggcccggcacgcgcctgtggctcggcaccttcggcacggccgaggaggcggccgtcgcgtacgaccgcgccgcgttccgcctgcgcggcgccaaggcgctgctcaacttcccgccggccgtgggggcgcacgaggcgcgccgcggcggcgccaatgaggcgaggtga

SEQ ID NO: 61 - DNA, 896 bp, Artificial Sequence
  CDS of TaERF922-2A-II
atgctgctgaacccggcctcggaggcgctggtgctcgacagcatccggcagcacctcatggaggacacggcggccgcggcggcgccggtggcggaggcgaggcggcagcggccggcctactgccgcagcgccagcttcgggagcctggtggcggaccagtggagcgagtcgctcccgttccgcgccgacgactccgacgacatggtcgtctacggcgccctccgcgacgccttctcctgcggctggctccccgacggctcctttgccgccgtcaagccggagcccctcctgccttccccggactctgcctacgcgtgcgacggcccgtcctgcttcggtttcctcgagccggagacgccccccgccacgtccccgggcgagggcgaggaggagggcgcggcgttcatgggcgaggcggccgccgcggtggcgaggggcaaacactaccgcggggtgcggcagcggccgtggggcaagttcgcggcggagatccgggacccggccaagaacggcgcgcgcgtgtggctcggcacctacgacaccgccgaggacgccgccgtggcctacgaccgcgccgcgtaccgcatgcgcggctcccgcgcgctcctcaacttcccgctccgcatcggctccgagatcgccgccgcgcacgccgcggccgcggccgccggggacaagcggccgtcacccgagcccgcgacctcggactcgtcatcctcgtcctcctcctccgggtccccgaaaaggaggaagaggggcgaggccgcggccgcgtcgatggccatggcgctggtgccgccgccctcgcagctgagccggccagcccacgcgtggtaccccgccgcgccggtggagcaggtggccatggcgccgcgcgcgcagcagctggtcagcta

SEQ ID NO: 62 - DNA, 872 bp, Artificial Sequence
  CDS of TaERF922-2B-II
atgctgctgaacccggcctcggaggcgatggtgctggacagcatcaggcagcacctcatggaggacacggcggctccggcgccggaggcgaggaggcagaggccggcctactgccgcagcgccagcttcgggagcctggtggccgaccagtggagcgagtcgctcccgttccgcgcggacgactccgacgacatggtcgtctacggcgcgctccgcgacgccttctcctgcggctggctccccgacggctccttcgccgccgtcaagccggagcccctcctgccttccccggactcctgctatgtcggcttcctcgagccggagacgcctccggccacgtccccgggcgggagcgaggaggaggccgccgcggcgttcatgggcgaggaggcggccgcggccgtggcgagggggaagcactaccgcggggtgaggcagcggccgtggggcaagttcgcggcggagatccgggacccggccaagaacggcgcccgcgtgtggctcggcacctacgacaccgccgaggacgccgccgtggcctacgaccgcgccgcgtaccgcatgcgcggctcccgcgcgctcctcaacttcccgctccgcatcggctccgagatcgccgccgcgcacgccgcggccgcggccgccggcgacaagcggccgtcgcccgagcccgcgacatcggactcgtcatcctcctcgtcctcctccgggtccccgaaaaggaggaagagaggcgaggccgcggccgcgtccatggccatggcgctggtgccgccgtcctcgcagttgagccggccggcccacgcgtggtaccccgccgtgccggcggagcaggtggccatggcgccgtgcgtgcagcagctggtcagcta

SEQ ID NO: 63 - DNA, 869 bp, Artificial Sequence
  CDS of TaERF922-2D-II
atgctgctgaacccggcctcggaggcgctggtgctcgacagcatcaggcagcacctcatggaggacacggcggcgccggcgccggaggcgaggaggcagcggccggcctactgccgcagcgccagcttcgggagcctggtggccgaccagtggagcgagtcgctcccgttccgcgccgacgactccgacgacatggtcgtctacggcgcgctccgcgacgccttctcctgcggctggctccccgacggctccttcgccgccgtcaagccggagcccctcctgccttccccggactcctgctacgtcggcttcctcgagccggagacgcctccggccacgtccccgggagagagcgaggaggaggccgccgcggcgttcatgggcgaggcggccgcggccgcggcgagggggaagcactaccgcggggtgaggcagcggccgtggggcaagttcgcggcggagatccgggacccggccaagaacggcgcgcgcgtgtggctcggcacctacgacaccgccgaggacgccgccgtggcctacgaccgcgccgcgtaccgcatgcgcggctcccgcgccctcctcaacttcccgctccgcatcggctccgagatcgccgccgcgcacgccgcggccgcggccgccggggacaagcggccttcccccgagccggcgacctcagactcgtcgtcgtcctcctcctcctccgggtccccgaaaagaaggaagaggggcgaggccgcggccgcgtccatggccatggcgctggtgccgccgccctcgcagctgagccggccggcccaggcgtggtaccccgcggcgccggtggagcaggtggccatggcgccgcgcgcgcagcagctggtcagcta

SEQ ID NO: 64 - DNA, 558 bp, Artificial Sequence
  CDS of ScERF922-I
atgcctctctcgctcggcgccgaccacctcgccacgcccggcgctcctgcgcgggtgggcgcgggcatgtgcatgggcatggacacgcccctctcgctgggcggcgccgctacccacggccacggccagtactacctcccgctcaacgagaacgactccctcgacatgctcctcttcgacgtcctccgcgaggcctccgccgtggccaccgccccgccctcctcgtcctctccctcgacgaagatggcgacggccattgccgcctctgatcccgccacgatcgagggcggcagggcatgcgcgcccgggagggccgcggagcggccgcactaccgcggggtgaggcggcggccgtgggggaagtacgcggcggagatccgggacccgtcaagggccggcgcgcgcctgtggctcggcaccttcggcacggccgaggaggcggccgtcgcgtacgaccgcgcggcgttccgcctgcgcggcgccaaggcgctgctcaacttcccgccggtcgtggcggcggacggggggcgccgcggcggcgccgatggggcgaggtga

SEQ ID NO: 65 - DNA, 894 bp, Artificial Sequence
  CDS of ScERF922-II
atgctgctgaacccggcctcggaggcgatggtgctggacagcatccggcagcacctcatggaggacacggcggccgcggcgccggcggcggaggcgaggaggcagaggccggcctactgccgcagcgccagcttcgggagcctggtggcggaccagtggagcgagtcgctcccgttccgcgccgacgactccgacgacatggtcgtctacggagcgctccgcgacgccttctcctgcggctggctccccgacggctccttcgccgccgtcaagccggagcccctcctgccttccccggactctgcctacgcgtgcggcggcccgtcgtgcttcggtttcctcgagccggagacggccccggccacgccccagggcgagggcgaggaggaggaggaggccgcggcgttcatgggcgaggcggccgccgcggtggcacggggcaagcactaccgtggggtgaggcagcggccgtggggcaagttcgcggcggagatccgggacccggccaagaacggcgcgcgcgtgtggctcggcacctacgacaccgccgaggacgccgccgtggcctacgaccgcgccgcctaccgcatgcgcggctcccgcgcgctcctcaacttcccgctccgcatcggctccgagatcgccgccgcgcacgctgcggccgcggccgccggcgacaagcggccgtcgcccgagcccgcaacctcggacacatcctcgtcatcttccgggtcgccgaaaagaaggaagagaggcgaggccgcggccgcgtcgatggccatggcgctggtgccgccgccctccctgctgagccggccggcccaggcgtggtaccccgccgcgccggtggagcaggtggccatggcgccgcgcgcgcagcagctggtcagctag

SEQ ID NO: 66 - DNA, 12783 bp, Artificial Sequence
  DNA sequence of ERF922 RNAi construct
aaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcaggggtacgtcgaggtcgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcgcgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacgtctcaaccgtgcggctgcatgaaatcctggccggtttgtctgatgccaagctcgcggcctggccggcgagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggcggcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggcgagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccggtgggtatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgtctaacaattcgttcaagccgacgccgcttcgcggcgcggcttaactcaagcgttagatgcactaagcacataattgctcacagccaaactatcaggtcaagtctgcttttattatttttaagcgtgcataataagccctacacaaattgggagatatatcatgaaaggctggctttttcttgttatcgcaatagttggcgaagtaatcgcaacatagcttgcttggtcgttccgcgtgaacgtcggctcgattgtacctgcgttcaaatactttgcgatcgtgttgcgcgcctgcccggtgcgtcggctgatctcacggatcgactgcttctctcgcaacgccatccgacggatgatgtttaaaagtcccatgtggatcactccgttgccccgtcgctcaccgtgttggggggaaggtgcacatggctcagttctcaatggaaattatctgcctaaccggctcagttctgcgtagaaaccaacatgcaagctccaccgggtgcaaagcggcagcggcggcaggatatattcaattgtaaatggcttcatgtccgggaaatctacatggatcagcaatgagtatgatggtcaatatggagaaaaagaaagagtaattaccaattttttttcaattcaaaaatgtagatgtccgcagcgttattataaaatgaaagtacattttgataaaacgacaaattacgatccgtcgtatttataggcgaaagcaataaacaaattattctaattcggaaatctttatttcgacgtgtctacattcacgtccaaatgggggcttagatgagaaacttcacgatcggctctagctagggataacagggtaatgcgtgctgcagggctgaagcggtgcgggaagagctgcaggctgaggtacaccaactacctgaggcccaacctcaagcacgagaacttcacgtacgcactggattttggttttaggaattagaaattttattgatagaagtattttacaaatacaaatacatactaagggtttcttatatgctcaacacatgagcgaaaccctataagaaccctaattcccttatctgggaactactcacacattattatagagagagatagatttgtagagagagactggtgatttcagcttcagatctttactccggcagcgttgtgtcgcccgcgttgagcggccgttgcatgagcaccgtgtcgatccagcggccgaacttgaagccgacggacttgagcacgcctgcctgcacgaagccgcaggcggcatgcagaccgagggacgccgtattgccgctgtcgccgacgttcgcaatcagttgccgccacgggccgccttcgcaacgcgcgatgagcgtgagcaacagcgtgcgccctacgccctgcccgatcgcatcgggggcgatatacaccgagtcttccagcgtgaaacggtaggcggagcgcggccggtaatgcgtggcgtatgcgtagccgagcagcttgccgtcgcgttcggcgacgagatacggcagtccggcgtcgagtaccttggcgcaacgcgcgcgcatctcggtgtcgtcgggtggcacctcttcgaacgatgccgtgccggtacgcacgtgatgggcgtagatggccgtgatggcgggcaggtcgtcgtcacgcacgacgcgcaccgtgcatgcgcaacgcgccagttcgacgccgacgcgttgcggcgcgtcgggagtggaggggtgagacgaagatgacggcatggtctgcagctcgagccgcagctgcacgggtccaggaaagcaatcgcatagtcaagctaaatcatcaagatgcaaacttttcgcccttgctaaacacggtaaaattcgaatggacatgtgtggagcagcaaaggccttacgtccgagaaacagggccactcaacgagttagttaaattcaaagaaagaaacgcctccttgcaagttgcaacattcctagatcatactaatgaaaatgacgtccttcattaaagaacagggaagatagatttttgctccatagatcgtatgatgtgttcagccagactgtcggatggaccacacggtaatagcagtgctggacgatgttacatcgagaaagattactagccttttcatgggagtaaaggatataaaagaaataagttcaccacgattgcaggatagcatacaagatcagcgccactgcggcactgttcatcgaaaaaaaactgtggacgaagctttccccaaaattactcaacgaatcataaaccaagattagtcagatcaagagacagaggagaaacaaggcggacctttgcagtcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgagactgtatctttgatattcttggagtagacgagagtgtcgtgctccaccatgtatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgaacgcgtgctgcagggctgaagcggtgcgggaagagctgcaggctgaggtacaccaactacctgaggcccaacctcaagcacgagaacttcacgtacgctagggataacagggtaatccaatggtctagaggccatggcggccggaagacaaagatagcccggggcctgcaggaccaatgcattgggggcccttggccggcctcgagcgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggaaattcgcgtacgcgcacgggcacggccagtacctccccctcaacgaggacgactccctggacatggtcctcttcgacgtgctccgcgaggcatccggctccgcacccggcgccacctaccagccgccctcgtcgtcgttgcctacgctgaaggcgctcgcttccggcgcctcgcgcaagggcggtggcaacaacggcaacggcagtagggccacctgaaattggtaaaagtagacttttagttagtactctatctatgagggccatcccttcttttcgaaaaaaaaactctataaattgtatcaggtacactactctttttcaggactcggaagctggaaaatttggcttatttagttttttggggaagtatgtcacatgatgaatagttagtgcatagaaaaataggcatttttttccatgtcacctgcacatgtgttatatgatatcccagctatattttgttaggcaactagataaccattttcaaattaaacaaatgcagtatgaagatacactatccatacctggccctactgccgttgccgttgttgccaccgcccttgcgcgaggcgccggaagcgagcgccttcagcgtaggcaacgacgacgagggcggctggtaggtggcgccgggtgcggagccggatgcctcgcggagcacgtcgaagaggaccatgtccagggagtcgtcctcgttgagggggaggtactggccgtgcccgtgcggcggccgctgaggtgcacgaagtaacaccaaacaacagggtgagcatcgacaaaagaaacagtaccaagcaaataaatagcgtatgaaggcagggctaaaaaaatccacatatagctgctgcatatgccatcatccaagtatatcaagatcaaaataattataaaacatacttgtttattataatagataggtactcaaggttagagcatatgaatagatgctgcatatgccatcatgtatatgcatcagtaaaacccacatcaacatgtatacctatcctagatcgatatttccatccatcttaaactcgtaactatgaagatgtatgacacacacatacagttccaaaattaataaatacaccaggtagtttgaaacagtattctactccgatctagaacgaatgaacgaccgcccaaccacaccacatcatcacaaccaagcgaacaaaaagcatctctgtatatgcatcagtaaaacccgcatcaacatgtatacctatcctagatcgatatttccatccatcatcttcaattcgtaactatgaatatgtatggcacacacatacagatccaaaattaataaatccaccaggtagtttgaaacagaattctactccgatctagaacgaccgcccaaccagaccacatcatcacaaccaagacaaaaaaaagcatgaaaagatgacccgacaaacaagtgcacggcatatattgaaataaaggaaaagggcaaaccaaaccctatgcaacgaaacaaaaaaaatcatgaaatcgatcccgtctgcggaacggctagagccatcccaggattccccaaagagaaacactggcaagttagcaatcagaacgtgtctgacgtacaggtcgcatccgtgtacgaacgctagcagcacggatctaacacaaacacggatctaacacaaacatgaacagaagtagaactaccgggccctaaccatggaccggaacgccgatctagagaaggtagagagggggggggggggaggacgagcggcgtaccttgaagcggaggtgccgacgggtggatttgggggagatccactagtcgaggtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgagactgtatctttgatattcttggagtagacgagagtgtcgtgctccaccatgtatcacatcaatccacttgctttgaagacgtggttggaacgtcttctttttccacgatgttcctcgtgggtgggggtccatctttgggaccactgtcggtagaggcatcttgaacgatagcctttcctttatcgcaatgatggcatttgtagaagccatcttccttttctactgtcctttcgatgaagtgacagatagctgggcaatggaatccgaggaggtttcccgatattaccctttgttgaaaagtctcaatagccctctggtcttctgaacgacgtcgggccccccctgcaggactagtggatttgtcttcaaggccttaagggccagatcttgggcccggtacccgatcagattgtcgtttcccgccttcggtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggagtgcttggcattccgtgcgataatgacttctgttcaaccacccaaacgtcggaaagcctgacgacggagcagcattccaaaaagatcccttggctcgtctgggtcggctagaaggtcgagtgggctgctgtggcttgatccctcaacgcggtcgcggacgtagcgcagcgccgaaaaatcctcgatcgcaaatccgacgctgtcgaaaagcgtgatctgcttgtcgctctttcggccgacgtcctggccagtcatcacgcgccaaagttccgtcacaggatgatctggcgcgagttgctggatctcgccttcaatccgggtctgtggcgggaactccacgaaaatatccgaacgcagcaagatatcgcggtgcatctcggtcttgcctgggcagtcgccgccgacgccgttgatgtggacgccg

SEQ ID NO: 67 - DNA, 24 bp, Artificial Sequence
  crGEP239 (gRNA targeting ERF922)
aggccgggttcagcagcatcctcg

SEQ ID NO: 68 - DNA, 24 bp, Artificial Sequence
  crGEP243 (gRNA targeting ERF922)
gctcaacgagaacgactccctcga

SEQ ID NO: 69 - DNA, 200 bp, Artificial Sequence
  TaCPL1_silencing_sequence_1
ccagtcgttcaaccagttgtcttagatccactgcaagcttctccaggtagagaagagggcgaggttccagagtctgagttagatcctgacacaaggagaaggcttctgatattacagcatggccaagacacaagagatcccacaccaccctttgctgcagaaccttctgtacaagcctcagttcctccagtgcaatctca

SEQ ID NO: 70 - DNA, 222 bp, Artificial Sequence
  TaCPL1_silencing_sequence_2
cagaagcagccaaatggagtcaggacgtcaatttgtacaatacacagagacatctggtgctgtattggaggagattgcagcgaagtgtggatttaaggtggagtacaggtcaacattatgtgataccgcagaactacagttctctattgaggtttggatagtgggagaaaaagtgggtgaaggcattggtagctcaaggaaagaagcacagcgtcaagctgc

SEQ ID NO: 71 - DNA, 24 bp, Artificial Sequence
  crGEP240 (gRNA targeting ERF922)
gaagctggcgctgcggcagtaggc

SEQ ID NO: 72 - DNA, 24 bp, Artificial Sequence
  crGEP241 (gRNA targeting ERF922)
gggaaggcaggaggggctccggct

SEQ ID NO: 73 - DNA, 24 bp, Artificial Sequence
  crGEP242 (gRNA targeting ERF922)
cctgctcgggtgggcgcggccatg

SEQ ID NO: 74 - DNA, 24 bp, Artificial Sequence
  crGEP244 (gRNA targeting ERF922)
tcgacatgctcctcttcgacgtcc

SEQ ID NO: 75 - DNA, 21 bp, Artificial Sequence
  Protospacer sequence
gaggacagaattgatgcactt

SEQ ID NO: 76 - DNA, 21 bp, Artificial Sequence
  Protospacer sequence
agatccactgcaaggttctcc

SEQ ID NO: 77 - DNA, 21 bp, Artificial Sequence
  Protospacer sequence
accgttgaatccgattctatg

SEQ ID NO: 78 - DNA, 21 bp, Artificial Sequence
  Protospacer sequence
gcttgagggaacatgccgagt

SEQ ID NO: 79 - DNA, 21 bp, Artificial Sequence
  Protospacer sequence
ggtggagtacaggtcaacatt

SEQ ID NO: 80 - protein, 932 aa, Artificial Sequence
  ZmCPL1.1 with D148A substitution
MFKSMVYYVNTSIGEVEVWPKGEASAGLTMAAWAREIRVERISPPSERCPPLAVMHTVAVGARCLVMESRPPVVADVVPPLVVMHTACLRENKTAVVPLGDEELHLVAMTSRRNLTNHACFWGYKLPFGLYNSCLTMLNLRCLGIVFALDETLIVANTSRSFEDRIDALQRKLSNETDPQRRNGMLSEIKRYQDDKSILKQYIEGDQVYDDGKVYKAQPEIVPPLSDNQQPMTRPVIRLQDKNIILTRINPLIRDTSVLVCLRPAWEDLRSYLIARGRKRFEVYVCTMAERDYALEMWRLLDPDSRLINSVQLHDRMVCVKSGLKKSLLNVFHDGSCHPGMALVIDDRLKVWDEKDQLRVHVVPAFTPYYAPQAEANCSIPVLCVARNVACNVRGGFFKDFDEGLLPRISNVHYEDEVNEISAPDVGNYLITDDENVALVNGNRDSLPFDGMADAEVERRMKEANAQSFHQTAGDFVMPVAPAQNFVSTSVASLAPPLGMMPSPFSQPVAPPGFSDSLQGSPAREEGEVPESELDPDTRRRLLILQHGQDTRDPTSPLPAIPPVQVPVPPVQPHGNWFPTEDGINQSNLNRGSAGFTVESDSIVYEKKQPPHPSFFHGGDSPMPSDRFGYQNQRFPSQLPHEDHPMMQNHAPPKYRSFSGEELASWHVPSSQRNNQIESGRHFAQYAGTSAGILEGIALKCGSKVEYKSALCDTAELQFSIEVWIVGEKVGEGIGRTRREAQRQAAEMSLRNLANKYLSSDPNKLSDMKENDFSSNRNVFGYSGNTRDDMLPLSSTSEESRFMKMENNNSRKTGSSVAALKELCTVEGYNLVFQACPSSADGLVGKESYAQVQVGGQILGKGVGLTWEEAKLQAAAEALGTLRSMLGQLGHKRSGSPRSLAPNFNKRFKPDFPRTVQRVPYGTYSRIEGHVP

SEQ ID NO: 81 - protein, 934 aa, Artificial Sequence
  ZmCPL1.2 with D149A substitution
MFKSMVYFVNISIGEVEVWPKGQASAGLAVAAWARDIRVDRLSPPSERCPPLAVMHTVAVAARCLVMESRPPVAADVASLPLVDMHAACLRDNKTAVVALGDEELHLVAMTSRRNLTNHACFWGYKLPFGLYNSCLTMLNLRCLGIVFALDETLVVANTSRSFEDRIDALQRKLSNETDPQRRNGMLSEIKRYQDDKSILKQYIEGDQVYDDGKVYKAQPEIVPPLSDNQQTMTRPVIRLQEKDIILTRINPLIRDTSVLVRLRPAWEDLRSYLIARGRKRFEVYVCTMAERDYALEMWRLLDPDSRLINSVRLHDRMVCVKSGLKKSLLNVFHDGSCHPGMALVIDDRLKVWDEKDQLRVHVVPAFTPYYAPQAEANCSIPVLCVARNVACNVRGGFFKDFDEGLLPRISNVHYEDEVNDISSAPDVGNYLITEDENVALVNGNRDSLPFDGMSDAEIERRMKEANAQAFHQTATNFVMPVAPAQNFVSSSVAPLAPPLSMMPPPFSQPVVQPGFSDPLQGSPAREEGEVPESELDPDTRRRLLILQHGQDTRDPTPPHPAIPPVQVPVPPVQPHGNWFPTEDGINPSNLSRGSAGFTVESDSMPYEKKQPPHPSFFHGGDSPMSSDRFGYQNQRFPSQLPHTEDHHMLQNHAPPKYRSFSGEELATRHVPSSQRNHIESGRHFSQYAGTSAGVLEGIAVKCGSKVEYRSTLCDTAELQFSIEVWIVGEKFGEGIGRTRREAQRQAAEMSLRNLANKYLSSDPNKLTDMKQDAFGSNRNIFGYSGNTRDDMLPLSSTSEESRFMKMEENNSRKTGDSVTALKELCTVEGYNLVFQACPSPADGLVGKESYAQVQIGRQILGKGVGLTWEEAKLQAADEALGTLRSMLGQLGHRRSGSPRSLAPNFNKRFKPDFPRTVQRVPYGTYSRIEGHAP

SEQ ID NO: 82 - protein, 1023 aa, Artificial Sequence
  Beta vulgaris g16374.t1 protein with D162A substitution
MIKSVVYEGENLLGEVEIYFQNNNNNKNLELMKGMRISHYSEMSERCPPLAVLHTITKSSGGICFKMMESSSHTSSNNNNNNKFYFQQQQQESQLLAMHSNCIRDNKTAVVPLGEQEIHLVALRSRRMAGVTPCFWGFSVAPGLYESCLGLLNLRCLGIVFALDETLIVANTLRSFEDRIEALQRKISVEADPQRIAGMVAEVKRYQEDKSILKQYAETDQVVDNGKVHKIQAEVIPALSDNHQTVIRPLIRLQDKNIVLTRINPQIRDTSVLVRLRPAWEDLRSYLTARGRKRFEVYVCTMAERDYALEMWRLLDPDSNLICARELLDRIVCVKSGSKKSLFNVFHGGICHPKMALVIDDRLKVWDEKDQPRVHVVPAFAPYYAPQAEANNAIPVLCVARNVACNVRGGFFKEFDEGLLQRVSEVSFEDDPRDIPSPPDVSNYLVSEDDGSGSNGIKESMTFDGMADAEVERRLKEAVLSSSSASPLPSANTPATVNFDHRLASSLPFAVATSALAIPQPAPQATITPYHNNLFSQAGPLARPLGNIGPQDIGLHNSPAREEGEVPESELDPDTRRRLLILQHGQDMREGPPNEPPFPARTPVQAPVTGPVPVSVPGPVPVPGPGPVSVPVPGPIPSPVPVPVSGTVPGPGPRVQSRGSWFPVEDHISQGPLSRVAAKEFPVAPDASPVEKQRPPPPSFPRKVESLGWSDRNYAEKQRLPREALRRDDRLRSNYSLPSHQSFRGDEISLSRSASSNKDFEVEPERGSSFAESPSIALHDIAMKCGTKVEFKTGLVATPELKFLLEAYFAGDKIGEGTGTTRREAQHRAAEAALMNLADKYLTHIKSDSSTPQSDTSRGHSPIDTGFVSDANSHGDGISRKEDIIPSSEMTGLDDSNVDGSKNSMGSVSVLKELCLREGLGVDFKGQSPTSTNSVDRDEIHAEVEINGQVLGKGTGLTWDEAKMQAAEMALTSLNSMIGQFNKRPSSPRLLQGMPNKRLKPEYPRVVDHLPSSRYPRNASPVP

SEQ ID NO: 83 - protein, 881 aa, Artificial Sequence
  Solanum tuberosum PGSC0003DMT400057635_protein with D143A substitution
MFKSTVVLYEGERLVGEVEIYCEKGVLWGEKVIRISHYSPSSERCPPLAVLHTVTTGLSFKLEPTKSKPLTQDSPLTLLHSTCLRDNKTAVMSLGREELHLVAMQSKNIGGQCPCFWGFKVASGLYDSCLTMLNLRCLGIVFALDETLIVANTMRSFEDRIEALQRKINSESDPQRASVMLAEVKRYQEDKIILKQYAENDQVVDNGKVIKSQSEVFPALSDNHQPIVRPLIRLQDRNIILTRINPMIRDTSVLVRLRPAWEDLRSYLTARGRKRFEVYVCTMAERDYALEMWRLLDPDSNLINSQELLDRIVCVKSGLRKSLFNVFQDGNCHPKMALVIDDRLKVWDDKDQPRVHVVPAFAPYFAPQAEGNNSVPVLCVARNVACNVRGGFFKDFDEGLLQRISEVAYEDDIKQVPSAPDVSNYLISEDDPSAVNGNKDSLGFDGMADSEVERRLKEAMLASTSVPSQMTNLDPRLVPALQYPVPPVISQPSIQSPVVPFPTQHLPQVTSVLKSSVTQISPQDTSLQSSPAREEGEVPESELDPDTRRRLLILQHGQDTRDQVSSEPKFPMGTPLQVSVPPRVQPHGWFPAEEEMSPRQLNRPLPPKEFPLNPESMHINKHRPPHPPFLPKMETSMPSDRVLFENQRLPKEVIPRDDRMRFSQSQPSFRPPGEEVPLGRSSSSNRVLDLEPGHYDPYLETPAGALQDIAFKCGAKVEFRSSFLSSPELQFSLEVLFAGEKVGEGTGRTRREAQRRAAEESLMYLADKYLSCIKPDSSSTQGDGFRFPNASDNGFVDNMSPFGYQDRVSHSFASEPPRVLDPRLEVFKKSVGSVGALRELCAIEGLGLAFQTQPQLSANPGQKSEIYAQVAHLLPFLLCYC

SEQ ID NO: 84 - protein, 956 aa, Artificial Sequence
  Glycine max Glyma.07g194800.1 protein with D141A substitution
MRMYKSVVYQGEVVVGEVDVYPEENNNYKNFHVKEIRISHFSQPSERCPPLAVLHTVTSCGVCFKMESKTQQQDGLFQLHSLCIRENKTAVMPLGGEEIHLVAMHSRNVDRPCFWGFIVALGLYDSCLVMLNLRCLGIVFALDETLIVANTMRSFEDRIDALQRKINSEVDPQRISGMQAEVKRYQDDKNILKQYAENDQVVDNGRVIKVQSEIVPALSDSHQPIVRPLIRLQDKNIILTRINPQIRDTSVLVRLRPAWEDLRSYLTARGRKRFEVYVCTMAERDYALEMWRLLDPDSNLINSKELLGRIVCVKSGLKKSLFNVFQDGLCHPKMALVIDDRLKVWDEKDQPRVHVVPAFAPYYAPQAEASNTIPVLCVARNVACNVRGGFFKDFDDGLLQKIPQIAYEDDIKDIPSPPDVSNYLVSEDDGSISNGHRDPFLFDGMADAEVERKLKDALSAASTIPVTTANLDPRLTSLQYTMVPSGSVPPPTAQASMMPFPHVQFPQPATLVKPMGQAAPSEPSLHSSPAREEGEVPESELDPDTRRRLLILQHGQDTRDHASAEPPFPVRHPVQTSAPHVPSSRGVWFPAEEEIGSQPLNRVVPKEFPVDSGPLGIAKPRPHHPSFFSKVESSISSDRILHDSHQRLPKEMYHRDDRPRLNHMLSSYRSFSGDDIPFSRSFSSHRDLDSESGHSVLHADTPVAVLQEIALKCGTKVDFISSLVASTELQFSMEAWFSGKKIGHRVGRTRKEAQNKAAEDSIKHLADIYLSSAKDEPGSTYGDVSGFPNVNDSGYMGIASSLGNQPLSKEDSASFSTASPSRVLDPRLDVSKRSMGSISSLKELCMMEGLDVNFLSAPAPVSTNSVQKDEVHAQVEIDGKVFGKGIGLTWDEAKMQAAEKALGSLRSKLGQSIQKRQSSPRPHQGFSNKRLKQEYPRPMQRMPSSARYPRNAPPIP

SEQ ID NO: 85 - protein, 948 aa, Artificial Sequence
  Triticum aestivum TraesCS2A02G373400.3 protein with D149A substitution
MIKSMVYFGHISIGEVELSPKGETNVAAAPWVREIRVDRLSPPSERCLPLAVLHTVSSGALCFVMESRPSPATADNEPPSSLVAMHTACLRDNKTAVFPLGAEEIHLVAMKPKSNLPNHACFWGYKVPLGLYSSCLSMLNLRCLGIVFALDETLIVANTTRSFEDRIDALQRKLSKETDPQRISGMLAEIKRYQEDRTMLKQYIDGDQVIDGGKMYKVQSEVVPPLADNHQPMIRPVIRLQDKSIILTRINPSIRDTSVLVRLRPAWDDLRSYLIARGRKRFEVYVCTMAERDYALEMWRLLDPDSRLINSVQLPHRLVCVKSGSKKSLLNVFHDGSCHPGMALVIDDRLKVWEEKDQCRVHVVPAFSPYYAPQAEANFPIPVLCVARNVACNVRGSFFKEFDEGLLPSISEVHFDDELDHVPSSPDVGNYLISEDENAASLNVNKDPMAFDGMADAEVERRMKEAVCSVQAADPVTTNVDVISVAANQQFATSSSIPLAPPLGVVPLNNDQGPQPPSVSWPDAQSGMVDPLQGSPAREEGEVPESELDPDTRRRLLILQHGQDTRDPTPPFAAEPSVQASVPPVQSQGNWFPVEDEMDPRNLNRTSTDFHLESDAVHSDKSQPPHQPYFPARDNPIFSDRLNHQNQRYSSQLPHSEDRQMLQNQAPTTYRSFSGEDMATQRFHPGNRSSQMESGRQFVQYTETSGAVLEEIAAKCGFKVEYRSTLCDTTELRFSIQIWIVGEKVGEGMGRTRKEAQRQAANISLRNLADRFLSFDPDKMTVPVDDGFSSNPNSFKYRGIDGDNIVPVASTSDGSRYMHERVDNSTKSAGSVAALKELCTAEGYNLVFQAQPSPSDSLRREEVHAQIEIGGQILGKGVGVTWEEAKVQAADGALGTLRYMLGQRPQKRSGSPRSFASNYNNKRYKPDFQPMVQRIPSGRYSRNDSRVP

SEQ ID NO: 86 - protein, 935 aa, Artificial Sequence
  Triticum aestivum TraesCS6B02G264000.4 protein with D147A substitution
MIKSMVYYGNTSIGEVEVWPKGDTNLGAAAWAREIRVDRLSPPSERCLPLAVMHTVAVGARCLVMESRPPKAADEPPPPLVAMHAACLRDNKTAVVPLGEEELHLVAMTSGRNLTNHACFWGYKVPFGLYNSCLTMLNLRCLGIVFALDETLIVANTTRTFEDRIDSLQRKLSNETDPQRMNGMLAEIKRYQDDRSILKQYIEGDQVYDDGKMYKVQPEIVPPLSDNHQSLTRPVIRLQEKNIILTRINPLIRDTSVLVRLRPAWEDLRSYLIARGRKRFEVYVCTMAERDYALEMWRLLDPDSRLINSVQLSDRMVCVKSGLKKSLLNVFHDRSCHPGMALVIDDRLKVWDEKDQSRVHVVPAFTPYYAPQAEANCSIPVLCVARNVACNVRGGFFKDFDEGLLPRITSVLYEDEIQDISSAPDVGNYLISEDENVAVVNGNRDSLAFDGMADAEGERRMKEASGSCSVLNPTMANMVMPVAPSQSFVPSSVAPFAPPLGMMPLSNNQVPPPVFSQPVVQPVVLDPLQASPGREEGEVPESELDPDTRRRLLILQHGQDTRDPTPPLPAVPPAQVSVPPVQSHGNWFPVEDGIGMNSNNLNRGSAGFPSESDTMHYDKKQPPQPSYFHGGDNNPVSSDRFSYQSQRFPSQVTHTEDHRMLQNHAPPRYRSFPGQRDNLIESGQSYARNVGTSVGILEEIALKSGSKVEYRSKLCDTAELQFSIEVWIVGEKVGEGIGSSRKEAQRQAAEISLRNLANKYLLSDPNKMTDVNEDGFGSNPNFFGYSENTRNDILPVASTSEESRFTKTGENNSRITGGSIAALKQLCTVEGYNLVFQARPSPLDGSGGKETYAQVEVGGQTLGKGVGITWEEAKLQAADEALGTLRSMLGQLAQKRSSSPRSLAPNYNKRFKPDFPRAVQRPPYGRYSRIEGHVP

SEQ ID NO: 87 - protein, 934 aa, Artificial Sequence; identical to SEQ ID NO: 4
  Sorghum bicolor Sobic.004G227600.1 protein with D149A substitution

(19) United States
(12) Patent Application Publication
(10) Pub. No.: US 2023/0357788 A1
(43) Pub. Date: Nov. 9, 2023

(54) ENHANCED DISEASE RESISTANCE OF CROPS BY DOWNREGULATION OF REPRESSOR GENES

(71) Applicant: KWS SAAT SE & Co. KGaA, Einbeck (DE)

(72) Inventors: Daniel Fabian STIRNWEIS, Gottingen (DE); Dietmar STAHL, Einbeck (DE); Urs Konrad FISCHER, Gottingen (DE); Christopher SCELONGE, Chesterfield, MO (US)

(73) Assignee: KWS SAAT SE & Co. KGaA, Einbeck (DE)

(21) Appl. No.: 17/768,696

(22) PCT Filed: Oct. 16, 2020
(86) PCT No.: PCT/EP2020/079174

§ 371 Date: Apr. 13, 2022

Related U.S. Application Data
(60) Provisional application No. 62/916,578, filed on Oct. 17, 2019.

(51) Int. Cl. C12N 15/82; C07K 14/415

(52) U.S. Cl. CPC C12N 15/8282; C07K 14/415

(57) ABSTRACT

The present invention relates to plants having increased resistance or tolerance to pathogens. Such plants have reduced expression levels of CPL1 and/or ERF922. The invention further relates to methods for producing such plants, as well as methods for identifying such plants.

### FIELD OF THE INVENTION

The invention relates to plants having increased resistance or tolerance to pathogens. The invention further relates to methods for producing such plants, as well as methods for identifying such plants.

### BACKGROUND OF THE INVENTION

Plants possess a highly efficient, two layered innate immune system that makes them resistant against most microbial pathogens (Jones and Dangl, 2006, “The plant immune system”, Nature, 444(7117):323-329).

The first layer of defence relies on the recognition of evolutionary conserved pathogen- or microbial-associated molecular patterns (PAMPs or MAMPs) by the so-called pattern recognition receptors (PRRs).

PAMPs or MAMPs are invariant structures broadly represented among microbial taxa and have essential roles in microbial physiology. Only an extremely select group of molecules have been found to function as PAMPs. It was also found that conserved molecules from nematodes can elicit plant defences and pathogen resistance. Accordingly, such molecules were defined as nematode-associated molecular patterns (NAMPs). Besides molecular patterns originating from the pathogen, plants can also sense molecular patterns associated with cell wall destruction or cell damage, the so-called danger/damage-associated molecular patterns (DAMPs).

PRRs are generally plasma membrane receptors which are often coupled to intracellular kinase domains or require a co-receptor to provide signalling function (Dangl et al., 2013, “Pivoting the plant immune system from dissection to deployment”, Science, 341(6147):746-751). Depending on the presence of the signal transduction domain the plant PRRs are classified either as receptor-like kinases (RLKs) or as receptor-like proteins (RLPs). Recognition of PAMPs, MAMPs, NAMPs or DAMPs in the apoplast by pattern recognition receptors (PRRs) initiates a complex signalling cascade leading to PRR-triggered immunity (PTI). Adapted pathogens may be able to suppress the first defence layer through the secretion of effector proteins that interfere with the signaling (Jones and Dangl, 2006).

The second layer of plant defence, the effector triggered immunity (ETI), relies on the specific recognition of effectors by disease resistance genes (Jones and Dangl, 2006). This recognition leads to a strong defence response which is often associated with a local programmed cell death, the hypersensitive reaction. Since effectors are generally species or isolate specific, this second layer of immunity is only efficient against isolates that carry the recognized effector, which is then called an avirulence gene.

Whether a potential pathogen is able to overcome the first layer of defence, the PTI, and to reproduce effectively, depends on its intrinsic ability to suppress PTI responses of the plant. But it also depends on the plants ability to efficiently and quickly induce and, if required, to maintain defence responses above a certain threshold for effective resistance (Jones and Dangl, 2006).

PTI responses are generally conserved and include the activation of mitogen-activated protein kinases (MAPKs), the generation of reactive oxygen species, the activation of salicylic acid (SA)- and jasmonic acid (JA)-signaling pathways and the enhanced expression of plant defence genes like pathogenesis-related proteins. Transcriptional activation can typically be measured within minutes or hours after infection and it is reduced after effective defence response.

Transcription of protein-coding genes in eukaryotes is intricately orchestrated by RNA polymerase II (RNAPII), general transcription factors, mediators, and gene-specific transcription factors. The multisubunit RNAPII is evolutionarily conserved from yeast to human. Its largest subunit Rpb1 contains a carboxyl-terminal domain (CTD) consisting of conserved heptapeptide repeats with the consensus sequence Y1S2P3T4S5P6S7 (Buratowski, 2009, “Progression through the RNA polymerase II CTD cycle”, Moll Cell, 36(4):541-546). The combinatorial complexity of CTD posttranslational modifications constitutes a ‘CTD code’ that is ‘read’ by CTD-binding proteins to regulate the transcription cycle, modify chromatin structure, and modulate RNA capplng, splicing, and polyadenylation. In particular, the CTD undergoes waves of Serine phosphorylation and dephosphorylation events regulated by various CTD kinases, often members of cyclin-dependent kinases (CDKs), and phosphatases during transcription initiation, elongation, and termination. The interplay between different CTD kinases and phosphatases provides a means for coupling and coordinating specific stages of transcription by recruiting other factors required for proper gene expression.

In Arabidopsis, there are five members of the CTD phosphatase-like protein family (CPL1-5) (Fukudome et al., 2014, “Arabidopsis CPL4 is an essential C-terminal domain phosphatase that suppresses xenobiotic stress responses”, Plant J, 80(1):27-39). They were shown to possess preferences for different phosphorylated serines in the heptapeptide repeats and they were shown to be involved in different biological processes.

Arabidopsis AtCPL1 is a negative regulator of stress-responsive gene expression under various abiotic stresses (cold, abscisic acid (ABA), salt treatment, and iron deficiency) (Koiwa et al., 2002, “C-terminal domain phosphatase-like family members (AtCPLs) differentially regulate Arabidopsis thaliana abiotic stress signalling, growth, and development”, PNAS, 99(16):10893-10898; Aksoy et al., 2013, “Loss of function of Arabidopsis C-terminal domain phosphatase-likel activates iron deficiency responses at the transcriptional level”, Plant Physiol, 161(1):330345), and negatively regulates wound-induced JA-biosynthesis genes. It was shown lately by Thatcher et al. (2018, “The Arabidopsis RNA Polymerase II Carboxyl Terminal Domain (CTD) Phosphatase-Like1 (CPL1) is a biotic stress susceptibility gene”, Sci Rep, 8:13454) that a complete knock-out of AtCPL1 in Arabidopsis leads to enhanced resistance against the necrotrophic fungal pathogens Fusarium oxysporum and Alternaria brassicicola, and reduced symptom development under aphid (Myzus persicae) infestation. In addition, a delayed flowering is shown for Arabidopsis AtCPL1 mutants with early stop codons (full AtCPL1 knock-out).

Plants with downregulation of AtCPL1 were not described by Thatcher et al. (Sci Rep; 2018). An effect of CPL genes on pathogen resistance from other plant species than Arabidopsis was not described so far.

ERF922 is a plant ethylene responsive factor (ERF), a subfamily of the APETELA2/ethylene response factor (AP2/ERF) transcription factor superfamily in plants. ERF922 mutants of rice were generated by CRISPR/Cas9 engineering which results in the improvement of rice blast resistance (Wang et al., 2016, “Enhanced Rice Blast Resistance by CRISPR/Cas9-Targeted Mutagenesis of the ERF Transcription Factor Gene OsERF922”, PLoS ONE, 11(4):e0154027). The publication shows that RNAi mediated down-regulation of ERF922 enhances resistance against the fungal pathogen Magnaporthae grisea and acts as a negative regulator of plant defence. OsERF922 is a transcriptional activator, binds to the GCC element of promoters and is induced by M. grisea infection. OsERF922 downregulates the expression of defence related genes including PR1 and P10.

### SUMMARY OF THE INVENTION

The present invention relates to induction or increase of pathogen resistance in plants, in particular by reducing or eliminating the expression level of negative regulators of plant defence genes or disease resistance genes. In particular embodiments, the invention relates to methods for inducing or increasing pathogen resistance in a plant by reducing or eliminating the expression level of CPL1 and/or ERF922, or otherwise affecting the activity or stability of these proteins. Indeed, both CPL1 and/or ERF922 have been found to be negative regulators of plant defence particularly suitable for the methods of the invention.

The present inventors hereto have identified previously unknown CPL1 and ERF922 genes in a variety of economically highly relevant crops, including corn, wheat, barley, rye, Sorghum, potato, soy, beet.

Moreover, in several of these crops, multiple CPL1 and/or ERF922 genes have been identified, in particular several homeologues and paralogues. Several of the homeologues/paralogues were moreover found to be differentially expressed. The presence of such homeologues or paralogues—and in particular when differentially expressed—obviously adds many levels of complexity. Advantageously however, the existence of different paralogues allows to better fine-tune pathogen resistance.

The inventors have found that reducing the expression (or otherwise reducing functionality) of CPL1 and/or ERF922—in particular of CPL1—rather than completely abolishing expression advantageously results in increased pathogen resistance or tolerance, without concomitant growth or developmental defects, such as growth retardation in case of ERF922 knockout or delayed flowering in case of CPL1 knockout.

The use of dominant negative forms of CPL1 and/or ERF922—in particular CPL1—advantageously can achieve the above effects, especially in cases where multiple homeologues and/or paralogues are present, as a single dominant negative form can simultaneously suppress all homeologues and paralogues.

Furthermore, the present inventors for the first time have demonstrated that reduced or eliminated expression (or otherwise reduced or eliminated functionality) of CPL1 and/or ERF922 is capable of increasing pathogen resistance or tolerance against biotrophic and hemibiotrophic pathogens (in particular fungi), which operate in an entirely different manner than necrotrophic pathogens.

Down-regulation of negative regulators of plant defence such as CPL1 and ERF922 increases the plant tolerance against diseases caused by fungal pathogens, bacteria, insects and nematodes. The downregulation can be done for instance by disruption of the coding sequence by CRISPR/nuclease or Tilling, the reduction of gene transcription by RNAi, miRNA and by generation of less functional alleles, such as by Tilling or CRISPR/nuclease.

The present invention is in particular captured by any one or any combination of one or more of the below numbered statements 1 to 73, with any other statement and/or embodiments.
1. A method for increasing resistance and/or tolerance to a pathogen in a plant, plant part, or plant population, comprising reducing or eliminating the expression, stability, and/or activity of a CPL1 and/or ERF922 protein and/or gene in a plant, plant part, or plant population.
2. The method according to statement 1, wherein a CPL1 and/or ERF922 gene is mutated.
3. The method according to statement 1 or 2, the coding sequence and/or a regulatory sequence of a CPL1 and/or ERF922 gene is mutated.
4. The method according to any of statements 1 to 3, wherein expression, stability, and/or activity of a CPL1 and/or ERF922 protein is reduced.
5. The method according to any of statements 1 to 4, comprising expressing mutated CPL1 and/or ERF922 protein.
6. The method according to statement 5, wherein said mutated CPL1 and/or ERF922 gene comprises a point mutation, preferably resulting an amino acid substitution in the CPL1 and/or ERF922 protein.
7. The method according to statement 5 or 6, wherein said mutated CPL1 and/or ERF922 protein is a dominant negative CPL1 and/or ERF922 protein.
8. The method according to any of statements 5 to 7, wherein said mutated CPL1 protein comprises a mutation in a DXDXT motif.
9. The method according to any of statements 5 to 8, wherein said mutated CPL1 protein comprises a mutation corresponding to D128X in AtCPL4, wherein X is an amino acid different from D.
10. The method according to any of statements 5 to 9, wherein said mutated CPL1 protein comprises a mutation corresponding to D128A in AtCPL4.
11. The method according to any of statements 5 to 10, wherein said mutated CPL1 protein comprises a mutation in the CPL1 protein corresponding to:
   D148X, preferably D148A, when said plant is from the genus Zea, preferably Zea mays;
   D149X, preferably D148A, when said plant is from the genus Zea, preferably Zea mays;
   D162X, preferably D162A, when said plant is from the genus Beta, preferably Beta vulgaris;
   D143X, preferably D143A, when said plant is from the genus Solanum, preferably Solanum tuberosum;
   D141X, preferably D141A, when said plant is from the genus Glycine, preferably Glycine max;
   D149X, preferably D149A, when said plant is from the genus Triticum, preferably Triticum aestivum;
   D147X, preferably D147A, when said plant is from the genus Triticum, preferably Triticum aestivum;
   D149X, preferably D149A, when said plant is from the genus Sorghum, preferably Sorghum bicolor;
   wherein X is an amino acid different from D.
12. The method according to any of statements 1 to 11, wherein the wild type CPL1 or ERF922 protein comprises a sequence which is respectively at least 95% identical, preferably over its entire length, to a sequence of any of SEQ ID NOs: 2-17 or 37-50 or which is encoded by a sequence which at least 95% identical, preferably over its entire length, to a sequence of any of SEQ ID NOs: 19-34, or 52-65.
13. The method according to any of statements 1 to 12, wherein said pathogen is selected from fungi, bacteria, viruses, nematodes, and insects.
14. The method according to any of statements 1 to 13, wherein said pathogen is a biotrophic or hemibiotrophic pathogen.
15. The method according to any of statements 1 to 14, wherein said plant comprises at least two CPL1 and/or ERF922 genes.
16. The method according to any of statements 1 to 15, wherein said plant comprises at least two homeologues or paralogues of CPL1 and/or ERF922.
17. The method according to any of statements 1 to 15, comprising reducing or eliminating the expression, stability, and/or activity of more than one CPL1 and/or more than one ERF922 protein in said plant.
18. The method according to any of statements 15 to 17, wherein said at least two CPL1 proteins are differentially expressed and/or wherein said at least two ERF922 proteins are differentially expressed.
19. The method according to any of statements 1 to 18, wherein said plant is a crop plant.
20. The method according to any of statements 1 to 19, wherein said plant is selected from the family of poaceae.
21. The method according to any of statements 1 to 20, wherein said plant is selected from the subfamily of pooideae.
22. The method according to any of statements 1 to 20, wherein said plant is selected from the genus Zea, Sorghum, Triticum, Hordeum, Secale, Beta, Glycine, or Solanum.
23. The method according to any of statements 1 to 20, wherein said plant is selected from the species Zea mays, Sorghum bicolor, Triticum aestivum, Hordeum vulgare, Secale cereale, Beta vulgaris, Glycine max, or Solanum tuberosum.
24. The method according to any of statements 1 to 23, wherein said CPL1 and/or ERF922 protein and/or gene expression, stability, and/or activity is reduced or eliminated by knocking out a CPL1 and/or ERF922 gene or knocking down said CPL1 and/or ERF922 protein.
25. The method according to any of statements 1 to 24, wherein said CPL1 and/or ERF922 protein and/or gene expression, stability, and/or activity is reduced or eliminated by mutagenesis, RNAi, or gene editing.
26. The method according to any of statements 1 to 24, wherein the method comprises (recombinantly or transgenically) introducing or introgressing into the genome of a plant or plant part a mutation in a CPL1 and/or ERF922 gene or a nucleotide sequence of a gene encoding CPL1 and/or ERF922 having a mutation, preferably a mutation leading to reduced or eliminated expression of the mRNA of the gene and/or the CPL1 and/or ERF922 protein, a mutation leading to a CPL1 and/or ERF922 protein having reduced or eliminated activity upon translation, or a mutation leading to a CPL1 and/or ERF922 protein having reduced stability.
27. The method according to any of statements 1 to 26, wherein the method comprises
(a) (recombinantly or transgenically) introducing or introgressing into a plant or plant part into a nucleotide sequence of a (endogenous (wild type)) gene encoding a CPL1 and/or ERF922 a mutation, preferably a mutation leading to reduced or eliminated expression of the (endogenous (full length)) mRNA of the gene and/or the (endogenous (full length)) CPL1 and/or ERF922 protein, a mutation leading to a CPL1 and/or ERF922 protein having reduced activity upon translation, or a mutation leading to a CPL1 and/or ERF922 protein having reduced stability;
(b) (recombinantly or transgenically) introducing or introgressing into a plant or a plant part an RNAi molecule directed against, targeting, or hybridizing with a nucleotide sequence encoding a CPL1 and/or ERF922 protein, or a polynucleotide sequence encoding an RNAi molecule directed against, targeting, or hybridizing with a nucleotide sequence encoding a CPL1 and/or ERF922 protein, or
(c) (recombinantly or transgenically) introducing or introgressing into a plant or a plant part an RNA-specific or DNA-specific CRISPR/Cas system directed against or targeting a nucleotide sequence encoding a CPL1 and/or ERF922 protein, or one or more polynucleotide sequence(s) encoding said RNA-specific CRISPR/Cas system, or
(d) (recombinantly or transgenically) introducing or introgressing into a plant or a plant part a chemical compound or an antibody altering the activity of a CPL1 and/or ERF922 protein upon interaction with said CPL1 and/or ERF922.
(e) (recombinantly or transgenically) introducing or introgressing into a plant or a plant part a dominant negative CPL1 and/or ERF922 protein or one or more nucleic acid encoding a dominant negative CPL1 and/or ERF922 protein.
(f) optionally, regenerating a plant from the plant part of any of (a) to (d).
28. The method according to any of statements 1 to 27, wherein said plant is transgenic.
29. A plant, plant part, or plant population obtainable by the method according to any of statements 1 to 28, or the progeny thereof.
30. A plant, plant part, or plant population having reduced or eliminated expression, stability, and/or activity of a CPL1 and/or ERF922 protein and/or gene compared to the expression, stability, and/or activity in a plant, plant part, or plant population of the same species without the reduced or eliminated expression, stability, and/or activity of a CPL1 and/or ERF922 protein and/or gene.
31. The plant, plant part, or plant population according to statement 29 or 30, which is mutagenized.
32. The plant, plant part, or plant population according to any of statements 29 to 31, which is transgenic or gene-edited.
33. The plant part according to any of statements 1 to 32, which is a cell, tissue, organ, fruit or seed.
34. The plant, plant part, or plant population according to any of statements 29 to 33, wherein said plant is a crop plant.
35. The plant, plant part, or plant population according to any of statements 29 to 34, wherein said plant is selected from the family of poaceae.
36. The plant, plant part, or plant population according to any of statements 29 to 35, wherein said plant is selected from the subfamily of pooideae.
37. The plant, plant part, or plant population according to any of statements 29 to 34, wherein said plant is selected from the genus Zea, Sorghum, Triticum, Hordeum, Secale, Beta, Glycine, or Solanum.
38. The plant, plant part, or plant population according to any of statements 29 to 34, wherein said plant is selected from the species Zea mays, Sorghum bicolor, Triticum aestivum, Hordeum vulgare, Secale cereale, Beta vulgaris, Glycine max, or Solanum tuberosum.
39. An (isolated) polynucleic acid comprising a sequence which is at least 90% identical, preferably over its entire length, to a sequence of any of SEQ ID NOs: 19-34, or 52-65; or which encodes a polypeptide which is at least 90% identical, preferably over its entire length, to a sequence of any of SEQ ID NOs: 2-17, 80-87, or 37-50.
40. An (isolated) polynucleic acid specifically hybridizing with the polynucleic acid of statement 39, the complement thereof, or the reverse complement thereof.
41. The (isolated) polynucleic acid according to statement 40, wherein said polynucleic acid is a primer or a probe.
42. The (isolated) polynucleic acid according to statement 40, wherein said polynucleic acid is an RNAi polynucleic acid, siRNA, or shRNA.
43. The (isolated) polynucleic acid according to statement 40, wherein said polynucleic acid is a guide RNA.
44. A method for generating a plant or plant part, comprising (a) providing a first plant according to any of statements 29 to 38, (b) crossing said first plant with a second plant, (c) selecting progeny plants having reduced or eliminated expression, stability, and/or activity of a CPL1 and/or ERF922 protein and/or gene compared to the expression, stability, and/or activity in a plant of the same species, and optionally (d) harvesting said plant part from said progeny.
45. Use of the (isolated) polynucleic acid according to any one of statements 39 to 43 for increasing resistance and/or tolerance to a pathogen in a plant, plant part, or plant population or for generating a plant or plant part, or plant population according to 29 to 38.
46. The use according to statement 45, wherein said (isolated) polynucleic acid encodes a polypeptide which is at least 90% identical, preferably over its entire length, to a sequence of any of SEQ ID NOs: 80-87.
47. A plant, plant part, or plant population comprising the polynucleic acid according to any of statements 39 to 43.
48. The plant, plant part, or plant population according to statement 47, wherein said plant, plant part, or plant population is transgenic.
49. The plant, plant part, or plant population according to statement 47, wherein said plant, plant part, or plant population recombinantly expresses said polynucleic acid.
50. The plant part according to any of statements 47 to 49, which is a cell, tissue, organ, fruit or seed.
51. Method for controlling pathogen infestation in a plant (population) comprising
   a) Providing (a) plant(s) according to any of statements 29 to 38 or 47 to 50 or growing from seeds (a) plant(s) according to any of statements 29 to 38 or 47 to 50,
   b) Cultivating the plant(s) of a) under conditions of pathogen infestation. 52. The method according to statement 51, wherein pathogen infestation is reduced. 53. The method according to statement 51 or 52, wherein pathogen symptoms are reduced.
54. The method according to any of statements 51 to 53, wherein said conditions of pathogen infestation comprise the presence of a pathogen.
55. The method or use according to any of statements 45 to 54, wherein said pathogen is selected from fungi, bacteria, viruses, nematodes, and insects.
56. The method or use according to any of statements 45 to 55, wherein said pathogen is a biotrophic or hemibiotrophic pathogen.
57. Use of a method according to anyone of statements 1 to 28 for increasing the yield (potential) of a plant, preferably under conditions of pathogen infestation.
58. The use according to statement 52, wherein the yield is biomass or seed yield.
59. The use according to statement 58, wherein said biomass is whole plant biomass or biomass of a plant part.
60. The use according to statement 59, wherein said plant part is a tissue, organ, fruit, or seed.
61. The use according to statement 59 or 60, wherein said plant part is a harvestable plant part.
62. Method for producing feed or food with reduced amount of fungal or bacterial toxins comprising
A) controlling pathogen infestation in a plant population by the method of any of statements 51 to 56,
B) harvesting plant material from the population, and C) producing feed or food from the harvested plant material.
63. Feed or food with reduced amount of fungal or bacterial toxins obtained by a method according to statement 62.
64. The method or use according to any of statements 45 to 63, wherein said plant is a crop plant.
65. The method or use according to any of statements 45 to 64, wherein said plant is selected from the family of poaceae.
66. The method or use according to any of statements 45 to 65, wherein said plant is selected from the subfamily of pooideae.
67. The method or use according to any of statements 45 to 66, wherein said plant is selected from the genus Zea, Sorghum, Triticum, Hordeum, Secale, Beta, Glycine, or Solanum.
68. The method or use according to any of statements 45 to 67, wherein said plant is selected from the species Zea mays, Sorghum bicolor, Triticum aestivum, Hordeum vulgare, Secale cereale, Beta vulgaris, Glycine max, or Solanum tuberosum.
69. A method for identifying a plant, plant part, or plant population having increased resistance and/or tolerance to a pathogen, comprising screening for and/or identifying a mutation as defined in any of statements 2 to 11.
71. The method according to statement 69, further comprising the step of selecting the plant, plant part, or plant population having a mutation as defined in any of statements 2 to 11.
72. A method for identifying a plant, plant part, or plant population having increased resistance and/or tolerance to a pathogen, comprising screening for and/or identifying reduced or eliminated expression, activity, and/or stability of CPL1 and/or ERF922 protein and/or gene in a plant, plant part, or plant population.
73. The method according to statement 72, further comprising the step of selecting the plant, plant part, or plant population having a reduced or eliminated expression, activity, and/or stability of CPL1 and/or ERF922.

### BRIEF DESCRIPTION OF THE FIGURES

FIG. 1: Sequence alignment between the different protein sequences of the discovered CPL1 genes and comparison to the Arabidopsis AtCPL1 protein sequence.

FIG. 2: Sequence alignment between the different coding sequences of the discovered CPL1 genes and comparison to the Arabidopsis AtCPL1 coding sequence.

FIG. 3: Vector map of the plasmid construct used for maize transformation to silence ZmCPL1 genes.

FIG. 4: Disease scores of Zymoseptoria tritici infected wheat leaves after VIGS mediated downregulation of TaCPL1

FIG. 5: Comparison of amino acid sequences of ERF922-I and ERF922-II from different crops.

FIG. 6: Phylogram of the ERF922-I and ERF922-II genes of different crops.

FIG. 7: Disease scores of Zymoseptoria tritici infected wheat leaves after VIGS mediated downregulation of TaERF922-I on chromosome 3 (TaERF922-3A-I, TaERF922-3B-I, TAERF922-3D-I) and TaERF922-II on chromosome 2 (TaERF922-2A-II, TaERF922-2B-II, TAERF922-2D-II)

FIG. 8: Disease scores of Fusarium graminearum infected wheat heads after VIGS mediated downregulation of TaERF922-I on chromosome 3 (TaERF922-3A-I, TaERF922-3B-I, TAERF922-3D-I) and TaERF922-II on chromosome 2 (TaERF922-2A-II, TaERF922-2B-II, TAERF922-2D-II)

FIG. 9: Vector map of the plasmid construct used for maize transformation to silence ZmERF922 genes.

### DETAILED DESCRIPTION OF THE INVENTION

Before the present system and method of the invention are described, it is to be understood that this invention is not limited to particular systems and methods or combinations described, since such systems and methods and combinations may, of course, vary. It is also to be understood that the terminology used herein is not intended to be limiting, since the scope of the present invention will be limited only by the appended claims.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. It will be appreciated that the terms “comprising”, “comprises” and “comprised of” as used herein comprise the terms “consisting of”, “consists” and “consists of”, as well as the terms “consisting essentially of”, “consists essentially” and “consists essentially of”.

The recitation of numerical ranges by endpoints includes all numbers and fractions subsumed within the respective ranges, as well as the recited endpoints.

The term “about” or “approximately” as used herein when referring to a measurable value such as a parameter, an amount, a temporal duration, and the like, is meant to encompass variations of +/−20% or less, preferably +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Whereas the terms “one or more” or “at least one”, such as one or more or at least one member(s) of a group of members, is clear per se, by means of further exemplification, the term encompasses inter alia a reference to any one of said members, or to any two or more of said members, such as, e.g., any or etc. of said members, and up to all said members.

All references cited in the present specification are hereby incorporated by reference in their entirety. In particular, the teachings of all references herein specifically referred to are incorporated by reference.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

Standard reference works setting forth the general principles of recombinant DNA technology include Molecular Cloning: A Laboratory Manual, 2nd ed., vol. 1-3, ed. Sambrook et al., Cold Spring Harbor Laboratory Press, Cold Spring Harbor, N.Y., 1989; Current Protocols in Molecular Biology, ed. Ausubel et al., Greene Publishing and Wiley-Interscience, New York, 1992 (with periodic updates) (“Ausubel et al. 1992”); the series Methods in Enzymology (Academic Press, Inc.); Innis et al., PCR Protocols: A Guide to Methods and Applications, Academic Press: San Diego, 1990; PCR 2: A Practical Approach (M. J. MacPherson, B. D. Hames and G. R. Taylor eds. (1995); Harlow and Lane, eds. (1988) Antibodies, a Laboratory Manual; and Animal Cell Culture (R. I. Freshney, ed. (1987). General principles of microbiology are set forth, for example, in Davis, B. D. et al., Microbiology, 3rd edition, Harper & Row, publishers, Philadelphia, Pa. (1980).

In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention, and form different embodiments, as would be understood by those in the art. For example, in the appended claims, any of the claimed embodiments can be used in any combination.

In the following detailed description of the invention, reference is made to the accompanying drawings that form a part hereof, and in which are shown by way of illustration only of specific embodiments in which the invention may be practiced. It is to be understood that other embodiments may be utilised and structural or logical changes may be made without departing from the scope of the present invention. The following detailed description, therefore, is not to be taken in a limiting sense, and the scope of the present invention is defined by the appended claims.

Preferred statements (features) and embodiments of this invention are set herein below. Each statements and embodiments of the invention so defined may be combined with any other statement and/or embodiments unless clearly indicated to the contrary. In particular, any feature indicated as being preferred or advantageous may be combined with any other feature or features or statements indicated as being preferred or advantageous.

In an aspect, the invention relates to a method for increasing resistance and/or tolerance to a pathogen and/or for increasing yield (potential) in a plant, plant part, or plant population, comprising reducing or eliminating the expression, stability, and/or activity of a negative regulator of a plant defence or plant pathogen resistance gene in a plant, plant part or plant population.

In one embodiment, the negative regulator is CPL1 and/or ERF922. Thus, in an aspect, the invention relates to a method for increasing resistance and/or tolerance to a pathogen and/or for increasing yield (potential) in a plant, plant part, or plant population, comprising reducing or eliminating the expression, stability, and/or activity of a CPL1 and/or ERF922 protein and/or gene in a plant, plant part, or plant population.

In particular embodiments, the invention encompasses a method for increasing resistance and/or tolerance to a pathogen and/or for increasing yield (potential) in a plant, plant part, or plant population, comprising reducing or eliminating the expression, stability, and/or activity of CPL1 protein and/or gene in a plant, plant part, or plant population.

In an aspect, the invention relates to a method for increasing resistance and/or tolerance to a pathogen and/or for increasing yield (potential) in a plant, plant part, or plant population, comprising reducing or eliminating the expression, stability, and/or activity of an ERF922 protein and/or gene in a plant, plant part, or plant population.

In an aspect, the invention relates to a method for increasing resistance and/or tolerance to a pathogen and/or for increasing yield (potential) in a plant, plant part, or plant population, comprising reducing or eliminating the expression or activity of a CPL1 and/or ERF922 protein and/or gene in a plant, plant part, or plant population.

In an aspect, the invention relates to a method for increasing resistance and/or tolerance to a pathogen and/or for increasing yield (potential) in a plant, plant part, or plant population, comprising reducing or eliminating the expression or activity of a CPL1 protein and/or gene in a plant, plant part, or plant population.

In an aspect, the invention relates to a method for increasing resistance and/or tolerance to a pathogen and/or for increasing yield (potential) in a plant, plant part, or plant population, comprising reducing or eliminating the expression or activity of a ERF922 protein and/or gene in a plant, plant part, or plant population.

In an aspect, the invention relates to a method for increasing resistance and/or tolerance to a pathogen and/or for increasing yield (potential) in a plant, plant part, or plant population, comprising reducing or eliminating the expression of a CPL1 and/or ERF922 protein and/or gene in a plant, plant part, or plant population.

In an aspect, the invention relates to a method for increasing resistance and/or tolerance to a pathogen and/or for increasing yield (potential) in a plant, plant part, or plant population, comprising reducing or eliminating the expression of a CPL1 protein and/or gene in a plant, plant part, or plant population.

In an aspect, the invention relates to a method for increasing resistance and/or tolerance to a pathogen and/or for increasing yield (potential) in a plant, plant part, or plant population, comprising reducing or eliminating the expression of a ERF922 protein and/or gene in a plant, plant part, or plant population.

In an aspect, the invention relates to a method for increasing resistance and/or tolerance to a pathogen and/or for increasing yield (potential) in a plant, plant part, or plant population, comprising reducing or eliminating the activity of a CPL1 and/or ERF922 protein and/or gene in a plant, plant part, or plant population.

In an aspect, the invention relates to a method for increasing resistance and/or tolerance to a pathogen and/or for increasing yield (potential) in a plant, plant part, or plant population, comprising reducing or eliminating the activity of a CPL1 protein and/or gene in a plant, plant part, or plant population.

In an aspect, the invention relates to a method for increasing resistance and/or tolerance to a pathogen and/or for increasing yield (potential) in a plant, plant part, or plant population, comprising reducing or eliminating the activity of a ERF922 protein and/or gene in a plant, plant part, or plant population.

In an aspect, the invention relates to a method for increasing resistance and/or tolerance to a pathogen and/or for increasing yield (potential) in a plant, plant part, or plant population, comprising expressing a CPL1 and/or ERF922 dominant negative protein and/or gene in a plant, plant part, or plant population.

In an aspect, the invention relates to a method for increasing resistance and/or tolerance to a pathogen and/or for increasing yield (potential) in a plant, plant part, or plant population, comprising expressing a CPL1 dominant negative protein and/or gene in a plant, plant part, or plant population.

In an aspect, the invention relates to a method for increasing resistance and/or tolerance to a pathogen and/or for increasing yield (potential) in a plant, plant part, or plant population, comprising expressing a ERF922 dominant negative protein and/or gene in a plant, plant part, or plant population.

In an aspect, the invention relates to a method for generating or obtaining a plant, plant part, or plant population, comprising reducing or eliminating the expression, stability, and/or activity of a negative regulator of a plant defence or plant pathogen resistance gene, more particularly of a CPL1 and/or ERF922 protein and/or gene in a plant, plant part, or plant population.

In an aspect, the invention relates to a method for generating or obtaining a plant, plant part, or plant population, comprising reducing or eliminating the expression, stability, and/or activity of a CPL1 protein and/or gene in a plant, plant part, or plant population.

In an aspect, the invention relates to a method for generating or obtaining a plant, plant part, or plant population, comprising reducing or eliminating the expression, stability, and/or activity of an ERF922 protein and/or gene in a plant, plant part, or plant population.

In an aspect, the invention relates to a method for generating or obtaining a plant, plant part, or plant population, comprising reducing or eliminating the expression or activity of a CPL1 and/or ERF922 protein and/or gene in a plant, plant part, or plant population.

In an aspect, the invention relates to a method for generating or obtaining a plant, plant part, or plant population, comprising reducing or eliminating the expression or activity of a CPL1 protein and/or gene in a plant, plant part, or plant population.

In an aspect, the invention relates to a method for generating or obtaining a plant, plant part, or plant population, comprising reducing or eliminating the expression or activity of an ERF922 protein and/or gene in a plant, plant part, or plant population.

In an aspect, the invention relates to a method for generating or obtaining a plant, plant part, or plant population, comprising reducing or eliminating the expression of a CPL1 and/or ERF922 protein and/or gene in a plant, plant part, or plant population.

In an aspect, the invention relates to a method for generating or obtaining a plant, plant part, or plant population, comprising reducing or eliminating the expression of a CPL1 protein and/or gene in a plant, plant part, or plant population.

In an aspect, the invention relates to a method for generating or obtaining a plant, plant part, or plant population, comprising reducing or eliminating the expression of a ERF922 protein and/or gene in a plant, plant part, or plant population.

In an aspect, the invention relates to a method for generating or obtaining a plant, plant part, or plant population, comprising reducing or eliminating the activity of a CPL1 and/or ERF922 protein and/or gene in a plant, plant part, or plant population.

In an aspect, the invention relates to a method for generating or obtaining a plant, plant part, or plant population, comprising reducing or eliminating the activity of a CPL1 protein and/or gene in a plant, plant part, or plant population.

In an aspect, the invention relates to a method for generating or obtaining a plant, plant part, or plant population, comprising reducing or eliminating the activity of an ERF922 protein and/or gene in a plant, plant part, or plant population.

In an aspect, the invention relates to a method for generating or obtaining a plant, plant part, or plant population, comprising expressing a CPL1 and/or ERF922 dominant negative protein and/or gene in a plant, plant part, or plant population.

In an aspect, the invention relates to a method for generating or obtaining a plant, plant part, or plant population, comprising expressing a CPL1 dominant negative protein and/or gene in a plant, plant part, or plant population.

In an aspect, the invention relates to a method for generating or obtaining a plant, plant part, or plant population, comprising expressing an ERF922 dominant negative protein and/or gene in a plant, plant part, or plant population.

The inventors have observed that in particular embodiments, the plant will comprise more than one CPL1 and/or ERF922 gene. Thus, in certain embodiments, the expression, activity, and/or stability of at least two CPL1 and/or at least two ERF922 proteins is reduced or eliminated.

In certain embodiments, the expression, activity, and/or stability of at least two CPL1 and at least two ERF922 proteins is reduced or eliminated.

In certain embodiments, the expression, activity, and/or stability of at least two CPL1 proteins is reduced or eliminated.

In certain embodiments, the expression, activity, and/or stability of at least two ERF922 proteins is reduced or eliminated.

One or more mutations in one or more genes encoding a negative regulator of a plant defence or plant pathogen resistance gene can result in reduced or eliminated expression of said negative regulator, increasing or inducing pathogen resistance and/or yield in said plant. In certain embodiments, a mutation in a CPL1 and/or ERF922 gene or a nucleotide sequence of a gene encoding CPL1 and/or ERF922 having a mutation, preferably a mutation leading to reduced or eliminated expression of the mRNA of the gene and/or the CPL1 and/or ERF922 protein, a mutation leading to a CPL1 and/or ERF922 protein having reduced or eliminated activity upon translation (including a dominant negative mutation), or a mutation leading to a CPL1 and/or ERF922 protein having reduced stability is (recombinantly or transgenically) introduced or introgressed into the genome of a plant or plant part. Different methods are envisaged for ensuring one or more mutations in one or more genes encoding a negative regulator of a plant defence or plant pathogen resistance gene. For instance, the mutations can be introduced by recombinant technology or can be introduced by introgression from another plant (carrying the one or more envisaged mutations).

In certain embodiments, the methods of the invention as described herein comprise:
(a) (recombinantly or transgenically) introducing or introgressing in a plant or plant part into a nucleotide sequence of a (endogenous (wild type)) gene encoding a CPL1 and/or ERF922 a mutation, preferably a mutation leading to reduced or eliminated expression of the (endogenous (full length)) mRNA of the gene and/or the (endogenous (full length)) CPL1 and/or ERF922 protein, a mutation leading to a CPL1 and/or ERF922 protein having reduced activity upon translation, or a mutation leading to a CPL1 and/or ERF922 protein having reduced stability;
(b) (recombinantly or transgenically) introducing or introgressing in a plant or a plant part one or more RNAi molecule directed against, targeting, or hybridizing with a nucleotide sequence encoding a CPL1 and/or a nucleotide sequence encoding a ERF922 protein, or a polynucleotide sequence encoding one or more RNAi molecule directed against, targeting, or hybridizing with a nucleotide sequence encoding a CPL1 and/or a nucleotide sequence encoding a ERF922 protein;
(c) (recombinantly or transgenically) introducing or introgressing in a plant or a plant part one or more RNA-specific or DNA-specific CRISPR/Cas system directed against or targeting a nucleotide sequence encoding a CPL1 and/or a nucleotide sequence encoding a ERF922 protein, or one or more polynucleotide sequence(s) encoding said RNA-specific or DNA-specific CRISPR/Cas system;
(d) (recombinantly or transgenically) introducing or introgressing into a plant or a plant part a chemical compound or an antibody (or polynucleic acid encoding such) altering the activity of a CPL1 and/or ERF922 protein upon interaction with said CPL1 and/or ERF922; or
(e) (recombinantly or transgenically) introducing or introgressing into a plant or a plant part a dominant negative CPL1 and/or ERF922 protein or one or more nucleic acid encoding a dominant negative CPL1 and/or ERF922 protein;
(f) optionally, regenerating a plant from the plant part of any of (a) to (d).

The methods described herein will result in a plant, plant part or plant population comprising one or more mutations in one or more genes encoding a negative regulator of a plant defence or plant pathogen resistance gene resulting in reduced or eliminated expression of said negative regulator in said plant, plant part or plant population, resulting in increased or induced pathogen resistance and/or yield in said plant, plant part or plant population. Thus, the invention provides plants, plant parts and plant populations having increased or induced pathogen resistance and/or yield.

Thus, in an aspect, the invention relates to a plant, plant part, or plant population obtained by any of the methods described above. In an aspect, the invention relates to a plant obtained by any of the methods described above. In an aspect, the invention relates to plant part obtained by any of the methods described above. In an aspect, the invention relates to a plant population obtained by any of the methods described above.

In an aspect, the invention relates to a plant, plant part, or plant population having reduced or eliminated expression, stability, and/or activity of a CPL1 and/or ERF922 protein and/or gene (such as compared to the expression, stability, and/or activity in a wild type plant, plant part, or plant population of the same species (or line or genotype) or a plant, plant part, or plant population of the same species (or line or genotype) not having the reduced or eliminated expression, stability, and/or activity of a CPL1 and/or ERF922 protein and/or gene.

In an aspect, the invention relates to a plant, plant part, or plant population comprising one or more polynucleotide sequence of a gene encoding a CPL1 and/or ERF922 having a mutation, preferably a mutation leading to reduced or absent expression of the mRNA and/or protein, or a mutation leading to a truncated or non-functional protein upon translation.

In an aspect, the invention relates to a plant, plant part, or plant population comprising one or more nucleotide sequence of a gene encoding a CPL1 and/or ERF922 having reduced or eliminated mRNA and/or protein expression.

The invention further provides plants, plant parts and plant populations comprising a modified sequence of one or more genes encoding a negative regulator of a plant defence gene or a plant pathogen resistance gene. In an aspect, the invention relates to a plant comprising:
(a) a nucleotide sequence of a (endogenous (wild type)) gene encoding a CPL1 and/or ERF922 a mutation, preferably a mutation leading to reduced or eliminated expression of the (endogenous (full length)) mRNA of the gene and/or the (endogenous (full length)) CPL1 and/or ERF922 protein, a mutation leading to a CPL1 and/or ERF922 protein having reduced activity upon translation, or a mutation leading to a CPL1 and/or ERF922 protein having reduced stability;
(b) one or more RNAi molecule directed against, targeting, or hybridizing with a nucleotide sequence encoding a CPL1 and/or a nucleotide sequence encoding a ERF922 protein, or a polynucleotide sequence encoding one or more RNAi molecule directed against, targeting, or hybridizing with a nucleotide sequence encoding a CPL1 and/or a nucleotide sequence encoding a ERF922 protein;
(c) one or more RNA-specific or DNA-specific CRISPR/Cas system directed against or targeting a nucleotide sequence encoding a CPL1 and/or a nucleotide sequence encoding a ERF922 protein, or one or more polynucleotide sequence(s) encoding said RNA-specific or DNA-specific CRISPR/Cas system;
(d) a chemical compound or an antibody (or polynucleic acid encoding such) altering the activity of a CPL1 and/or ERF922 protein upon interaction with said CPL1 and/or ERF922; or
(e) a dominant negative CPL1 and/or ERF922 protein or one or more nucleic acid encoding a dominant negative CPL1 and/or ERF922 protein.

In particular embodiments, plants comprising a knockout and/or knockdown of one or more negative regulators of a plant pathogen resistance gene or a pathogen defence gene are provided. Alternatively, the mutation in the negative regulator is a dominant negative mutation, such that the gene product negatively affects the function of the wildtype negative regulator. In an aspect, the invention relates to a plant comprising a knockout mutation of CPL1 and/or ERF922. In an aspect, the invention relates to a plant comprising a knockout mutation of CPL1 and ERF922. In an aspect, the invention relates to a plant comprising a knockout mutation of CPL1. In an aspect, the invention relates to a plant comprising a knockout mutation of ERF922. In certain embodiments, the mutation is homozygous. In certain embodiments, the mutation is heterozygous.

In an aspect, the invention relates to a plant comprising a knockdown mutation of CPL1 and/or ERF922. In an aspect, the invention relates to a plant comprising a knockdown mutation of CPL1 and ERF922. In an aspect, the invention relates to a plant comprising a knockdown mutation of CPL1. In an aspect, the invention relates to a plant comprising a knockdown mutation of ERF922. In certain embodiments, the mutation is homozygous. In certain embodiments, the mutation is heterozygous.

In an aspect, the invention relates to a plant expressing a dominant negative CPL1 and/or ERF922 protein. In an aspect, the invention relates to a plant expressing a dominant negative CPL1 and ERF922 protein. In an aspect, the invention relates to a plant expressing a dominant negative CPL1. In an aspect, the invention relates to a plant expressing a dominant negative ERF922 protein. In certain embodiments, the mutation is homozygous. In certain embodiments, the mutation is heterozygous.

The term “plant” includes whole plants, including descendants or progeny thereof. The term “plant part” includes any part or derivative of the plant, including particular plant tissues or structures, plant cells, plant protoplast, plant cell or tissue culture from which plants can be regenerated, plant calli, plant clumps and plant cells that are intact in plants or parts of plants, such as seeds, kernels, cobs, fruits, flowers, cotyledons, leaves, stems, buds, roots, root tips, stover, and the like. Plant parts may include processed plant parts or derivatives, including flower, oils, extracts etc. In certain embodiments, the plant part or derivative as referred to herein is a cell, tissue, or seed (or grain). In certain embodiments, the plant part or derivative as referred to herein is a seed (or grain). In certain embodiments, the plant part is a harvestable plant part, such as seeds or grains, fruits, roots, leaves, stalks, flowers, tubers, bulbs, cobs, etc.

In certain embodiments, the plant is a crop plant, such as a cash crop or substinence crop, such as food or non-food crops, including agriculture, horticulture, floriculture, or industrial crops. The term crop plant has its ordinary meaning as known in the art. By means of further guidance, and without limitation, a crop plant is a plant grown by humans for food and other resources, and can be grown and harvested extensively for profit or subsistence, typically in an agricultural setting or context.

In certain embodiments, the plant is from the family of Poaceae. As used herein, the term Poaceae refers to the family of grasses, or Gramineae. Preferably, the Poaceae are cereals (or cereal grasses), which are in particular cultivated for the edible components of its grain.

In certain embodiments, the plant is from the subfamily of Pooideae. As used herein, the term Pooideae refers to the subfamily of Poaceae in the Poaceae family. Preferably, the Pooideae are cereals (or cereal grasses), which are in particular cultivated for the edible components of its grain. In certain embodiments, the plant is from the genus Zea, preferably Zea mays. In certain embodiments, the plant is from the genus Sorghum, preferably Sorghum bicolor. In certain embodiments, the plant is from the genus Triticum, preferably Triticum aestivum. In certain embodiments, the plant is from the genus Hordeum, preferably Hordeum vulgare. In certain embodiments, the plant is from the genus Secale, preferably Secale cereale. In certain embodiments, the plant is from the genus Beta, preferably Beta vulgaris. In certain embodiments, the plant is from the genus Glycine, preferably Glycine max. In certain embodiments, the plant is from the genus Solanum, preferably Solanum tuberosum. In certain embodiments, the plant from the genus Oryza, preferably Oryza sativa.

In certain embodiments, the plant part is or comprises propagation material. In certain embodiments, the plant part or derivative is not (functional) propagation material, such as germplasm, a seed, or plant embryo or other material from which a plant can be regenerated. In certain embodiments, the plant part or derivative does not comprise (functional) male and female reproductive organs. In certain embodiments, the plant part or derivative is or comprises propagation material, but propagation material which does not or cannot be used (anymore) to produce or generate new plants, such as propagation material which have been chemically, mechanically or otherwise rendered non-functional, for instance by heat treatment, acid treatment, compaction, crushing, chopping, etc.

As used herein, the term “plant population” may be used interchangeably with population of plants. A plant population preferably comprises a multitude of individual plants, such as preferably at least 10, such as 20, 30, 40, 50, 60, 70, 80, or 90, more preferably at least 100, such as 200, 300, 400, 500, 600, 700, 800, or 900, even more preferably at least 1000, such as at least 10000 or at least 100000.

As used herein the terms “increased pathogen tolerance” and “increased pathogen resistance” relate to any relief from, reduced presentation of, improvement of, or any combination thereof of any symptom (such as damage or loss in biomass) of an infection by a pathogen. Increased pathogen resistance or tolerance as referred to herein may also relate to the ability to which a plant maintains for instance its biomass production (such as harvestable biomass production, such as seed yield) upon or during pathogen infection. A pathogen resistant or tolerant plant, plant cell or plant part may refer herein to a plant, plant cell or plant part, respectively, having increased resistance/tolerance to a pathogen compared to a parent plant from which they are derived (and not having reduced or eliminated expression, stability, and/or activity of a CPL1 and/or ERF922 protein and/or gene). Resistance may relate herein to the plant's ability to limit pathogen multiplication. Tolerance may relate herein to a plant's ability to reduce the effect of infection on its fitness regardless of the level of pathogen multiplication. Methods of determining pathogen resistance/tolerance are known to the person of skill in the art, such as visual scoring of pathogen infection or pathogen-induced damage, determination of biomass (yield), etc. As used herein, the terms “increased pathogen tolerance” and “increased pathogen resistance” may be used interchangeably with “reduced sensitivity” or “reduced susceptibility” towards pathogens. Accordingly, a plant, plant part, or plant population according to the invention which is more resistant or more tolerant towards a pathogen is considered less sensitive toward such pathogen. Less sensitive or less susceptible when used herein may be seen as “more tolerant” or “more resistant”. Similarly, “more tolerant” or “more resistant” may, vice versa, be seen as “less sensitive” or “less susceptible”. More sensitive or more susceptible when used herein may, vice versa, be seen as “less tolerant” or “less resistant”. Similarly, “less tolerant” or “less resistant” may, vice versa, be seen as “more sensitive” or “more susceptible”.

The plants, plant parts or plant populations as described herein having increased pathogen resistance or tolerance can be used to control pathogen infestation or infection. Accordingly, in an aspect, the invention relates to the use of such plants for controlling pathogen infestation or infection. Preferably pathogen infestation or infection is controlled by reduction of pathogen infestation or infection or as reduction in the symptoms of pathogen infestation or infection, at the plant, plant part, or plant population level, such as further described below.

In certain embodiments, an increased resistance or tolerance may present itself as a reduction of infection or infestation (e.g. the amount of pathogens (e.g. per plant area or per plant biomass), the multiplication (rate) or spread (rate)/distribution of pathogens, as well as the speed of pathogen spreading such as at a specific time during the (growth) season) at the (sub) plant level (such as for instance particular cells, organs, or tissues, for instance harvestable parts of a plant or for instance leaves, stalks, fruits, or seeds) or at the population level, such as a reduction of at least 5%, preferably at least 10%, such as at least 20%, at least 30%, at least 40%, at least 50%, at least 60%, at least 70%, at least 80%, at least 90%, or (about) 100%. At the population level, an increased resistance or tolerance may present itself as a reduction of infection or infestation as described above, but also for instance as a reduction in the amount of infected plants (or a combination), such as a reduction of at least 5%, preferably at least 10%, such as at least 20%, at least 30%, at least 40%, at least 50%, at least 60%, at least 70%, at least 80%, at least 90%, or (about) 100%. It will be understood that such reduction of infection or infestation can be relative to a reference plant (part) or population (such as a corresponding wild type plant not according to the invention).

In certain embodiments, an increased resistance or tolerance may present itself as a reduction in the loss of biomass or yield in general or of a particular (harvestable) plant part (such as seed or fruit amount or weight) due to or as a consequence of pathogen infection. In certain embodiments, the resistant or tolerant plants exhibit a loss in biomass production (such as expressed in g/day or kg/ha or kg/ha/day, such as expressed as dry matter for instance expressed as weight percent) under pathogen infection which is at least 1%, preferably at least 2%, such as at least 3%, at least 4%, at least 5%, such as at least 10%, at least 15%, or at least 20% or more, lower than corresponding control plants, such as plants which are less resistant or tolerant, or plants not according to the invention as described herein. In certain embodiments, the resistant or tolerant plants exhibit biomass production (such as expressed in g/day or kg/ha or kg/ha/day, such as expressed as dry matter for instance expressed as weight percent) under pathogen infection which is at least 1%, preferably at least 2%, such as at least 3%, at least 4%, at least 5%, such as at least 10%, at least 15%, or at least 20% or more, higher than corresponding control plants, such as plants which are less resistant or tolerant, or plants not according to the invention as described herein.

As used herein, the term “yield potential” refers to maximum yield obtainable at harvest.

The term “pathogen” as used herein generally refers to any type of infectious agent capable of causing an (infectious) disease, and includes without limitation a virus, bacterium, protozoan, prion, viroid, or fungus (including yeasts). Also parasites, such as insects or worms, but also parasitic plants or algae are generally encompassed by the term pathogen as used herein.

In certain embodiments, the pathogen is a biotrophic pathogen. In certain embodiments, the pathogen is a hemibiotrophic pathogen. In certain embodiments, the pathogen is not a necrotrophic pathogen.

Biotrophs derive energy from living cells; they are found on or in living plants, can have very complex nutrient requirements and do not kill host plants (rapidly). In contrast, necrotrophs derive energy from killed cells; they invade and kill plant tissue rapidly and then live saprotrophically on the dead remains. Hemibiotrophs have an initial period of biotrophy followed by necrotrophy.

As used herein, reduced (protein and/or gene/mRNA) expression levels may refer to decreased expression levels of about at least 10%, preferably at least 30%, more preferably at least 50%, such as at least 20%, 40%, 60%, 80% or more, such as at least 85%, at least 90%, at least 95%, or more. Expression is (substantially) absent or eliminated if expression levels are reduced at least 80%, preferably at least 90%, more preferably at least 95%. In certain embodiments, expression is (substantially) absent, if no protein and/or mRNA, in particular the wild type or native protein and/or mRNA, can be detected. In certain embodiments, protein and/or gene/mRNA expression levels are reduced between 20 and 80%, such as between 30 and 70% or between 40 and 60%, such as (about) 50%, such as compared to the expression level in a reference plant, which may be a wild type plant, or a plant not comprising a mutated CPL1 and/or ERF822 or any of the (genetic) events leading to reduced expression, as described herein elsewhere. Expression levels can be determined by any means known in the art, such as by standard detection methods, including for instance (quantitative) PCR, northern blot, western blot, ELISA, etc.

Reduced expression levels may result from increased turnover of mRNA or protein, such as increased breakdown or decreased stability. Reduced expression levels may result from reduced expression rate or reduced transcription rate. Reduced expression levels may result from reduced copy number (e.g. heterozygous wild type and mutant CPL1 and/or ERF922).

As used herein, reduced expression rate may refer to a reduction in the expression rate of a nucleotide sequence by more than 10%, 15%, 20%, 25% or 30%, preferably by more than 40%, 45%, 50%, 55%, 60% or 65%, more preferably by more than 70%, 75%, 80%, 85%, 90%, 92%, 94%, 96% or 98% in comparison to the specified reference, such as a plant not comprising the genetic or otherwise modifications according to the invention as described herein elsewhere, or a reference plant, such as a wild type plant. However, it may also mean that the expression rate of a nucleotide sequence is reduced by 100%. The reduction in the expression rate preferably leads to a change of the phenotype of a plant in which the expression rate is reduced.

As used herein, reduced transcription rate may refer to a reduction in the transcription rate of a nucleotide sequence by more than 10%, 15%, 20%, 25% or 30%, preferably by more than 40%, 45%, 50%, 55%, 60% or 65%, more preferably by more than 70%, 75%, 80%, 85%, 90%, 92%, 94%, 96% or 98% in comparison to the specified reference, such as a plant not comprising the genetic or otherwise modifications according to the invention as described herein elsewhere, or a reference plant, such as a wild type plant. However, it may also mean that the transcription rate of a nucleotide sequence is reduced by 100%. The reduction in the transcription rate preferably leads to a change of the phenotype of a plant in which the transcription rate is reduced.

As used herein, reduced (protein) activity may refer to decreased activity of about at least 10%, preferably at least 30%, more preferably at least 50%, such as at least 20%, 40%, 60%, 80% or more, such as at least 85%, at least 90%, at least 95%, or more. Activity is (substantially) absent or eliminated if activity is reduced at least 80%, preferably at least 90%, more preferably at least 95%. In certain embodiments, activity is (substantially) absent, if no activity, in particular the wild type or native protein activity, can be detected. (Protein) activity levels can be determined by any means known in the art, depending on the type of protein, such as by standard detection methods, including for instance enzymatic assays (for enzymes), transcription assays (for transcription factors), assays to analyse a phenotypic output, etc.

As used herein, reduced stability may refer to reduced protein stability or reduced RNA, such as mRNA stability. Stability of proteins or RNA can be determined by means known in the art, such as determination of protein/RNA half-life. Reduced protein or RNA stability in certain embodiments means a reduction of stability of about at least 10%, preferably at least 30%, more preferably at least 50%, such as at least 20%, 40%, 60%, 80% or more, such as at least 85%, at least 90%, at least 95. Reduced protein or RNA stability in certain embodiments means a reduction in half-life of about at least 10%, preferably at least 30%, more preferably at least 50%, such as at least 20%, 40%, 60%, 80% or more, such as at least 85%, at least 90%, at least 95%, such as a two-fold, three-fold, four-fold, 5-fold or more decrease in half-life. Stability may be compared to a reference as defined above.

Expression levels, stability, or activity may be compared between different plants (or plant parts), such as a plant (part) having reduced CLP1 and/or ERF922 expression, stability, or activity according to the invention and a reference plant (part), such as a wild type plant (part). Expression levels, stability, or activity may be compared between different conditions. Expression levels, stability, or activity may be compared with a predetermined threshold. Such predetermined threshold may for instance correspond to expression levels, stability, or activity in a particular genotype or under particular conditions.

In certain embodiments, the CPL1 and/or ERF922 protein and/or gene expression, stability, and/or activity is reduced or eliminated by knocking out a CPL1 and/or ERF922 gene or knocking down said CPL1 and/or ERF922 protein, as described herein elsewhere. In certain embodiments, the CPL1 and/or ERF922 protein and/or gene expression, stability, and/or activity is reduced or eliminated by mutagenesis, RNAi, or gene editing, as described herein elsewhere.

It will be understood that reduced expression, activity, or stability preferably refers to reduced expression, activity, or stability of the wild type (functional) CPL1 or ERF922. For instance, a knockout generated by genomic insertion or creation of a premature stop codon may lead to expression of a truncated mRNA/protein of which the expression levels are similar to the expression levels of the unmutated mRNA/protein. However, the resulting protein has affected functionality and hence expression (and activity in this case) are thus considered reduced. Similarly, introduction of a dominant negative variant of a protein may not impact expression of the endogenous wild type (functional) protein. Nevertheless, at least wild type (functional) protein activity is reduced to the presence of the dominant negative variant.

As used herein, the term “dominant negative” has its ordinary meaning known in the art. By means of further guidance, and without limitation, a dominant negative protein comprises a mutation whose gene product adversely affects the normal, wild-type gene product within the same cell. This usually occurs if the product can still interact with the same elements as the wild-type product, but block some aspect of its function, for example: a mutation in a transcription factor that removes the activation domain, but still contains the DNA binding domain (this product can then block the wild-type transcription factor from binding the DNA site leading to reduced levels of gene activation); a mutation in an enzyme that abolishes catalytic activity (this product can then block the wild-type by binding and hence tittering out the substrate without catalytic conversion); a protein that is functional as a dimer (a mutation that removes the functional domain, but retains the dimerization domain would cause a dominate negative phenotype, because some fraction of protein dimers would be missing one of the functional domains); etc.

Reduction of expression, activity, and/or stability as described herein may be constitutive or conditional, such as inducible and/or tissue-specific (e.g. in reproductive organs). Means for conditional, such as inducible or tissue-specific manipulation are well known in the art.

When used herein, the term “polypeptide” or “protein” (both terms are used interchangeably herein) means a peptide, a protein, or a polypeptide which encompasses amino acid chains of a given length, wherein the amino acid residues are linked by covalent peptide bonds. However, peptidomimetics of such proteins/polypeptides wherein amino acid(s) and/or peptide bond(s) have been replaced by functional analogs are also encompassed by the invention as well as other than the 20 gene-encoded amino acids, such as selenocysteine. Peptides, oligopeptides and proteins may be termed polypeptides. The term polypeptide also refers to, and does not exclude, modifications of the polypeptide, e.g., glycosylation, acetylation, phosphorylation and the like. Such modifications are well described in basic texts and in more detailed monographs, as well as in the research literature.

Amino acid substitutions encompass amino acid alterations in which an amino acid is replaced with a different naturally-occurring amino acid residue. Such substitutions may be classified as “conservative<1>, in which an amino acid residue contained in the wild-type protein is replaced with another naturally-occurring amino acid of similar character, for example Gly< >Ala, Val< >Ile< >Leu, Asp< >Glu, Lys< >Arg, Asn< >Gln or Phe< >Trp< >Tyr.

Substitutions encompassed by the present invention may also be “non-conservative”, in which an amino acid residue which is present in the wild-type protein is substituted with an amino acid with different properties, such as a naturally-occurring amino acid from a different group (e.g. substituting a charged or hydrophobic amino acid with alanine. “Similar amino acids”, as used herein, refers to amino acids that have similar amino acid side chains, i.e. amino acids that have polar, non-polar or practically neutral side chains. “Non-similar amino acids”, as used herein, refers to amino acids that have different amino acid side chains, for example an amino acid with a polar side chain is non-similar to an amino acid with a non-polar side chain. Polar side chains usually tend to be present on the surface of a protein where they can interact with the aqueous environment found in cells (“hydrophilic” amino acids). On the other hand, “non-polar” amino acids tend to reside within the center of the protein where they can interact with similar non-polar neighbours (“hydrophobic” amino acids”). Examples of amino acids that have polar side chains are arginine, asparagine, aspartate, cysteine, glutamine, glutamate, histidine, lysine, serine, and threonine (all hydrophilic, except for cysteine which is hydrophobic). Examples of amino acids that have non-polar side chains are alanine, Glycine, isoleucine, leucine, methionine, phenylalanine, proline, and tryptophan (all hydrophobic, except for Glycine which is neutral).

The term “gene” when used herein refers to a polymeric form of nucleotides of any length, either ribonucleotides or desoxyribonucleotides. The term includes double- and single-stranded DNA and RNA. It also includes known types of modifications, for example, methylation, “caps”, substitutions of one or more of the naturally occurring nucleotides with an analog. Preferably, a gene comprises a coding sequence encoding the herein defined polypeptide. A “coding sequence” is a nucleotide sequence which is transcribed into mRNA and/or translated into a polypeptide when placed or being under the control of appropriate regulatory sequences. The boundaries of the coding sequence are determined by a translation start codon at the 5′-terminus and a translation stop codon at the 3′-terminus. A coding sequence can include, but is not limited to mRNA, cDNA, recombinant nucleic acid sequences or genomic DNA, while introns may be present as well under certain circumstances.

A used herein, the term “endogenous” refers to a gene or allele which is present in its natural genomic location. The term “endogenous” can be used interchangeably with “native”. This does not however exclude the presence of one or more nucleic acid differences with the wild-type allele. In particular embodiments, the difference with a wild-type allele can be limited to less than 9 preferably less than 6, more particularly less than 3 nucleotide differences. More particularly, the difference with the wildtype sequence can be in only one nucleotide.

Preferably, the endogenous allele encodes a modified protein having less than 9, preferably less than 6, more particularly less than 3 and even more preferably only one amino acid difference with the wild-type protein. In certain embodiments, the endogenous gene or allele is the wild type gene or allele. In certain embodiments, the endogenous gene is mutagenized, as described herein elsewhere. In certain embodiments, expression, activity, and/or stability of the endogenous gene is modified, as described herein elsewhere.

As used herein, the term “homozygote” refers to an individual cell or plant having the same alleles at one or more or all loci. When the term is used with reference to a specific locus or gene, it means at least that locus or gene has the same alleles. As used herein, the term “homozygous” means a genetic condition existing when identical alleles reside at corresponding loci on homologous chromosomes. As used herein, the term “heterozygote” refers to an individual cell or plant having different alleles at one or more or all loci. When the term is used with reference to a specific locus or gene, it means at least that locus or gene has different alleles. As used herein, the term “heterozygous” means a genetic condition existing when different alleles reside at corresponding loci on homologous chromosomes. In certain embodiments, the CPL1 and/or ERF922 gene as described herein is homozygous (e.g. mutated CPL1 and/or ERF922 on all homologous or homeologous chromosomes). In certain embodiments, the CPL1 and/or ERF922 gene as described herein is heterozygous (e.g. (at least) one mutated CPL1 and/or ERF922 and (at least) one wild type CPL1 and/or ERF922 on homologous or homeologous chromosomes).

A “polymorphism” is a variation in the DNA between two or more individuals within a population. A polymorphism preferably has a frequency of at least 1% in a population. A useful polymorphism can include a single nucleotide polymorphism (SNP), a simple sequence repeat (SSR), or an insertion/deletion polymorphism, also referred to herein as an “indel”. The term “indel” refers to an insertion or deletion, wherein one line may be referred to as having an inserted nucleotide or piece of DNA relative to a second line, or the second line may be referred to as having a deleted nucleotide or piece of DNA relative to the first line.

The term “sequence” when used herein relates to nucleotide sequence(s), polynucleotide(s), nucleic acid sequence(s), nucleic acid(s), nucleic acid molecule, peptides, polypeptides and proteins, depending on the context in which the term “sequence” is used. The terms “nucleotide sequence(s)”, “polynucleotide(s)”, “nucleic acid sequence(s)”, “nucleic acid(s)”, “nucleic acid molecule” are used interchangeably herein and refer to nucleotides, either ribonucleotides or deoxyribonucleotides or a combination of both, in a polymeric unbranched form of any length. Nucleic acid sequences include DNA, cDNA, genomic DNA, RNA, synthetic forms and mixed polymers, both sense and antisense strands, or may contain non-natural or derivatized nucleotide bases, as will be readily appreciated by those skilled in the art.

As used herein, the term “sequence identity” refers to the degree of identity between any given nucleic acid sequence and a target nucleic acid sequence. Percent sequence identity is calculated by determining the number of matched positions in aligned nucleic acid sequences, dividing the number of matched positions by the total number of aligned nucleotides, and multiplying by 100. A matched position refers to a position in which identical nucleotides occur at the same position in aligned nucleic acid sequences. Percent sequence identity also can be determined for any amino acid sequence. To determine percent sequence identity, a target nucleic acid or amino acid sequence is compared to the identified nucleic acid or amino acid sequence using the BLAST 2 Sequences (Bl2seq) program from the stand-alone version of BLASTZ containing BLASTN and BLASTP. This stand-alone version of BLASTZ can be obtained from Fish & Richardson's web site (World Wide Web at fr.com/blast) or the U.S. government's National Center for Biotechnology Information web site (World Wide Web at ncbi.nlm.nih.gov). Instructions explaining how to use the Bl2seq program can be found in the readme file accompanying BLASTZ. Bl2seq performs a comparison between two sequences using either the BLASTN or BLASTP algorithm.

BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq l .txt); -j is set to a file containing the second nucleic acid sequence to be compared (e.g., C:\seq2.txt); -p is set to blastn; -o is set to any desired file name (e.g., C :\output.txt); -q is set to—1; -r is set to 2; and all other options are left at their default setting. The following command will generate an output file containing a comparison between two sequences: C:\B12seq -i c:\seql.txt -j c:\seq2.txt -p blastn -o c:\output.txt -q—1 -r 2. If the target sequence shares homology with any portion of the identified sequence, then the designated output file will present those regions of homology as aligned sequences. If the target sequence does not share homology with any portion of the identified sequence, then the designated output file will not present aligned sequences. Once aligned, a length is determined by counting the number of consecutive nucleotides from the target sequence presented in alignment with the sequence from the identified sequence starting with any matched position and ending with any other matched position. A matched position is any position where an identical nucleotide is presented in both the target and identified sequences. Gaps presented in the target sequence are not counted since gaps are not nucleotides. Likewise, gaps presented in the identified sequence are not counted since target sequence nucleotides are counted, not nucleotides from the identified sequence. The percent identity over a particular length is determined by counting the number of matched positions over that length and dividing that number by the length followed by multiplying the resulting value by 100. For example, if (i) a 500-base nucleic acid target sequence is compared to a subject nucleic acid sequence, (ii) the Bl2seq program presents 200 bases from the target sequence aligned with a region of the subject sequence where the first and last bases of that 200-base region are matches, and (iii) the number of matches over those 200 aligned bases is 180, then the 500-base nucleic acid target sequence contains a length of 200 and a sequence identity over that length of 90% (i.e., 180/200×100=90). It will be appreciated that different regions within a single nucleic acid target sequence that aligns with an identified sequence can each have their own percent identity. It is noted that the percent identity value is rounded to the nearest tenth. For example, 78.11, 78.12, 78.13, and 78.14 are rounded down to 78.1, while 78.15, 78.16, 78.17, 78.18, and 78.19 are rounded up to 78.2. It also is noted that the length value will always be an integer.

An “isolated nucleic acid” is understood to be a nucleic acid isolated from its natural or original environment. The term also includes a synthetic manufactured nucleic acid. An “isolated nucleic acid sequence” or “isolated DNA” refers to a nucleic acid sequence which is no longer in the natural environment from which it was isolated, e.g. the nucleic acid sequence in a bacterial host cell or in the plant nuclear or plastid genome. When referring to a “sequence” herein, it is understood that the molecule having such a sequence is referred to, e.g. the nucleic acid molecule. A “host cell” or a “recombinant host cell” or “transformed cell” are terms referring to a new individual cell (or organism) arising as a result of at least one nucleic acid molecule, having been introduced into said cell. The host cell is preferably a plant cell or a bacterial cell. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

When reference is made to a nucleic acid sequence (e.g. DNA or genomic DNA) having “substantial sequence identity to” a reference sequence or having a sequence identity of at least 80%>, e.g. at least 85%, 90%, 95%, 98%> or 99%>nucleic acid sequence identity to a reference sequence, in one embodiment said nucleotide sequence is considered substantially identical to the given nucleotide sequence and can be identified using stringent hybridisation conditions. In another embodiment, the nucleic acid sequence comprises one or more mutations compared to the given nucleotide sequence but still can be identified using stringent hybridisation conditions. “Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

“Stringent hybridisation conditions” can be used to identify nucleotide sequences, which are substantially identical to a given nucleotide sequence. Stringent conditions are sequence dependent and will be different in different circumstances. Generally, stringent conditions are selected to be about 5° C. lower than the thermal melting point (Tm) for the specific sequences at a defined ionic strength and pH. The Tm is the temperature (under defined ionic strength and pH) at which 50% of the target sequence hybridises to a perfectly matched probe. Typically stringent conditions will be chosen in which the salt concentration is about 0.02 molar at pH 7 and the temperature is at least 60° C. Lowering the salt concentration and/or increasing the temperature increases stringency. Stringent conditions for RNA-DNA hybridisations (Northern blots using a probe of e.g. 100 nt) are for example those which include at least one wash in 0.2×SSC at 63° C. for 20 min, or equivalent conditions. Stringent conditions for DNA-DNA hybridisation (Southern blots using a probe of e.g. 100 nt) are for example those which include at least one wash (usually 2) in 0.2×SSC at a temperature of at least 50° C., usually about 55° C., for 20 min, or equivalent conditions. See also Sambrook et al. (1989) and Sambrook and Russell (2001).

A “functional fragment” of a nucleotide sequence as used herein means a segment of a nucleotide sequence which has the functionality identical or comparable to the complete nucleotide sequence from which the functional fragment originates. As such, the functional fragment may possess a nucleotide sequence which is identical or homologous to the complete nucleotide sequence over a length of at least 50%, 55%, 60%, 65%, 70%, 75%, 80%, 85%, 90%, 92%, 94% 96%, 97%, 98% or 99%. Furthermore, a “functional fragment” of a nucleotide sequence may also mean a segment of a nucleotide sequence which alters the functionality of the total nucleotide sequence, e.g., in the course of post-transcriptional gene silencing. As such, the functional fragment of a nucleotide sequence may include at least 14, 15, 16, 17, 18, 19, 20, 21, 22, 23, 24 or 25, preferably at least 30, 35, 40, 45, 50, 60, 70, 80, 90, 100, 120 or 140, more preferably at least 160, 180, 200, 250, 300, 350, 400, 450, 500, 600, 700, 800, 900 or 1000 successive nucleotides of the complete nucleotide sequence.

A “functional part” of a protein means a segment of a protein, or a section of the amino acid sequence, that encodes for the protein, wherein the segment may exert functionality identical or comparable to the entire protein in a plant cell. A functional part of a protein has, over a length of at least 50%, 55%, 60%, 65%, 70%, 75%, 80%, 85%, 90%, 92%, 94%, 96%, 97%, 98%, or 99%, an identical or—under conservative and semi-conservative amino acid exchanges—similar amino acid sequence to that of the protein from which the functional part originates.

As used herein “CPL1” refers to RNA polymerase II C-terminal domain phosphatase-like 1. By means of example, and without limitation, CPL1 as used herein may refer to any orthologue, homologue, homeologue, or paralogue of Arabidopsis thaliana CPL1 as represented by the protein sequence of SEQ ID NO: 1 or the coding sequence/cDNA sequence of SEQ ID NO: 18.

In certain embodiments, the plant (or plant part or population) comprises more than one CPL1 gene, which may be homeologous or paralogous, or both. In certain embodiments, the plant (or plant part or population) comprises two or more CPL1 genes, which may be homeologous or paralogous, or both. In certain embodiments, the different CPL1 homeologues or paralogues are differentially expressed. Differential expression can entail expression in different cells, tissues, or organs; expression at different growths stages; or both.

As used herein, “ERF922” refers to ethylene response factor 922, also known as ethylene responsive factor 922. By means of example, and without limitation, ERF922 as used herein may refer to any orthologue, homologue, homeologue, or paralogue of Oryza sativa ERF922 as represented by the protein sequence of SEQ ID NO: 36 or the coding sequence/cDNA sequence of SEQ ID NO: 51.

In certain embodiments, the plant (or plant part or population) comprises more than one ERF922 gene, which may be homeologous or paralogous, or both. In certain embodiments, the plant (or plant part or population) comprises two or more ERF922 genes, which may be homeologous or paralogous, or both.

In certain embodiments, the different ERF922 homeologues or paralogues are differentially expressed. Differential expression can entail expression in different cells, tissues, or organs; expression at different growths stages; or both.

In certain embodiments of the methods, plants (or parts or population), uses, polynucleic acids, or polypeptides according to the invention as described herein, a CPL1 and/or ERF922 gene is mutated.

In certain embodiments of the methods, plants (or parts or population), uses, polynucleic acids, or polypeptides according to the invention as described herein, a CPL1 and an ERF922 gene is mutated.

In certain embodiments of the methods, plants (or parts or population), uses, polynucleic acids, or polypeptides according to the invention as described herein, a CPL1 gene is mutated.

In certain embodiments of the methods, plants (or parts or population), uses, polynucleic acids, or polypeptides according to the invention as described herein, a ERF922 gene is mutated.

In certain embodiments of the methods, plants (or parts or population), uses, polynucleic acids, or polypeptides according to the invention as described herein, at least two, such as two, CPL1 and/or at least two, such as two, ERF922 genes are mutated. In certain embodiments of the methods, plants (or parts or population), uses, polynucleic acids, or polypeptides according to the invention as described herein, at least two, such as two, CPL1 and at least two, such as two, ERF922 genes are mutated. In certain embodiments of the methods, plants (or parts or population), uses, polynucleic acids, or polypeptides according to the invention as described herein, at least two, such as two, CPL1 genes are mutated. In certain embodiments of the methods, plants (or parts or population), uses, polynucleic acids, or polypeptides according to the invention as described herein, at least two, such as two, ERF922 genes are mutated.

The skilled person will understand that the wild type or unmutated gene product is a functional gene product having (substantially) unaltered functionality, such as enzymatic activity or transcriptional activity, as defined herein elsewhere. The skilled person will further understand that sequence variations described above for the wild type genes do not include frame shift or nonsense mutations.

As used herein, the mutated CPL1 and/or ERF922 or the mutation in the CPL1 and/or ERF922 may comprise or may refer to any type of CPL1 and/or ERF922 mutation. In certain embodiments the mutation alters expression of the wild type or native CPL1 and/or ERF922 protein and/or mRNA. In certain embodiments the mutation reduces or eliminates expression of the (wild type or native) CPL1 and/or ERF922 protein and/or mRNA, as described herein elsewhere. Mutations may affect transcription and/or translation. Mutations may occur in exons or introns. Mutations may occur in regulatory elements, such as promotors, enhancers, terminators, insulators, etc, as well as in 5′ and/or 3′ UTR coding regions. Mutations may occur in coding sequences. Mutations may occur in splicing signal sites, such as splice donor or splice acceptor sites. Mutations may be frame shift mutations. Mutations may be nonsense mutations. Mutations may be point mutations. Mutations may be insertion or deletion of one or more nucleotides, internally and/or terminally. Mutations may be non-conservative mutations (in which one or more wild type amino acids are replaced with one or more non-wild type amino acids). Mutations may result in a truncated protein. Mutations may affect or alter the function of the CPL1 and/or ERF922 protein, such as enzymatic or transcriptional activity. Mutations may reduce or (substantially) eliminate the function of the CPL1 and/or ERF922 protein, such as enzymatic activity or transcriptional activity. Reduced function, such as reduced enzymatic activity or transcriptional activity, may refer to a reduction of about at least 10%, preferably at least 30%, more preferably at least 50%, such as at least 20%, 40%, 60%, 80% or more, such as at least 85%, at least 90%, at least 95%, or more. (Substantially) eliminated function, such as (substantially) eliminated enzymatic activity or transcriptional activity, may refer to a reduction of at least 80%, preferably at least 90%, more preferably at least 95%. Mutations may be dominant negative mutations.

In certain embodiments, only a single allele is mutated (e.g. the plant, plant part, or plant population comprises only one mutant allele). In certain embodiments, multiple alleles are mutated. In certain embodiments, the mutation is homozygous. In certain embodiments, the mutation is heterozygous. In certain embodiments, one or both alleles of one or more paralogues genes are mutated. In certain embodiments, one or both alleles of one or more homeologous genes are mutated. In certain embodiments, one or both alleles of one or more paralogues and homeologous genes are mutated.

In certain embodiments, a dominant negative CPL1 and/or ERF922 is expressed in a plant. In certain embodiments, a dominant negative CPL1 and/or ERF922 is expressed in a plant, whereby the dominant negative is derived from the same species as the species it is expressed in. In certain embodiments, a dominant negative CPL1 and/or ERF922 is expressed in a plant, whereby the dominant negative is derived from the different species as the species it is expressed in (i.e. derived from an othologue).

In certain embodiments, an endogenous CPL1 and/or ERF922 gene is mutated to result in expression of a dominant negative protein. If all alleles are mutated (of all homeologues or paralogues), such dominant negative may be functionally equivalent to a knockout. If only a fraction of the alleles is mutated, such dominant negative may be functionally equivalent to a knockdown. In certain embodiments, the dominant negative is homozygous. In certain embodiments, the dominant negative is heterozygous.

In certain embodiments, the dominant negative CPL1 and/or ERF922 is exogenous (i.e. recombinant or transgenic), although it may originate from the same species as the species it is introduced.

As used herein, the term “CPL1 activity” may refer to the enzymatic activity of CPL1 or non-enzymatic activity of CPL1. The term “having reduced CPL1 activity” in the context of variant CPL1 as described above in certain preferred embodiments refers to a CPL1 of which the enzymatic or non-enzymatic activity is affected, in particular reduced compared to wild type or native CPL1. In certain embodiments, the (enzymatic) activity is at most 50% of the wild type CPL1 activity, preferably at most 40%, more preferably at most 30%, even more preferably at most 20%, most preferably at most 10%, such as at most 5%. (Enzymatic) activity can be measured by means known in the art.

As used herein, the term “ERF922 activity” may refer to the transcriptional activity of ERF922 or non-transcriptional activity of ERF922. The term “having reduced ERF922 activity” in the context of variant ERF922 as described above in certain preferred embodiments refers to a ERF922 of which the transcriptional or non-transcriptional activity is affected, in particular reduced compared to wild type or native ERF922. In certain embodiments, the (transcriptional) activity is at most 50% of the wild type ERF922 activity, preferably at most 40%, more preferably at most 30%, even more preferably at most 20%, most preferably at most 10%, such as at most 5%. (Transcriptional) activity can be measured by means known in the art.

In certain embodiments, the CPL1 and/or ERF922 mutation is an insertion of one or more nucleotides in the coding sequence. In certain embodiments, the CPL1 and/or ERF922 mutation is a nonsense mutation. In certain embodiments, the CPL1 and/or ERF922 mutation results in reduced expression of the CPL1 and/or ERF922 gene. In certain embodiments, the CPL1 and/or ERF922 mutation results in knockout of the CPL1 and/or ERF922 gene or knockdown of the CPL1 and/or ERF922 mRNA and/or protein. In certain embodiments, the mutation results in a frame shift of the coding sequence of CPL1 and/or ERF922. In certain embodiments, the mutation results in an altered protein sequence encoded by the CPL1 and/or ERF922 gene.

In certain embodiments, the CPL1 and/or ERF922 mutation is an insertion, preferably in an exon, preferably an insertion in the first exon, of one or more nucleotides, preferably a frame shift insertion.

CPL1 and/or ERF922 mRNA and/or protein expression may be reduced or eliminated by mutating the CPL1 and/or ERF922 gene itself (including coding, non-coding, and regulatory element). Methods for introducing mutations are described herein elsewhere. Alternatively, CPL1 and/or ERF922 mRNA and/or protein expression may be reduced or eliminated by (specifically) interfering with transcription and/or translation, such as to decrease or eliminate mRNA and/or protein transcription or translation. Alternatively, CPL1 and/or ERF922 mRNA and/or protein expression may be reduced or eliminated by (specifically) interfering with mRNA and/or protein stability, such as to reduce mRNA and/or protein stability. By means of example, mRNA (stability) may be reduced by means of RNAi, as described herein elsewhere. Also miRNA can be used to affect mRNA (stability). In certain embodiments, a reduced CPL1 and/or ERF922 expression which is achieved by reducing mRNA or protein stability is also encompassed by the term “mutated” CPL1 and/or ERF922. In certain embodiments, a reduced CPL1 and/or ERF922 expression which is achieved by reducing mRNA or protein stability is not encompassed by the term “mutated” CPL1 and/or ERF922.

As used herein, a “mutation” refers to a modification at the DNA level, and includes changes in the genetics and/or epigenetics. An alteration in the genetics may include an insertion, a deletion, an introduction of a stop codon, a base change (e.g. transition or transversion), or an alteration in splice junctions. These alterations may arise in coding or non-coding regions (e.g. promoter regions, exons, introns or splice junctions) of the endogenous DNA sequence. For example, an alteration in the genetics may be the exchange of at least one nucleobase in the endogenous DNA sequence or in a regulatory sequence of the endogenous DNA sequence. If such a nucleobase exchange takes place in a promoter, for example, this may lead to an altered activity of the promoter, since, for example, cis-regulator elements are modified such that the affinity of a transcription factor to the mutated cis-regulatory elements is altered in comparison to the wild-type promoter, so that the activity of the promoter with the mutated cis-regulatory elements is increased or reduced, depending upon whether the transcription factor is a repressor or inductor, or whether the affinity of the transcription factor to the mutated cis-regulatory elements is intensified or weakened. If such a nucleobase exchange occurs, e.g., in an encoding region of the endogenous DNA sequence, this may lead to an amino acid exchange in the encoded protein, which may produce an alteration in the activity or stability of the protein, in comparison to the wild-type protein. An alteration in the epigenetics may take place via an altered methylation pattern of the DNA.

Mutagenesis may be performed in accordance with any of the techniques known in the art. As used herein, “mutagenization” or “mutagenesis” includes both conventional mutagenesis and location-specific mutagenesis or “genome editing” or “gene editing”. In conventional mutagenesis, modification at the DNA level is not produced in a targeted manner. The plant cell or the plant is exposed to mutagenic conditions, such as TILLING, via UV light exposure or the use of chemical substances (Till et al., 2004). An additional method of random mutagenesis is mutagenesis with the aid of a transposon. Location-specific mutagenesis enables the introduction of modification at the DNA level in a target-oriented manner at predefined locations in the DNA. For example, TALENS, meganucleases, homing endonucleases, zinc finger nucleases, or a CRISPR/Cas System as further described herein may be used for this.

As used herein, the terms “introgression”, “introgressed” and “introgressing” refer to both a natural and artificial process whereby chromosomal fragments or genes of one species, variety or cultivar are moved into the genome of another species, variety or cultivar, by crossing those species. The process may optionally be completed by backcrossing to the recurrent parent. For example, introgression of a desired allele at a specified locus can be transmitted to at least one progeny via a sexual cross between two parents of the same species, where at least one of the parents has the desired allele in its genome. Alternatively, for example, transmission of an allele can occur by recombination between two donor genomes, e.g., in a fused protoplast, where at least one of the donor protoplasts has the desired allele in its genome. The desired allele can be, e.g., detected by a marker that is associated with a phenotype, at a QTL, a transgene, or the like. In any case, offspring comprising the desired allele can be repeatedly backcrossed to a line having a desired genetic background and selected for the desired allele, to result in the allele becoming fixed in a selected genetic background. The process of “introgressing” is often referred to as “backcrossing” when the process is repeated two or more times. “Introgression fragment” or “introgression segment” or “introgression region” refers to a chromosome fragment (or chromosome part or region) which has been introduced into another plant of the same or related species either artificially or naturally such as by crossing or traditional breeding techniques, such as backcrossing, i.e. the introgressed fragment is the result of breeding methods referred to by the verb “to introgress” (such as backcrossing). It is understood that the term “introgression fragment” never includes a whole chromosome, but only a part of a chromosome. The introgression fragment can be large, e.g. even three quarter or half of a chromosome, but is preferably smaller, such as about 15 Mb or less, such as about 10 Mb or less, about 9 Mb or less, about 8 Mb or less, about 7 Mb or less, about 6 Mb or less, about 5 Mb or less, about 4 Mb or less, about 3 Mb or less, about 2.5 Mb or 2 Mb or less, about 1 Mb (equals 1,000,000 base pairs) or less, or about 0.5 Mb (equals 500,000 base pairs) or less, such as about 200,000 bp (equals 200 kilo base pairs) or less, about 100,000 bp (100 kb) or less, about 50,000 bp (50 kb) or less, about 25,000 bp (25 kb) or less.

The term “locus” (loci plural) means a specific place or places or a site on a chromosome where for example a QTL, a gene or genetic marker is found. As used herein, the term “quantitative trait locus” or “QTL” has its ordinary meaning known in the art. By means of further guidance, and without limitation, a QTL may refer to a region of DNA that is associated with the differential expression of a quantitative phenotypic trait in at least one genetic background, e.g., in at least one breeding population. The region of the QTL encompasses or is closely linked to the gene or genes that affect the trait in question. An “allele of a QTL” can comprise multiple genes or other genetic factors within a contiguous genomic region or linkage group, such as a haplotype. An allele of a QTL can denote a haplotype within a specified window wherein said window is a contiguous genomic region that can be defined, and tracked, with a set of one or more polymorphic markers. A haplotype can be defined by the unique fingerprint of alleles at each marker within the specified window. A QTL may encode for one or more alleles that affect the expressivity of a continuously distributed (quantitative) phenotype. In certain embodiments, the QTL as described herein may be homozygous. In certain embodiments, the QTL as described herein may be heterozygous.

As used herein, the term “allele” or “alleles” refers to one or more alternative forms, i.e. different nucleotide sequences, of a locus.

As used herein, the term “mutant alleles” or “mutation” of alleles include alleles having one or more mutations, such as insertions, deletions, stop codons, base changes (e.g., transitions or transversions), or alterations in splice junctions, which may or may not give rise to altered gene products. Modifications in alleles may arise in coding or non-coding regions (e.g. promoter regions, exons, introns or splice junctions).

A genetic element, an introgression fragment, or a gene or allele conferring a trait (such as increased pathogen tolerance or resistance) is said to be “obtainable from” or can be “obtained from” or “derivable from” or can be “derived from” or “as present in” or “as found in” a plant or plant part as described herein elsewhere if it can be transferred from the plant in which it is present into another plant in which it is not present (such as a line or variety) using traditional breeding techniques without resulting in a phenotypic change of the recipient plant apart from the addition of the trait conferred by the genetic element, locus, introgression fragment, gene or allele. The terms are used interchangeably and the genetic element, locus, introgression fragment, gene or allele can thus be transferred into any other genetic background lacking the trait. Not only pants comprising the genetic element, locus, introgression fragment, gene or allele can be used, but also progeny/descendants from such plants which have been selected to retain the genetic element, locus, introgression fragment, gene or allele, can be used and are encompassed herein. Whether a plant (or genomic DNA, cell or tissue of a plant) comprises the same genetic element, locus, introgression fragment, gene or allele as obtainable from such plant can be determined by the skilled person using one or more techniques known in the art, such as phenotypic assays, whole genome sequencing, molecular marker analysis, trait mapping, chromosome painting, allelism tests and the like, or combinations of techniques. It will be understood that transgenic plants may also be encompassed.

As used herein the terms “genetic engineering”, “transformation” and “genetic modification” are all used herein as synonyms for the transfer of isolated and cloned genes into the DNA, usually the chromosomal DNA or genome, of another organism.

“Transgenic” or “genetically modified organisms” (GMOs) as used herein are organisms whose genetic material has been altered using techniques generally known as “recombinant DNA technology”. Recombinant DNA technology encompasses the ability to combine DNA molecules from different sources into one molecule ex vivo (e.g. in a test tube). This terminology generally does not cover organisms whose genetic composition has been altered by conventional cross-breeding or by “mutagenesis” breeding, as these methods predate the discovery of recombinant DNA techniques. “Non-transgenic” as used herein refers to plants and food products derived from plants that are not “transgenic” or “genetically modified organisms” as defined above.

“Transgene” or “chimeric gene” refers to a genetic locus comprising a DNA sequence, such as a recombinant gene, which has been introduced into the genome of a plant by transformation, such as Agrobacterium mediated transformation. A plant comprising a transgene stably integrated into its genome is referred to as “transgenic plant”.

“Gene editing” or “genome editing” refers to genetic engineering in which in which DNA or RNA is inserted, deleted, modified or replaced in the genome of a living organism. Gene editing may comprise targeted or non-targeted (random) mutagenesis. Targeted mutagenesis may be accomplished for instance with designer nucleases, such as for instance with meganucleases, zinc finger nucleases (ZFNs), transcription activator-like effector-based nucleases (TALEN), and the clustered regularly interspaced short palindromic repeats (CRISPR/Cas9) system. These nucleases create site-specific double-strand breaks (DSBs) at desired locations in the genome. The induced double-strand breaks are repaired through nonhomologous end-joining (NHEJ) or homologous recombination (HR), resulting in targeted mutations or nucleic acid modifications. The use of designer nucleases is particularly suitable for generating gene knockouts or knockdowns. In certain embodiments, designer nucleases are developed which specifically induce a mutation in the CPL1 and/or ERF922 gene, as described herein elsewhere, such as to generate a mutated CPL1 and/or ERF922 or a knockout of the CPL1 and/or ERF922 gene. In certain embodiments, designer nucleases, in particular RNA-specific CRISPR/Cas systems are developed which specifically target the CPL1 and/or ERF922 mRNA, such as to cleave the CPL1 and/or ERF922 mRNA and generate a knockdown of the CPL1 and/or ERF922 gene/mRNA/protein. Delivery and expression systems of designer nuclease systems are well known in the art.

In certain embodiments, the nuclease or targeted/site-specific/homing nuclease is, comprises, consists essentially of, or consists of a (modified) CRISPR/Cas system or complex, a (modified) Cas protein, a (modified) zinc finger, a (modified) zinc finger nuclease (ZFN), a (modified) transcription factor-like effector (TALE), a (modified) transcription factor-like effector nuclease (TALEN), or a (modified) meganuclease. In certain embodiments, said (modified) nuclease or targeted/site-specific/homing nuclease is, comprises, consists essentially of, or consists of a (modified) RNA-guided nuclease. It will be understood that in certain embodiments, the nucleases may be codon optimized for expression in plants. As used herein, the term “targeting” of a selected nucleic acid sequence means that a nuclease or nuclease complex is acting in a nucleotide sequence specific manner. For instance, in the context of the CRISPR/Cas system, the guide RNA is capable of hybridizing with a selected nucleic acid sequence. As used herein, “hybridization” or “hybridizing” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. Hybridization is a process in which a single-stranded nucleic acid molecule attaches itself to a complementary nucleic acid strand, i.e. agrees with this base pairing. Standard procedures for hybridization are described, for example, in Sambrook et al. (Molecular Cloning. A Laboratory Manual, Cold Spring Harbor Laboratory Press, 3rd edition 2001). Preferably this will be understood to mean an at least 50%, more preferably at least 55%, 60%, 65%, 70%, 75%, 80% or 85%, more preferably 90%, 91%, 92%, 93%, 94%, 95%, 96%, 97%, 98% or 99% of the bases of the nucleic acid strand form base pairs with the complementary nucleic acid strand. A hybridization reaction may constitute a step in a more extensive process, such as the initiation of PGR, or the cleavage of a polynucleotide by an enzyme. A sequence capable of hybridizing with a given sequence is referred to as the “complement” of the given sequence.

Gene editing may involve transient, inducible, or constitutive expression of the gene editing components or systems. Gene editing may involve genomic integration or episomal presence of the gene editing components or systems. Gene editing components or systems may be provided on vectors, such as plasmids, which may be delivered by appropriate delivery vehicles, as is known in the art. Preferred vectors are expression vectors.

Gene editing may comprise the provision of recombination templates, to effect homology directed repair (HDR). For instance a genetic element may be replaced by gene editing in which a recombination template is provided. The DNA may be cut upstream and downstream of a sequence which needs to be replaced. As such, the sequence to be replaced is excised from the DNA. Through HDR, the excised sequence is then replaced by the template. In certain embodiments, the QTL allele of the invention as described herein may be provided on/as a template. By designing the system such that double strand breaks are introduced upstream and downstream of the corresponding region in the genome of a plant not comprising the QTL allele, this region is excised and can be replaced with the template comprising the QTL allele of the invention. In this way, introduction of the QTL allele of the invention in a plant need not involve multiple backcrossing, in particular in a plant of specific genetic background. Similarly, the mutated CPL1 and/or ERF922 of the invention may be provided on/as a template. More advantageously however, the mutated CPL1 and/or ERF922 of the invention may be generated without the use of a recombination template, but solely through the endonuclease action leading to a double strand DNA break which is repaired by NHEJ, resulting in the generation of indels.

In certain embodiments, the nucleic acid modification or mutation is effected by a (modified) transcription activator-like effector nuclease (TALEN) system. Transcription activator-like effectors (TALEs) can be engineered to bind practically any desired DNA sequence. Exemplary methods of genome editing using the TALEN system can be found for example in Cermak T. Doyle E L. Christian M. Wang L. Zhang Y. Schmidt C, et al. Efficient design and assembly of custom TALEN and other TAL effector-based constructs for DNA targeting. Nucleic Acids Res. 2011; 39:e82; Zhang F. Cong L. Lodato S. Kosuri S. Church G M. Arlotta P Efficient construction of sequence-specific TAL effectors for modulating mammalian transcription. Nat Biotechnol. 2011; 29:149-153 and U.S. Pat. Nos. 8,450,471, 8,440,431 and 8,440,432, all of which are specifically incorporated by reference. By means of further guidance, and without limitation, naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, or “TALE monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X1-11-(X12X13)-X14-33 or 34 or 35, where the subscript indicates the amino acid position and X represents any amino acid. X12X13 indicate the RVDs. In some polypeptide monomers, the variable amino acid at position 13 is missing or absent and in such polypeptide monomers, the RVD consists of a single amino acid. In such cases the RVD may be alternatively represented as X*, where X represents X12 and (*) indicates that X13 is absent. The DNA binding domain comprises several repeats of TALE monomers and this may be represented as (X1-11-(X12X13)-X14-33 or 34 or 35)z, where in an advantageous embodiment, z is at least 5 to 40. In a further advantageous embodiment, z is at least 10 to 26. The TALE monomers have a nucleotide binding affinity that is determined by the identity of the amino acids in its RVD. For example, polypeptide monomers with an RVD of NI preferentially bind to adenine (A), polypeptide monomers with an RVD of NG preferentially bind to thymine (T), polypeptide monomers with an RVD of HD preferentially bind to cytosine (C) and polypeptide monomers with an RVD of NN preferentially bind to both adenine (A) and guanine (G). In yet another embodiment of the invention, polypeptide monomers with an RVD of IG preferentially bind to T. Thus, the number and order of the polypeptide monomer repeats in the nucleic acid binding domain of a TALE determines its nucleic acid target specificity. In still further embodiments of the invention, polypeptide monomers with an RVD of NS recognize all four base pairs and may bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011), each of which is incorporated by reference in its entirety.

In certain embodiments, the nucleic acid modification or mutation is effected by a (modified) zinc-finger nuclease (ZFN) system. The ZFN system uses artificial restriction enzymes generated by fusing a zinc finger DNA-binding domain to a DNA-cleavage domain that can be engineered to target desired DNA sequences. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference. By means of further guidance, and without limitation, artificial zinc-finger (ZF) technology involves arrays of ZF modules to target new DNA-binding sites in the genome. Each finger module in a ZF array targets three DNA bases. A customized array of individual zinc finger domains is assembled into a ZF protein (ZFP). ZFPs can comprise a functional domain. The first synthetic zinc finger nucleases (ZFNs) were developed by fusing a ZF protein to the catalytic domain of the Type IIS restriction enzyme Fokl. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to FokI cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms.

In certain embodiments, the nucleic acid modification is effected by a (modified) meganuclease, which are endodeoxyribonucleases characterized by a large recognition site (double-stranded DNA sequences of 12 to 40 base pairs). Exemplary method for using meganucleases can be found in U.S. Pat. Nos. 8,163,514; 8,133,697; 8,021,867; 8,119,361; 8,119,381; 8,124,369; and 8,129,134, which are specifically incorporated by reference.

In certain embodiments, the nucleic acid modification is effected by a (modified) CRISPR/Cas complex or system. With respect to general information on CRISPR/Cas Systems, components thereof, and delivery of such components, including methods, materials, delivery vehicles, vectors, particles, and making and using thereof, including as to amounts and formulations, as well as Cas9CRISPR/Cas-expressing eukaryotic cells, Cas-9 CRISPR/Cas expressing eukaryotes, reference is made to: U.S. Pat. Nos. 8,999,641, 8,993,233, 8,697,359, 8,771,945, 8,795,965, 8,865,406, 8,871,445, 8,889,356, 8,889,418, 8,895,308, 8,906,616, 8,932,814, 8,945,839, 8,993,233 and 8,999,641; US Patent Publications US 2014-0310830 (U.S. application Ser. No. 14/105,031), US 2014-0287938 A1 (U.S. application Ser. No. 14/213,991), US 2014-0273234 A1 (U.S. application Ser. No. 14/293,674), US2014-0273232 A1 (U.S. application Ser. No. 14/290,575), US 2014-0273231 (U.S. application Ser. No. 14/259,420), US 2014-0256046 A1 (U.S. application Ser. No. 14/226,274), US 2014-0248702 A1 (U.S. application Ser. No. 14/258,458), US 2014-0242700 A1 (U.S. application Ser. No. 14/222,930), US 2014-0242699 A1 (U.S. application Ser. No. 14/183,512), US 2014-0242664 A1 (U.S. application Ser. No. 14/104,990), US 2014-0234972 A1 (U.S. application Ser. No. 14/183,471), US 2014-0227787 A1 (U.S. application Ser. No. 14/256,912), US 2014-0189896 A1 (U.S. application Ser. No. 14/105,035), US 2014-0186958 (U.S. application Ser. No. 14/105,017), US 2014-0186919 A1 (U.S. application Ser. No. 14/104,977), US 2014-0186843 A1 (U.S. application Ser. No. 14/104,900), US 2014-0179770 A1 (U.S. application Ser. No. 14/104,837) and US 2014-0179006 A1 (U.S. application Ser. No. 14/183,486), US 2014-0170753 (U.S. application Ser. No. 14/183,429); US 2015-0184139 (U.S. application Ser. No. 14/324,960); Ser. No. 14/054,414 European Patent Applications EP 2 771 468 (EP13818570.7), EP 2 764 103 (EP13824232.6), and EP 2 784 162 (EP14170383.5); and PCT Patent Publications WO 2014/093661 (PCT/US2013/074743), WO 2014/093694 (PCT/US2013/074790), WO 2014/093595 (PCT/US2013/074611), WO 2014/093718 (PCT/US2013/074825), WO 2014/093709 (PCT/US2013/074812), WO 2014/093622 (PCT/US2013/074667), WO 2014/093635 (PCT/US2013/074691), WO 2014/093655 (PCT/US2013/074736), WO 2014/093712 (PCT/US2013/074819), WO 2014/093701 (PCT/US2013/074800), WO 2014/018423 (PCT/US2013/051418), WO 2014/204723 (PCT/US2014/041790), WO 2014/204724 (PCT/US2014/041800), WO 2014/204725 (PCT/US2014/041803), WO 2014/204726 (PCT/US2014/041804), WO 2014/204727 (PCT/US2014/041806), WO 2014/204728 (PCT/US2014/041808), WO 2014/204729 (PCT/US2014/041809), WO 2015/089351 (PCT/US2014/069897), WO 2015/089354 (PCT/US2014/069902), WO 2015/089364 (PCT/US2014/069925), WO 2015/089427 (PCT/US2014/070068), WO 2015/089462 (PCT/US2014/070127), WO 2015/089419 (PCT/US2014/070057), WO 2015/089465 (PCT/US2014/070135), WO 2015/089486 (PCT/US2014/070175), PCT/US2015/051691, PCT/US2015/051830. Reference is also made to U.S. provisional patent applications 61/758,468; 61/802,174; 61/806,375; 61/814,263; 61/819,803 and 61/828,130, filed on Jan. 30, 2013; Mar. 15, 2013; Mar. 28, 2013; Apr. 20, 2013; May 6, 2013 and May 28, 2013 respectively. Reference is also made to U.S. provisional patent application 61/836,123, filed on Jun. 17, 2013. Reference is additionally made to U.S. provisional patent applications 61/835,931, 61/835,936, 61/835,973, 61/836,080, 61/836,101, and 61/836,127, each filed Jun. 17, 2013. Further reference is made to U.S. provisional patent applications 61/862,468 and 61/862,355 filed on Aug. 5, 2013; 61/871,301 filed on Aug. 28, 2013; 61/960,777 filed on Sep. 25, 2013 and 61/961,980 filed on Oct. 28, 2013. Reference is yet further made to: PCT/US2014/62558 filed Oct. 28, 2014, and U.S. Provisional Patent Applications Ser. Nos. 61/915,148, 61/915,150, 61/915,153, 61/915,203, 61/915,251, 61/915,301, 61/915,267, 61/915,260, and 61/915,397, each filed Dec. 12, 2013; 61/757,972 and 61/768,959, filed on Jan. 29, 2013 and Feb. 25, 2013; 62/010,888 and 62/010,879, both filed Jun. 11, 2014; 62/010,329, 62/010,439 and 62/010,441, each filed Jun. 10, 2014; 61/939,228 and 61/939,242, each filed Feb. 12, 2014; 61/980,012, filed Apr. 15, 2014; 62/038,358, filed Aug. 17, 2014; 62/055,484, 62/055,460 and 62/055,487, each filed Sep. 25, 2014; and 62/069,243, filed Oct. 27, 2014. Reference is made to PCT application designating, inter alia, the United States, application No. PCT/US14/41806, filed Jun. 10, 2014. Reference is made to U.S. provisional patent application 61/930,214 filed on Jan. 22, 2014. Reference is made to PCT application designating, inter alia, the United States, application No. PCT/US14/41806, filed Jun. 10, 2014. Mention is also made of U.S. application 62/180,709, 17 Jun. 2015, PROTECTED GUIDE RNAS (PGRNAS); U.S. application 62/091,455, filed, 12 Dec. 2014, PROTECTED GUIDE RNAS (PGRNAS); U.S. application 62/096,708, 24 Dec. 2014, PROTECTED GUIDE RNAS (PGRNAS); U.S. applications 62/091,462, 12 Dec. 2014, 62/096,324, 23 Dec. 2014, 62/180,681, 17 Jun. 2015, and 62/237,496, 5 Oct. 2015, DEAD GUIDES FOR CRISPR TRANSCRIPTION FACTORS; U.S. application 62/091,456, 12 Dec. 2014 and 62/180,692, 17 Jun. 2015, ESCORTED AND FUNCTIONALIZED GUIDES FOR CRISPR-CAS SYSTEMS; U.S. application 62/091,461, 12 Dec. 2014, DELIVERY, USE AND THERAPEUTIC APPLICATIONS OF THE CRISPR-CAS SYSTEMS AND COMPOSITIONS FOR GENOME EDITING AS TO HEMATOPOETIC STEM CELLS (HSCs); U.S. application 62/094,903, 19 Dec. 2014, UNBIASED IDENTIFICATION OF DOUBLE-STRAND BREAKS AND GENOMIC REARRANGEMENT BY GENOME-WISE INSERT CAPTURE SEQUENCING; U.S. application 62/096,761, 24 Dec. 2014, ENGINEERING OF SYSTEMS, METHODS AND OPTIMIZED ENZYME AND GUIDE SCAFFOLDS FOR SEQUENCE MANIPULATION; U.S. application 62/098,059, 30 Dec. 2014, 62/181,641, 18 Jun. 2015, and 62/181,667, 18 Jun. 2015, RNA-TARGETING SYSTEM; U.S. application 62/096,656, 24 Dec. 2014 and 62/181,151, 17 Jun. 2015, CRISPR HAVING OR ASSOCIATED WITH DESTABILIZATION DOMAINS; U.S. application 62/096,697, 24 Dec. 2014, CRISPR HAVING OR ASSOCIATED WITH AAV; U.S. application 62/098,158, 30 Dec. 2014, ENGINEERED CRISPR COMPLEX INSERTIONAL TARGETING SYSTEMS; U.S. application 62/151,052, 22 Apr. 2015, CELLULAR TARGETING FOR EXTRACELLULAR EXOSOMAL REPORTING; U.S. application 62/054,490, 24 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING PARTICLE DELIVERY COMPONENTS; U.S. application 61/939,154, 12 Feb. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/055,484, 25 Sep. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/087,537, 4 Dec. 2014, SYSTEMS, METHODS AND COMPOSITIONS FOR SEQUENCE MANIPULATION WITH OPTIMIZED FUNCTIONAL CRISPR-CAS SYSTEMS; U.S. application 62/054,651, 24 Sep. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. application 62/067,886, 23 Oct. 2014, DELIVERY, USE AND THERAPEUTIC APPLICATIONS OF THE CRISPR-CAS SYSTEMS AND COMPOSITIONS FOR MODELING COMPETITION OF MULTIPLE CANCER MUTATIONS IN VIVO; U.S. applications 62/054,675, 24 Sep. 2014 and 62/181,002, 17 Jun. 2015, DELIVERY, USE AND THERAPEUTIC APPLICATIONS OF THE CRISPR-CAS SYSTEMS AND COMPOSITIONS IN NEURONAL CELLS/TISSUES; U.S. application 62/054,528, 24 Sep. 2014, DELIVERY, USE AND THERAPEUTIC APPLICATIONS OF THE CRISPR-CAS SYSTEMS AND COMPOSITIONS IN IMMUNE DISEASES OR DISORDERS; U.S. application 62/055,454, 25 Sep. 2014, DELIVERY, USE AND THERAPEUTIC APPLICATIONS OF THE CRISPR-CAS SYSTEMS AND COMPOSITIONS FOR TARGETING DISORDERS AND DISEASES USING CELL PENETRATION PEPTIDES (CPP); U.S. application 62/055,460, 25 Sep. 2014, MULTIFUNCTIONAL-CRISPR COMPLEXES AND/OR OPTIMIZED ENZYME LINKED FUNCTIONAL-CRISPR COMPLEXES; U.S. application 62/087,475, 4 Dec. 2014 and 62/181,690, 18 Jun. 2015, FUNCTIONAL SCREENING WITH OPTIMIZED FUNCTIONAL CRISPR-CAS SYSTEMS; U.S. application 62/055,487, 25 Sep. 2014, FUNCTIONAL SCREENING WITH OPTIMIZED FUNCTIONAL CRISPR-CAS SYSTEMS; U.S. application 62/087,546, 4 Dec. 2014 and 62/181,687, 18 Jun. 2015, MULTIFUNCTIONAL CRISPR COMPLEXES AND/OR OPTIMIZED ENZYME LINKED FUNCTIONAL-CRISPR COMPLEXES; and U.S. application 62/098,285, 30 Dec. 2014, CRISPR MEDIATED IN VIVO MODELING AND GENETIC SCREENING OF TUMOR GROWTH AND METASTASIS. Mention is made of U.S. applications 62/181,659, 18 Jun. 2015 and 62/207,318, 19 Aug. 2015, ENGINEERING AND OPTIMIZATION OF SYSTEMS, METHODS, ENZYME AND GUIDE SCAFFOLDS OF CAS9 ORTHOLOGS AND VARIANTS FOR SEQUENCE MANIPULATION. Mention is made of U.S. applications 62/181,663, 18 Jun. 2015 and 62/245,264, 22 Oct. 2015, NOVEL CRISPR ENZYMES AND SYSTEMS, U.S. applications 62/181,675, 18 Jun. 2015, and Attorney Docket No. 46783.01.2128, filed 22 Oct. 2015, NOVEL CRISPR ENZYMES AND SYSTEMS, U.S. application 62/232,067, 24 Sep. 2015, U.S. application 62/205,733, 16 Aug. 2015, U.S. application 62/201,542, 5 Aug. 2015, U.S. application 62/193,507, 16 Jul. 2015, and U.S. application 62/181,739, 18 Jun. 2015, each entitled NOVEL CRISPR ENZYMES AND SYSTEMS and of U.S. application 62/245,270, 22 Oct. 2015, NOVEL CRISPR ENZYMES AND SYSTEMS. Mention is also made of U.S. application 61/939,256, 12 Feb. 2014, and WO 2015/089473 (PCT/US2014/070152), 12 Dec. 2014, each entitled ENGINEERING OF SYSTEMS, METHODS AND OPTIMIZED GUIDE COMPOSITIONS WITH NEW ARCHITECTURES FOR SEQUENCE MANIPULATION. Mention is also made of PCT/US2015/045504, 15 Aug. 2015, U.S. application 62/180,699, 17 Jun. 2015, and U.S. application 62/038,358, 17 Aug. 2014, each entitled GENOME EDITING USING CAS9 NICKASES. European patent application EP3009511. Reference is further made to Multiplex genome engineering using CRISPR/Cas systems. Cong, L., Ran, F. A., Cox, D., Lin, S., Barretto, R., Habib, N., Hsu, P. D., Wu, X., Jiang, W., Marraffini, L. A., & Zhang, F. Science February 15; 339(6121):819-23 (2013); RNA-guided editing of bacterial genomes using CRISPR-Cas systems. Jiang W., Bikard D., Cox D., Zhang F, Marraffini L A. Nat Biotechnol March; 31(3):233-9 (2013); One-Step Generation of Mice Carrying Mutations in Multiple Genes by CRISPR/Cas-Mediated Genome Engineering. Wang H., Yang H., Shivalila C S., Dawlaty M M., Cheng A W., Zhang F., Jaenisch R. Cell May 9; 153(4):910-8 (2013); Optical control of mammalian endogenous transcription and epigenetic states. Konermann S, Brigham M D, Trevino A E, Hsu P D, Heidenreich M, Cong L, Platt R J, Scott D A, Church G M, Zhang F. Nature. 2013 Aug. 22; 500(7463):472-6. doi: 10.1038/Nature12466. Epub 2013 Aug. 23; Double Nicking by RNA-Guided CRISPR Cas9 for Enhanced Genome Editing Specificity. Ran, F A., Hsu, P D., Lin, C Y., Gootenberg, J S., Konermann, S., Trevino, A E., Scott, D A., Inoue, A., Matoba, S., Zhang, Y., & Zhang, F. Cell August 28. pii: S0092-8674(13)01015-5. (2013); DNA targeting specificity of RNA-guided Cas9 nucleases. Hsu, P., Scott, D., Weinstein, J., Ran, F A., Konermann, S., Agarwala, V., Li, Y., Fine, E., Wu, X., Shalem, O., Cradick, T J., Marraffini, L A., Bao, G., & Zhang, F. Nat Biotechnol doi:10.1038/nbt.2647 (2013); Genome engineering using the CRISPR-Cas9 system. Ran, F A., Hsu, P D., Wright, J., Agarwala, V., Scott, D A., Zhang, F. Nature Protocols November; 8(11):2281-308. (2013); Genome-Scale CRISPR-Cas9 Knockout Screening in Human Cells. Shalem, O., Sanjana, N E., Hartenian, E., Shi, X., Scott, D A., Mikkelson, T., Heckl, D., Ebert, B L., Root, D E., Doench, J G., Zhang, F. Science Dec. 12. (2013). [Epub ahead of print]; Crystal structure of cas9 in complex with guide RNA and target DNA. Nishimasu, H., Ran, F A., Hsu, P D., Konermann, S., Shehata, S I., Dohmae, N., Ishitani, R., Zhang, F., Nureki, O. Cell Feb. 27. (2014). 156(5):935-49; Genome-wide binding of the CRISPR endonuclease Cas9 in mammalian cells. Wu X., Scott D A., Kriz A J., Chiu A C., Hsu P D., Dadon D B., Cheng A W., Trevino A E., Konermann S., Chen S., Jaenisch R., Zhang F., Sharp P A. Nat Biotechnol. (2014) Apr. 20. doi: 10.1038/nbt.2889; CRISPR-Cas9 Knockin Mice for Genome Editing and Cancer Modeling, Platt et al., Cell 159(2): 440-455 (2014) DOI: 10.1016/j.cell.2014.09.014; Development and Applications of CRISPR-Cas9 for Genome Engineering, Hsu et al, Cell 157, 1262-1278 (Jun. 5, 2014) (Hsu 2014); Genetic screens in human cells using the CRISPR/Cas9 system, Wang et al., Science. 2014 Jan. 3; 343(6166): 80-84. doi:10.1126/science.1246981; Rational design of highly active sgRNAs for CRISPR-Cas9-mediated gene inactivation, Doench et al., Nature Biotechnology 32(12):1262-7 (2014) published online 3 Sep. 2014; doi:10.1038/nbt.3026, and In vivo interrogation of gene function in the mammalian brain using CRISPR-Cas9, Swiech et al, Nature Biotechnology 33, 102-106 (2015) published online 19 Oct. 2014; doi:10.1038/nbt.3055, Cpf1 Is a Single RNA-Guided Endonuclease of a Class 2 CRISPR-Cas System, Zetsche et al., Cell 163, 1-13 (2015); Discovery and Functional Characterization of Diverse Class 2 CRISPR-Cas Systems, Shmakov et al., Mol Cell 60(3): 385-397 (2015); C2c2 is a single-component programmable RNA-guided RNA-targeting CRISPR effector, Abudayyeh et al, Science (2016) published online Jun. 2, 2016 doi: 10.1126/science.aaf5573. Each of these publications, patents, patent publications, and applications, and all documents cited therein or during their prosecution (“appln cited documents”) and all documents cited or referenced in the appln cited documents, together with any instructions, descriptions, product specifications, and product sheets for any products mentioned therein or in any document therein and incorporated by reference herein, are hereby incorporated herein by reference, and may be employed in the practice of the invention. All documents (e.g., these patents, patent publications and applications and the appln cited documents) are incorporated herein by reference to the same extent as if each individual document was specifically and individually indicated to be incorporated by reference.

In certain embodiments, the CRISPR/Cas system or complex is a class 2 CRISPR/Cas system. In certain embodiments, said CRISPR/Cas system or complex is a type II, type V, or type VI CRISPR/Cas system or complex. The CRISPR/Cas system does not require the generation of customized proteins to target specific sequences but rather a single Cas protein can be programmed by an RNA guide (gRNA) to recognize a specific nucleic acid target, in other words the Cas enzyme protein can be recruited to a specific nucleic acid target locus (which may comprise or consist of RNA and/or DNA) of interest using said short RNA guide.

In general, the CRISPR/Cas or CRISPR system is as used herein foregoing documents refers collectively to transcripts and other elements involved in the expression of or directing the activity of CRISPR-associated (“Cas”) genes, including sequences encoding a Cas gene and one or more of, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g. CRISPR RNA and, where applicable, transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides.

In certain embodiments, the gRNA is a chimeric guide RNA or single guide RNA (sgRNA). In certain embodiments, the gRNA comprises a guide sequence and a tracr mate sequence (or direct repeat). In certain embodiments, the gRNA comprises a guide sequence, a tracr mate sequence (or direct repeat), and a tracr sequence. In certain embodiments, the CRISPR/Cas system or complex as described herein does not comprise and/or does not rely on the presence of a tracr sequence (e.g. if the Cas protein is Cpf1).

As used herein, the term “crRNA” or “guide RNA” or “single guide RNA” or “sgRNA” or “one or more nucleic acid components” of a CRISPR/Cas locus effector protein, as applicable, comprises any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). The ability of a guide sequence (within a nucleic acid-targeting guide RNA) to direct sequence-specific binding of a nucleic acid-targeting complex to a target nucleic acid sequence may be assessed by any suitable assay.

A guide sequence, and hence a nucleic acid-targeting guide RNA may be selected to target any target nucleic acid sequence. The target sequence may be DNA. The target sequence may be genomic DNA. The target sequence may be mitochondrial DNA. The target sequence may be any RNA sequence. In some embodiments, the target sequence may be a sequence within a RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within a RNA molecule selected from the group consisting of mRNA, pre-mRNA, and rRNA. In some preferred embodiments, the target sequence may be a sequence within a RNA molecule selected from the group consisting of ncRNA, and lncRNA. In some more preferred embodiments, the target sequence may be a sequence within an mRNA molecule or a pre-mRNA molecule.

In certain embodiments, the gRNA comprises a stem loop, preferably a single stem loop. In certain embodiments, the direct repeat sequence forms a stem loop, preferably a single stem loop. In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27-30 nt, e.g., 27, 28, 29, or 30 nt, from 30-35 nt, e.g., 30, 31, 32, 33, 34, or 35 nt, or 35 nt or longer. In particular embodiments, the CRISPR/Cas system requires a tracrRNA. The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and gRNA sequence are contained within a single transcript, such that hybridization between the two produces a transcript having a secondary structure, such as a hairpin. In an embodiment of the invention, the transcript or transcribed polynucleotide sequence has at least two or more hairpins. In preferred embodiments, the transcript has two, three, four or five hairpins. In a further embodiment of the invention, the transcript has at most five hairpins. In a hairpin structure the portion of the sequence 5′ of the final “N” and upstream of the loop may correspond to the tracr mate sequence, and the portion of the sequence 3′ of the loop then corresponds to the tracr sequence. In a hairpin structure the portion of the sequence 5′ of the final “N” and upstream of the loop may alternatively correspond to the tracr sequence, and the portion of the sequence 3′ of the loop corresponds to the tracr mate sequence. In alternative embodiments, the CRISPR/Cas system does not require a tracrRNA, as is known by the skilled person.

In certain embodiments, the guide RNA (capable of guiding Cas to a target locus) may comprise (1) a guide sequence capable of hybridizing to a target locus and (2) a tracr mate or direct repeat sequence (in 5′ to 3′ orientation, or alternatively in 3′ to 5′ orientation, depending on the type of Cas protein, as is known by the skilled person). In particular embodiments, the CRISPR/Cas protein is characterized in that it makes use of a guide RNA comprising a guide sequence capable of hybridizing to a target locus and a direct repeat sequence, and does not require a tracrRNA. In particular embodiments, where the CRISPR/Cas protein is characterized in that it makes use of a tracrRNA, the guide sequence, tracr mate, and tracr sequence may reside in a single RNA, i.e. an sgRNA (arranged in a 5′ to 3′ orientation or alternatively arranged in a 3′ to 5′ orientation), or the tracr RNA may be a different RNA than the RNA containing the guide and tracr mate sequence. In these embodiments, the tracr hybridizes to the tracr mate sequence and directs the CRISPR/Cas complex to the target sequence.

Typically, in the context of an endogenous nucleic acid-targeting system, formation of a nucleic acid-targeting complex (comprising a guide RNA hybridized to a target sequence and complexed with one or more nucleic acid-targeting effector proteins) results in modification (such as cleavage) of one or both DNA or RNA strands in or near (e.g., within 1, 2, 3, 4, 5, 6, 7, 8, 9, 10, 20, 50, or more base pairs from) the target sequence. As used herein the term “sequence(s) associated with a target locus of interest” refers to sequences near the vicinity of the target sequence (e.g. within 1, 2, 3, 4, 5, 6, 7, 8, 9, 10, 20, 50, or more base pairs from the target sequence, wherein the target sequence is comprised within a target locus of interest). The skilled person will be aware of specific cut sites for selected CRISPR/Cas systems, relative to the target sequence, which as is known in the art may be within the target sequence or alternatively 3′ or 5′ of the target sequence.

In some embodiments, the unmodified nucleic acid-targeting effector protein may have nucleic acid cleavage activity. In some embodiments, the nuclease as described herein may direct cleavage of one or both nucleic acid (DNA, RNA, or hybrids, which may be single or double stranded) strands at the location of or near a target sequence, such as within the target sequence and/or within the complement of the target sequence or at sequences associated with the target sequence. In some embodiments, the nucleic acid-targeting effector protein may direct cleavage of one or both DNA or RNA strands within about 1, 2, 3, 4, 5, 6, 7, 8, 9, 10, 15, 20, 25, 50, 100, 200, 500, or more base pairs from the first or last nucleotide of a target sequence. In some embodiments, the cleavage may be blunt (e.g. for Cas9, such as SaCas9 or SpCas9). In some embodiments, the cleavage may be staggered (e.g. for Cpf1), i.e. generating sticky ends. In some embodiments, the cleavage is a staggered cut with a 5′ overhang. In some embodiments, the cleavage is a staggered cut with a 5′ overhang of 1 to 5 nucleotides, preferably of 4 or 5 nucleotides. In some embodiments, the cleavage site is upstream of the PAM. In some embodiments, the cleavage site is downstream of the PAM. In some embodiments, the nucleic acid-targeting effector protein that may be mutated with respect to a corresponding wild-type enzyme such that the mutated nucleic acid-targeting effector protein lacks the ability to cleave one or both DNA or RNA strands of a target polynucleotide containing a target sequence. As a further example, two or more catalytic domains of a Cas protein (e.g. RuvC I, RuvC II, and RuvC III or the HNH domain of a Cas9 protein) may be mutated to produce a mutated Cas protein substantially lacking all DNA cleavage activity. In some embodiments, a nucleic acid-targeting effector protein may be considered to substantially lack all DNA and/or RNA cleavage activity when the cleavage activity of the mutated enzyme is about no more than 25%, 10%, 5%, 1%, 0.1%, 0.01%, or less of the nucleic acid cleavage activity of the non-mutated form of the enzyme; an example can be when the nucleic acid cleavage activity of the mutated form is nil or negligible as compared with the non-mutated form. As used herein, the term “modified” Cas generally refers to a Cas protein having one or more modifications or mutations (including point mutations, truncations, insertions, deletions, chimeras, fusion proteins, etc.) compared to the wild type Cas protein from which it is derived. By derived is meant that the derived enzyme is largely based, in the sense of having a high degree of sequence homology with, a wildtype enzyme, but that it has been mutated (modified) in some way as known in the art or as described herein.

In certain embodiments, the target sequence should be associated with a PAM (protospacer adjacent motif) or PFS (protospacer flanking sequence or site); that is, a short sequence recognized by the CRISPR complex. The precise sequence and length requirements for the PAM differ depending on the CRISPR enzyme used, but PAMs are typically 2-5 base pair sequences adjacent the protospacer (that is, the target sequence). Examples of PAM sequences are given in the examples section below, and the skilled person will be able to identify further PAM sequences for use with a given CRISPR enzyme. Further, engineering of the PAM Interacting (PI) domain may allow programing of PAM specificity, improve target site recognition fidelity, and increase the versatility of the Cas, e.g. Cas9, genome engineering platform. Cas proteins, such as Cas9 proteins may be engineered to alter their PAM specificity, for example as described in Kleinstiver B P et al. Engineered CRISPR-Cas9 nucleases with altered PAM specificities. Nature. 2015 Jul. 23; 523(7561):481-5. doi: 10.1038/nature14592. In some embodiments, the method comprises allowing a CRISPR complex to bind to the target polynucleotide to effect cleavage of said target polynucleotide thereby modifying the target polynucleotide, wherein the CRISPR complex comprises a CRISPR enzyme complexed with a guide sequence hybridized to a target sequence within said target polynucleotide, wherein said guide sequence is linked to a tracr mate sequence which in turn hybridizes to a tracr sequence. The skilled person will understand that other Cas proteins may be modified analogously.

The Cas protein as referred to herein, such as without limitation Cas9, Cpf1 (Cas12a), C2c1 (Cas12b), C2c2 (Cas13a), C2c3, Cas13b protein, may originate from any suitable source, and hence may include different orthologues, originating from a variety of (prokaryotic) organisms, as is well documented in the art. In certain embodiments, the Cas protein is (modified) Cas9, preferably (modified) Staphylococcus aureus Cas9 (SaCas9) or (modified) Streptococcus pyogenes Cas9 (SpCas9). In certain embodiments, the Cas protein is (modified) Cpf1, preferably Acidaminococcus sp., such as Acidaminococcus sp. BV3L6 Cpf1 (AsCpf1) or Lachnospiraceae bacterium Cpf1, such as Lachnospiraceae bacterium MA2020 or Lachnospiraceae bacterium MD2006 (LbCpf1). In certain embodiments, the Cas protein is (modified) C2c2, preferably Leptotrichia wadei C2c2 (LwC2c2) or Listeria newyorkensis FSL M6-0635 C2c2 (LbFSLC2c2). In certain embodiments, the (modified) Cas protein is C2c1. In certain embodiments, the (modified) Cas protein is C2c3. In certain embodiments, the (modified) Cas protein is Cas13b.

In certain embodiments, the nucleic acid modification is effected by random mutagenesis. Cells or organisms may be exposed to mutagens such as UV radiation or mutagenic chemicals (such as for instance such as ethyl methanesulfonate (EMS)), and mutants with desired characteristics are then selected. Mutants can for instance be identified by TILLING

(Targeting Induced Local Lesions in Genomes). The method combines mutagenesis, such as mutagenesis using a chemical mutagen such as ethyl methanesulfonate (EMS) with a sensitive DNA screening-technique that identifies single base mutations/point mutations in a target gene. The TILLING method relies on the formation of DNA heteroduplexes that are formed when multiple alleles are amplified by PCR and are then heated and slowly cooled. A “bubble” forms at the mismatch of the two DNA strands, which is then cleaved by a single stranded nucleases. The products are then separated by size, such as by HPLC. See also McCallum et al. “Targeted screening for induced mutations”; Nat Biotechnol. 2000 April; 18(4):455-7 and McCallum et al. “Targeting induced local lesions IN genomes (TILLING) for plant functional genomics”; Plant Physiol. 2000 June; 123(2):439-42.

RNA interference (RNAi) is a biological process in which RNA molecules inhibit gene expression or translation, by neutralizing targeted mRNA molecules. Two types of small ribonucleic acid (RNA) molecules—microRNA (miRNA) and small interfering RNA (siRNA)—are central to RNA interference. RNAs are the direct products of genes, and these small RNAs can bind to other specific messenger RNA (mRNA) molecules and either increase or decrease their activity, for example by preventing an mRNA from being translated into a protein. The RNAi pathway is found in many eukaryotes, including animals, and is initiated by the enzyme Dicer, which cleaves long double-stranded RNA (dsRNA) molecules into short double-stranded fragments of about 21 nucleotide siRNAs (small interfering RNAs). Each siRNA is unwound into two single-stranded RNAs (ssRNAs), the passenger strand and the guide strand. The passenger strand is degraded and the guide strand is incorporated into the RNA-induced silencing complex (RISC). Mature miRNAs are structurally similar to siRNAs produced from exogenous dsRNA, but before reaching maturity, miRNAs must first undergo extensive post-transcriptional modification. A miRNA is expressed from a much longer RNA-coding gene as a primary transcript known as a pri-miRNA which is processed, in the cell nucleus, to a 70-nucleotide stem-loop structure called a pre-miRNA by the microprocessor complex. This complex consists of an RNase III enzyme called Drosha and a dsRNA-binding protein DGCR8. The dsRNA portion of this pre-miRNA is bound and cleaved by Dicer to produce the mature miRNA molecule that can be integrated into the RISC complex; thus, miRNA and siRNA share the same downstream cellular machinery. A short hairpin RNA or small hairpin RNA (shRNA/Hairpin Vector) is an artificial RNA molecule with a tight hairpin turn that can be used to silence target gene expression via RNA interference. The most well-studied outcome is post-transcriptional gene silencing, which occurs when the guide strand pairs with a complementary sequence in a messenger RNA molecule and induces cleavage by Argonaute 2 (Ago2), the catalytic component of the RISC. As used herein, an RNAi molecule may be an siRNA, shRNA, or a miRNA. In will be understood that the RNAi molecules can be applied as such to/in the plant, or can be encoded by appropriate vectors, from which the RNAi molecule is expressed. Delivery and expression systems of RNAi molecules, such as siRNAs, shRNAs or miRNAs are well known in the art.

In certain embodiments, the methods for obtaining plants or plant parts as described herein according to the invention, such as the methods for obtaining plants or plant parts having increased pathogen resistance or tolerance, involve or comprise transgenesis and/or gene editing, such as including CRISPR/Cas, TALEN, ZFN, meganucleases; (induced) mutagenesis, which may or may not be random mutagenesis, such as TILLING. In certain embodiments, the methods for obtaining plants or plant parts as described herein according to the invention, such as the methods for obtaining plants or plant parts having increased pathogen resistance or tolerance, involve or comprise RNAi applications, which may or may not be, comprise, or involve transgenic applications. By means of example, non-transgenic applications may for instance involve applying RNAi components such as double stranded siRNAs to plants or plant surfaces, such as for instance as a spray. Stable integration into the plant genome is not required.

In certain embodiments, the methods for obtaining plants or plant parts as described herein according to the invention, such as the methods for obtaining plants or plant parts having increased pathogen resistance or tolerance, do not involve or comprise transgenesis, gene editing, and/or mutagenesis.

In certain embodiments, the methods for obtaining plants or plant parts as described herein according to the invention, such as the methods for obtaining plants or plant parts having increased pathogen resistance or tolerance, involve, comprise or consist of breeding and selection.

In certain embodiments, the methods for obtaining plants or plant parts as described herein according to the invention, such as the methods for obtaining plants or plant parts having increased pathogen resistance or tolerance, do not involve, comprise or consist of breeding and selection.

In an aspect, the invention relates to a plant or plant part obtained or obtainable by the methods of the invention as described herein, such as the methods for obtaining plants or plant parts having increased pathogen resistance or tolerance.

In certain embodiments of the methods, uses, plants, plant parts, plant populations, nucleic acids, or proteins of the invention as described herein, CPL1 is mutated. In certain embodiments, said mutation is a dominant negative mutation. In certain embodiments, the mutation is in a acylphosphatase signature motif. In certain embodiments, said mutation is a mutation in a DXDXT motif, wherein D is aspactic acid, T is threonine, and X is any amino acid. By means of example, a DXDXT motif can be found in the Arabidopsis thaliana CPL4 protein at position 128 to 134. A corresponding DXDXT motif can be found in the Arabidopsis thaliana CPL1 protein at position 161 to 165, such as position 161 to 165 of SEQ ID NO: 1. Corresponding positions can be identified in orthologues, homologues, homeologues, or paralogues. In certain embodiments, one or both of the aspartic acid resisues is mutated. In certain embodiments the threonine residue is mutated. In certain preferred embodiments, the first aspartic acid residue (i.e. corresponding to the aspartic residue at position 161 of Arabidopsis thaliana CPL1) is mutated. In certain preferred embodiments, the mutation is a non-conservative mutation. In certain embodiments, the mutation is a mutation to a (small) non-polar amino acid. In certain embodiments, the mutation is a mutation to an alanine residue.

In certain embodiments, the CPL1 protein comprises one of the following mutations corresponding to:
D148X, preferably D148A, when said plant is from the genus Zea, preferably Zea mays, preferably wherein said Zea mays wild type sequence has a sequence as set forth in SEQ ID NO: 2;
D149X, preferably D148A, when said plant is from the genus Zea, preferably Zea mays, preferably wherein said Zea mays wild type sequence has a sequence as set forth in SEQ ID NO: 3;
D162X, preferably D162A, when said plant is from the genus Beta, preferably Beta vulgaris, preferably wherein said Beta vulgaris wild type sequence has a sequence as set forth in SEQ ID NO: 16;
D143X, preferably D143A, when said plant is from the genus Solanum, preferably Solanum tuberosum, preferably wherein said Beta vulgaris wild type sequence has a sequence as set forth in SEQ ID NO: 17;
D140X, D141X or D145X, preferably D140A, D141A or D145A, when said plant is from the genus Glycine, preferably Glycine max, preferably wherein said Glycine max wild type sequence has a sequence respectively as set forth in SEQ ID NO: 14/15, 12 or 13;
D149X, preferably D149A, when said plant is from the genus Triticum, preferably Triticum aestivum, preferably wherein said Beta vulgaris wild type sequence has a sequence as set forth in SEQ ID NO: 6, 7, or 8;
D147X, preferably D147A, when said plant is from the genus Triticum, preferably Triticum aestivum, preferably wherein said Beta vulgaris wild type sequence has a sequence as set forth in SEQ ID NO: 9, 10, or 11;
D149X or D144X, preferably D149A or D144A, when said plant is from the genus Sorghum, preferably Sorghum bicolor, preferably wherein said Sorghum bicolor wild type sequence has a sequence respectively as set forth in SEQ ID NO: 4 or 5;

wherein X is an amino acid different from D.

In certain embodiments, the mutated CPL1 protein has a sequence as set forth in any of SEQ ID NOs: 80 to 87.

In certain embodiments, the wild type CPL1 gene has or comprises:
(i) a nucleotide sequence having the cDNA or coding sequence of any of SEQ ID NOs: 19-34;
(ii) a nucleotide sequence encoding for a polypeptide having the amino acid sequence of any of SEQ ID NOs: 2-17;
(iii) a nucleotide sequence having at least 60% identity to the sequence of any of SEQ ID NOs: 19-34; such as at least 65%, 70%, 75%, 80%, 85%, 90%, 95% or more sequence identity, preferably at least 85% sequence identity, more preferably at least 90% sequence identity or at least 95% sequence identity;
(iv) a nucleotide sequence encoding for a polypeptide having at least 60% identity to the sequence of any of SEQ ID NOs: 2-17; such as at least 65%, 70%, 75%, 80%, 85%, 90%, 95% or more sequence identity, preferably at least 85% sequence identity, more preferably at least 90% sequence identity or at least 95% sequence identity;
(v) a nucleotide sequence hybridizing with the reverse complement of a nucleotide sequence as defined in (i) or (ii) under stringent hybridization conditions; and
(vi) a nucleotide sequence encoding a protein derived from the polypeptide encoded by the nucleotide sequence of any of (i) to (v) by way of substitution, deletion and/or addition of one or more amino acid(s).

In certain embodiments, the wild type ERF922 gene has or comprises:
(i) a nucleotide sequence having the cDNA or coding sequence of any of SEQ ID NOs: 52-65;
(ii) a nucleotide sequence encoding for a polypeptide having the amino acid sequence of any of SEQ ID NOs: 37-50;
(iii) a nucleotide sequence having at least 60% identity to the sequence of any of SEQ ID NOs: 52-65; such as at least 65%, 70%, 75%, 80%, 85%, 90%, 95% or more sequence identity, preferably at least 85% sequence identity, more preferably at least 90% sequence identity or at least 95% sequence identity;
(iv) a nucleotide sequence encoding for a polypeptide having at least 60% identity to the sequence of any of SEQ ID NOs: 37-50; such as at least 65%, 70%, 75%, 80%, 85%, 90%, 95% or more sequence identity, preferably at least 85% sequence identity, more preferably at least 90% sequence identity or at least 95% sequence identity;
(v) a nucleotide sequence hybridizing with the reverse complement of a nucleotide sequence as defined in (i) or (ii) under stringent hybridization conditions; and
(vi) a nucleotide sequence encoding a protein derived from the polypeptide encoded by the nucleotide sequence of any of (i) to (v) by way of substitution, deletion and/or addition of one or more amino acid(s).

In certain embodiments, the wild type CPL1 or ERF922 protein comprises a sequence which is respectively at least 95% identical, preferably over its entire length, to a sequence of any of SEQ ID NOs: 2-17 or 37-50 or which is encoded by a sequence which at least 95% identical, preferably over its entire length, to a sequence of any of SEQ ID NOs: 19-34, or 52-65.

In an aspect, the invention provides a nucleic acid which, after transcription or expression in a plant or after silencing in a plant, is suitable for increasing pathogen resistance or tolerance, such as caused by a mutated CPL1 and/or ERF922 gene. On the other hand, an endogenous DNA sequence in the genome of a plant, or in the genome of a plant haploid inductor, which is identical to one of the nucleic acids according to the invention, may also be modified such that the property of pathogen resistance or tolerance is mediated, or pathogen resistance or tolerance is increased, after transcription or expression of the endogenous DNA sequence.

The nucleic acid of the present invention is preferably an isolated nucleic acid which is extracted from its natural or original environment (genetic context). A nucleic acid may be double-stranded or single-stranded, and linear or circular. It may thereby be genomic DNA, synthetic DNA, cDNA, or an RNA type (for example, lncRNA, siRNA, or miRNA), wherein the nucleobase uracil occurs in RNA instead of the nucleobase thymine.

The nucleic acid according to the invention may be used as a transgene. On the other hand, an endogenous DNA sequence in the genome of a plant which is identical to one of the nucleic acids according to the invention, may also be modified such that the pathogen resistance or tolerance, such as caused by a mutated CPL1 and/or ERF922 gene can be enhanced, after transcription or expression of the endogenous DNA sequence or after silencing of the endogenous DNA sequence.

In an aspect, the invention relates to a (isolated) polynucleic acid comprising or consisting of a sequence as set forth in any of SEQ ID NOs: 19-35 or 52-79, the complement, or the reverse complement thereof.

In an aspect, the invention relates to a polynucleic acid encoding a polypeptide having a sequence as set forth in any of SEQ ID NOs: 2-17, 37-50, or 80-87, the complement, or the reverse complement thereof.

In an aspect, the invention relates to a polynucleic acid comprising:
(i) a nucleotide sequence having the sequence of any of SEQ ID NOs: 19-35 or 52-79, the complement or reverse complement thereof;
(ii) a nucleotide sequence encoding for a polypeptide having the amino acid sequence of any of SEQ ID NOs: 2-17, 37-50, or 80-87, the complement or reverse complement thereof;
(iii) a nucleotide sequence having at least 60% identity (preferably over the entire length) to the sequence of any of SEQ ID NOs: 19-35 or 52-79; such as at least 65%, 70%, 75%, 80%, 85%, 90%, 95% or more sequence identity, preferably at least 85% sequence identity, more preferably at least 90% sequence identity or at least 95% sequence identity, the complement or reverse complement thereof;
(iv) a nucleotide sequence encoding for a polypeptide having at least 60% identity (preferably over the entire length) to the sequence of any of SEQ ID NOs: 2-17, 37-50, or 80-87; such as at least 65%, 70%, 75%, 80%, 85%, 90%, 95% or more sequence identity, preferably at least 85% sequence identity, more preferably at least 90% sequence identity or at least 95% sequence identity, the complement or reverse complement thereof;
(v) a nucleotide sequence hybridizing with the reverse complement of a nucleotide sequence as defined in (i) or (ii) under stringent hybridization conditions, the complement or reverse complement thereof;
(vi) a nucleotide sequence encoding a protein derived from the polypeptide encoded by the nucleotide sequence of any of (i) to (v) by way of substitution, deletion and/or addition of one or more amino acid(s), the complement or reverse complement thereof; and
(vii) a functional fragment of any of (i) to (vi).

In an aspect, the invention relates to a polynucleic acid comprising:
(i) a nucleotide sequence having the sequence of any of SEQ ID NOs: 19-34 or 52-65, the complement or reverse complement thereof;
(ii) a nucleotide sequence encoding for a polypeptide having the amino acid sequence of any of SEQ ID NOs: 2-17 or 37-50, the complement or reverse complement thereof;
(iii) a nucleotide sequence having at least 60% identity (preferably over the entire length) to the sequence of any of SEQ ID NOs: 19-34 or 52-65; such as at least 65%, 70%, 75%, 80%, 85%, 90%, 95% or more sequence identity, preferably at least 85% sequence identity, more preferably at least 90% sequence identity or at least 95% sequence identity, the complement or reverse complement thereof, preferably wherein said nucleotide sequence encodes a functional polypeptide;
(iv) a nucleotide sequence encoding for a polypeptide having at least 60% identity (preferably over the entire length) to the sequence of any of SEQ ID NOs: 2-17 or 37-50; such as at least 65%, 70%, 75%, 80%, 85%, 90%, 95% or more sequence identity, preferably at least 85% sequence identity, more preferably at least 90% sequence identity or at least 95% sequence identity, the complement or reverse complement thereof, preferably wherein said nucleotide sequence encodes a functional polypeptide;
(v) a nucleotide sequence hybridizing with the reverse complement of a nucleotide sequence as defined in (i) or (ii) under stringent hybridization conditions, the complement or reverse complement thereof, preferably wherein said nucleotide sequence encodes a functional polypeptide;
(vi) a nucleotide sequence encoding a protein derived from the polypeptide encoded by the nucleotide sequence of any of (i) to (v) by way of substitution, deletion and/or addition of one or more amino acid(s), the complement or reverse complement thereof, preferably wherein said nucleotide sequence encodes a functional polypeptide; and
(vii) a functional fragment of any of (i) to (vi).

In an aspect, the invention relates to a polynucleic acid comprising:
(i) a nucleotide sequence encoding for a polypeptide having the amino acid sequence of any of SEQ ID NOs: 80-87, the complement or reverse complement thereof;
(ii) a nucleotide sequence encoding for a polypeptide having at least 60% identity (preferably over the entire length) to the sequence of any of SEQ ID NOs: 80-87; such as at least 65%, 70%, 75%, 80%, 85%, 90%, 95% or more sequence identity, preferably at least 85% sequence identity, more preferably at least 90% sequence identity or at least 95% sequence identity, the complement or reverse complement thereof, preferably wherein said nucleotide sequence encodes a dominant negative polypeptide;
(iii) a nucleotide sequence hybridizing with the reverse complement of a nucleotide sequence as defined in (i) or (ii) under stringent hybridization conditions, the complement or reverse complement thereof, preferably wherein said nucleotide sequence encodes a dominant negative polypeptide;
(iv) a nucleotide sequence encoding a protein derived from the polypeptide encoded by the nucleotide sequence of any of (i) to (iii) by way of substitution, deletion and/or addition of one or more amino acid(s), the complement or reverse complement thereof, preferably wherein said nucleotide sequence encodes a dominant negative polypeptide; and
(v) a functional fragment of any of (i) to (iv).

In an aspect, the invention relates to an (isolated) polynucleic acid comprising a mutated CPL1 and/or ERF922 as described herein elsewhere, such as encoding a dominant negative CPL1 and/or ERF922, or the complement, or the reverse complement thereof.

In certain embodiments, the polynucleic acid is DNA. In certain embodiments, the polynucleic acid is RNA. In certain embodiments, the polynucleic acid is single stranded. In certain embodiments, the polynucleic acid is double stranded. In certain embodiments, the polynucleic acid is single stranded DNA. In certain embodiments, the polynucleic acid is single stranded RNA. In certain embodiments, the polynucleic acid is double stranded DNA. In certain embodiments, the polynucleic acid is double stranded RNA.

In an aspect, the invention relates to a polynucleic acid specifically hybridizing with a polynucleic acid as described above, or the complement, or the reverse complement thereof. In certain embodiments, such polynucleic acids are at least 80% identical (i.e. complementary) (preferably over the entire length), preferably at least 90% identical, such as at least 95%, 96%, 97%, 98%, 99%, or 100% identical. In certain embodiments such polynucleic acids are 100% identical (i.e. complementary).

In an aspect, the invention relates to a first polynucleic acid comprising a second polynucleic acid specifically hybridizing with a polynucleic acid as described above (such as a gRNA, shRNA, siRNA), or the complement, or the reverse complement thereof. In certain embodiments, the second polynucleic acid and the polynucleic acid described above are at least 80% identical (i.e. complementary) (preferably over the entire length), preferably at least 90% identical, such as at least 95%, 96%, 97%, 98%, 99%, or 100% identical. In certain embodiments such polynucleic acids are 100% identical (i.e. complementary).

In certain embodiments, the invention relates to an RNAi molecule comprising a polynucleic acid having a sequence as set forth in any of SEQ ID NOs: 35, 69, or 70, the complement, or reverse complement thereof.

In certain embodiments, the invention relates to a gRNA comprising a sequence as set forth in any of SEQ ID NOs: 67, 68, or 71-79.

In certain embodiments, the nucleic acid molecule as described herein comprises less than 50000 nucleotides. In certain embodiments, the nucleic acid molecule as described herein comprises less than 40000 nucleotides. In certain embodiments, the nucleic acid molecule as described herein comprises less than 30000 nucleotides. In certain embodiments, the nucleic acid molecule as described herein comprises less than 25000 nucleotides. In certain embodiments, the nucleic acid molecule as described herein comprises less than 20000 nucleotides. In certain embodiments, the nucleic acid molecule as described herein comprises less than 15000 nucleotides. In certain embodiments, the nucleic acid molecule as described herein comprises less than 10000 nucleotides. In certain embodiments, the nucleic acid molecule as described herein comprises less than 5000 nucleotides. In certain embodiments, the nucleotide molecule as described herein comprises at least 100 nucleotides. In certain embodiments, the nucleic acid molecule as described herein comprises at least 100 nucleotides and less than 50000 nucleotides. In certain embodiments, the nucleic acid molecule as described herein comprises at least 100 nucleotides and less than 40000 nucleotides. In certain embodiments, the nucleic acid molecule as described herein comprises at least 100 nucleotides and less than 30000 nucleotides. In certain embodiments, the nucleic acid molecule as described herein comprises at least 100 nucleotides and less than 25000 nucleotides. In certain embodiments, the nucleic acid molecule as described herein comprises at least 100 nucleotides and less than 20000 nucleotides. In certain embodiments, the nucleic acid molecule as described herein comprises at least 100 nucleotides and less than 15000 nucleotides. In certain embodiments, the nucleic acid molecule as described herein comprises at least 100 nucleotides and less than 10000 nucleotides. In certain embodiments, the nucleic acid molecule as described herein comprises at least 100 nucleotides and less than 5000 nucleotides.

In certain embodiments, the polynucleic acid comprises at least 15 nucleotides, such as 16, 17, 18, 19, 20, 21, 22, 23, 24, or 25 nucleotides, such as at least 30, 35, 40, 45, or 50 nucleotides, such as at least 100, 200, 300, or 500 nucleotides.

In certain embodiments, a polynucleic acid as described herein is a primer or a probe. In certain embodiments, a polynucleic acid is a primer of between (and including) 10 and 80 nucleotides, such as between (and including) 15 and 50 nucleotides or 15 and 25 nucleotides. In certain embodiments, a polynucleic acid is a probe of between (and including) 10 and 500 nucleotides, such as between (and including) 50 and 400 nucleotides or 100 and 250 nucleotides.

In certain embodiments, the primer or probe is capable of specifically detecting a polynucleic acid of the invention, such as to specifically hybridise with a polynucleic acid of the invention.

In certain embodiments, the primer or probe is capable of hybridising with a unique nucleotide fragment or section of any of SEQ ID NOs: 19-34 or 52-68, or the complement or the reverse complement. In certain embodiments, the primer or probe is capable of hybridising with a unique nucleotide fragment or section of a polynucleic acid encoding a protein of any of SEQ ID NOs: 2-17, 37-50, or 80-87, or the complement or the reverse complement. In certain embodiments, the primer or probe is capable of specifically hybridizing with or capable of specifically detecting a polynucleic acid encoding a dominant negative CPL1 or ERF922 protein (i.e. capable of discriminating between wild type and dominant negative protein encoding sequences).

In will be understood that in embodiments relating to a set of forward and reverse primers, only one of both primers (forward or reverse) may need to be capable of discriminating between a wild type and mutant, and hence may be unique. The other primer may or may not be capable of discriminating between a wild type and mutant, and hence may be unique.

In certain embodiments, a polynucleic acid as described herein is an RNAi polynucleic acid, siRNA, or shRNA.

In certain embodiments, a polynucleic acid as described herein is a guide RNA (gRNA).

In an aspect, the invention relates to a vector comprising a polynucleic acid of the invention.

As used herein, a “vector” has its ordinary meaning in the art, and may for instance be a plasmid, a cosmid, a phage or an expression vector, a transformation vector, shuttle vector, or cloning vector; it may be double- or single-stranded, linear or circular; or it may transform a prokaryotic or eukaryotic host, either via integration into its genome or extrachromosomally. The nucleic acid according to the invention is preferably operatively linked in a vector with one or more regulatory sequences which allow the transcription, and, optionally, the expression, in a prokaryotic or eukaryotic host cell. A regulatory sequence—preferably, DNA—may be homologous or heterologous to the nucleic acid according to the invention. For example, the nucleic acid is under the control of a suitable promoter or terminator. Suitable promoters may be promoters which are constitutively induced (example: 35S promoter from the “Cauliflower mosaic virus” (Odell et al., 1985); those promoters which are tissue-specific are especially suitable (example: Pollen-specific promoters, Chen et al. (2010), Zhao et al. (2006), or Twell et al. (1991)), or are development-specific (example: blossom-specific promoters). Suitable promoters may also be synthetic or chimeric promoters which do not occur in nature, are composed of multiple elements, and contain a minimal promoter, as well as—upstream of the minimum promoter—at least one cis-regulatory element which serves as a binding location for special transcription factors. Chimeric promoters may be designed according to the desired specifics and are induced or repressed via different factors. Examples of such promoters are found in Gurr & Rushton (2005) or Venter (2007). For example, a suitable terminator is the nos-terminator (Depicker et al., 1982). The vector may be introduced via conjugation, mobilization, biolistic transformation, agrobacteria-mediated transformation, transfection, transduction, vacuum infiltration, or electroporation.

As used herein, the term “operatively linked” or “operably linked” means connected in a common nucleic acid molecule in such a manner that the connected elements are positioned and oriented relative to one another such that a transcription of the nucleic acid molecule may occur. A DNA which is operatively linked with a promoter is under the transcriptional control of this promoter.

In certain embodiments, the vector is a conditional expression vector. In certain embodiments, the vector is a constitutive expression vector. In certain embodiments, the vector is a tissue-specific expression vector, such as a pollen-specific expression vector. In certain embodiments, the vector is an inducible expression vector. All such vectors are well-known in the art.

Methods for preparation of the described vectors are commonplace to the person skilled in the art (Sambrook et al., 2001).

Also envisaged herein is a host cell, such as a plant cell, which comprises a nucleic acid as described herein, or a vector as described herein. The host cell may contain the nucleic acid as an extra-chromosomally (episomal) replicating molecule, or comprises the nucleic acid integrated in the nuclear or plastid genome of the host cell, or as introduced chromosome, e.g. minichromosome.

The host cell may be a prokaryotic (for example, bacterial) or eukaryotic cell (for example, a plant cell or a yeast cell). For example, the host cell may be an Agrobacterium, such as Agrobacterium tumefaciens or Agrobacterium rhizogenes. Preferably, the host cell is a plant cell.

A nucleic acid described herein or a vector described herein may be introduced in a host cell via well-known methods, which may depend on the selected host cell, including, for example, conjugation, mobilization, biolistic transformation, agrobacteria-mediated transformation, transfection, transduction, vacuum infiltration, or electroporation. In particular, methods for introducing a nucleic acid or a vector in an Agrobacterium cell are well-known to the skilled person and may include conjugation or electroporation methods. Also methods for introducing a nucleic acid or a vector into a plant cell are known (Sambrook et al., 2001) and may include diverse transformation methods such as biolistic transformation and Agrobacterium-mediated transformation.

In an aspect, the invention relates to a kit comprising polynucleic acids, such as primers (comprising forward and/or reverse primers) and/or probes, vectors, or host cell of the invention as described herein. The kit may further comprise instructions for use.

In an aspect, the invention relates to a method for identifying a plant, plant part, or plant population having increased resistance and/or tolerance to a pathogen, comprising screening for and/or identifying reduced or eliminated expression, activity, and/or stability of a CPL1 and/or ERF922 protein and/or gene or screening for and/or identifying a mutation as described herein elsewhere, in a plant, plant part, or plant population.

In an aspect, the invention relates to a method for identifying a plant, plant part, or plant population having increased resistance and/or tolerance to a pathogen, comprising isolating genetic material (such as genomic DNA or mRNA) from a plant, plant part, or plant population and screening for and/or identifying reduced or eliminated expression, activity, and/or stability of a CPL1 and/or ERF922 protein and/or gene or screening for and/or identifying a mutation as described herein elsewhere, in a plant, plant part, or plant population.

In an aspect, the invention relates to a method for selecting a plant, plant part, or plant population having increased resistance and/or tolerance to a pathogen, comprising screening for and/or identifying reduced or eliminated expression, activity, and/or stability of a CPL1 and/or ERF922 protein and/or gene or screening for and/or identifying a mutation as described herein elsewhere, in a plant, plant part, or plant population; and selecting a plant, plant part, or plant population having reduced or eliminated expression, activity, and/or stability of a CPL1 and/or ERF922 protein or having a mutation as described herein elsewhere.

In an aspect, the invention relates to a method for selecting a plant, plant part, or plant population having increased resistance and/or tolerance to a pathogen, comprising isolating genetic material (such as genomic DNA or mRNA) from a plant, plant part, or plant population and screening for and/or identifying reduced or eliminated expression, activity, and/or stability of a CPL1 and/or ERF922 protein and/or gene or screening for and/or identifying a mutation as described herein elsewhere, in a plant, plant part, or plant population; and selecting a plant, plant part, or plant population having reduced or eliminated expression, activity, and/or stability of a CPL1 and/or ERF922 protein or having a mutation as described herein elsewhere.

Methods for screening for the presence of a CPL1 and/or ERF922 gene having reduced expression, activity, or stability or the mutant CPL1 and/or ERF922 genes as described herein are known in the art. Without limitation, screening may encompass or comprise sequencing, hybridization based methods (such as (dynamic) allele-specific hybridization, molecular beacons, SNP microarrays), enzyme based methods (such as PCR, KASP (Kompetitive Allele Specific PCR), RFLP, ALFP, RAPD, Flap endonuclease, primer extension, 5′-nuclease, oligonucleotide ligation assay), post-amplification methods based on physical properties of DNA (such as single strand conformation polymorphism, temperature gradient gel electrophoresis, denaturing high performance liquid chromatography, high-resolution melting of the entire amplicon, use of DNA mismatch-binding proteins, SNPlex, surveyor nuclease assay), etc.

The aspects and embodiments of the invention are further supported by the following non-limiting examples. The following examples, including the experiments conducted and the results achieved, are provided for illustrative purposes only and are not constructed as limiting the present invention.

### EXAMPLES

### Example 1: CPL1 Gene Identification

Using homology searches with the Arabidopsis AtCPL1 protein sequence (SEQ ID NO: 1) CPL1-coding genes in multiple crop plants (SEQ ID NOs: 2-17) have been identified.

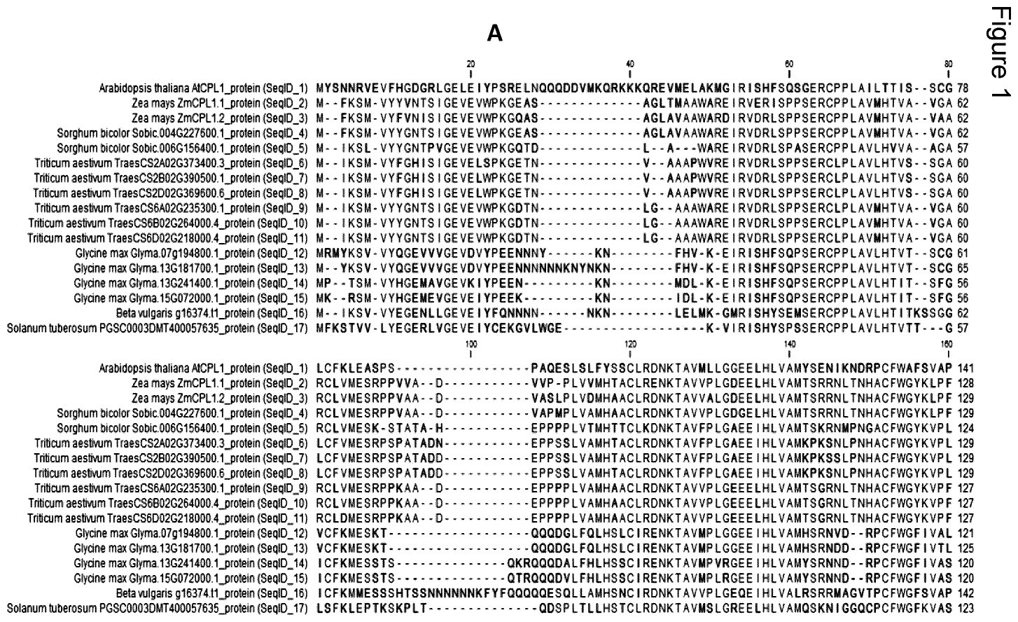

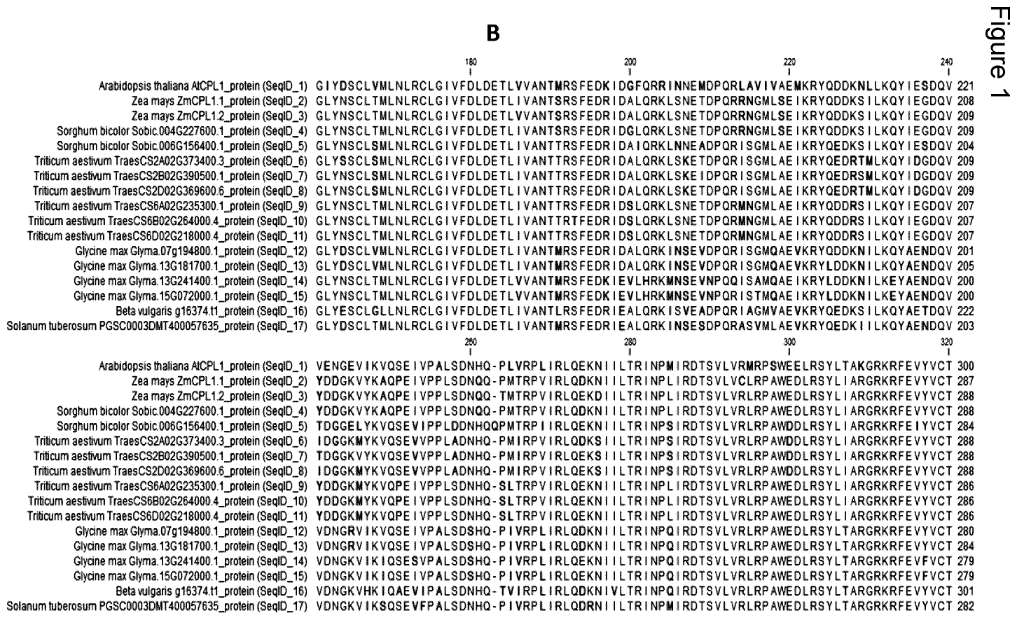

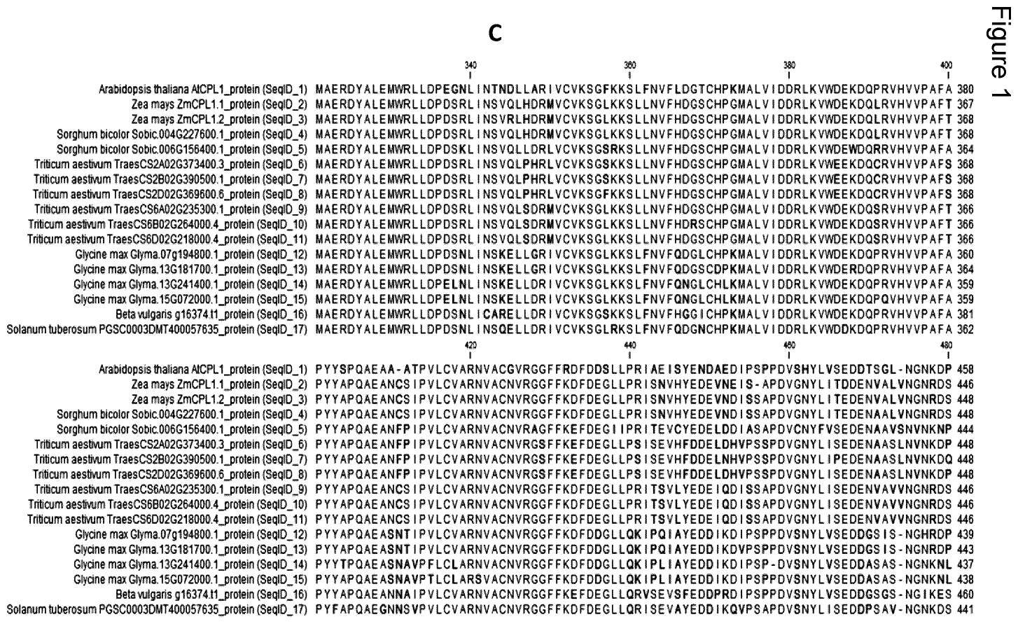

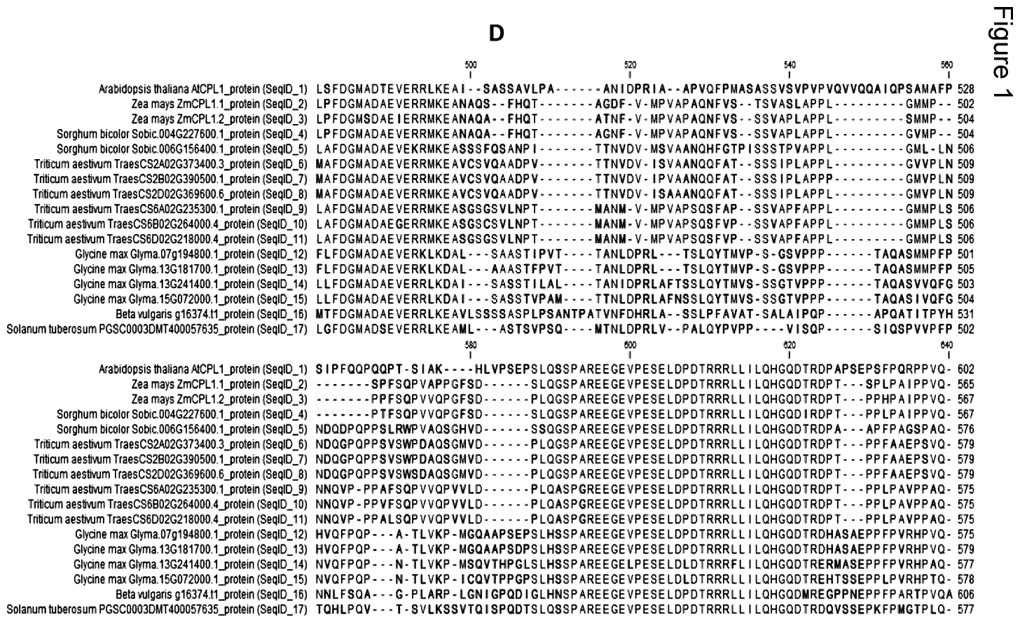

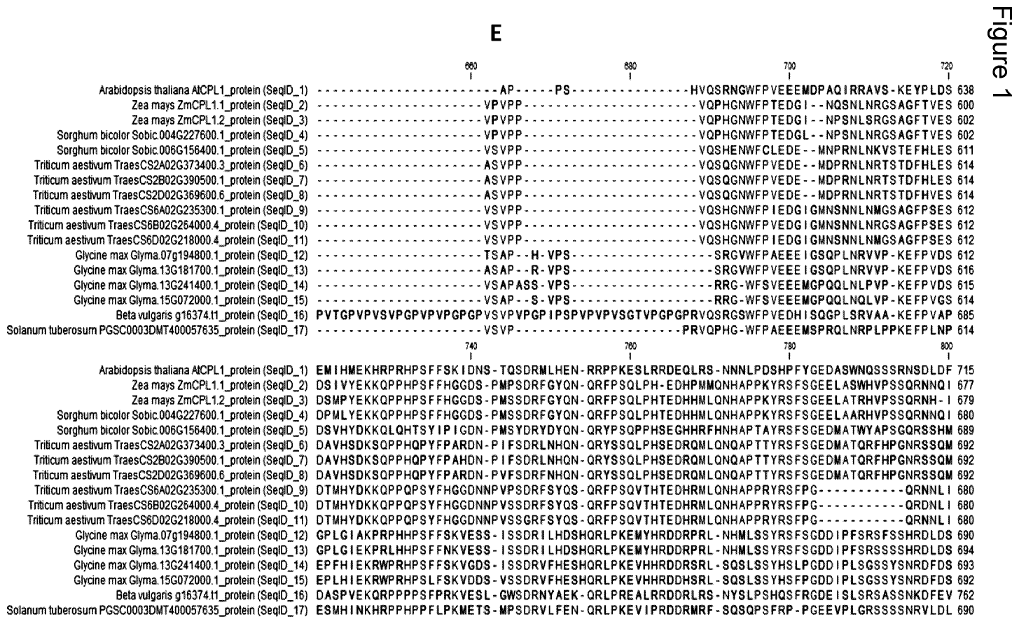

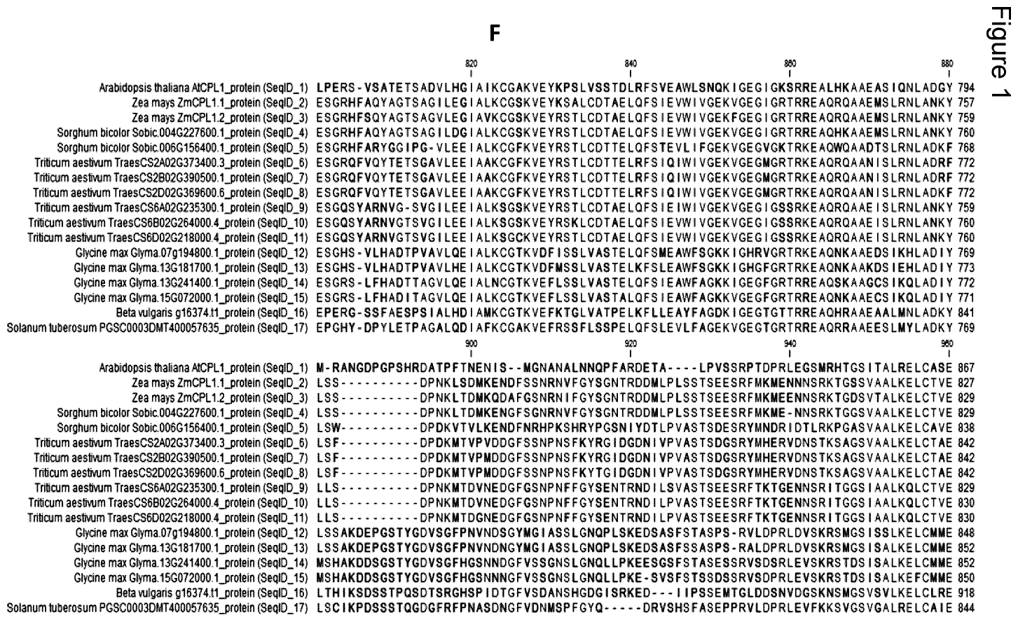

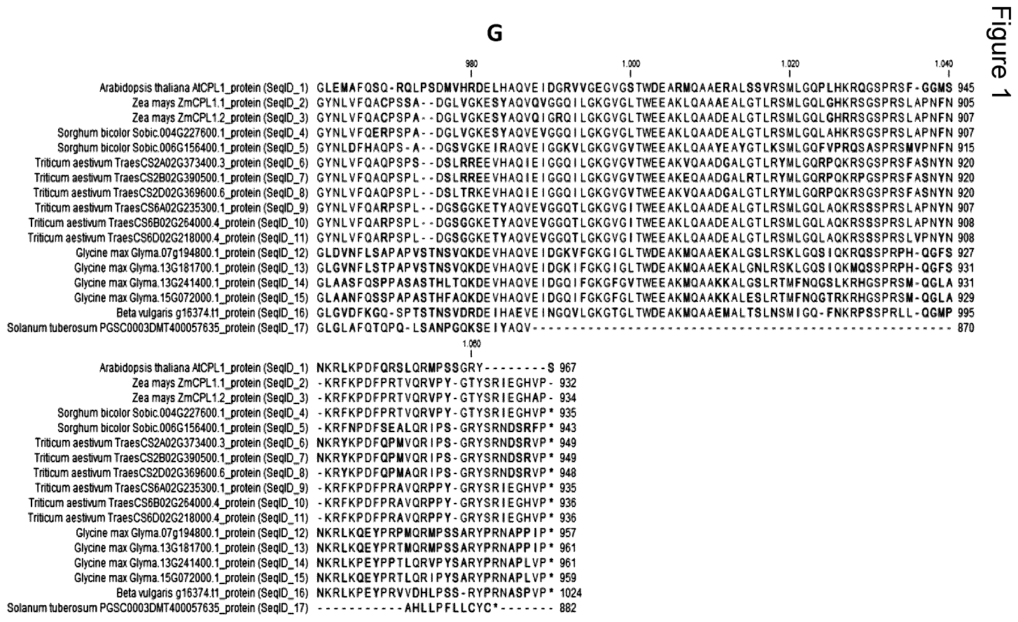

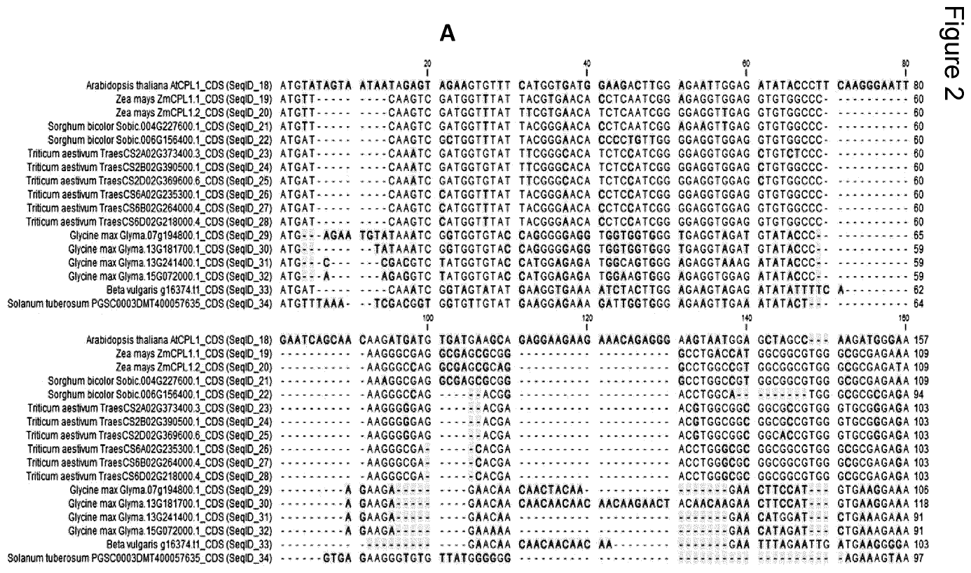

The identities of the selected CPL1 proteins (SEQ ID NOs 2-17) to the Arabidopsis AtCPL1 protein are between 46% and 60% at amino acid level (see FIG. 1; Table 1). The identities of the coding sequences (CDS) of the selected CPL1 genes (SEQ ID NOs 19-34) to the Arabidopsis AtCPL1 coding sequence (SEQ ID NO: 18) are between 53% and 66% at nucleotide level (see also FIG. 2; Table 2). Soybean (Glycine max) contains four paralogues CPL1 sequences, in maize (Zea mays) and Sorghum bicolor there are two CPL1 paralogues, each. In wheat (Triticum aestivum) there is one CPL1 gene on each of the chromosomes 2A, 2B, 2D, 6A, 6B, and 6D.

TABLE 1
Percent Identity between the different protein sequences of the discovered CPL1
genes and comparison to the Arabidopsis AtCPL1 protein sequence

| SEQ | | | | | | | | | | | | | | | | | |
|---|---|---|---|---|---|---|---|---|---|---|---|---|---|---|---|---|---|
| ID | | | | | | | | | | | | | | | | | |
| NOS | 1 | 2 | 3 | 4 | 5 | 6 | 7 | 8 | 9 | 10 | 11 | 12 | 13 | 14 | 15 | 16 | 17 |
| 1 | 100 | 46.8 | 46.8 | 47.6 | 46.1 | 47.9 | 48.0 | 48.1 | 46.9 | 67.9 | 46.9 | 59.9 | 59.4 | 57.9 | 58.4 | 51.4 | 51.8 |
| 2 | — | 100 | 92.2 | 93.9 | 67.7 | 67.9 | 68.1 | 68.4 | 80.2 | 80.2 | 80.0 | 51.2 | 51.1 | 49.7 | 50.3 | 47.6 | 48.9 |
| 3 | — | — | 100 | 93.7 | 67.8 | 68.6 | 69.0 | 69.3 | 80.5 | 80.8 | 80.5 | 51.1 | 50.9 | 49.8 | 50.4 | 47.6 | 48.7 |
| 4 | — | — | — | 100 | 68.9 | 69.9 | 69.9 | 70.1 | 82.4 | 82.5 | 82.4 | 52.3 | 52.2 | 50.6 | 51.4 | 48.6 | 49.3 |
| 5 | — | — | — | — | 100 | 73.6 | 73.4 | 73.9 | 69.6 | 69.5 | 69.9 | 51.0 | 50.8 | 48.4 | 49.1 | 47.1 | 48.2 |
| 6 | — | — | — | — | — | 100 | 97.8 | 98.0 | 70.6 | 70.4 | 70.3 | 52.5 | 52.7 | 50.5 | 50.8 | 49.5 | 48.3 |
| 7 | — | — | — | — | — | — | 100 | 97.3 | 70.7 | 70.5 | 70.4 | 52.3 | 52.5 | 50.4 | 50.9 | 49.5 | 48.2 |
| 8 | — | — | — | — | — | — | — | 100 | 71.4 | 71.2 | 71.1 | 52.4 | 52.6 | 50.5 | 51.1 | 49.1 | 48.4 |
| 9 | — | — | — | — | — | — | — | — | 100 | 98.5 | 98.8 | 52.6 | 52.0 | 50.5 | 51.4 | 48.2 | 48.2 |
| 10 | — | — | — | — | — | — | — | — | — | 100 | 98.4 | 52.5 | 52.0 | 50.5 | 51.3 | 48.0 | 47.9 |
| 11 | — | — | — | — | — | — | — | — | — | — | 100 | 52.5 | 51.9 | 50.5 | 51.3 | 48.0 | 48.1 |
| 12 | — | — | — | — | — | — | — | — | — | — | — | 100 | 95.3 | 76.3 | 77.2 | 59.7 | 58.7 |
| 13 | — | — | — | — | — | — | — | — | — | — | — | — | 100 | 75.3 | 76.3 | 59.3 | 58.4 |
| 14 | — | — | — | — | — | — | — | — | — | — | — | — | — | 100 | 92.6 | 56.9 | 57.2 |
| 15 | — | — | — | — | — | — | — | — | — | — | — | — | — | — | 100 | 57.4 | 57.9 |
| 16 | — | — | — | — | — | — | — | — | — | — | — | — | — | — | — | 100 | 53.3 |
| 17 | — | — | — | — | — | — | — | — | — | — | — | — | — | — | — | — | 100 |

TABLE 2
Percent Identity between the different coding sequences of the discovered CPL1
genes and comparison to the Arabidopsis AtCPL1 coding sequence

| SEQ | | | | | | | | | | | | | | | | | |
|---|---|---|---|---|---|---|---|---|---|---|---|---|---|---|---|---|---|
| ID | | | | | | | | | | | | | | | | | |
| NOS | 18 | 19 | 20 | 21 | 22 | 23 | 24 | 25 | 26 | 27 | 28 | 29 | 30 | 31 | 32 | 33 | 34 |
| 18 | 100 | 54.6 | 54.5 | 55.0 | 54.2 | 55.9 | 55.4 | 55.8 | 53.8 | 53.6 | 53.76 | 66.2 | 66.0 | 65.4 | 65.2 | 57.7 | 57.2 |
| 19 | — | 100 | 93.6 | 95.1 | 73.3 | 74.2 | 74.2 | 74.6 | 82.0 | 81.7 | 81.6 | 58.6 | 58.5 | 57.8 | 58.1 | 53.5 | 53.9 |
| 20 | — | — | 100 | 94.8 | 73.2 | 74.6 | 74.6 | 75.0 | 82.1 | 81.9 | 81.7 | 58.7 | 58.6 | 57.8 | 58.2 | 53.8 | 53.2 |
| 21 | — | — | — | 100 | 73.7 | 75.0 | 74.9 | 75.3 | 82.8 | 82.6 | 82.4 | 58.9 | 58.8 | 58.1 | 58.4 | 54.0 | 53.8 |
| 22 | — | — | — | — | 100 | 80.2 | 79.9 | 80.4 | 74.1 | 74.0 | 74.2 | 59.4 | 59.5 | 58.2 | 58.6 | 53.8 | 53.7 |
| 23 | — | — | — | — | — | 100 | 98.3 | 98.6 | 76.3 | 76.1 | 76.3 | 59.6 | 60.0 | 59.1 | 59.3 | 53.8 | 53.7 |
| 24 | — | — | — | — | — | — | 100 | 98.1 | 76.0 | 75.8 | 76.1 | 59.5 | 59.8 | 59.2 | 59.3 | 53.6 | 53.6 |
| 25 | — | — | — | — | — | — | — | 100 | 76.5 | 76.2 | 76.5 | 59.7 | 60.1 | 59.2 | 59.4 | 53.8 | 53.9 |
| 26 | — | — | — | — | — | — | — | — | 100 | 99.0 | 99.0 | 58.5 | 58.3 | 57.9 | 58.4 | 53.2 | 53.0 |
| 27 | — | — | — | — | — | — | — | — | — | 100 | 98.7 | 58.5 | 58.2 | 57.9 | 58.3 | 53.2 | 52.8 |
| 28 | — | — | — | — | — | — | — | — | — | — | 100 | 58.5 | 58.4 | 58.0 | 58.4 | 53.3 | 52.9 |
| 29 | — | — | — | — | — | — | — | — | — | — | — | 100 | 95.8 | 82.4 | 82.9 | 63.1 | 61.7 |
| 30 | — | — | — | — | — | — | — | — | — | — | — | — | 100 | 81.4 | 82.1 | 62.9 | 61.5 |
| 31 | — | — | — | — | — | — | — | — | — | — | — | — | — | 100 | 95.1 | 62.6 | 61.5 |
| 32 | — | — | — | — | — | — | — | — | — | — | — | — | — | — | 100 | 62.6 | 61.3 |
| 33 | — | — | — | — | — | — | — | — | — | — | — | — | — | — | — | 100 | 56.3 |
| 34 | — | — | — | — | — | — | — | — | — | — | — | — | — | — | — | — | 100 |

### Example 2: Fungal Resistance in Corn (Zea mays) by RNAi or Mutated CPL1 Genes

An RNAi-based silencing construct against the maize ZmCPL1.1 and ZmCPL1.2 genes (FIG. 1-2) has been developed and stably transformed into maize plants of the A188 genotype.

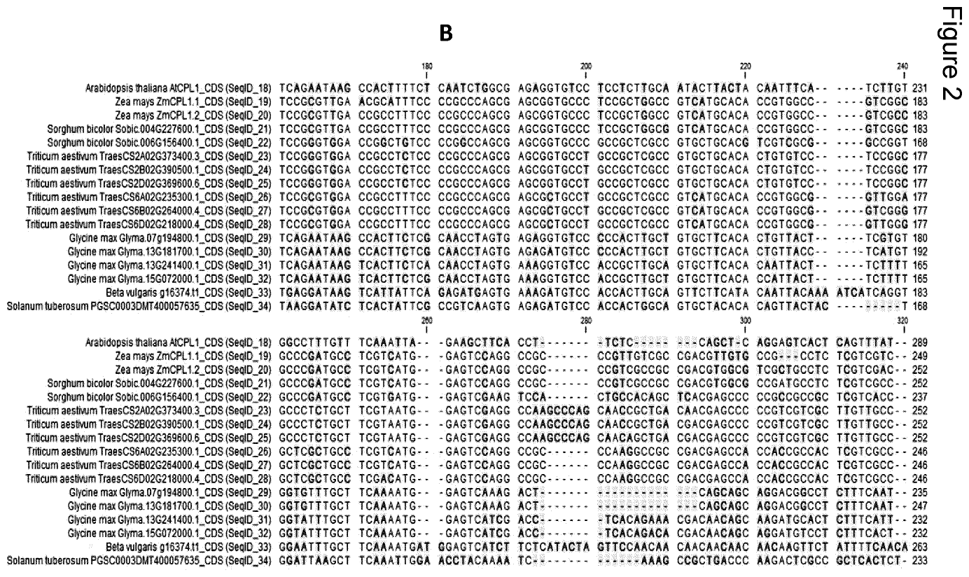

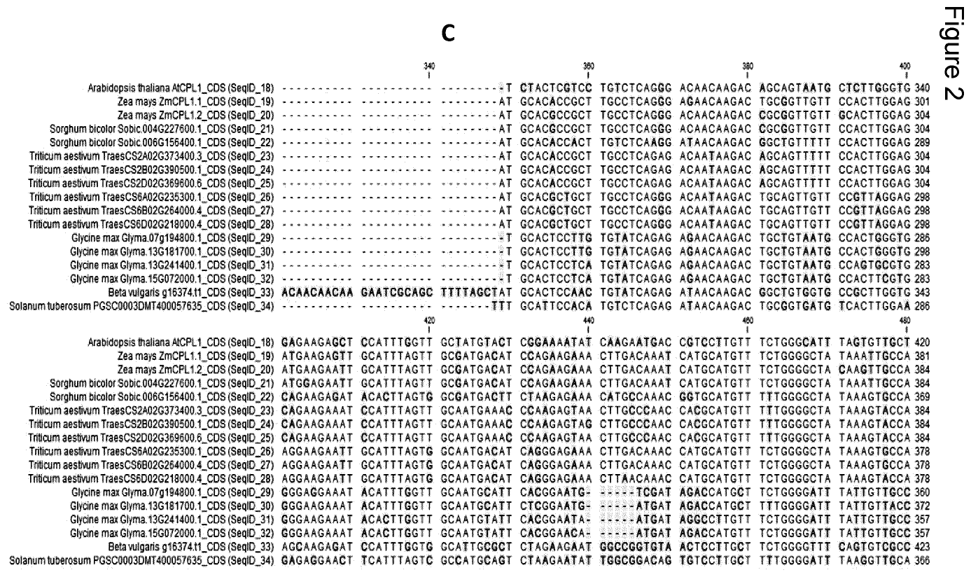

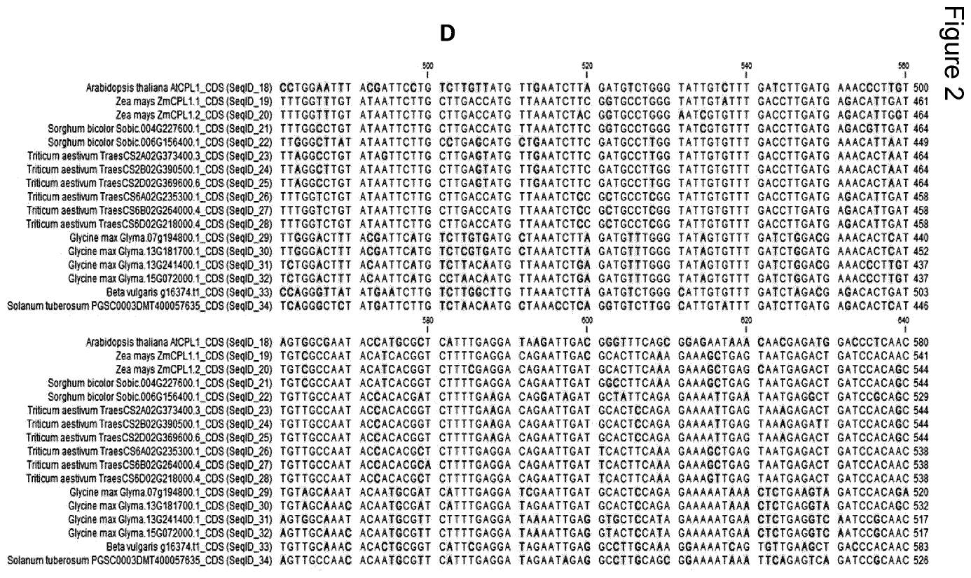

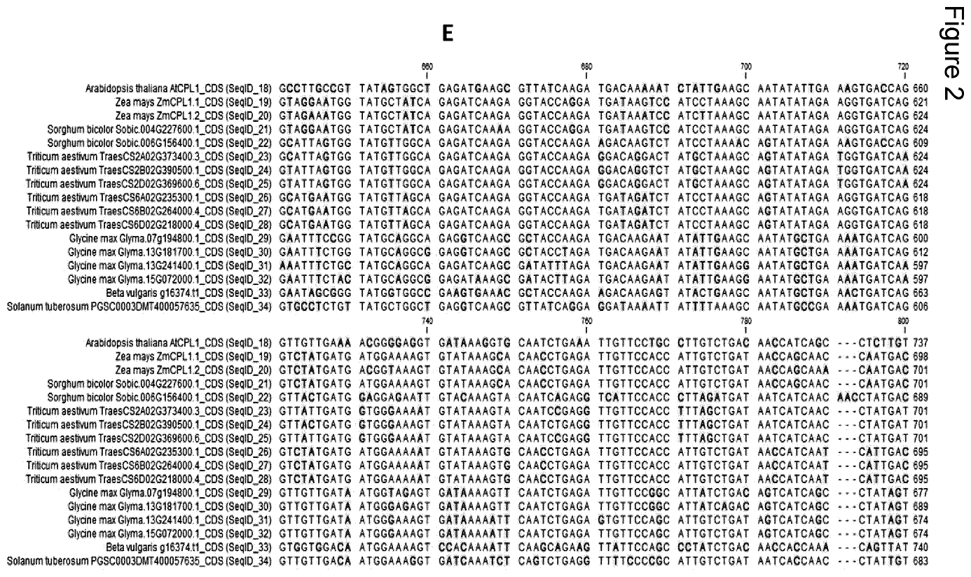

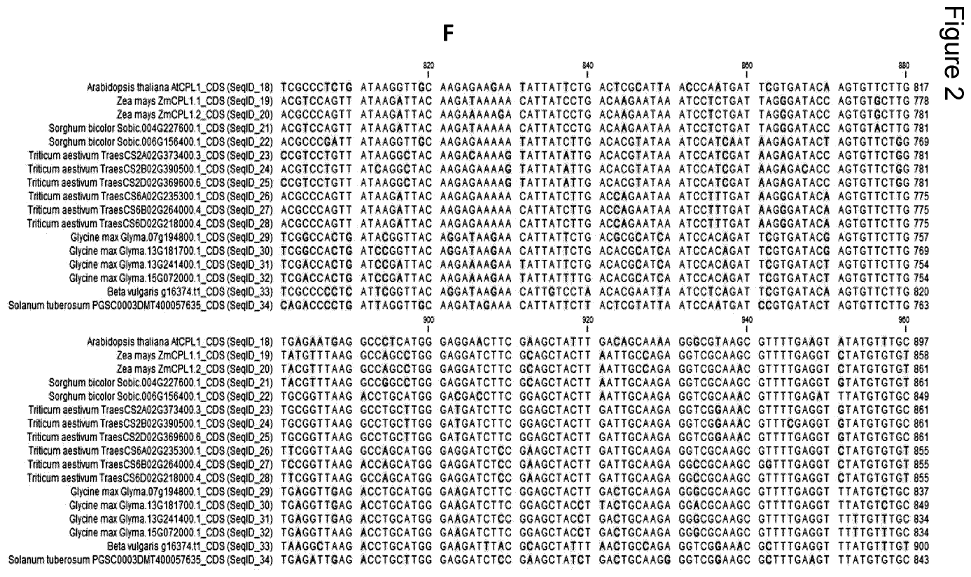

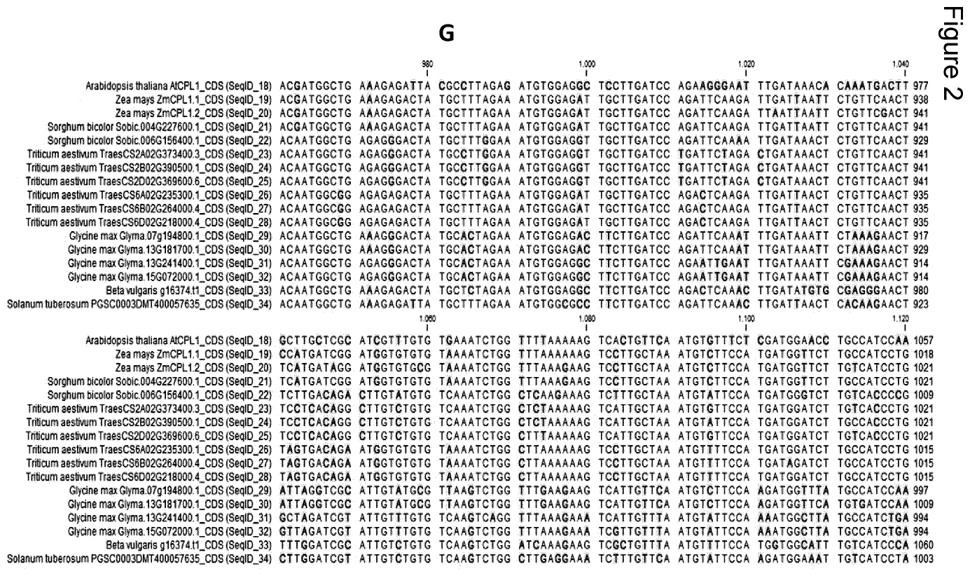

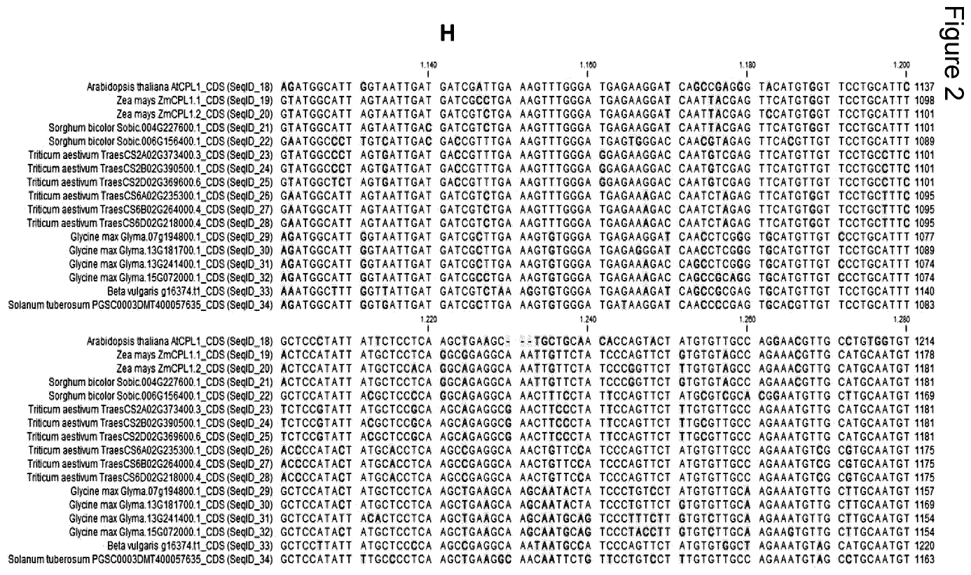

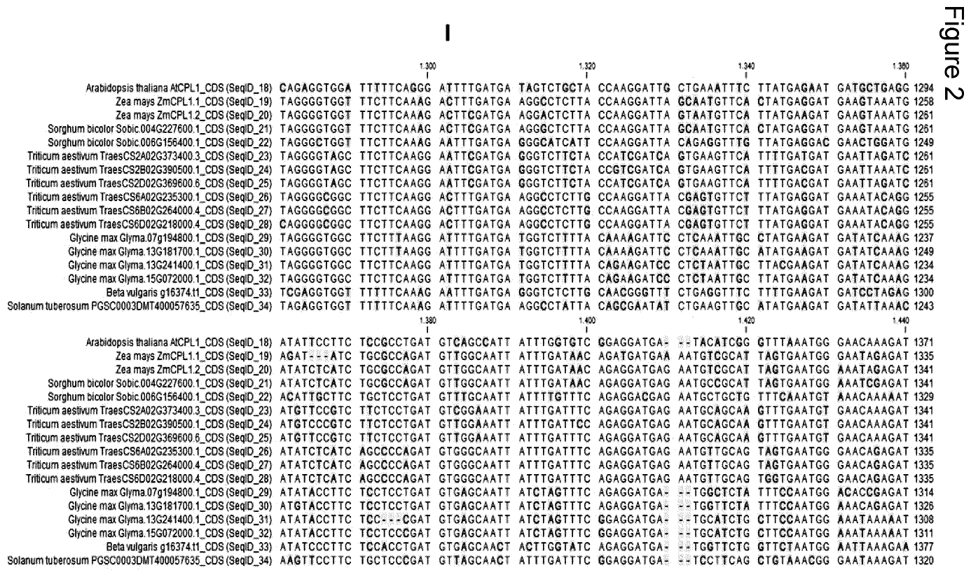

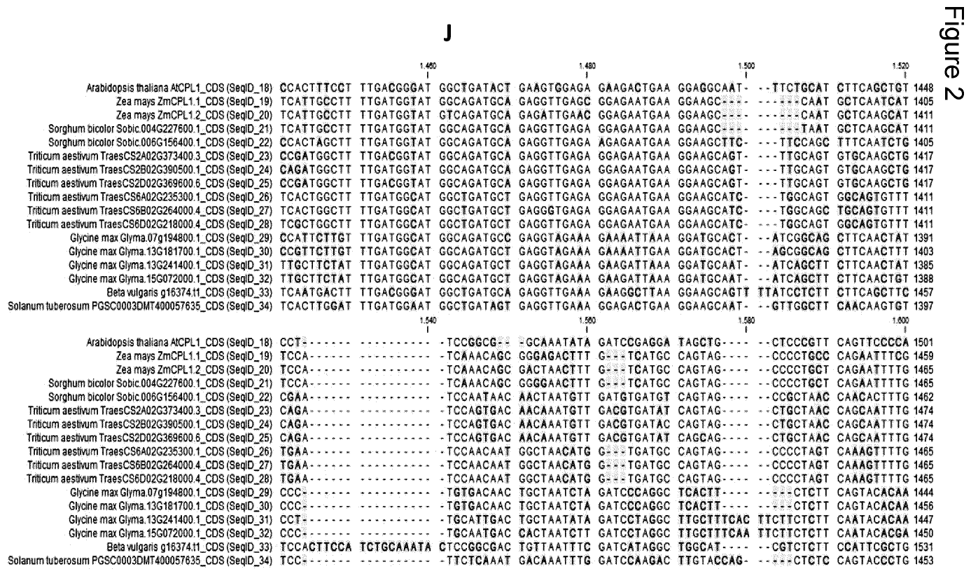

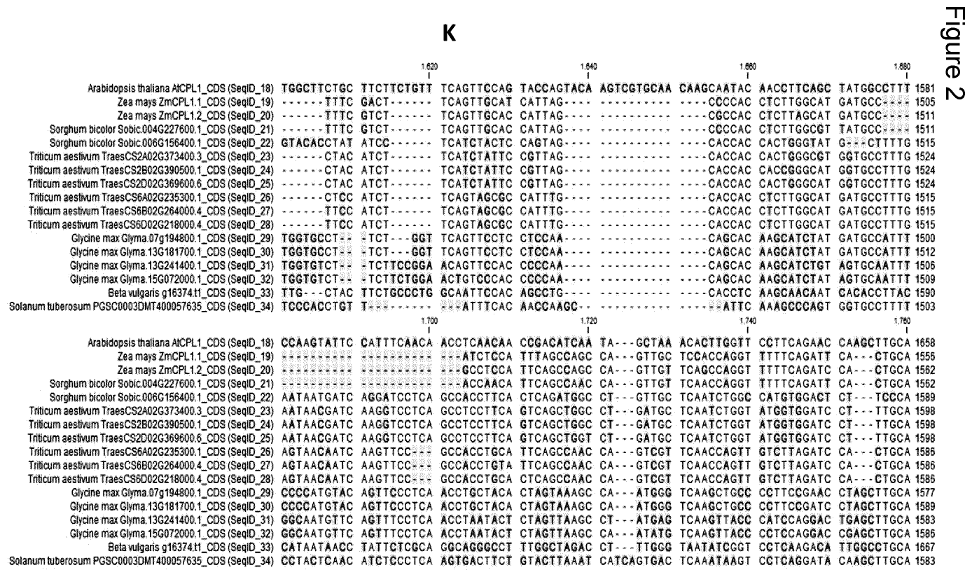

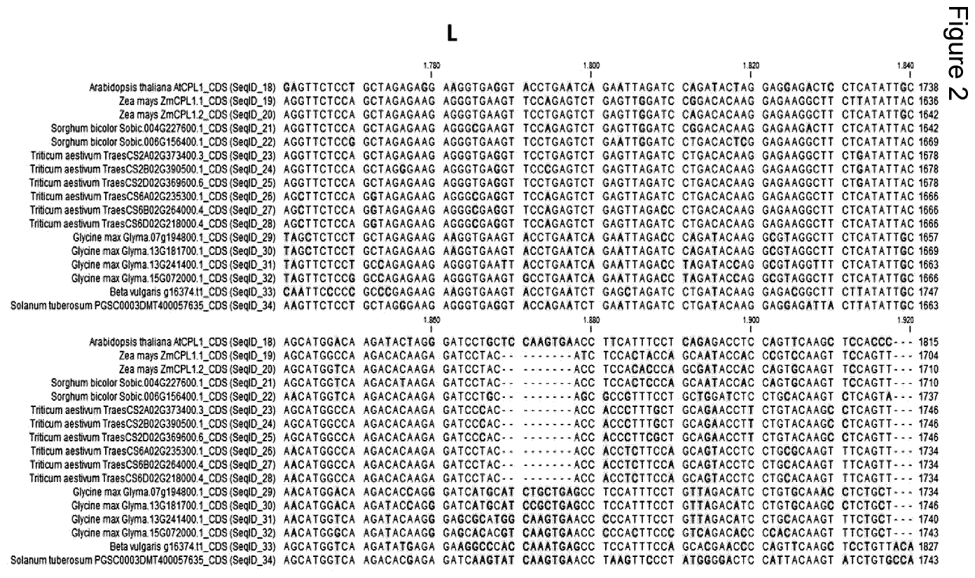

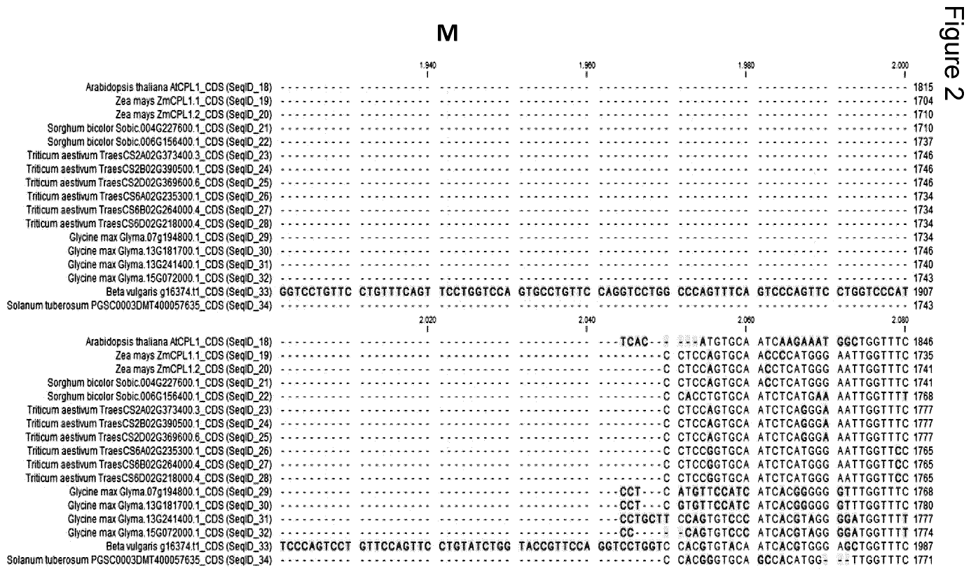

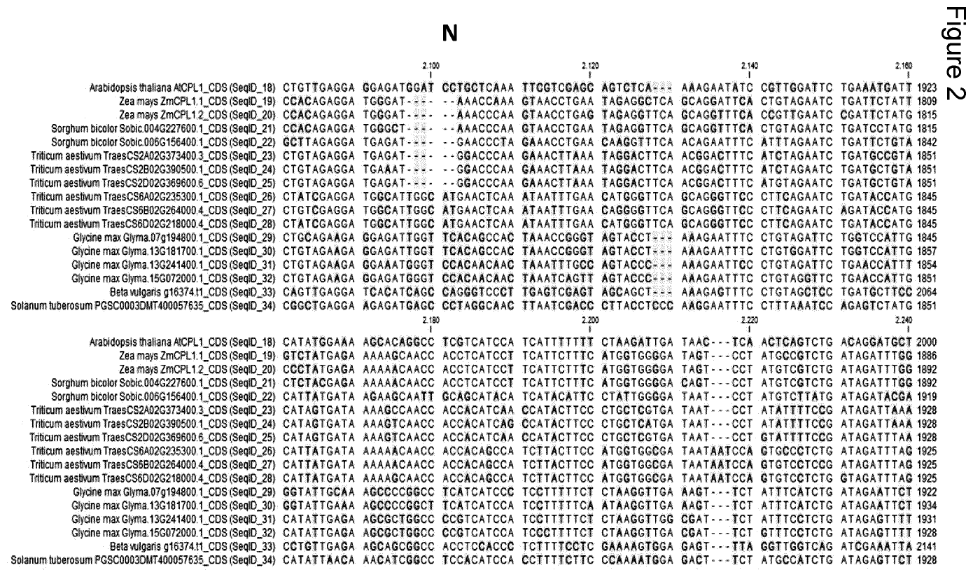

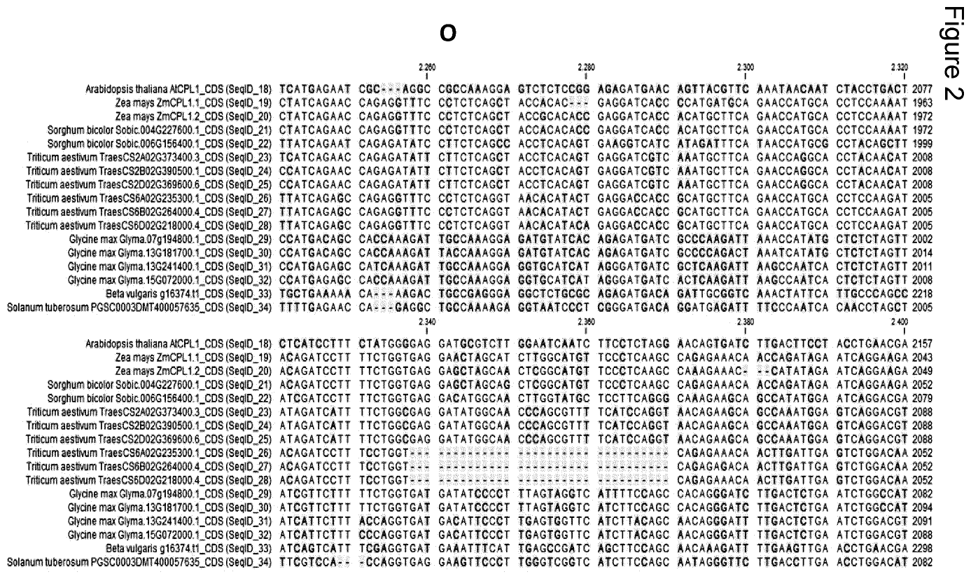

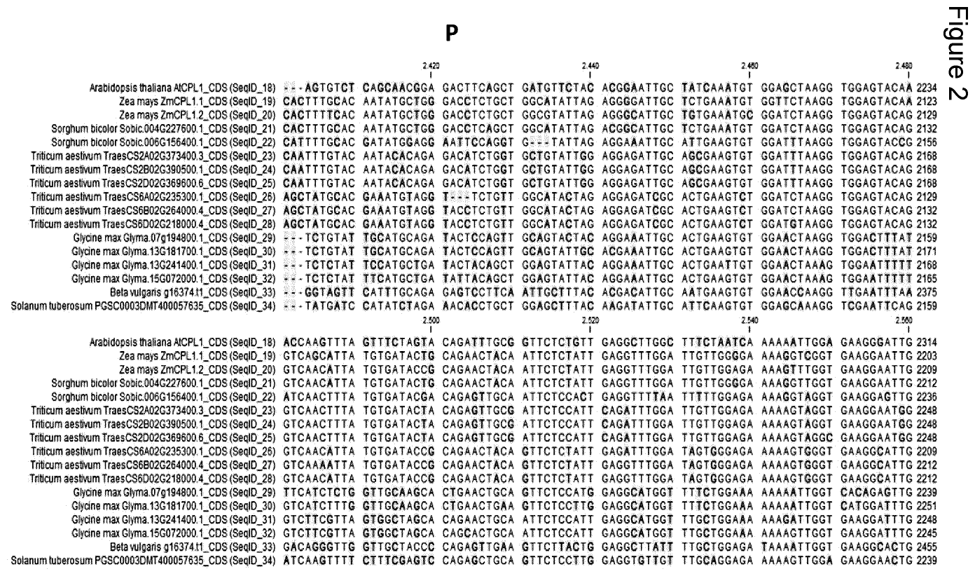

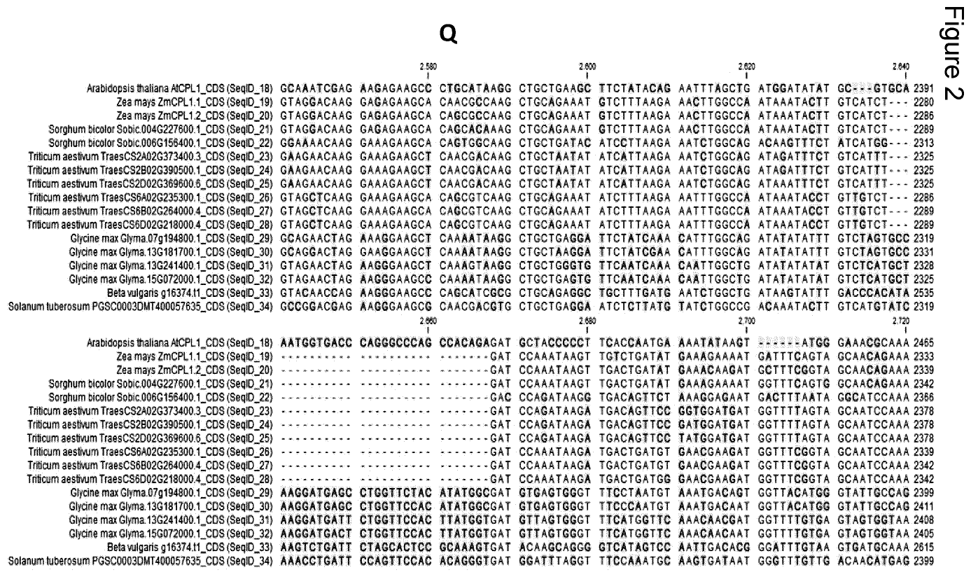

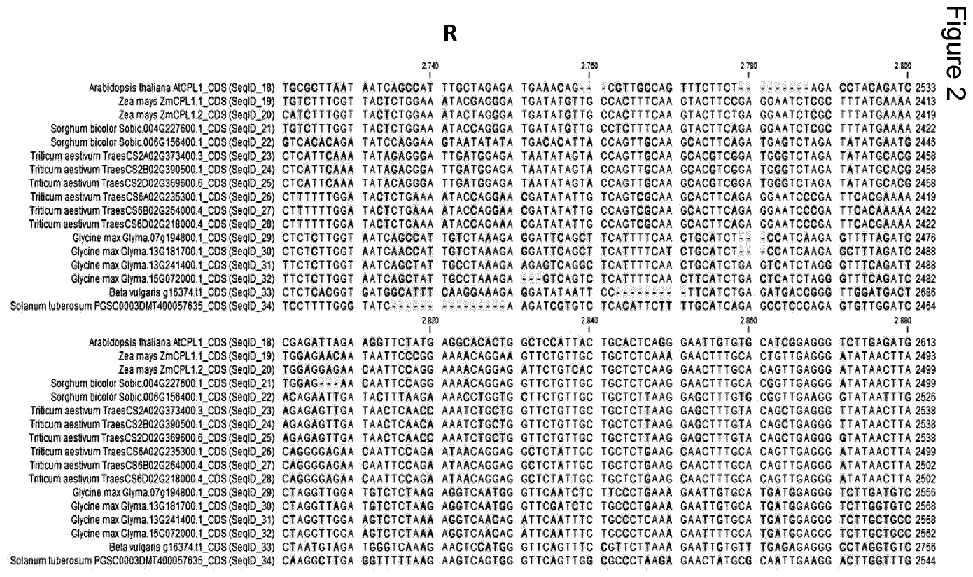

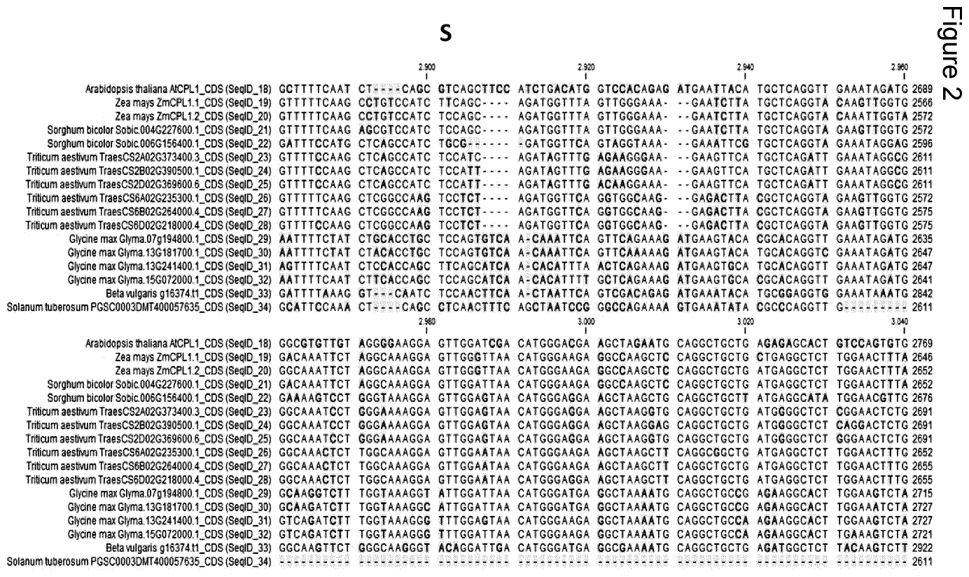

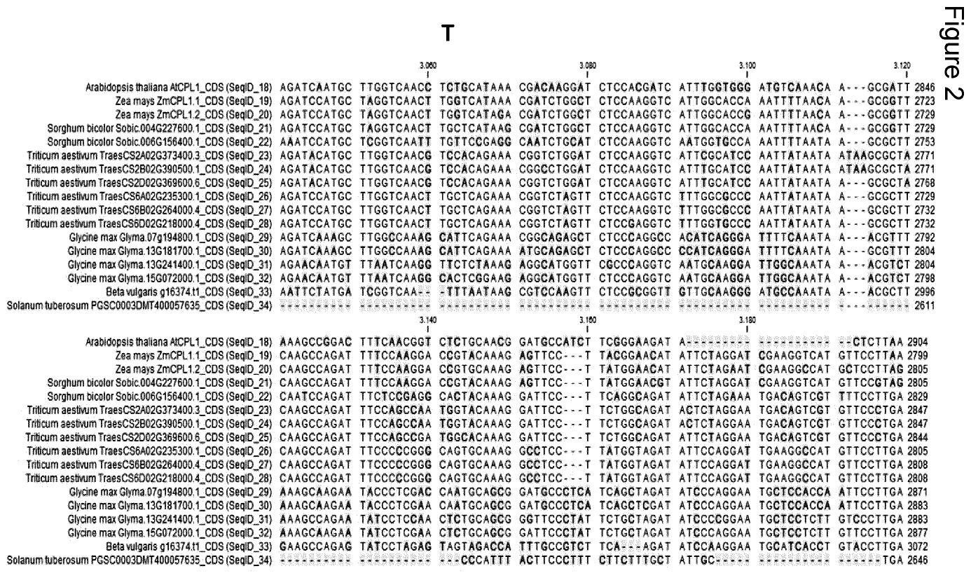

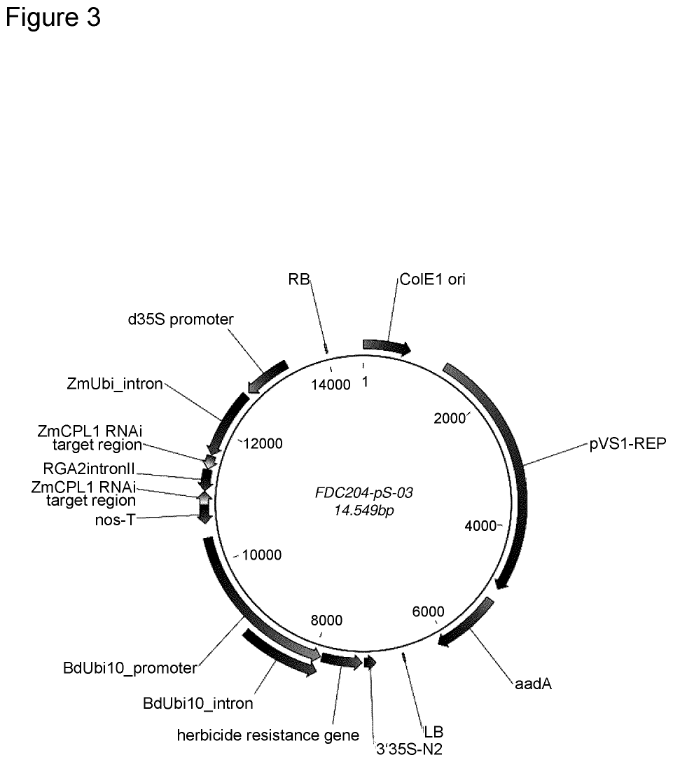

The ZmCPL1 silencing sequence (SEQ ID NO: 35) was specifically selected for having very high homology to the selected ZmCPL1 genes (100% identity to ZmCPL1.1 and >95% identity to ZmCPL1.2) and, at the same time, to avoid large stretches of homology (<20 bp) to other sequences in the maize genome. The vector construct used for transformation is shown in FIG. 3. Transgenic maize plants of the segregating T1 generation as well as homozygous T2 lines along with the respective azygous sister lines (null-segregants) are tested for resistance against Setosphaeria turcica (Northern Corn Leaf Blight (NCLB)) in the greenhouse and Fusarium resistance in the field.

These experiments show that the downregulation of the ZmCPL1 genes leads to increased resistance against a hemibiotrophic fungus and that downregulation of ZmCPL1 does not cause any growth retardation.

There are multiple ways to downregulate the expression of a gene. Among them are for example the expression of an RNAi construct or a microRNA construct or the modification of the promoter or other regulatory elements of a gene. For downregulation of CPL1 genes, in addition to the aforementioned methods, a dominant-negative allele of CPL1 has been expressed. It has been shown that the point mutation D128A in the conserved DXDXT motif of the catalytic phosphatase domain resulted in a dominant-negative form of the Arabidopsis protein AtCPL4, that is involved in normal growth and plant development (Fukudome et al.; 2014). Overexpression of the dominant negative allele AtCPL4_D128A in Arabidopsis was lethal and resembled the phenotype of AtCPL4 knock-out lines that are homozygous lethal. Arabidopsis plants with AtCPL4_RNAi constructs were viable with mild toxicity phenotype. This shows that strong overexpression of a dominant-negative AtCPL4 allele resembles a complete knock out of AtCPL4.

To achieve downregulation of CPL1 genes a dominant negative allele under the control of a strong overexpressing promoter, or under control of a weaker promoter for example the native promoter is used to gain dominant negative alleles of the selected CPL1 genes. In one embodiment mutations in the DXDXT motif similar to D128A in AtCPL4 are used. Examples for DXDXT motif modifications are D148A in ZmCPL1.1 (SEQ ID NO: 80) and D149A in ZmCPL1.2 (SEQ ID NO: 81). In other agronomically important crops these position corresponds to D162A in Beta vulgaris g16374.t1_protein (CPL1 homologue in sugarbeet; SEQ ID NO: 82), D143A in Solanum tuberosum PGSC0003DMT400057635_protein (CPL1 homologue in potato; SEQ ID NO: 83), D141A in Glycine max Glyma.07g194800.1_protein (CPL1 homologue in soybean; SEQ ID NO: 84), D149A in Triticum aestivum TraesCS2A02G373400.3_protein (CPL1 homologue in wheat; SEQ ID NO: 85), D147A in Triticum aestivum TraesCS6B02G264000.4_protein (CPL1 homologue in wheat; SEQ ID NO: 86), and D149A in Sorghum bicolor Sobic.004G227600.1_protein (CPL1 homologue in Sorghum; SEQ ID NO: 87).

Consequently, downregulation of CPL1 genes can also be achieved by introducing a point mutation in a native CPL1 gene that leads to a dominant-negative allele and to keep this mutation in a heterozygous state. The resistance effect of a dominant-negative allele of CPL1 would be genetically dominant which has benefits in breeding of resistant hybrid crops as compared to recessive mutations in the promoter, for example, that would need to be present in both parents of the hybrid. If paralogues CPL1 genes are present, like in maize, wheat, soybean, and Sorghum, using a dominant-negative CPL1 allele for engineering resistant plants requires less effort than, for example, downregulating all CPL1 paralogs by promoter modifications at the same time.

### Example 3: Fungal Resistance in Wheat (Triticum aestivum) Via VIGS Approach Against CPL1

Two silencing constructs targeting all six paralogs/homeologs of CPL1 for virus induced gene silencing (VIGS) experiments in wheat have been developed. The silencing sequences were specifically designed for having high homology (>83% identity) to all six wheat CPL1 homologs and, at the same time, to avoid large stretches of homology (<20 bp) to other coding sequences in the wheat genome. For the VIGS experiments an appropriate vector system was used. Here, the TaCPL1 silencing sequences (SEQ ID NOs: 69 and 70) were inserted individually. The virus particles carrying the silencing sequences were assembled by Agrobacterium co-infiltration-based transformation into Nicotiana benthamiana. Leaves of wheat cultivar Passat (KWS, Einbeck, Germany) are subsequently transfected with sap extracted from the infiltrated N. benthamiana leaves. 14 days after transfection with the virus variant, the wheat plants are infected with a pathogen suspension, e.g. fungal spores of Zymoseptoria tritici, the causal agent of Septoria blotch. Subsequently, the plants are kept under conditions that support the pathogen infection and the pathogen infection is scored.

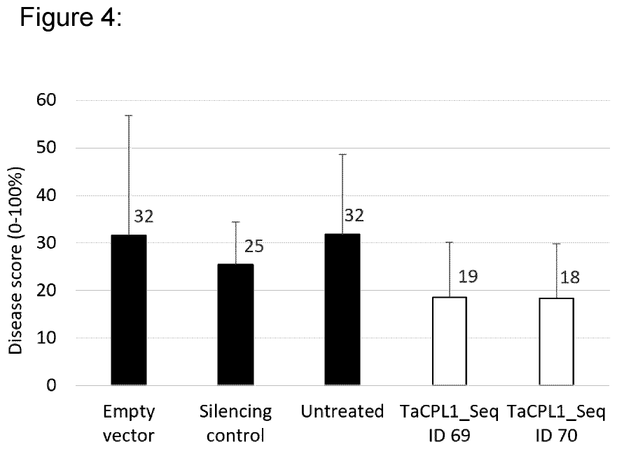

In comparison to wheat plants that were mock-inoculated, untreated or infected with an empty vector control, the wheat plants infected with CPL1-silencing constructs show a significant reduction of fungal pathogen (FIG. 4).

This demonstrates that silencing of all CPL1 homeologues in wheat leads to increased resistance against insects or pathogens (among them hemibiotrophic fungal pathogens).

Complementary to the fungal pathogen assay, an insect resistance assay is performed. It is expected that silencing of CPL1 also affects the susceptibility of plants, in particular wheat plants, towards insect infestation, i.e. a modification of the CPL1 protein or the reduction of the expression of CPL1 gene(s) increases the insect resistance of plants. Therefore, CPL1-silencing construct as well as above mentioned dominant-negative allele(s) can also be used as approach to increase insect resistance.

### Example 4: Fungal Resistance in Wheat (Triticum aestivum) Via KO Approach of CPL1 Genes

To confirm the relevance of the CPL1 genes for fungal resistance (knock-out) mutations in this gene are introduced by a TILLING approach using EMS or ENU as a mutagen. The selected plants are subsequently self-pollinated to create homozygous mutants. The homozygous CPL1 mutants are analyzed for increased resistance to fungal pathogens. If multiple paralogues are present in a plant, additive effects of a knock-out of multiple paralogues genes in parallel are expected. A full knock-out of the CPL1 genes and not only a reduced expression of them, leads to delayed flowering. Thus, a downregulation of CPL1 gene expression might be the preferred technical teaching.

As an alternative to the TILLING approach, genome editing is used to create knock-out mutations in CPL1 genes. Modifications of regulatory elements of CPL1 genes by genome editing are used for the downregulation of CPL1 gene expression. Concerning a delayed flowering in CPL1 full-knock-out plants, a downregulation of expression prevents this side effect of the CPL1 resistance approach.

For genome editing of CPL1 a target validation has been performed. Corn protoplasts were co-transfected with two constructs. One carrying a constitutively expressed MAD7 gene with nuclease activity if expressed, and the second carrying a constitutively expressed single guide RNA's (crRNA) for each target site. Following transfection, the protoplasts were cultured for 24 hrs.

Protoplast samples were measured for transfection efficiency using expression of a fluorescent marker gene carried on the same plasmid as MAD7 gene. The protoplasts were counted using a flow cytometer.

Protoplast samples were extracted for genomic DNA and DNA samples were PCR amplified for an amplicon flanking the targeted sequence. Amplicon deep sequencing was used to determine the cutting rate at each site. INDEL frequencies are presented in Table 3. Each treatment was replicated three times and data is shown in the table below. The INDEL frequency and Std. Error are based on raw data. The second set of values is adjusted based on the protoplast transfection efficiency.

TABLE 3

| | | | | | INDEL | |
|---|---|---|---|---|---|---|
| | | | | | frequency | |
| | | | | | normalized to | |
| PAM | | | INDEL | Standard | transfection | Standard |
| sequence | Protospacer sequence | replicates | frequency | error | efficiency | error |
| TTTC | GAGGACAGAATTGATGCACTT | 3 | 5.19 | 0.45 | 22.72 | 1.31 |
| | (SEQ ID NO: 75) | | | | | |
| TTTC | AGATCCACTGCAAGGTTCTCC | 3 | 6.11 | 0.49 | 23.09 | 1.99 |
| | (SEQ ID NO: 76) | | | | | |
| TTTC | ACCGTTGAATCCGATTCTATG | 3 | 2.69 | 0.17 | 10.72 | 0.59 |
| | (SEQ ID NO: 77) | | | | | |
| TTTG | GCTTGAGGGAACATGCCGAGT | 3 | 6.91 | 1.49 | 26.60 | 4.55 |
| | (SEQ ID NO: 78) | | | | | |
| TTTA | GGTGGAGTACAGGTCAACATT | 3 | 11.84 | 0.83 | 45.02 | 3.28 |
| | (SEQ ID NO: 79) | | | | | |

### Example 5: ERF922 Gene Identification

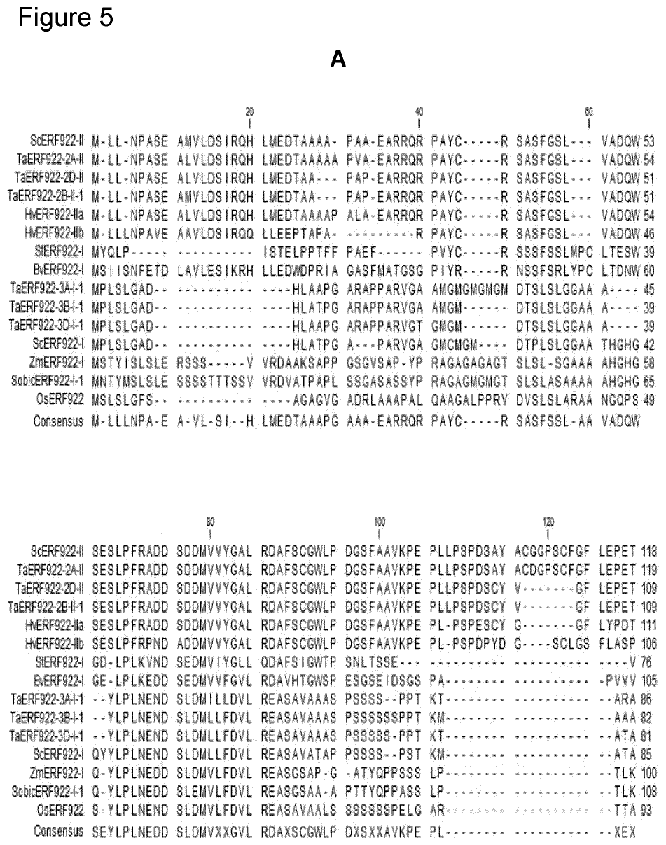

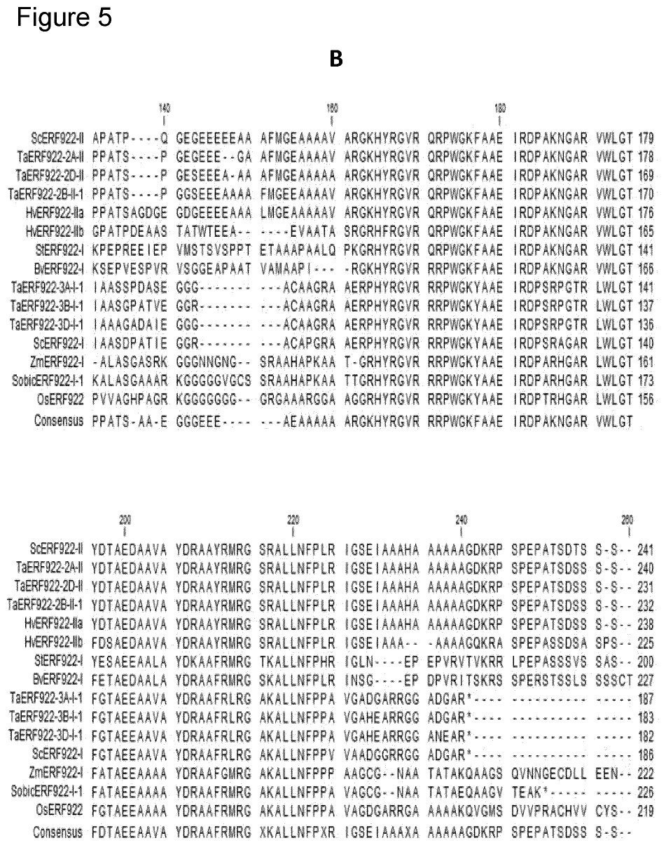

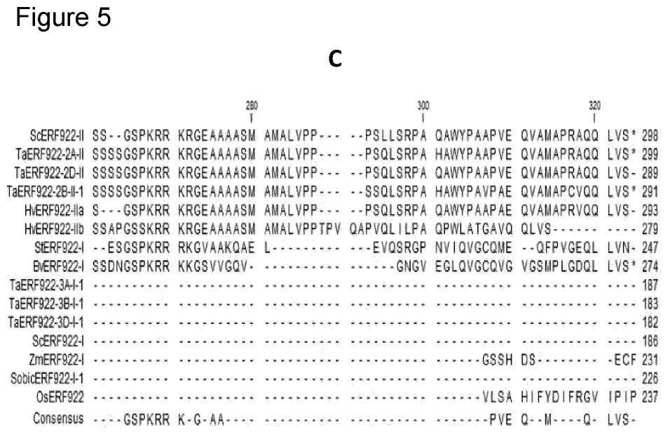

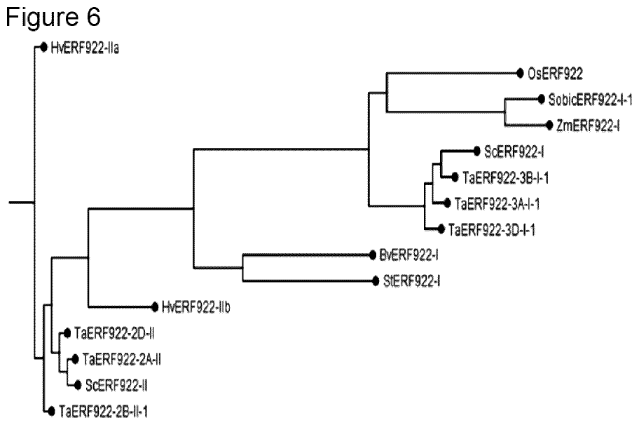

Using homology searches with the rice ERF922 protein sequence ERF922-coding genes in multiple crop plants have been identified, which encode ERF922 protein sequences (SEQ ID NOs: 36-50). Corresponding DNA coding region are shown (SEQ ID NOs: 51-65). In corn (Zea mays), Sorghum (Sorghum bicolor), sugar beet (Beta vulgaris) and potato (Solanum tuberosum) the protein of ERF922 gene is encoded by one gene ERF922-I. In the poaceae wheat (Triticum aestivum) and rye (Secale cereale) beside ERF922-I a second ERF992 like gene exist that shows also high homology to OsERF922 but belongs to a second ERF922-like protein named ERF922-II. In wheat the ERF922-I and ERF922-II genes are located as single alleles in the A, B and D genome on chromosome 3 and 2 each (FIGS. 5 and 6). The presence of a distinct but related ERF922 allele in the rye and wheat plant is shown in FIG. 5.

Transcription of OsERF922 is induced by fungal infection of rice leaves and abiotic stress (salt and drought). Analysis of mRNA seq data of wheat revealed that the A, B and D alleles of ERF922-I are expressed exclusively in wheat heads after Fusarium infection. ERF922-I expression in non-infected wheat was only detectable in the rachis and florets of wheat heads and absent in other tissues in contrast to ERF922-II. ERF922-II transcription was detectable in seedlings, roots, shoot, flag leaf and the spike, rachis and florets of the inflorescence. After fungal infection of leaves or stress-treated wheat plants expression of ERF922-I alleles was not detectable. However, the 3 alleles of ERF922-II were induced in all tissues by biotic and abiotic stress. ERF922-II transcripts were induced by Fusarium in wheat heads and by Septoria tritici, Puccinia and powdery mildew infection in wheat leaves. In addition, the ERF922-II expression was induced by cold. The ERF922 genes in the rye and wheat are paralogue. To improve the resistance of wheat against leaf disease the expression of ERF922-II must be reduced. Either reduction of ERF922-I or ERF922-II increases Fusarium head scab resistance of wheat.

### Example 6: Fungal Resistance in Wheat (Triticum aestivum) Via VIGS Approach Against ERF922-I and ERF922-II

Two silencing constructs targeting all six alleles of TaERF922-I on chromosome 3 (TaERF922-3A-I, TaERF922-3B-I, TAERF922-3D-I) and two silencing constructs targeting all six alleles of TaERF922-II on chromosome 2 (TaERF922-2A-II, TaERF922-2B-II, TAERF922-2D-II) have been developed for virus induced gene silencing (VIGS) experiments in wheat. The silencing sequences were specifically designed for having either high homology (>92% identity) to all six wheat ERF922-I homologs or to all six wheat ERF922-II homologs and, at the same time, to avoid large stretches of homology (<20 bp) to other coding sequences in the wheat genome. For the VIGS experiments an appropriate vector system was used.

Here, the TaERF922-I silencing sequences of fragment TaERF922-3A_fragA and TaERF922-3A_fragB and the TaERF922-II silencing sequences TaERF922-2A_fragA and TaERF922-2A_fragB were inserted individually. The sequences of TaERF922-3A_fragA and TaERF922-3A_fragB are identical to the positons 70-288 and 368-558 of the SED ID NO: 58-60. The sequences of TaERF922-2A_fragA and TaERF922-2A_fragB are identical to the positons 21-179 and 611-766 of the SED ID NO: 61-63. Virus particles carrying the silencing sequences were assembled by Agrobacterium co-infiltration-based transformation into Nicotiana benthamiana. Leaves of wheat cultivar Taifun (KWS, Einbeck, Germany) are subsequently transfected with sap extracted from the infiltrated N. benthamiana leaves. 14 days after transfection with the virus variant, the leaves of wheat plants are infected with fungal spores of Zymoseptoria tritici, the causal agent of Septoria blotch and the wheat heads with spores of Fusarium graminearum, the causal agent of Fusarium graminearum. Subsequently, the plants are kept under conditions that support the pathogen infection and the pathogen infection is scored.

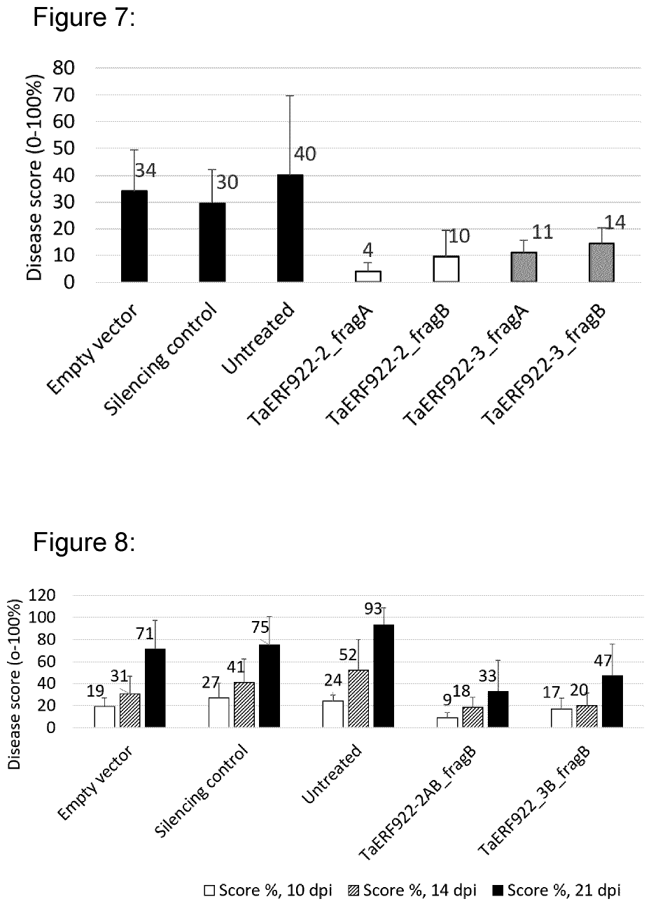

In comparison to wheat plants that were mock-inoculated, untreated or infected with an empty vector control, the wheat plants infected with ERF922-I or ERF922-II silencing constructs show a significant reduction of Septoria blotch (Figure. 7) and Fusarium head scab (Figure. 8).

This demonstrates that silencing of all ERF922 homeologues in wheat leads to increased resistance against fungal pathogens (among them hemibiotrophic and necrotrophic fungal pathogens).

### Example 7: Fungal Resistance in Corn (Zea mays) Via RNAi Approach Against ERF922

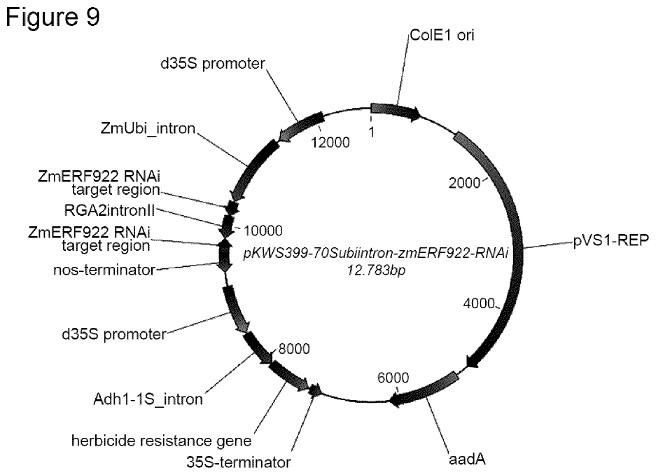

Resistance improvement was demonstrated by downregulation of expression of an ERF922-I gene after selecting the maize ZmERF922 gene for testing. An RNAi-based silencing construct against the ERF922-I gene of corn (FIG. 9, SEQ ID NO: 66) was constructed and stably transformed into the A188 genotype of corn by Agrobacterium tumefaciens-mediated transformation. Transgenic maize plants of line MTR554-T-015 along with the respective azygous sister line MTR554-T-015 (non-transgenic) of the segregating T1 generation were identified by PCR. The MTR554-T015 plants were tested with the genotype A188 for resistance against Setosphaeria turcica, the causal agent of Northern Corn Leaf Blight (NCLB), in the greenhouse. The height of the plants was measured additionally before inoculation and no difference was detectable (Table 4).

TABLE 4
ERF922 RNAi line MTR554-T-015 shows enhanced Northern corn leaf
blight resistance compared to the transformation genotype A188 and
the azygous MTR554-T-015 (non-transgenic)

| | | | | disease | disease | fungal |
|---|---|---|---|---|---|---|
| | | mean | mean | score | score | biomass in |
| | | plant | leaf | (0-100% | (0-100%) | infected leaf |
| | plant | size | width | 5^{th} leaf | 5^{th} leaf | (mg/g) |
| line | number | (cm)¹ | (cm)¹ | 18 dpi | 23 dpi | 24 dpi |
| A188 | 35 | 80.7 | 4.3 | 47.5 | 73 | 106.2 |
| MTR554-T-015 | 25 | 80.1 | 4.8 | 33.6 | 55.6 | 97.3 |
| (transgenic) | | | | | | |
| MTR554-T-015 | 5 | 77.8 | 5.1 | 48 | 72 | 116.9 |
| (non-transgenic) | | | | | | |

¹Plant size measured before inoculation

In the resistance assay the 5th leaf of each plant was spray inoculated with 5.000 spores/ml of a mixture of two S. turcia isolates (race 0). The transgenic ERF922 RNAi plants of line MTR554-T-015 showed an enhanced resistance after visual disease scoring of the 5th leaf 18 and 23 days after inoculation (dpi). Compared to the transformation genotype A188 and the azygous sister line MTR554-T-015 (non-transgenic) the ERF922 RNAi plants showed 30% and 23% less symptoms after 18 and 23 dpi, respectively. At 24 dpi the 5th leaf of each plant has been sampled, was dried and fungal biomass was determined from isolated genomic DNA by qPCR. The fungal biomass of the transgenic MTR554-T-015 line was 14% reduced compared to the mean of fungal biomass in A188 and MTR554-T-015 (non-transgenic). These experiments revealed that the downregulation of the ZmERF922 gene leads to increased resistance against a hemibiotrophic fungus in corn and that downregulation of ZmERF922 does not cause growth retardation.

### Example 8: Fungal Resistance in Wheat (Triticum aestivum) Via Genome Editing of ERF922

Genome editing was applied to downregulate the expression of ERF922-I and ERF922-II in wheat. Wheat protoplasts were co-transfected with two constructs; one carrying a constitutively expressed RR-Cpf1 gene and the second carrying a constitutively expressed single guide RNA for each target site. The proto-spacer sequences of the target sites, crGEP239 and crGEP243, are shown as SEQ ID NOs: 67 and 68. Following transfection, the protoplasts were cultured for 24 hrs. Protoplast samples were measured for transfection efficiency using expression of a fluorescent marker gene carried on the same plasmid as RR-Cpf1 gene. The protoplasts were counted using a flow cytometer.

Protoplast samples were extracted for genomic DNA and DNA samples were PCR amplified for an amplicon flanking the targeted sequence. Amplicon deep sequencing was used to determine the cutting rate at each site of the A and B genomes of wheat. INDEL frequencies are presented in Table 5.

TABLE 5
gRNA (pGE745-750) tested for ERF922 gene disruption
in wheat. InDel efficiency is given in %

| | | | | INDEL |
|---|---|---|---|---|
| Gene | Construct | Target | Target comments | Frequency (%) |
| Ta-ERF922 | pGEP745 | crGEP239 | A, B1 only | A4.0, B2.4 |
| Ta-ERF922 | pGEP746 | crGEP240 | A, B1 only | A1.2, B3.5 |
| Ta-ERF922 | pGEP747 | crGEP241 | A, B1 only | A0.0, B0.0 |
| Ta-ERF922 | pGEP748 | crGEP242 | B2 only | 0.0 |
| Ta-ERF922 | pGEP749 | crGEP243 | B2 only | 0.6 |
| Ta-ERF922 | pGEP750 | crGEP244 | B2 only | 0.1 |

1. A method for increasing resistance and/or tolerance to a pathogen in a plant, plant part, or plant population, comprising reducing or eliminating the expression, stability, and/or activity of ERF922 and/or CPL1 protein and/or gene in a plant, plant part, or plant population.

2. The method according to claim 1, wherein an ERF922 and/or CPL1 gene is mutated, preferably the coding sequence and/or a regulatory sequence of an ERF922 and/or CPL1 gene is mutated and/or expression, stability, and/or activity of an ERF922 and/or CPL1 protein is reduced.

3. The method according to claim 2, wherein said mutated ERF922 and/or CPL1 gene comprises a point mutation, preferably resulting an amino acid substitution in the ERF922 and/or CPL1 protein, preferably wherein said mutated ERF922 and/or CPL1 protein is a dominant negative ERF922 and/or CPL1 protein and/or said mutated CPL1 protein comprises a mutation in a DXDXT motif.

4. The method according to claim 2, wherein said mutated CPL1 protein comprises a mutation corresponding to D128X in AtCPL4, wherein X is an amino acid different from D, preferably wherein said mutated CPL1 protein comprises a mutation corresponding to D128A in AtCPL4, more preferably wherein said mutated CPL1 protein comprises a mutation in the CPL1 protein corresponding to:
D148X, preferably D148A, when said plant is from the genus Zea, preferably Zea mays;
D149X, preferably D149A, when said plant is from the genus Zea, preferably Zea mays;
D162X, preferably D162A, when said plant is from the genus Beta, preferably Beta vulgaris;
D143X, preferably D143A, when said plant is from the genus Solanum, preferably Solanum tuberosum;
D141X, preferably D141A, when said plant is from the genus Glycine, preferably Glycine max;
D149X, preferably D149A, when said plant is from the genus Triticum, preferably Triticum aestivum;
D147X, preferably D147A, when said plant is from the genus Triticum, preferably Triticum aestivum;
D149X, preferably D149A, when said plant is from the genus Sorghum, preferably Sorghum bicolor;
wherein X is an amino acid different from D.

5. The method according to claim 1, wherein the wild type CPL1 or ERF922 protein comprises a sequence which is respectively at least 95% identical, preferably over its entire length, to a sequence of any of SEQ ID NOs: 2-17 or 37-50 or which is encoded by a sequence which at least 95% identical, preferably over its entire length, to a sequence of any of SEQ ID NOs: 19-34, or 52-65.

6. The method according to claim 1, wherein said plant is selected from the Poaceae family, preferably said plant is selected from the Pooidae subfamily, more preferably said plant is selected from the genus Zea, Sorghum, Triticum, Hordeum, Secale, Beta, Glycine, or Solanum, even more preferably said plant is selected from the species Zea mays, Sorghum bicolor, Triticum aestivum, Hordeum vulgare, Secale cereale, Beta vulgaris, Glycine max, or Solanum tuberosum.

7. The method according to claim 1, wherein said ERF922 and/or CPL1 protein and/or gene expression, stability, and/or activity is reduced or eliminated by knocking out an ERF922 and/or CPL1 gene or knocking down said ERF922 and/or CPL1 protein and/or said ERF922 and/or CPL1 protein and/or gene expression, stability, and/or activity is reduced or eliminated by mutagenesis, RNAi, or gene editing.

8. The method according to claim 1, wherein the method comprises recombinantly or transgenically introducing or introgressing into the genome of a plant or plant part a mutation in an ERF922 and/or CPL1 gene or a nucleotide sequence of a gene encoding ERF922 and/or CPL1 having a mutation, preferably a mutation leading to reduced or eliminated expression of the mRNA of the gene and/or the ERF922 and/or CPL1 protein, a mutation leading to an ERF922 and/or CPL1 protein having reduced or eliminated activity upon translation, or a mutation leading to an ERF922 and/or CPL1 protein having reduced stability.

9. The method according to claim 1, wherein the method comprises
(a) recombinantly or transgenically introducing or introgressing into a plant or plant part into a nucleotide sequence of a endogenous wild type gene encoding an ERF922 and/or CPL1 a mutation, preferably a mutation leading to reduced or eliminated expression of the endogenous full length mRNA of the gene and/or the endogenous full length ERF922 and/or CPL1 protein, a mutation leading to an ERF922 and/or CPL1 protein having reduced activity upon translation, or a mutation leading to an ERF922 and/or CPL1 protein having reduced stability;
(b) recombinantly or transgenically introducing or introgressing into a plant or a plant part an RNAi molecule directed against, targeting, or hybridizing with a nucleotide sequence encoding an ERF922 and/or CPL1 protein, or a polynucleotide sequence encoding an RNAi molecule directed against, targeting, or hybridizing with a nucleotide sequence encoding an ERF922 and/or CPL1 protein, or
(c) recombinantly or transgenically introducing or introgressing into a plant or a plant part an RNA-specific or DNA-specific CRISPR/Cas system directed against or targeting a nucleotide sequence encoding an ERF922 and/or CPL1 protein, or one or more polynucleotide sequence(s) encoding said RNA-specific CRISPR/Cas system, or
(d) recombinantly or transgenically introducing or introgressing into a plant or a plant part a chemical compound or an antibody altering the activity of an ERF922 and/or CPL1 protein upon interaction with said ERF922 and/or CPL1 or
(e) recombinantly or transgenically introducing or introgressing into a plant or a plant part a dominant negative ERF922 and/or CPL1 protein or one or more nucleic acid encoding a dominant negative ERF922 and/or CPL1 protein.
(f) optionally, regenerating a plant from the plant part of any of (a) to (d).

10. A plant, plant part, or plant population obtained or obtainable by the method according to claim 1, or the progeny thereof, or having reduced or eliminated expression, stability, and/or activity of an ERF922 and/or CPL1 protein and/or gene compared to the expression, stability, and/or activity in a plant, plant part, or plant population of the same species without the reduced or eliminated expression, stability, and/or activity of an ERF922 and/or CPL1 protein and/or gene, preferably the plant, plant part, or plant population according is transgenic, mutagenized or gene-edited.

11. The plant, plant part, or plant population according to claim 10, wherein said plant is selected from the Poaceae family, preferably said plant is selected from the Pooidae subfamily, more preferably said plant is selected from the genus Zea, Sorghum, Triticum, Hordeum, Secale, Beta, Glycine, or Solanum, even more preferably said plant is selected from the species Zea mays, Sorghum bicolor, Triticum aestivum, Hordeum vulgare, Secale cereale, Beta vulgaris, Glycine max, or Solanum tuberosum.

12. A vector comprising a polynucleic acid comprising
A) a sequence which is at least 90% identical, preferably over its entire length, to a sequence of any of SEQ ID NOs: 19-34, or 52-65; or which encodes a polypeptide which is at least 90% identical, preferably over its entire length, to a sequence of any of SEQ ID NOs: 2-17, 80-87, or 37-50, or
B) a polynucleic acid specifically hybridizing with the polynucleic acid of a), the complement thereof, or the reverse complement thereof.

13. A method for generating a plant or plant part, comprising (a) providing a first plant according to claim 10, (b) crossing said first plant with a second plant, (c) selecting progeny plants having reduced or eliminated expression, stability, and/or activity of a ERF922 and/or CPL1 protein and/or gene compared to the expression, stability, and/or activity in a plant of the same species, and optionally (d) harvesting said plant part from said progeny.

14. A method of using the polynucleic acid as defined in claim 12 for increasing resistance and/or tolerance to a pathogen in a plant, plant part, or plant population or for generating a plant or plant part, or plant population having reduced or eliminated expression, stability, and/or activity of an ERF922 and/or CPL1 protein and/or gene compared to the expression, stability, and/or activity in a plant, plant part, or plant population of the same species without the reduced or eliminated expression, stability, and/or activity of an ERF922 and/or CPL1 protein and/or gene.

15. A plant, plant part, or plant population comprising the polynucleic acid as defined in claim 12.

16. A method for controlling pathogen infestation in a plant (population) comprising
a) Providing (a) plant(s) according to claim 10 or growing from seeds (a) plant(s) according to claim 10,
b) Cultivating the plant(s) of a) under conditions of pathogen infestation.

17. A method for producing feed or food with reduced amount of fungal or bacterial toxins comprising
A) controlling pathogen infestation in a plant population by the method of claim 16,
B) harvesting plant material from the population, and
C) producing feed or food from the harvested plant material.

18. A feed or food with reduced amount of fungal or bacterial toxins obtained by a method according to claim 17.

19. A method for identifying a plant, plant part, or plant population having increased resistance and/or tolerance to a pathogen, comprising screening for and/or identifying a mutation as defined in claim 2, or reduced or eliminated expression, activity, and/or stability of CPL1 and/or ERF922 protein and/or gene in a plant, plant part, or plant population.

20. The method according to claim 19, further comprising the step of selecting the plant, plant part, or plant population having the mutation, or the step of selecting the plant, plant part, or plant population having a reduced or eliminated expression, activity, and/or stability of ERF922 and/or CPL1.
